SEQUENCE LISTING (6 sequences)

SEQ ID NO: 1 - protein, 150 aa, Homo sapiens
HMADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 2 - protein, 107 aa, Oryctolagus cuniculus
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 3 - protein, 5037 aa, Oryctolagus cuniculus [sequence omitted]

SEQ ID NO: 4 - DNA, 34 bp, synthetic construct
  source 1..34: mol_type = other DNA
gaacatccatgaactcgcggcgctgacgcgcatt

SEQ ID NO: 5 - DNA, 34 bp, synthetic construct
  source 1..34: mol_type = other DNA
gaatgggcataacgtggcggcacgagaccgagtg

SEQ ID NO: 6 - DNA, 38 bp, synthetic construct
  source 1..38: mol_type = other DNA
caagaggctgcacccggcactagtgaacttccacagcc

(19) United States
(12) Patent Application Publication
(10) Pub. No.: US 2023/0386615 A1
(43) Pub. Date: Nov. 30, 2023

(54) BINDING SITE IN TYPE 1 RYANODINE RECEPTOR

(71) Applicant: The Trustees of Columbia University in the City of New York, New York, NY (US)

(72) Inventors: Andrew R. MARKS, New York, NY (US); Zephan MELVILLE, New York, NY (US)

(21) Appl. No.: 18/050,423

(22) Filed: Oct. 27, 2022

Related U.S. Application Data
(60) Provisional application No. 63/272,570, filed on Oct. 27, 2021.

(51) Int. Cl. G16C 20/50; G16C 20/70; G01N 33/53; G01N 33/566; G01N 33/68; G01N 23/2251

(52) U.S. Cl. CPC G16C 20/50; G16C 20/70; G01N 33/5308; G01N 33/566; G01N 33/6872; G01N 23/2251

(57) ABSTRACT

The present disclosure relates to methods and compositions useful for the identification of a ryanodine receptor modulator binding site in ryanodine receptor type 1 (RyR1). The present disclosure also provides compositions useful for the analysis of the ryanodine receptor modulator binding site in RyR1 via cryoEM. The present disclosure further provides computational methods for identifying compounds that bind to RyR1.

### CROSS REFERENCE

This application claims the benefit of U.S. Provisional Patent Application No. 63/272,570, filed on Oct. 27, 2021, the content of which is incorporated by reference herein in its entirety.

### STATEMENT AS TO FEDERALLY SPONSORED RESEARCH

This present disclosure was made with government support under R01HL145473, R01DK118240, R01HL142903, R01HL140934, R01AR070194 and T32 HL120826, awarded by the National Institutes of Health (NIH). The government has certain rights in the disclosure.

### SEQUENCE LISTING

The instant application contains a Sequence Listing which has been submitted electronically in XML format and is hereby incorporated by reference in its entirety. Said XML copy, created on Oct. 27, 2022, is named 44010-105US-PAT.xml and is 11,637 bytes in size.

### BACKGROUND

The ryanodine receptor (RyR) is required for excitation-contraction coupling. Although RyR is tightly regulated, inherited mutations and stress-induced post-translational modifications can result in a Ca²⁺ leak in skeletal myopathies, heart failure, and exercise-induced sudden death. Compounds known as Rycals® repair the leaky RyR and are effective in preventing and treating disease symptoms and restoring normal RyR function.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, the present disclosure provides a composition comprising a complex suspended in a solid medium, wherein the complex comprises a protein and a synthetic compound, wherein the protein is a ryanodine receptor 1 protein (RyR1) or mutant thereof.

In some embodiments, the present disclosure provides a method for predicting a docked position of a target ligand in a binding site of a biomolecule, the method comprising:
receiving a template ligand-biomolecule structure, the template ligand-biomolecule structure comprising a template ligand docked in the binding site of the biomolecule;
comparing a pharmacophore model of the template ligand to a pharmacophore model of the target ligand;
overlapping the pharmacophore model of the target ligand with the pharmacophore model of the template ligand while the template ligand is in the binding site of the biomolecule; and
predicting the docked position of the target ligand in the binding site of the biomolecule based on a position of the pharmacophore model of the target ligand when overlapped with the pharmacophore model of the template ligand,
wherein the biomolecule is a RYT&2 domain of RyR1, and wherein the template ligand-biomolecule structure is obtained by a process comprising subjecting a complex of the biomolecule and the template ligand to single-particle cryogenic electron microscopy analysis.

In some embodiments, the template ligand is a RyR1 modulator. In some embodiments, the template ligand can bind to leaky RyR channels and repair the Ca²⁺ leak, restoring normal channel function. In some embodiments, the target ligand is a RyR1 modulator. In some embodiments, the target ligand can bind to leaky RyR channels and repair the Ca²⁺ leak, restoring normal channel function.

In some embodiments, the present disclosure provides a method of identifying a plurality of potential lead compounds, the method comprising the steps of:
(a) analyzing, using a computer system, an initial lead compound known to bind to a biomolecular target, the analyzing comprising partitioning, by providing a database of known reactions, the initial lead compound into atoms defining partitioned lead compound comprising a lead compound core and atoms defining a lead compound non-core, wherein the initial lead compound is partitioned using a computational retrosynthetic analysis of the initial lead compound;
(b) identifying, using the computer system, a plurality of alternative cores to replace the lead compound core in the initial lead compound, thereby generating a plurality of potential lead compounds each having a respective one of the plurality of alternative cores;
(c) calculating, using the computer system, a difference in binding free energy between the partitioned lead compound and each potential lead compound;
(d) predicting, using the computer system, whether each potential lead compound will bind to the biomolecular target and identifying a predicted active set of potential lead compounds based on the prediction;
(e) obtaining a synthesized set of at least some of the potential leads of the predicted active set to establish a first of potential lead compounds; and
(f) determining, empirically, an activity of each of the first set of synthesized potential lead compounds,
wherein the biomolecular target is a RY1&2 domain of RyR1, and the structure of the biomolecular target used in the predicting of (d) is obtained by a process comprising subjecting a complex of the biomolecular target and the initial lead compound to single-particle cryogenic electron microscopy analysis.

In some embodiments, the present disclosure provides a computer-implemented method of quantifying binding affinity between a ligand and a receptor molecule, the method comprising:
receiving by one or more computers, data representing a ligand molecule, receiving by one or more computers, data representing a receptor molecule domain, using the data representing the ligand molecule and the data representing the receptor molecule domain in computer analysis to identify ring structure within the ligand, the ring structure being an entire ring or a fused ring;
using the data representative of the identified ligand ring structure to designate a first ring face and a second ring face opposite to the first ring face, and classifying the ring structure by:
a) determining proximity of receptor atoms to atoms on the first face of the ligand ring; and
b) determining proximity of receptor atoms to atoms on the second face of the ligand ring;
c) determining solvation of the first face of the ligand ring and solvation of the second face of the ligand ring;
classifying the identified ligand ring structure as buried, solvent exposed or having a single face exposed to solvent based on receptor atom proximity to and solvation of the first ring face and receptor atom proximity to and solvation of the second ring face; quantifying the binding affinity between the ligand and the receptor molecule domain based at least in part on the classification of the ring structure; and
displaying, via computer, information related to the classification of the ring structure,
wherein the receptor molecule domain is a RY1&2 domain of RyR1, wherein the data representing a ligand molecule and the data representing a receptor molecule domain are obtained by a process comprising subjecting a complex comprising the ligand molecule and the receptor molecule domain to single-particle cryogenic electron microscopy analysis.

In some embodiments, the present disclosure provides a method comprising:
(a) determining an open probability (Pₒ) of a first RyR1 protein, wherein the first RyR1 protein is treated with both an agent capable of phosphorylating, nitrosylating or oxidizing the first RyR1 protein, and a test compound; and
(b) determining an open probability (Pₒ) of a second RyR1 protein, wherein the second RyR1 protein is treated with the agent and not treated with the test compound.

In some embodiments, the present disclosure provides a method comprising:
(a) contacting a first RyR1 protein with an agent capable of phosphorylating, nitrosylating or oxidizing the RyR1 protein, and a test compound;
(b) contacting a second RyR1 protein with the agent and not with the test compound;
(c) subsequent to the contacting the first RyR1 protein with the agent and the test compound, measuring an open probability (Pₒ) of the first RyR1 protein; and
(d) subsequent to the contacting the first RyR1 protein with the agent and the test compound, measuring an open probability (Pₒ) of the second RyR1 protein.

In some embodiments, the present disclosure provides a method of identifying a compound having RyR1 modulatory activity, the method comprising:
(a) determining open probability (Pₒ) of a RyR1 protein, wherein the RyR1 protein is a mutant RyR1 protein, a post-translationally modified RyR1 protein, or a combination thereof,
(b) contacting the RyR1 protein with a test compound;
(c) determining open probability (Pₒ) of the RyR1 protein in the presence of the test compound; and
(d) determining a difference between the Pₒ of the RyR1 protein in the presence and absence of the test compound;
wherein a reduction in the Pₒ of the RyR1 protein in the presence of the test compound compared with the Pₒ of the RyR1 protein in the absence of the test compound is indicative of the compound having RyR1 modulatory activity.

In some embodiments, the present disclosure provides a method for identifying a compound having RyR1 modulatory activity, comprising:
(a) contacting a RyR1 protein with a ligand having known RyR1 modulatory activity to create a mixture, wherein the RyR1 protein is a mutant RyR1 protein, a post-translationally modified RyR1 protein, or a combination thereof;
(b) contacting the mixture of step (a) with a test compound; and
(c) determining the ability of the test compound to displace the ligand from the RyR1 protein.

In some embodiments, provided is a method for identifying a compound that preferentially binds to leaky RyR1, comprising:
(a) determining binding affinity of a test compound to a first RyR1 protein, wherein the first RyR1 protein is a wild-type RyR1 protein;
(b) determining binding affinity of a test compound to a second RyR1 protein, wherein second RyR1 protein is a leaky RyR1, the leaky RyR comprising mutant RyR1 protein, a post-translationally modified RyR1 protein, or a combination thereof; and
(c) selecting a compound having a higher binding affinity to the second RyR1 protein relative to the first RyR1 protein.

In some embodiments, the method further comprises determining the effect of the test compound on binding affinity of RyR1 to calstabin1. In some embodiments, the method further comprises determining the effect of the test compound on Kₒₙ (association) and K_{off} (dissociation) of calstabin1 and RyR1 protein.

In some embodiments, the agent capable of post-translationally modifying the RyR1 (e.g., phosphorylating, nitrosylating or oxidizing) is an oxidant. In some embodiments, the agent is a nitrosylating agent. In some embodiments, the agent is a phosphorylating agent (e.g., PKA and/or CaMKII).

In some embodiments, the RyR1 is a mutant RyR1. In some embodiments, the RyR1 is a post-translationally modified RyR1. In some embodiments, the RyR1 is in a primed state. In some embodiments, the RyR1 is a leaky RyR1, wherein Ca²⁺ leak from the RyR1 is associated with a disease.

In some embodiments, the test compound is a RyR1 modulator. In some embodiments, the test compound can bind to leaky RyR channels and repair the Ca³⁺ leak, restoring normal channel function.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides GSFSC curves for the structure of RyR1 with Compound 1 & ATP as determined by cryogenic electron microscopy (cryoEM).

FIG. 2 is a ribbon diagram of the binding site of Compound 1 in the RY1&2 domain of RyR1 where residues close enough to form hydrophobic or hydrogen bonding interactions with Compound 1 are highlighted.

FIG. 3, Panel A depicts the RY1&2 domain of RyR1 in the presence (light grey) and absence (dark grey) of Compound 1. Panel B depicts the pore of the RyR1 channel in the closed conformation.

FIG. 4A depicts 3D variability slices of the RyR1 structure with Compound 1.

FIG. 4B provides a reaction coordinate scatterplot of the eigenvectors from the 3D variability slices of the RyR1 structure with Compound 1.

FIG. 5, Panel A provides postulated interactions of Compound 1 and ATP with RY1&2 domain binding site in RyR1. Panel B is a ribbon diagram of residues 3,472-3,479 of RyR1 as determined by cryoEM. Panel C is a ribbon diagram of residues 4224-4254 of RyR1 as determined by cryoEM.

FIG. 6 provides a sideview of two RyR1 protomers, (Panel A), SPRY domain beta-sheets and calstabin (Panel B), and bridging solenoid helices and calmodulin (Panel C).

FIG. 7, left panel is a ribbon diagram of the calmodulin binding site in RyR1, and FIG. 7, right panel is a ribbon diagram that shows the conformation change in calmodulin as a result of Ca²⁺ binding.

FIG. 8 provides traces obtained from single-channel recordings of wild-type (WT) and mutant RyR1 reconstituted in planar lipid bilayers.

FIG. 9, Panel A is a chart that illustrates quantification of single channel current open probability (Pₒ) of RyR1 (data are means±SEMs; 1-way-ANOVA shows *p<0.05 versus WT). Panel B provides quantification of caffeine-induced calcium release in RyR1 in response to 10 mM caffeine.

FIG. 10A, Panel A is a chart that illustrates the effects of PKA phosphorylation and oxidation of RyR1 on S107 binding as measured in a ligand binding study. Panel B is a chart that illustrates the effects of ATP on S107 binding to purified RyR1 as determined by a ligand binding study. Panel C illustrates S107-Compound 1 competition in a ligand binding study performed with PKA/H₂O₂ treated microsomes, 500 nM of ³H-S107, and varied concentrations (1-10,000 nM) of unlabeled Compound 1. Panel D illustrates S107 binding in the presence of increasing concentrations of ATP or ADP as measured in a ligand binding study. Panel E illustrates S107 binding to recombinant RyR1-WT and RyR1-W882A mutant in microsomes treated with PKA and H₂O₂ as measured in a ligand binding study. Panel F illustrates ³²P-ATP binding to WT and W882A RyR1 as measured in a ligand binding study.

FIG. 10B, Panel G illustrates S107 binding to recombinant RyR1-WT and RyR1-W996A as measured in a ligand binding study. Panel H illustrates ³²P-ATP binding to WT and W996A RyR1 as measured in a ligand binding study. Panels I-L illustrate radioligand binding to WT and mutant channels with ADP in place of ATP as measured in a ligand binding study.

### DETAILED DESCRIPTION

Located on the sarco/endoplasmic reticulum (SR/ER) membrane, the ryanodine receptor (RyR) is the largest known ion channel, at over two megadaltons, and is the primary mediator of the Ca²⁺ release required for excitation-contraction coupling in cardiac and skeletal muscle. RyR is required for excitation-contraction coupling. RyR1 is the primary isoform in skeletal muscle while RyR2 is the predominant cardiac isoform. RyR1 and RyR2 are also found in neurons. RyR3 is present where RyR1 and RyR2 are each present, but with significantly lower expression levels. Beyond their expression pattern, RyR1 and RyR2 are unique in how each is activated. In skeletal muscle, RyR1 is activated by the direct, mechanical interaction with the dihydropyridine receptor (DHPR). RyR2 is instead activated by Ca²⁺ in the process termed calcium-induced calcium release (CICR) in which Ca²⁺ binding to RyR2 creates a cascade effect as the release of Ca²⁺ through the RyR creates a high local concentration of Ca²⁺, which can cause neighboring RyR channels to open. RyR, a tetramer, forms tetrads in muscle tissue and under normal conditions, undergoes cooperative activation through the process termed coupled gating.

The correct activation of RyR, and thus activation of the appropriate downstream Ca²⁺ signaling pathways, is regulated by multiple ligands and protein interactions. Aside from Ca²⁺, ATP, and caffeine, RyR also binds calmodulin (CaM). CaM is an inhibitor of ryanodine receptor type 2 (RyR2). CaM can act as either an activator of ryanodine receptor type 1 (RyR1) under low Ca²⁺ conditions (˜150 nM), such as those at rest, or an inhibitor of RyR1 under high Ca²⁺ conditions (>1 μM). High Ca²⁺ conditions occur locally following intracellular Ca²⁺ release. Calstabin, a second accessory protein, also binds the RyR. This interaction stabilizes the closed state of the channel. In disease states, RyR can be nitrosylated, oxidized and/or phosphorylated to cause calstabin to dissociate from the channel. This dissociation results in Ca²⁺ leaking into the cytosol and inappropriate triggering of downstream Ca²⁺ signaling pathways.

RyR comprises three major segments, each composed of several domains. The first, the cytosolic shell, consists of the N-terminal domain (NTD) with two segments (A & B) and an N-terminal solenoid, three SPRY domains, two RYR domains (RY1&2 and RY3&4), and the junctional and bridging solenoids (J-Sol and Br-Sol). The cytosolic shell also houses the calstabin binding site, which binds in a pocket formed by the Br-Sol and the SPRY domains, specifically SPRY1, and calmodulin, which binds on the other side of the Br-Sol from calstabin, with the N-terminal domain of CaM binding along the face of the Br-Sol while the C-terminal domain binds a peptide within a pocket of the Br-Sol.

Although RyR is tightly regulated, inherited mutations and stress-induced post-translational modifications (e.g., phosphorylation, nitrosylation and oxidation) can result in a Ca²⁺ leak. As a key player in Ca²⁺ signaling, leaky RyR channels are associated with a wide variety of disease states including skeletal muscle myopathies such as RyR-related myopathy (RYR-RM), dystrophies such as muscular dystrophy (e.g., Duchenne Muscular Dystrophy), cardiac diseases such as heart failure and catecholaminergic polymorphic ventricular tachycardia (CPVT), diabetes, and neurological disorders such as post-traumatic stress disorders (PTSD) and Alzheimer's disease.

Compounds known as ryanodine receptor modulators (also known as Rycals®) can repair leaky RyR and are effective in preventing and treating disease symptoms and restoring normal RyR function. Ryanodine receptor modulators can have efficacy in a host of diseases, both in vitro and in vivo using animal models. Ryanodine receptor modulators can repair the Ca²⁺ leak by preferentially binding to leaky RyR compared to normal RyR, and causing reassociation of calstabin, thus restabilizing the closed state of the channel. Mutations in RyR have been linked to rare genetic forms of cardiac and skeletal muscle disorders and ryanodine receptor modulators be effective in animal models in these disorders.

Given the structure of several ryanodine receptor modulator compounds, which contain aromatics and charged groups, ryanodine receptor modulators were initially hypothesized to bind near the caffeine binding site based on early cryo-electron microscopy (cryoEM) structures with limited resolution. Advances in cryoEM, and particularly direct detection cameras and novel processing methods including local refinement, have dramatically improved the resolution of cryoEM maps, allowing unambiguous identification of ligand binding sites, including identification of a novel ATP binding site as described herein, and binding sites for Ca²⁺, and caffeine.

In some embodiments, the present disclosure utilizes cryoEM techniques to generate a high resolution model of RyR1. In some embodiments, a high resolution model of RyR1 includes a ryanodine receptor modulator (e.g., Compound 1) bound to a ryanodine receptor modulator binding site in the RY1&2 domain of RyR1. In some embodiments, a ryanodine receptor modulator compound binds cooperatively with ATP and stabilizes the closed state of RyR1.

As demonstrated herein, ryanodine receptor modulator binding to RyR1 increases when the RyR1 channel is made leaky (e.g., by oxidation, nitrosylation and/or phosphorylation of the channel), mimicking the condition of RyR in disease states. Thus, in some embodiments, Ryanodine receptor modulator compounds can bind preferentially to leaky RyR channels, for example at least 2-fold, at least 3-fold, at least 4-fold, at least 5-fold, at least 6-fold, at least 7-fold, at least 8-fold, at least 9-fold, at least 10-fold, or even greater, as compared to non-leaky RyR channels.

Ca²⁺, ATP, and caffeine are known to bind within the C-terminal domain (CTD) of RyR1. Disclosed herein is the identification of an additional ATP-binding site, in the periphery of the cytosolic shell of the RyR, in the RY1&2 domain that is comprised within the SPRY domain. In some embodiments, this region is also the ryanodine receptor modulator (Rycal) binding site. As demonstrated herein, ryanodine receptor modulator binding to RyR1 can increase in the presence of ATP. In some embodiments, Compound 1 binds in the RY1&2 domain cooperatively with ATP and stabilizes the closed state of the RyR1 channel despite the presence of activating ligands (Ca²⁺, ATP, and caffeine). These results were confirmed functionally using site-directed mutagenesis and electrophysiology. This identifies ryanodine receptor modulators such as Compound 1 as allosteric modulators of the RyR channels.

The present disclosure relates to methods and compositions useful for the identification of a binding site for ryanodine receptor modulators (Rycals) in ryanodine receptor type 1 (RyR1). The present disclosure also provides compositions useful for the analysis of the ryanodine receptor modulator binding site in RyR1 via cryoEM. The present disclosure further provides methods (e.g., computational methods) for identifying compounds that bind to RyR1. The present disclosure further provides methods for screening for compounds that bind to RyR1 by utilizing a cryoEM model of RyR1.

### Methods of Structural Determination.

Cryogenic electron microscopy (cryoEM) is a cryomicroscopy technique applied on samples cooled to cryogenic temperatures and embedded in an environment of vitreous water. An aqueous sample solution is applied to a grid-mesh and plunge-frozen in a cryogenic fluid such as liquid ethane or a mixture of liquid ethane and propane.

The structures of the disclosure can be determined using cryo-EM with a sample frozen at a temperature of from about −40° C. to about −280° C. In some embodiments, the cryo-EM used for structural determination uses a sample frozen at a temperature of from about −40° C. to about −100° C., from about −100° C., to about −150° C., from about −150° C. to about −175° C., from about −175° C. to about −200° C., from about −200° C. to about −225° C., from about −225° C. to about −250° C., or from about −250° C. to about −280° C. In some embodiments, the cryo-EM used for structural determination uses a sample frozen at a temperature of from about −40° C. to about −100° C. In some embodiments, the cryo-EM used for structural determination uses a sample frozen at a temperature of from about −150° C. to about −175° C. In some embodiments, the cryo-EM used for structural determination uses a sample frozen at a temperature of from about −175° C. to about −200° C. In some embodiments, the cryo-EM used for structural determination uses a sample frozen at a temperature of from about −250° C. to about −280° C. In some embodiments, the cryo-EM used for structural determination uses a sample frozen at a temperature of about −150° C., about −175° C., about −200° C., about −250° C., or about −280° C. In some embodiments, the cryo-EM used for structural determination uses a sample frozen at a temperature of about −175° C. In some embodiments, the cryo-EM used for structural determination uses a sample frozen at a temperature of about −200° C. In some embodiments, the cryo-EM used for structural determination uses a sample frozen in liquid nitrogen. In some embodiments, the cryo-EM used for structural determination uses a sample frozen in liquid helium. In some embodiments, the cryo-EM used for structural determination uses a sample frozen in liquid ethane. In some embodiments, the cryo-EM used for structural determination uses a sample frozen in liquid propane. In some embodiments, the cryo-EM used for structural determination uses a sample frozen in mixture of liquid nitrogen and liquid propane.

The structures of the disclosure can be determined using a protein concentration of from about 50 nM to about 5 μM. In some embodiments, a structure of the disclosure can be determined using a protein concentration of from about 50 nM to about 250 nM, from about 250 nM to about 500 nM, from about 500 nM to about 750 nM, from about 750 nM to about 1 μM, or from about 1 μM to about 5 μM. In some embodiments, a structure of the disclosure can be determined using a protein concentration of from about 50 nM to about 250 nM. In some embodiments, a structure of the disclosure can be determined using a protein concentration of from about 250 nM to about 500 nM. In some embodiments, a structure of the disclosure can be determined using a protein concentration of from about 500 nM to about 750 nM. In some embodiments, a structure of the disclosure can be determined using a protein concentration of from about 750 nM to about 1 μM. In some embodiments, a structure of the disclosure can be determined using a protein concentration of from about 1 μM to about 5 μM.

The structures of the disclosure can be determined using a sample solution with a pH of about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In some embodiments, the sample solution has a pH of about 7.0. In some embodiments, the sample solution has a pH of about 7.1. In some embodiments, the sample solution has a pH of about 7.2. In some embodiments, the sample solution has a pH of about 7.3. In some embodiments, the sample solution has a pH of about 7.4. In some embodiments, the sample solution has a pH of about 7.5.

The structures of the disclosure (e.g., compositions comprising RyR1 and a ryanodine receptor modulator such as compound 1 bound to a ryanodine receptor modulator binding site on RyR1, and optionally an ATP molecule bound to an ATP binding site on the RyR1) can be determined at a resolution of from about 15 Å to about 2 Å. In some embodiments, the structures of the disclosure can be determined at a resolution of from about 15 Å to about 12 Å, from about 12 Å to about 9 Å, from about 9 Å to about 6 Å, from about 6 Å to about 5 Å, from about 5 Å to about 4 Å, from about 4 Å to about 3 Å, or from about 3 Å to about 2 Å. In some embodiments, the structures of the disclosure can be determined at a resolution of about 2.45 Å. In some embodiments, the structures of the disclosure is determined at a resolution of about 3.1 Å. In some embodiments, the structures of the disclosure is determined at a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

### Compositions Containing Complexes of RyR1.

In some embodiments, the present disclosure provides compositions useful for the determination of the ryanodine receptor modulator binding site in RyR1 via methods such as cryoEM. In some embodiments, the present disclosure provides a composition comprising a complex suspended in a solid medium, wherein the complex comprises a biomolecule (e.g., a protein) and a synthetic compound, wherein the protein is a ryanodine receptor 1 protein (RyR1) or a mutant thereof.

In some embodiments, the composition is prepared by a process comprising vitrifying an aqueous solution applied to an electron microscopy grid, wherein the aqueous solution comprises the protein and the synthetic compound.

An electron microscopy grid is a support structure used to insert specimens, for example, for use in an electron microscope. The grid structures can be flat with various suitable materials (e.g., copper, gold, rhodium, nickel, molybdenum, ceramic, etc.) for the grids themselves. In some cases, the grid structure can have plating (e.g., rhodium), coating (e.g., carbon, gold, plastic, silicon nitride, etc.), a suitable thickness (e.g., from 20 to 50 micron), and a suitable diameter (e.g., 3 mm). The grid structures generally have crossing bars and spacings/holes between the bars (e.g., nanometer to micrometer scale holes). The bars can come in various suitable sizes or pitch, patterns (e.g., regular or irregular), and shapes (e.g., numbers or letters built into the grid bars).

In some embodiments, prior to the vitrifying, the aqueous solution is applied to the electron microscopy grid, and excess aqueous solution is removed from the electron microscopy grid by blotting the excess aqueous solution.

In some embodiments, the aqueous solution is dispensed onto the electron microscopy grid from a dispensing apparatus located on the side of the electron microscopy grid opposed to the side abutting blotting material. Once the liquid sample is dispensed onto the cryoEM grid, the blotting material can pull excess solution through the electron microscopy grid to produce a thin liquid film of the aqueous solution on the electron microscopy grid.

In some embodiments, the vitrifying comprises plunge freezing the aqueous solution applied to the electron microscopy grid into liquid ethane chilled with liquid nitrogen.

In some embodiments, the aqueous solution further comprises a buffering agent. Suitable buffering agents can include, for example, zwitterionic amines, such as TAPS ([tris(hydroxymethyl)methylamino]propanesulfonic acid), Bicine (2-(bis(2-hydroxyethyl)amino)acetic acid), Tris (2-amino-2-(hydroxymethyl)propane-1,3-diol), and Tricine (N-[tris(hydroxymethyl)methyl]glycine), as well as zwitterionic sulfonic acids, such as TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (piperazine-N,N′-bis(2-ethanesulfonic acid)), and MES (2-(N-morpholino)ethanesulfonic acid). In some embodiments, the buffering agent is HEPES. In some embodiments, the buffering agent is EGTA.

In some embodiments, the aqueous solution further comprises a phospholipid. In some embodiments, the phospholipid is a phosphatidylcholine, such as, for example, 1,2-dioleoyl-sn-glycero-3-phosphocholine (DOPC), 1,2-dilinoleoyl-sn-glycero-3-phosphocholine (DLPC), 1,2-dihexanoyl-sn-glycero-3-phosphocholine (DHPC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), or 1,2-distearoyl-sn-glycero-3-phosphocholine (DSPC). In some embodiments, the phospholipid is a phosphatidylserine, such as, for example, 1-palmitoyl-2-oleoyl-sn-glycero-3-phospho-L-serine (sodium salt) (POPS), or 1,2-dioleoyl-sn-glycero-3-phospho-L-serine (sodium salt) (DOPS). In some embodiments, the phospholipid is DOPS.

In some embodiments, the aqueous solution further comprises a surfactant. Surfactants can be used in a composition disclosed herein to increase the solubility of a protein (e.g. RyR1). In some embodiments, the surfactant is a zwitterionic surfactant, such as, for example, 3-[(3-cholamidopropyl)dimethylammonio]-1-propanesulfonate (CHAPS) or 3-([3-Cholamidopropyl]dimethylammonio)-2-hydroxy-1-propanesulfonate (CHAPSO). In some embodiments, the zwitterionic surfactant is CHAPS.

In some embodiments, the aqueous solution further comprises a disulfide-reducing agent, which can be, for example, tris (2-carboxyethyl) phosphine hydrochloride (TCEP), beta-mercaptoethanol (BME), tributylphosphine (TBP). or dithiothreitol (DTT). In some embodiments, the disulfide-reducing agent is TCEP.

In some embodiments, the aqueous solution further comprises a protease inhibitor. Suitable protease inhibitors can include, for example, 4-(2-aminoethyl)benzenesulfonyl fluoride (AEBSF), phenylmethylsulfonyl fluoride (PMSF), leupeptin, N-ethylmaleimide, antipain, pepstatin, alpha 2-macro-globulin, EDTA, bestatin, amastatin, and benzamidine. In some embodiments, the protease inhibitor is AEBSF. In some embodiments, the protease inhibitor is benzamidine hydrochloride.

In some embodiments, the aqueous solution further comprises caffeine. The concentration of caffeine in the aqueous solution can be, for example, about 1 mM to about 15 mM, about 1 mM to about 50 mM, about 1 mM to about 30 mM, about 1 mM to about 10 mM, about 2 mM to about 10 mM, about 3 mM to about 10 mM, about 3 mM to about 7 mM, or about 4 mM to about 6 mM. In some embodiments, caffeine is present at a concentration of from about 3 mM to about 7 mM.

In some embodiments, caffeine is present at a concentration of about 1 mM, about 2 mM, about 3 mM, about 4 mM, about 5 mM, about 6 mM, about 7 mM, about 8 mM, about 9 mM, or about 10 mM. In some embodiments, caffeine is present at a concentration of about 5 mM.

In some embodiments, the aqueous solution further comprises dissolved Ca²⁺. The concentration of dissolved Ca²⁺ in the aqueous solution can be, for example, about 1 μM to about 200 μM, about 1 μM to about 150 μM, about 1 μM to about 100 μM, about 5 μM to about 100 μM, about 5 μM to about 75 μM, about 5 μM to about 50 μM, about 5 μM to about 40 μM, about 10 μM to about 40 μM, about 15 μM to about 40 μM, or about 20 μM to about 40 μM. In some embodiments, dissolved Ca²⁺ is present at a concentration from about 5 μM to about 100 μM. In some embodiments, dissolved Ca²⁺ is present at a concentration from about 20 μM to about 40 μM.

In some embodiments, Ca²⁺ is present at a concentration of about 10 μM, about 20 μM, about 30 μM, about 40 μM, about 50 μM, about 60 μM, about 70 μM, about 80 μM, about 90 μM, or about 100 μM. In some embodiments, dissolved Ca²⁺ is present at a concentration of about 30 μM.

The concentration of the protein in the aqueous solution can be, for example about 1 μM to about 100 μM, about 1 μM to about 75 μM, about 1 μM to about 50 μM, about 1 μM to about 45 μM, about 1 μM to about 40 μM, about 1 μM to about 35 μM, about 1 μM to about 30 μM, about 1 μM to about 25 μM, about 1 μM to about 20 μM, about 5 μM to about 30 μM, about 5 μM to about 25 μM, or about 5 μM to about 20 μM. In some embodiments, the protein is present at a concentration from about 1 μM to about 100 μM. In some embodiments, the protein is present at a concentration from about 1 μM to about 45 μM.

In some embodiments, the protein is present at a concentration of about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM about 24 μM, about 25 μM, about 26 μM, about 27 μM, about 28 μM, about 29 μM, about 30 μM, about 35 μM, about 40 μM, about 45 μM, about 50 μM, about 75 μM, or about 100 μM. In some embodiments, the protein is present at a concentration of about 15 μM.

In some embodiments, the aqueous solution further comprises sodium adenosine triphosphate (NaATP). The concentration of NaATP in the aqueous solution can be, for example, about 1 mM to about 15 mM, about 1 mM to about 50 mM, about 1 mM to about 30 mM, about 1 mM to about 30 mM, about 2 mM to about 30 mM, about 3 mM to about 30 mM, about 4 mM to about 30 mM, about 5 mM to about 30 mM, about 6 mM to about 30 mM, about 7 mM to about 30 mM, about 8 mM to about 30 mM, about 9 mM to about 30 mM, about 10 mM to about 30 mM, 1 mM to about 15 mM, about 2 mM to about 15 mM, about 3 mM to about 15 mM, about 4 mM to about 15 mM, or about 5 mM to about 15 mM. In some embodiments, NaATP is present at a concentration from about 3 mM to about 15 nM.

In some embodiments, NaATP is present at a concentration of about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, or about 50 mM. In some embodiments, the concentration of NaATP is about 10 mM.

In some embodiments, the aqueous solution is substantially free of cellular membrane. Prior to adding protein to the solution, the protein can be separated from cellular membranes by homogenization of cells containing the protein, and subjecting the resulting homogenate to chromatography.

In some embodiments, the aqueous solution further comprises calmodulin. In some embodiments, the calmodulin is human calmodulin. In some embodiments, the calmodulin is rabbit calmodulin. In some embodiments, the calmodulin has a sequence according to SEQ ID NO: 1.

The aqueous solution can be prepared or stored in a vessel. In some embodiments, the vessel is a vial, ampule, test tube, or microwell plate.

In some embodiments, the complex further comprises a nucleoside-containing molecule. In some embodiments, the nucleoside-containing molecule is a purine nucleoside-containing molecule. In some embodiments, the nucleoside-containing molecule is a nucleotide or nucleoside polyphosphate. In some embodiments, the nucleoside-containing molecule is an adenosine triphosphate (ATP) molecule.

In some embodiments, the nucleoside-containing molecule and the synthetic compound bind a RYR domain of the protein. In some embodiments, the ATP molecule forms a pi-stacking interaction with W996 of the protein. In some embodiments, the RYR domain is a RYT&2 domain. In some embodiments, the RY1&2 domain has a three-dimensional structure according to TABLE 2. In some embodiments, the synthetic compound has a three-dimensional conformation according to TABLE 3. In some embodiments, the ATP molecule has a three-dimensional conformation according to TABLE 4. In some embodiments, the ATP molecule forms a pi-stacking interaction with the synthetic compound. In some embodiments, the ATP molecule binds the protein and the synthetic compound. In some embodiments, the synthetic compound binds cooperatively with the ATP molecule in the RY 1&2 domain of RyR1. In some embodiments, the synthetic compound is a ryanodine receptor modulator, e.g., Compound 1.

In some embodiments, the nucleoside-containing molecule is an adenosine diphosphate (ADP) molecule. In some embodiments, the complex further comprises a second ADP molecule, wherein both ADP molecules bind a common RYR domain of the protein.

In some embodiments, the complex further comprises a second binding site for a nucleoside-containing molecule. In some embodiments, the complex further comprises a second nucleoside-containing molecule. In some embodiments, the second nucleoside-containing molecule binds a C-terminal domain of the RyR1 protein. In some embodiments, the second nucleoside-containing molecule is a nucleotide or nucleoside polyphosphate. In some embodiments, the second nucleoside-containing molecule is a second ATP molecule.

In some embodiments, the complex further comprises calmodulin. In some embodiments, the calmodulin is human calmodulin. In some embodiments, the calmodulin is rabbit calmodulin.

In some embodiments, the complex further comprises calstabin (i.e., peptidyl-prolyl cis-trans isomerase). In some embodiments, the calstabin is rabbit calstabin. In some embodiments, the calstabin is human calstabin. In some embodiments, the calstabin has a sequence according to SEQ ID NO: 2.

In some embodiments, the RyR1 protein is in a resting (closed) state. In some embodiments, the RyR1 protein is in the primed state. In some embodiments, a primed state comprises a higher distribution of open probability (Pₒ) as compared to a RyR1 in a resting (closed) state. In some embodiments, a primed state RyR1 comprises about 30% to about 60% of the RyR channel in an open state. In some embodiments, a primed state RyR1 comprises about 30%, about 35%, about 40%, about 45%, about 50%, about 55% or about 60% of the RyR channel in an open state.

In some embodiments, the complex further comprises a caffeine molecule. In some embodiments, the complex further comprises a Ca²⁺ ion.

In some embodiments, the solid medium comprises vitreous ice. In some embodiments, the solid medium is substantially free of crystalline ice.

In some embodiments, the composition is substantially free of cellular membrane. In some embodiments, the RyR1 is a purified RyR1. In some embodiments, the RyR1 is a semi-purified RyR1 that is substantially free of cellular membrane.

In some embodiments, the composition further comprises additional complexes, wherein each of the additional complexes independently comprises the protein and the synthetic compound.

In some embodiments, the synthetic compound binds a RYR domain of the protein. In some embodiments, the RYR domain is a RYT&2 domain. In some embodiments, the synthetic compound forms a pi-stacking interaction with W882 of the protein. In some embodiments, the synthetic compound forms a salt bridge with H879 of the protein.

In some embodiments, the protein is wild type RyR1. In some embodiments, the protein is mutant RyR1. In some embodiments, the mutant RyR1 is W882A RyR1, W882A RyR1, or C906A RyR1. In some embodiments, the protein is human RyR1. In some embodiments, the protein is rabbit RyR1. In some embodiments, the protein is a tetramer of rabbit RyR1 monomers, wherein each rabbit RyR1 monomer is a peptide according to SEQ ID NO: 3. In some embodiments, the RyR1 protein is C4-symmetrical. In some embodiments, the protein comprises four RYT&2 domains, each with a three-dimensional conformation according to TABLE 2.

### Compounds of the Disclosure.

The synthetic compound in the compositions described herein can be a ryanodine receptor modulator compound, such as a benzothiazepane derivative. Some benzothiazepine compounds are voltage-gated Ca²⁺ channel blockers, but ryanodine receptor modulator compounds can be free of any channel blocking activity. The inability of certain ryanodine receptor modulator compounds to block Ca²⁺ channels can be associated with the mechanism of stabilizing the closed state of the RyR without inhibiting the channel. In some embodiments, a ryanodine receptor modulator compounds are modulators of the RyR channel. In some embodiments, ryanodine receptor modulator compounds are allosteric modulators of the RyR channel.

Ryanodine receptor modulator compounds of the disclosure can be used as therapeutics because in some disease states, RyR leaks Ca²⁺ due to destabilization of the closed state of the channel after post-translational modifications such as nitrosylation, oxidation and phosphorylation. In other disease states, Ca²⁺ leak is present due to inherited mutations. The genetic mutations can predispose the RyR channel to post-translational modifications such as oxidation and nitrosylation, further exacerbating the leak. These mutations and post-translational modifications cause the stabilizing subunit, calstabin, to dissociate from the channel, increasing the open probability of the channel, resulting in Ca²⁺ leak. In disease models involving leaky RyR in cells, animals, and patients, treatment with a ryanodine receptor modulator compound can reverse the leak and restore calstabin binding.

In some embodiments, the synthetic compound comprises a benzazepane or benzothiazepane (e.g., 2,3,4,5-tetrahydro-1,4-benzothiazepine) moiety. In some embodiments, the synthetic compound comprises a benzothiazepane moiety. In some embodiments, the synthetic compound comprises a benzothiazepine moiety. In some embodiments, the synthetic compound comprises a 1,4-benzothiazepine moiety. In some embodiments, the synthetic compound comprises a benzothiazepane moiety, wherein the benzothiazepane moiety forms the pi-stacking interaction with W882 of the protein.

### Chemical Groups.

The term “alkyl” as used herein refers to a linear or branched, saturated hydrocarbon having from 1 to 6 carbon atoms. Representative alkyl groups include, but are not limited to, methyl, ethyl, propyl, isopropyl, butyl, sec-butyl, tert-butyl, pentyl, isopentyl, neopentyl, hexyl, isohexyl, and neohexyl. The term “C1-C4 alkyl” refers to a straight or branched chain alkane (hydrocarbon) radical containing from 1 to 4 carbon atoms, such as methyl, ethyl, propyl, isopropyl, n-butyl, t-butyl, and isobutyl.

The term “alkenyl” as used herein refers to a linear or branched hydrocarbon having from 2 to 6 carbon atoms and having at least one carbon-carbon double bond. In one embodiment, the alkenyl has one or two double bonds. The alkenyl moiety may exist in the E or Z conformation and the compounds of the present invention include both conformations.

The term “alkynyl” as used herein refers to a linear or branched hydrocarbon having from 2 to 6 carbon atoms and having at least one carbon-carbon triple bond.

The term “aryl” as used herein refers to an aromatic group containing 1 to 3 aromatic rings, either fused or linked.

The term “cyclic group” as used herein includes a cycloalkyl group and a heterocyclic group.

The term “cycloalkyl” as used herein refers to a three- to seven-membered saturated or partially unsaturated carbon ring. Any suitable ring position of the cycloalkyl group may be covalently linked to the defined chemical structure. Examples of cycloalkyl groups include cyclopropyl, cyclobutyl, cyclopentyl, cyclohexyl, and cycloheptyl.

The term “halogen” as used herein refers to fluorine, chlorine, bromine, and iodine.

The term “heterocyclic group” or “heterocyclic” or “heterocyclyl” or “heterocyclo” as used herein refers to fully saturated, or partially or fully unsaturated, including aromatic (i.e., “heteroaryl”) cyclic groups (for example, 4 to 7 membered monocyclic, 7 to 11 membered bicyclic, or 10 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached to the remainder of the molecule at any heteroatom or carbon atom of the ring or ring system. Examples of heterocyclic groups include, but are not limited to, azepanyl, azetidinyl, aziridinyl, dioxolanyl, furanyl, furazanyl, homo piperazinyl, imidazolidinyl, imidazolinyl, isothiazolyl, isoxazolyl, morpholinyl, oxadiazolyl, oxazolidinyl, oxazolyl, oxazolidinyl, pyrimidinyl, phenanthridinyl, phenanthrolinyl, piperazinyl, piperidinyl, pyranyl, pyrazinyl, pyrazolidinyl, pyrazolinyl, pyrazolyl, pyridazinyl, pyridooxazolyl, pyridoimidazolyl, pyridothiazolyl, pyridinyl, pyrimidinyl, pyrrolidinyl, pyrrolinyl, quinuclidinyl, tetrahydrofuranyl, thiadiazinyl, thiadiazolyl, thienyl, thienothiazolyl, thienooxazolyl, thienoimidazolyl, thiomorpholinyl, thiophenyl, triazinyl, and triazolyl. Examples of bicyclic heterocyclic groups include indolyl, isoindolyl, benzothiazolyl, benzoxazolyl, benzoxadiazolyl, benzothienyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Examples of tricyclic heterocyclic groups include carbazolyl, benzidolyl, phenanthrolinyl, acridinyl, phenanthridinyl, xanthenyl and the like.

The term “phenyl” as used herein refers to a substituted or unsubstituted phenyl group.

The aforementioned terms “alkyl,” “alkenyl,” “alkynyl,” “aryl,” “phenyl,” “cyclic group,” “cycloalkyl,” “heterocyclyl,” “heterocyclo,” and “heterocycle” can further be optionally substituted with one or more substituents. Examples of substituents include but are not limited to one or more of the following groups: hydrogen, halogen, CF₃, OCF₃, cyano, nitro, N₃, oxo, cycloalkyl, alkenyl, alkynyl, heterocycle, aryl, alkylaryl, heteroaryl, OR^{a}, SR^{a}, S(═O)R^{e}, S(═O)₂R^{e}, P(═O)₂R^{e}, S(═O)₂OR^{a}, P(═O)₂OR^{a}, NR^{b}R^{c}, NR^{b}S(═O)₂R^{e}, NR^{b}P(═O)₂R^{e}, S(═O)₂NR^{b}R^{c}, P(═O)₂NR^{b}R^{c}, C(═O)OR^{a}, C(═O)R^{a}, C(═O)NR^{b}R^{c}, OC(═O)R^{a}, OC(═O)NR^{b}R^{c}, NR^{b}C(═O)OR^{a}, NR^{d}C(═O)NR^{b}R^{c}, NR^{d}S(═O)₂NR^{b}R^{c}, NR^{d}P(═O)₂NR^{b}R^{c}, NR^{b}C(═O)R^{a}, or NR^{b}P(═O)₂R^{e}, wherein R^{a} is hydrogen, alkyl, cycloalkyl, alkenyl, alkynyl, alkylaryl, heteroaryl, heterocycle, or aryl; R^{b}, R^{c} and R^{d} are independently hydrogen, alkyl, cycloalkyl, alkylaryl, heteroaryl, heterocycle, aryl, or said R^{b} and R^{c}, together with the N to which R^{b} and R^{c} are bonded optionally form a heterocycle; and R^{e} is alkyl, cycloalkyl, alkenyl, cycloalkenyl, alkynyl, alkylaryl, heteroaryl, heterocycle, or aryl. In the aforementioned examples of substituents, groups such as alkyl, cycloalkyl, alkenyl, alkynyl, cycloalkenyl, alkylaryl; heteroaryl, heterocycle and aryl can themselves be optionally substituted.

Example substituents can further optionally include at least one labeling group, such as a fluorescent, a bioluminescent, a chemiluminescent, a colorimetric and a radioactive labeling group. A fluorescent labeling group can be selected from bodipy, dansyl, fluorescein, rhodamine, Texas red, cyanine dyes, pyrene, coumarins, Cascade Blue™, Pacific Blue, Marina Blue, Oregon Green, 4′,6-Diamidino-2-phenylindole (DAPI), indopyra dyes, lucifer yellow, propidium iodide, porphyrins, arginine, and variants and derivatives thereof. For example, ARM118 of the present invention contains a labeling group BODIPY, which is a family of fluorophores based on the 4,4-difluoro-4-bora-3a,4a-diaza-s-indacene moiety. For further information on fluorescent label moieties and fluorescence techniques, see, e.g., Handbook of Fluorescent Probes and Research Chemicals, by Richard P. Haughland, Sixth Edition, Molecular Probes, (1996), which is hereby incorporated by reference in its entirety. One of skill in the art can readily select a suitable labeling group, and conjugate such a labeling group to any of the compounds of the invention, without undue experimentation.

### Pharmaceutically Acceptable Salts.

The disclosure provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, or pyrazine.

In some embodiments, an ammonium salt is a triethyl amine salt, a trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, trifluoroacetic acid, mandelic acid, cinnamic acid, aspartic acid, stearic acid, palmitic acid, glycolic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a trifluoroacetate salt, a mandelate salt, a cinnamate salt, an aspartate salt, a stearate salt, a palmitate salt, a glycolate salt, a propionate salt, a butyrate salt, a fumarate salt, a hemifumarate salt, a succinate salt, a methanesulfonate salt, an ethanesulfonate salt, a benzenesulfonate salt, a p-toluenesulfonate salt, a citrate salt, an oxalate salt, or a maleate salt.

### Compounds.

In some embodiments, a compound capable of binding RyR1 is a compound of Formula I:

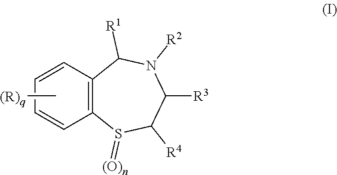

wherein,
n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
t is 1, 2, 3, 4, 5, or 6;
m is 1, 2, 3, or 4;
or a pharmaceutically-acceptable salt thereof.

In some embodiments, R² is unsubstituted alkyl.

In some embodiments, the present disclosure provides compounds of Formula I-a:

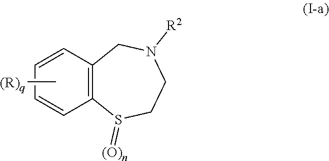

wherein:
n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)R¹², NH(C═O)R¹², —O(C═O)R¹², or —P(═O)R¹³R¹⁴;
m is 0, 1, 2, 3, or 4;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
or a pharmaceutically-acceptable salt thereof.

In some embodiments, the present disclosure provides a compound of formula I-a, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-a,

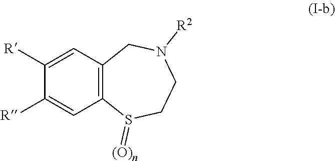

wherein
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰; and
n is 0, 1, or 2;
or a pharmaceutically-acceptable salt thereof.

In some embodiments, the present disclosure provides a compound of formula I-b, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-b, wherein R² is —C═O(R⁵), —C═S(R⁶), —SO₂R⁷, —P(═O)R₈R₉, or —(CH₂)ₘ—R¹⁰.

In some embodiments, the present disclosure provides a compound formula of I-c:

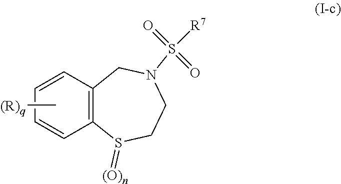

n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X; or a pharmaceutically-acceptable salt thereof.

In some embodiments, the present disclosure provides a compound of formula I-c, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-c, wherein R⁷ is alkyl, alkenyl, aryl, cycloalkyl, cycloalkylalkyl, heterocyclyl, or heterocyclylalkyl, each of which is independently substituted or unsubstituted; or —OH or —NR¹⁵R¹⁶.

In some embodiments, the present disclosure provides a compound of formula of I-d:

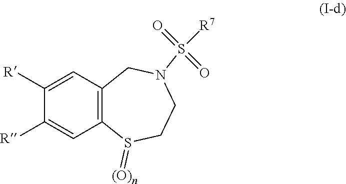

n is 0, 1, or 2;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X, or a pharmaceutically-acceptable salt thereof.

In some embodiments, the present disclosure provides a compound of formula wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-d, wherein R⁷ is alkyl, alkenyl, aryl, cycloalkyl, cycloalkylalkyl, heterocyclyl, or heterocyclylalkyl, each of which is independently substituted or unsubstituted; or —OH, or —NR¹⁵R¹⁶.

In some embodiments, the present disclosure provides a compound of formula of I-e:

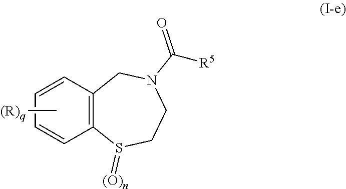

n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃; and
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group, or a pharmaceutically-acceptable salt thereof.

In some embodiments, the present disclosure provides a compound of formula I-e, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-e, wherein R⁵ is alkyl, alkenyl, aryl, cycloalkyl, cycloalkylalkyl, heterocyclyl, or heterocyclylalkyl, each of which is independently substituted or unsubstituted; or —NR¹⁵R¹⁶, —NHOH, —OR¹⁵, or —CH₂X.

In some embodiments, the present disclosure provides a compound of formula of I-f

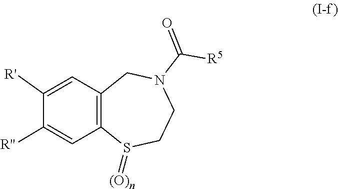

n is 0, 1, or 2;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group, or a pharmaceutically-acceptable salt thereof.

In some embodiments, the present disclosure provides a compound of formula I-f, wherein R′ and R″ are each independently H, halogen, —OH, OMe, —NH₂, —NO₂, —CN, —CF₃, —OCF₃, —N₃, —S(═O)₂C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-f, wherein R₅ is alkyl, alkenyl, aryl, cycloalkyl, cycloalkylalkyl, heterocyclyl, or heterocyclylalkyl, each of which is independently substituted or unsubstituted; or —NR¹⁵R¹⁶, —NHOH, —OR⁵, or —CH₂X.

In some embodiments, the present disclosure provides a compound of formula of I-g:

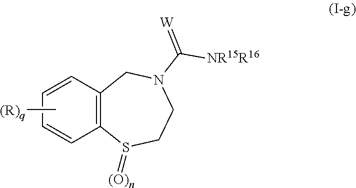

wherein
n is 0, 1, or 2;
q is 0, 1, 2, 3, or 4;
W is S or O;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded may form a heterocycle that is substituted or unsubstituted,
or a pharmaceutically-acceptable salt thereof.

In some embodiments, the present disclosure provides a compound of formula I-g, wherein each R is independently selected from the group consisting of H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl and propenyl; and n is 0, 1, or 2.

In some embodiments, the present disclosure provides a compound of formula I-g, wherein R¹⁵ and R¹⁶ are each independently alkyl, alkylamino, aryl, cycloalkyl, cycloalkylalkyl, heterocyclyl, and heterocyclylalkyl, each of which is independently substituted or unsubstituted; or H, OH, or NH₂; or R¹⁵ and R¹⁶ together with the N to which they are bonded form a heterocycle that is substituted or unsubstituted.

In some embodiments, the present disclosure provides a compound of formula I-g, wherein W is O or S.

In some embodiments, the present disclosure provides a compound of formula of I-h:

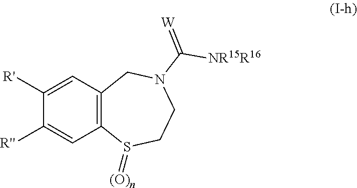

n is 0, 1, or 2;
W is S or O;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃, or a pharmaceutically-acceptable salt thereof.

In some embodiments, the present disclosure provides a compound of formula wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-h, wherein R¹⁵ and R¹⁶ are each independently alkyl, alkylamino, aryl, cycloalkyl, cycloalkylalkyl, heterocyclyl, or heterocyclylalkyl, each of which is independently substituted or unsubstituted; or H, OH, NH₂; or R¹⁵ and R¹⁶ together with the N to which R″ and R¹⁶ are bonded form a heterocycle that is substituted or unsubstituted.

In some embodiments, the present disclosure provides a compound of formula I-g, wherein W is O or S.

In some embodiments, the present disclosure provides a compound of formula of I-i:

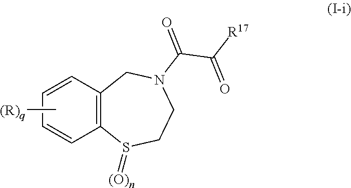

wherein
R¹⁷ is alkenyl, aryl, cycloalkyl, cycloalkylalkyl, heterocyclyl, and heterocyclylalkyl, each of which is independently substituted or unsubstituted; or —NR¹⁵R¹⁶, —NHNR¹⁵R¹⁶, —NHOH, —OR¹⁵, or —CH₂X;
n is 0, 1, or 2;
q is 0, 1, 2, 3, or 4; and
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃,
or a pharmaceutically-acceptable salt thereof.

In some embodiments, the present disclosure provides a compound of formula I-i, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-i, wherein R¹⁷ is —NR¹⁵R¹⁶ or —OR¹⁵. In some embodiments, R¹⁷ is —OH, —OMe, —Net, —NHEt, —NHPh, —NH₂, or —NHCH₂pyridyl.

In some embodiments, the present disclosure provides a compound of formula of I-j:

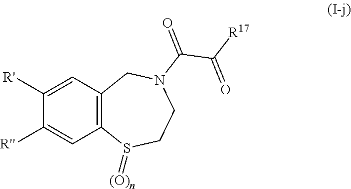

R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R¹⁷ is selected from the group consisting of —NR¹⁵R¹⁶, —NHOH, —OR¹⁵, —CH₂X, alkenyl, aryl, cycloalkyl, cycloalkylalkyl, heterocyclyl, and heterocyclylalkyl; wherein each alkenyl, aryl, cycloalkyl, cycloalkylalkyl, heterocyclyl, and heterocyclylalkyl may be substituted or unsubstituted;
n is 0, 1, or 2,
or a pharmaceutically-acceptable salt thereof.

In some embodiments, the present disclosure provides a compound of formula I-j, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-j, wherein R₁₇ is —NR₁₅R₁₆ or —OR₁₅. In some embodiments, R¹⁷ is —OH, —OMe, —Net, —NHEt, —NHPh, —NH₂, or —NHCH₂pyridyl.

In some embodiments, the present disclosure provides a compound of formula I-k or I-k-1.

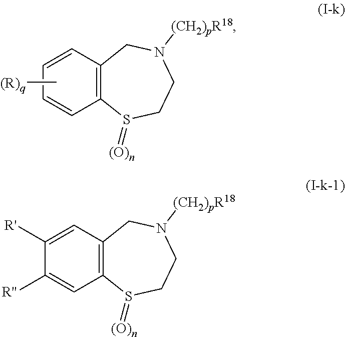

each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroaryl amino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R₁₈ is alkyl, aryl, cycloalkyl, or heterocyclyl, each of which is independently substituted or unsubstituted; or —NR₁₅R₁₆, —C(═O)NR₁₅R₁₆, —(C═O)OR₁₅, or —OR₁₅;
q is 0, 1, 2, 3, or 4;
p is 1, 2, 3, 4, 5, 6, 7, 8 9, or 10; and
n is 0, 1, or 2,
or a pharmaceutically-acceptable salt thereof.

In some embodiments, the present disclosure provides a compound of formula I-k, wherein each R is independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R is OMe at position 7 of the benzothiazepine ring.

In some embodiments, the present disclosure provides a compound of formula I-k-1, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-k or I-k-1, wherein R₁₈ is —NR¹⁵R¹⁶, —(C═O)OR¹⁵, —OR¹⁵, alkyl that is substituted or unsubstituted, or aryl that is substituted or unsubstituted. In some embodiments, m is 1, and R¹⁸ is Ph, C(═O)OMe, C(═O)OH, aminoalkyl, NH₂, NHOH, or NHCbz. In other embodiments, m is 0, and R¹⁸ is C₁-C₄ alkyl. In other embodiments, R¹⁸ is Me, Et, propyl, and butyl. In some embodiments, m is 2, and R¹⁸ is pyrrolidine, piperidine, piperazine, or morpholine. In some embodiments, m is 3, 4, 5, 5, 7, or 8, and R¹⁸ is a fluorescent labeling group selected from bodipy, dansyl, fluorescein, rhodamine, Texas red, cyanine dyes, pyrene, coumarins, Cascade Blue™, Pacific Blue, Marina Blue, Oregon Green, 4′,6-Diamidino-2-phenylindole (DAPI), indopyra dyes, lucifer yellow, propidium iodide, porphyrins, arginine, and variants and derivatives thereof.

In some embodiments, the present disclosure provides a compound of formula of I-l or I-l-1.

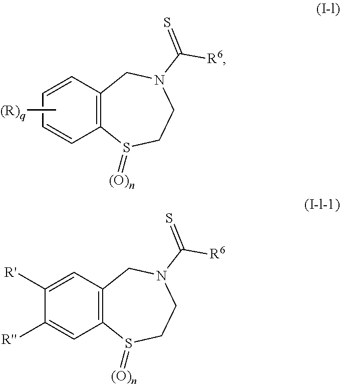

wherein
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
q is 0, 1, 2, 3, or 4; and
n is 0, 1, or 2,
or a pharmaceutically acceptable salt thereof.

In some embodiments, the present disclosure provides a compound of formula I-l, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R is OMe at position 7 of the benzothiazepine ring.

In some embodiments, the present disclosure provides a compound of formula I-l-1, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-1 or I-1-1, wherein R⁶ is acyl, alkenyl, alkyl, aryl, cycloalkyl, cycloalkylalkyl, heterocyclyl, and heterocyclylalkyl, each of which is independently substituted or unsubstituted; or —NR¹⁵R¹⁶, —NHNR¹⁵R¹⁶, —OR¹⁵, —NHOH, or —CH₂X. In some embodiments, R⁶ is —NR¹⁵R¹⁶. In some embodiments, R⁶ is —NHPh, pyrrolidine, piperidine, piperazine, morpholine. In some embodiments, R⁶ is alkoxyl. In some embodiments, R⁶ is —O-tBu.

In some embodiments, the present disclosure provides a compound of formula I-m or I-m-1.

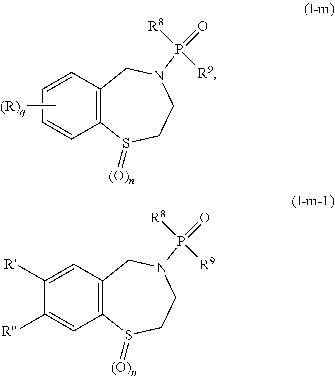

wherein
n is 0, 1, or 2;
q is 0, 1, 2, 3, or 4;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃; and
R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH,
or a pharmaceutically-acceptable salt thereof.

In some embodiments, the present disclosure provides a compound of formula I-m, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R is OMe at position 7 of the benzothiazepine ring.

In some embodiments, the present disclosure provides a compound of formula I-m-1, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-m or I-m-1, wherein R⁸ and R⁹ are each independently alkyl, aryl, —OH, alkoxyl, or alkylamino. In some embodiments, R⁸ is C₁-C₄alkyl. In some embodiments, R⁸ is Me, Et, propyl or butyl. In some embodiments, R⁹ is aryl. In some embodiments, R⁹ is phenyl.

In some embodiments, the present disclosure provides a compound of formula I-n,

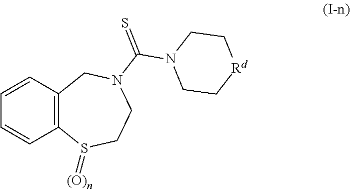

wherein:
R^{d} is CH₂, or NR^{a}; and
R^{a} is H, alkoxy, —(C₁-C₆ alkyl)-aryl, wherein the aryl is a disubstituted phenyl or a benzo[1,3]dioxo-5-yl group, or a Boc group.
or a pharmaceutically-acceptable salt thereof.

In some embodiments, R^{a} is H.

Representative compounds of Formula I-n include without limitation S101, S102, S103, and S114.

In some embodiments, the present disclosure provides a compound of Formula I-o:

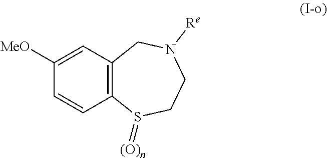

wherein:
R^{e} is —(C₁-C₆ alkyl)-phenyl, —(C₁-C₆ alkyl)-C(O)R^{b}, or substituted or unsubstituted —C₁-C₆ alkyl; and
R^{b} is —OH or —O—(C₁-C₆ alkyl),
wherein the phenyl or the substituted alkyl is substituted with one or more of halogen, hydroxyl, —C₁-C₆ alkyl, —O—(C₁-C₆ alkyl), —NH₂, —NH(C₁-C₆ alkyl), —N(C₁-C₆ alkyl)₂, cyano, or dioxolane,
or a pharmaceutically-acceptable salt thereof.

Representative compounds of Formula I-o include without limitation S107, S110, S111, S120, and S121.

In some embodiments, the present disclosure provides a compound of Formula I-p:

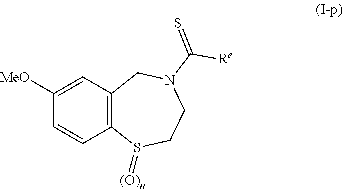

wherein:
R^{c} is —(C₁-C₆ alkyl)-NH₂, —(C₁-C₆ alkyl)-OR^{f}, wherein R_{f} is H or —C(O)—(C₁-C₆)alkyl, or —(C₁-C₆ alkyl)-NHR^{g}, wherein R^{g} is carboxybenzyl.

In some embodiments, the present disclosure provides compounds of Formula II or Formula III:

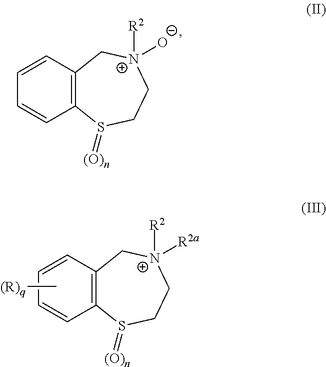

wherein:
n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
each R² and R^{2a} is independently alkyl, aryl, alkylaryl, heteroaryl, cycloalkyl, cycloalkylalkyl, or heterocyclyl, each of which is independently substituted or unsubstituted; or H, —C(═O)R, —C(═S)R⁶, —SO₂R⁷, —P(═O)R⁸R⁹, or —(CH₂)ₘ—R¹⁰;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)R¹², NH(C═O)R¹², —O(C═O)R¹², or —P(═O)R¹³R¹⁴;
m is 0, 1, 2, 3, or 4;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
or a pharmaceutically acceptable salt thereof.

In some embodiments, the compound of formula (I) is selected from:

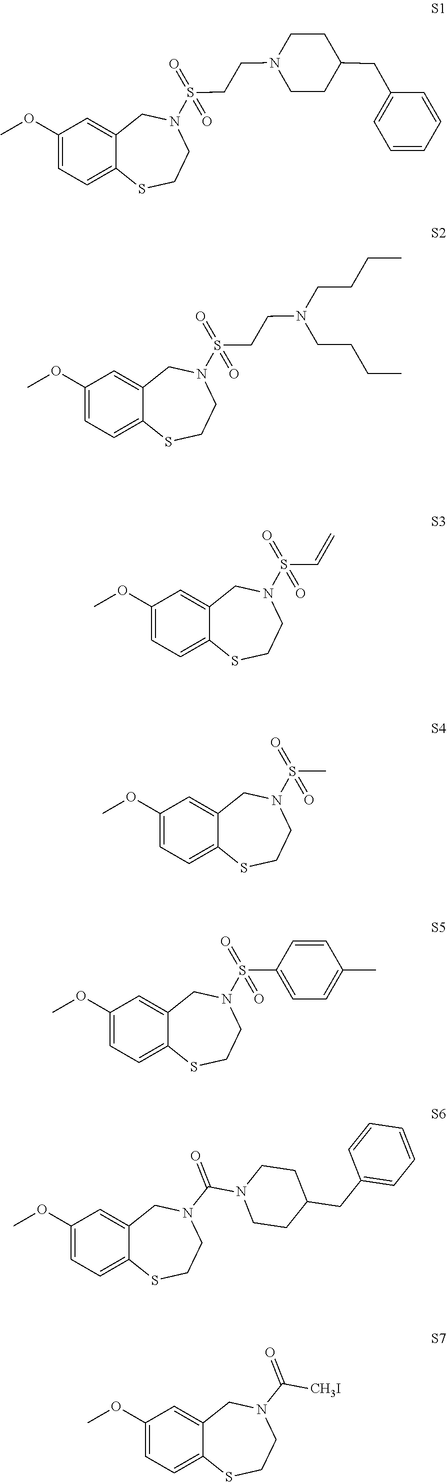

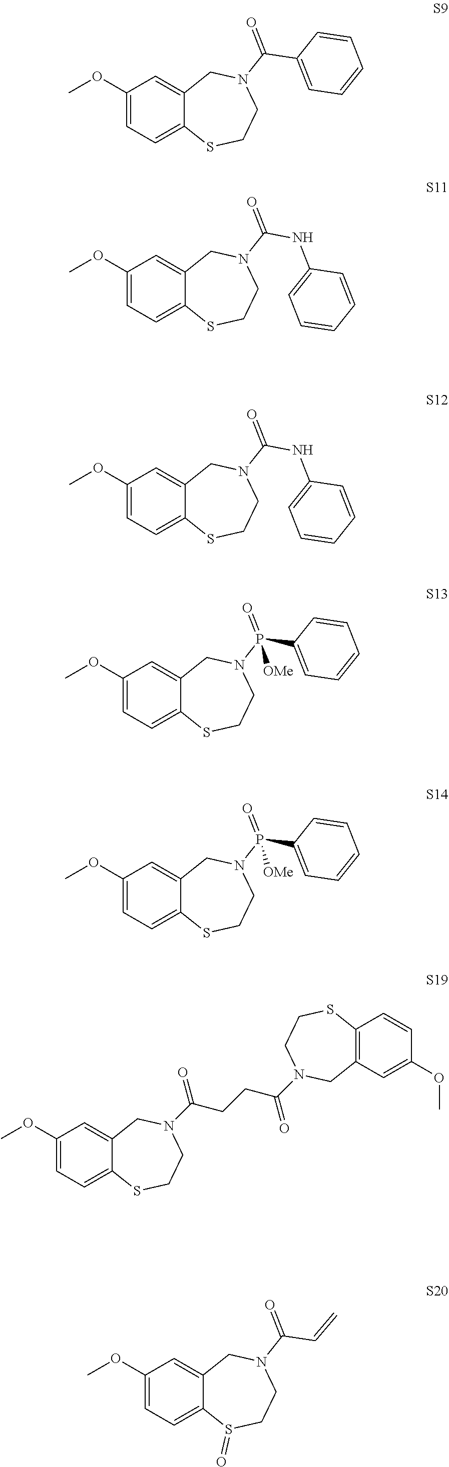

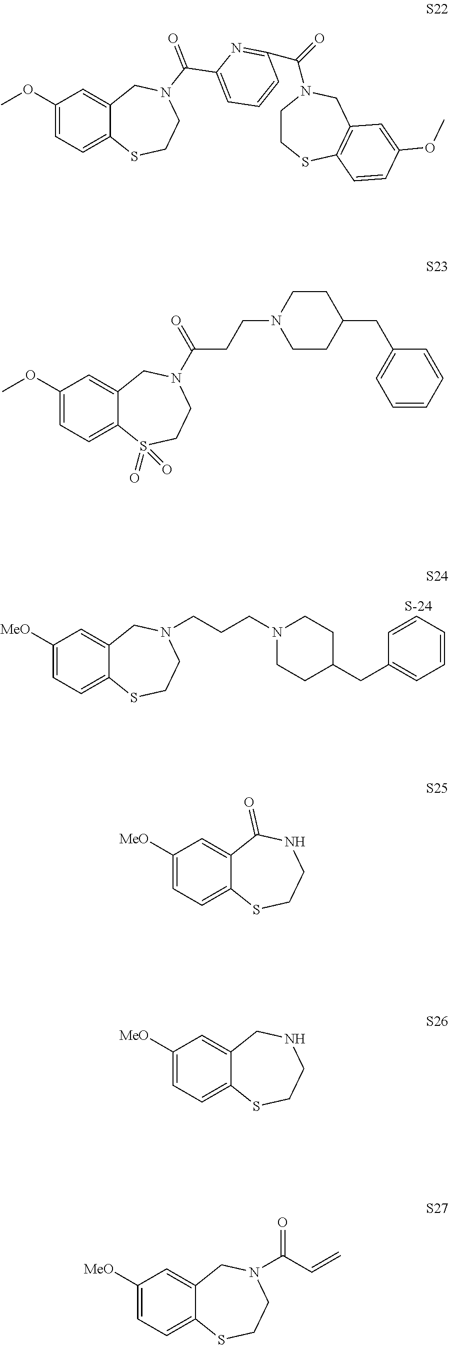

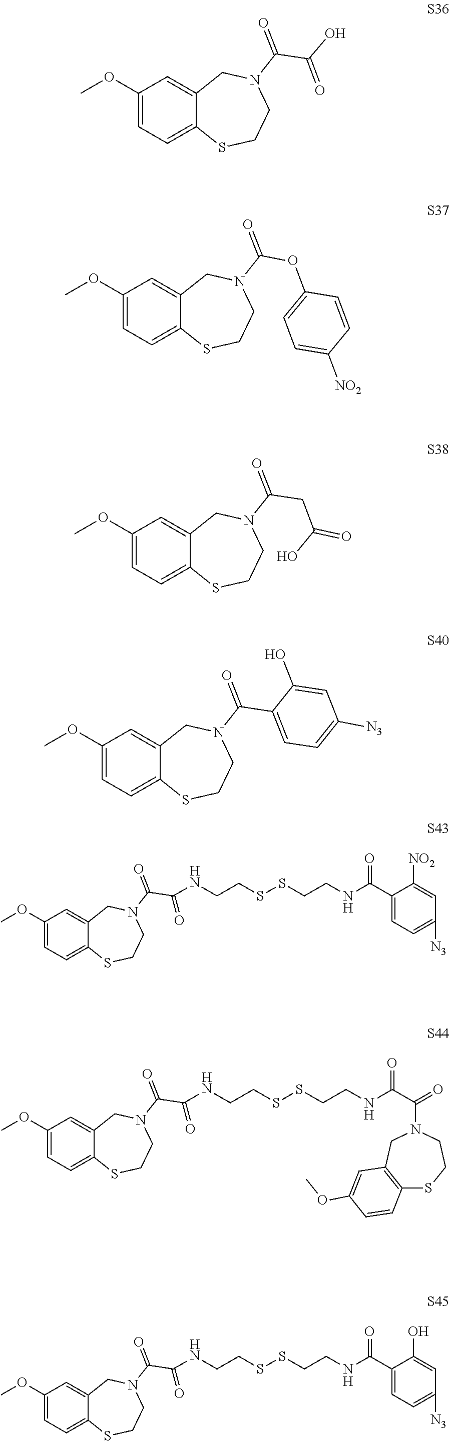

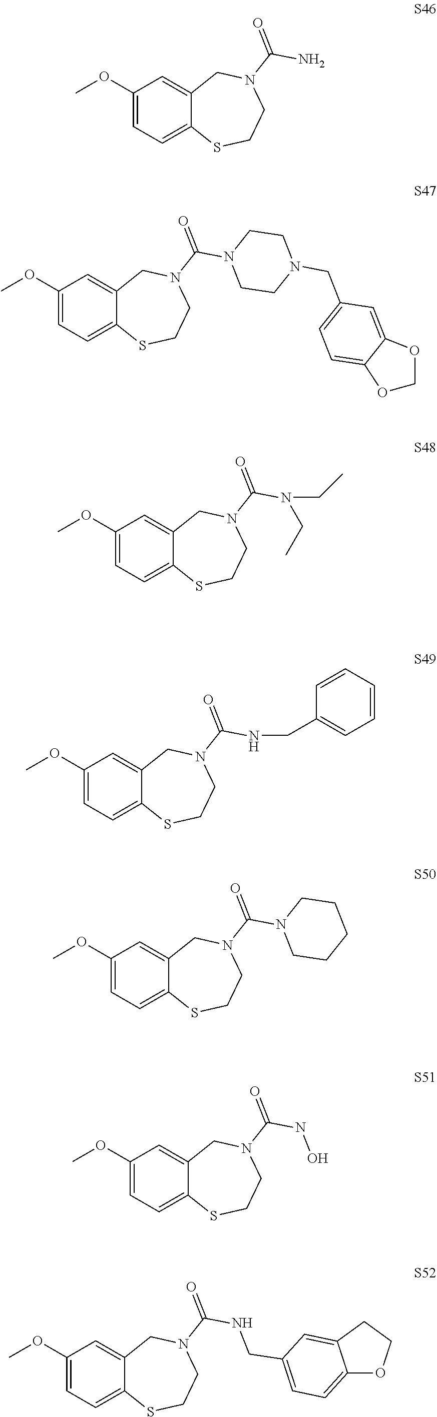

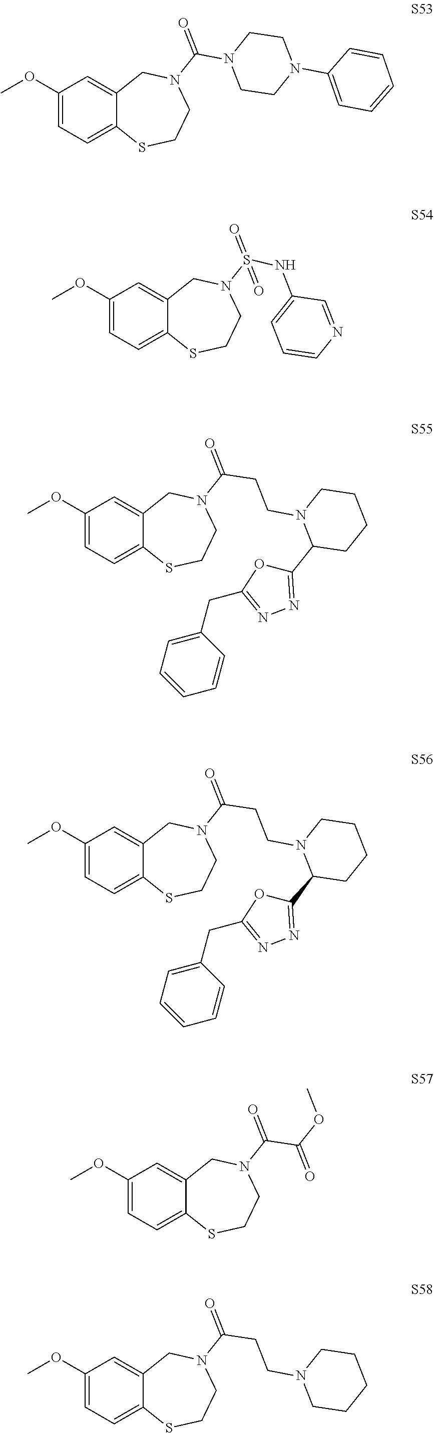 [SMILES: COc1ccc2c(c1)CN(C(=O)N1CCN(c3ccccc3)CC1)CCS2 ; COc1ccc2c(c1)CN(S(=O)(=O)Nc1cccnc1)CCS2 ; COc1ccc2c(c1)CN(C(=O)CCN1CCCCC1c1nnc(Cc3ccccc3)o1)CCS2 ; COc1ccc2c(c1)CN(C(=O)CCN1CCCC[C@H]1c1nnc(Cc3ccccc3)o1)CCS2 ; COC(=O)C(=O)N1CCSc2ccc(OC)cc2C1 ; COc1ccc2c(c1)CN(C(=O)CCN1CCCCC1)CCS2]

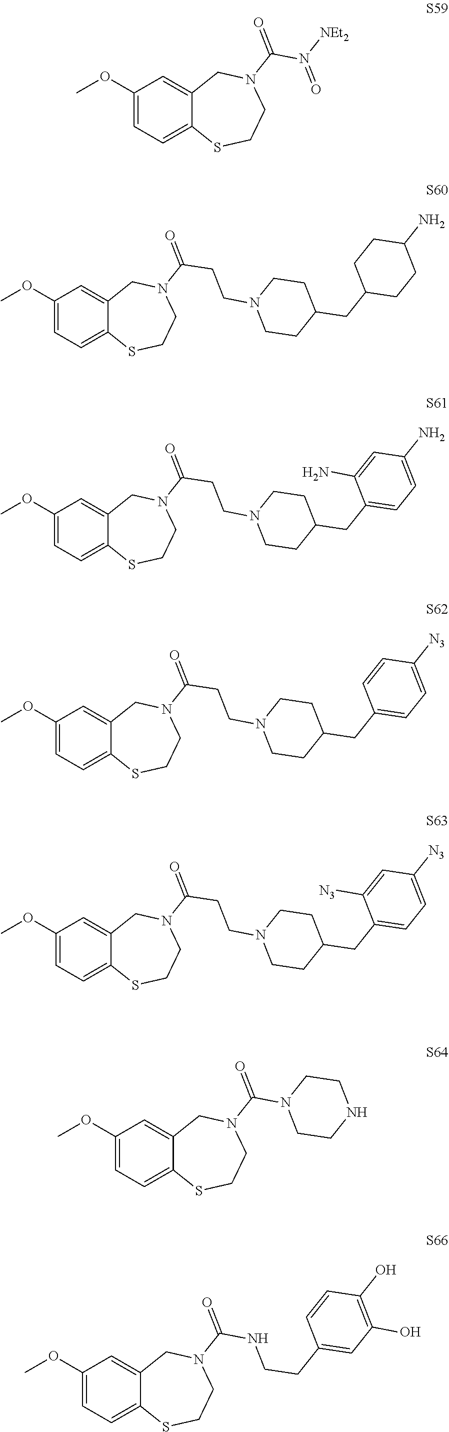

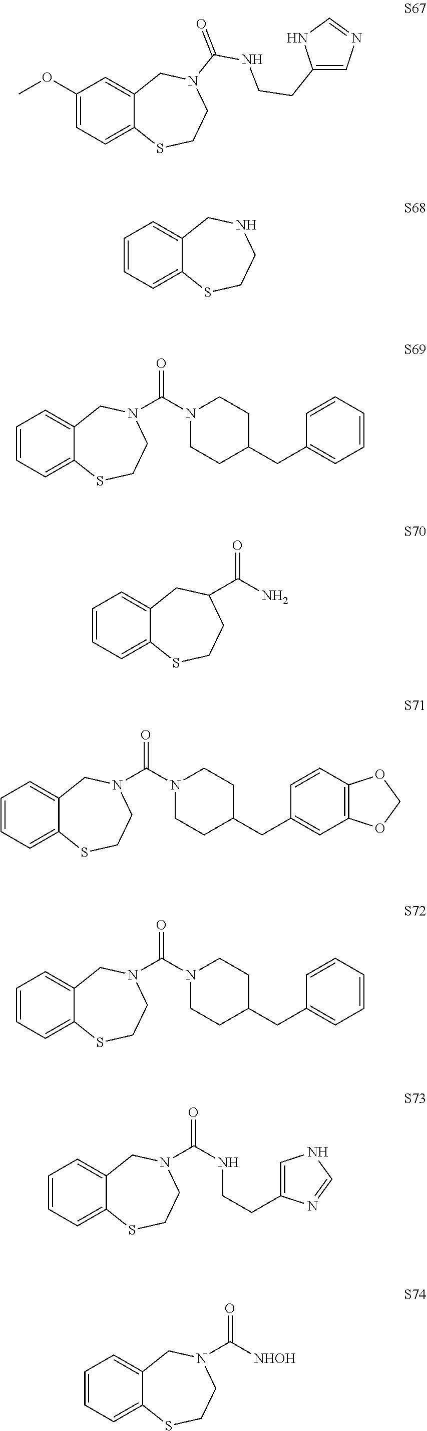

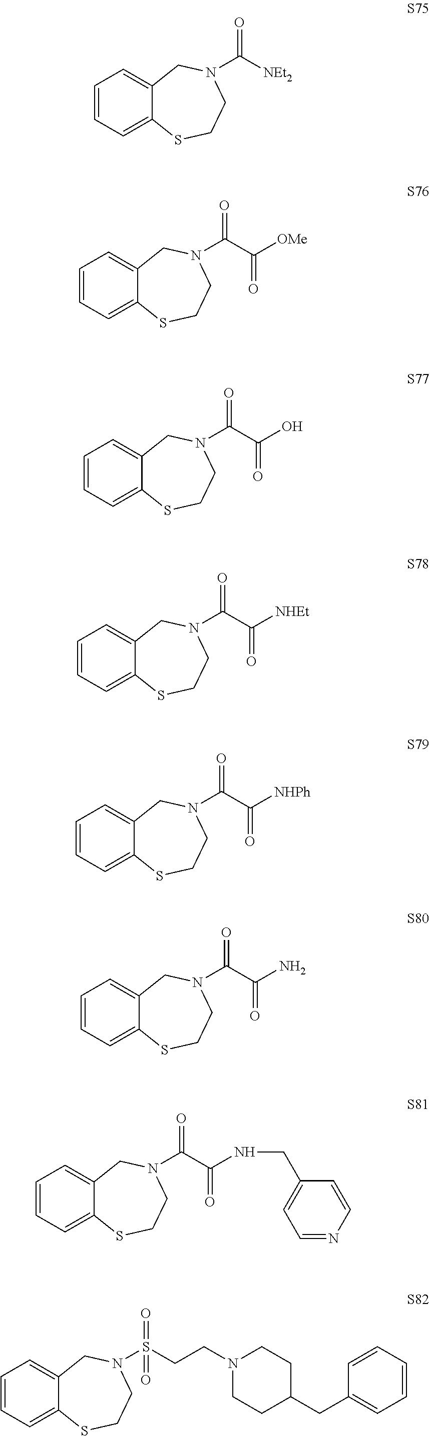

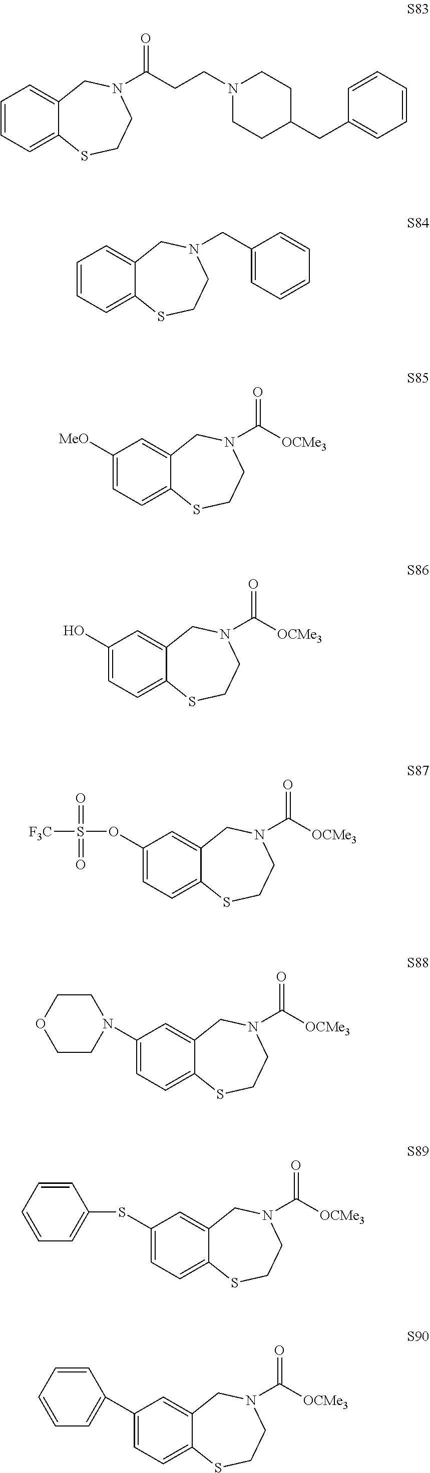

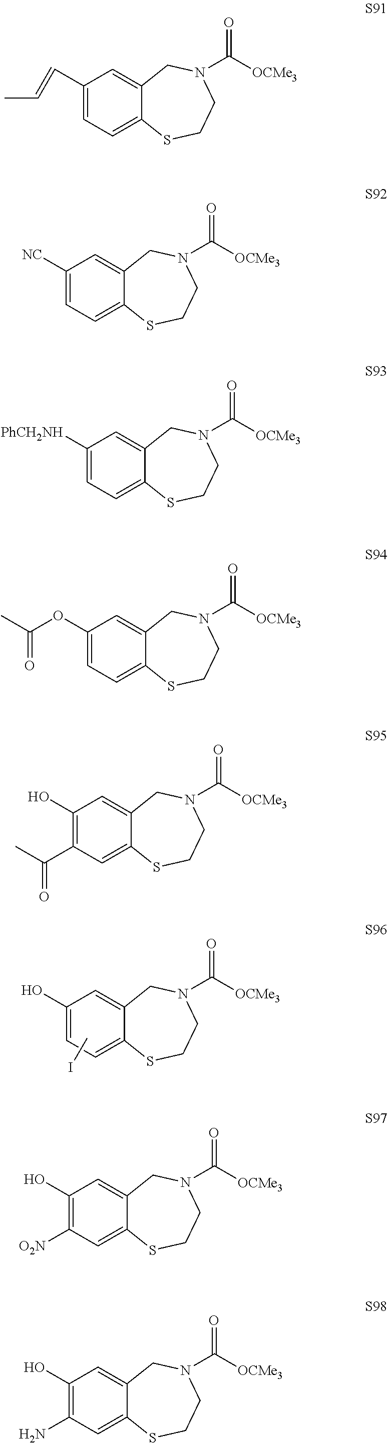

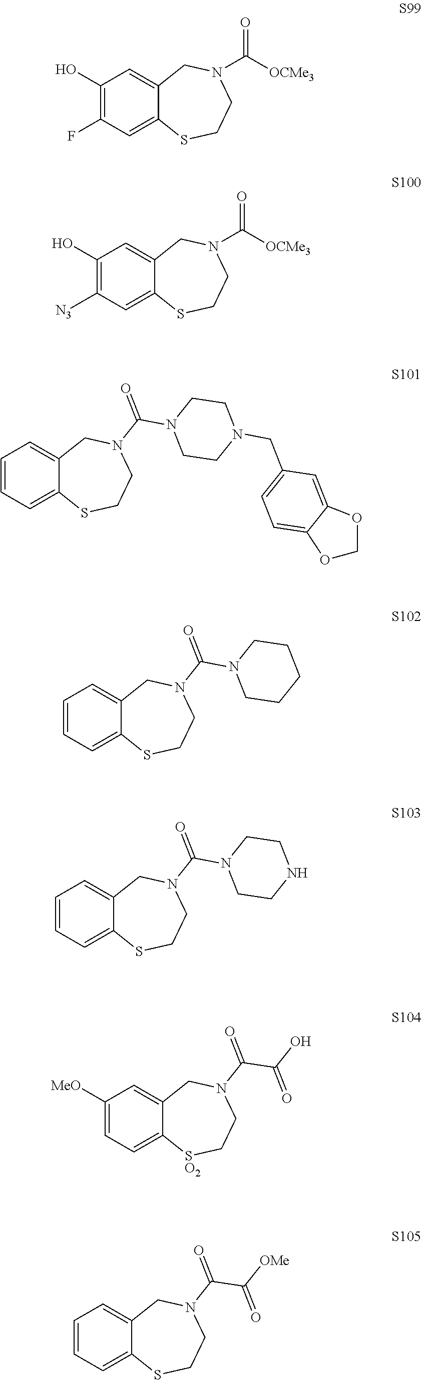

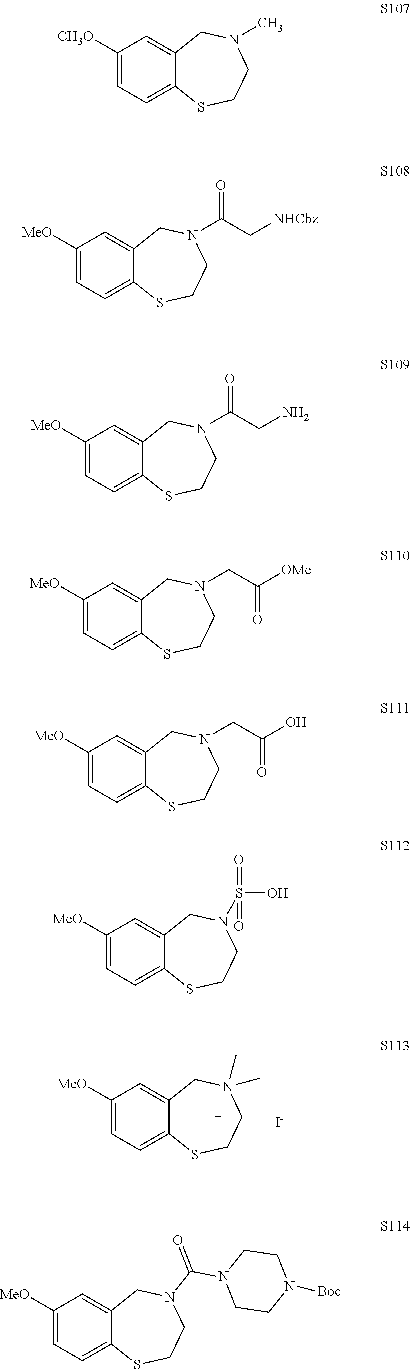

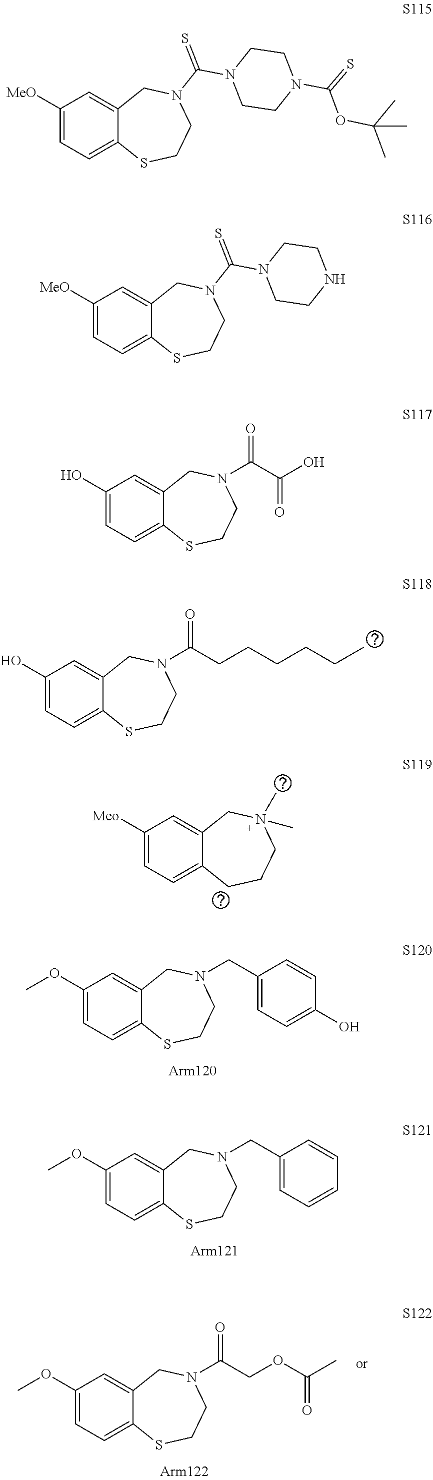

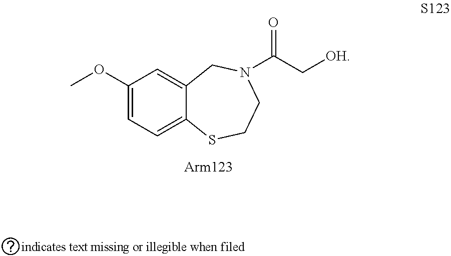 [SMILES: COc1ccc2c(c1)CN(C(=O)CO)CCS2]

In some embodiments, the synthetic compound is a compound of Formula (I), (I-a), (I-b), (I-c), (I-d), (I-e), (I-f), (I-g), (I-h), (I-i), (I-j) (I-k), (I-k-1), (I-l), (I-l-1), (I-m), (I-m-1), (I-n), (I-o), (I-p), (II), or (III). In some embodiments, the synthetic compound is S1, S2, S3, S4, S5, S6, S7, S9, S11, S12, S13, S14, S19, S20, S22, S23, S24, S25, S26, S27, S36, S37, S38, S40, S43, S44, S45, S46, S47, S48, S49, S50, S51, S52, S53, S54, S55, S56, S57, S58, S59, S60, S61, S62, S63, S64, S66, S67, S68, S69, S70, S71, S72, S73, S74, S75, S76, S77, S78, S79, S80, S81, S82, S83, S84, S85, S86, S87, S88, S89, S90, S91, S92, S93, S94, S95, S96, S97, S98, S99, S100, S101, S102, S103, S104, S105, S107, S108, S109, S110, S111, S112, S113, S114, S115, S116, S117, S118, S119, S120, S121, S122, or S123, as herein defined.

In some embodiments, the synthetic compound is:

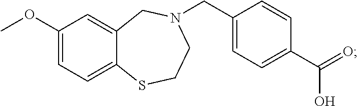 [SMILES: COc1ccc2c(c1)CN(Cc1ccc(C(=O)O)cc1)CCS2]

or a pharmaceutically-acceptable salt thereof or an ionized form thereof.

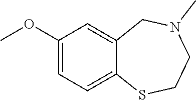

In some embodiments, the synthetic compound is: S or a pharmaceutically-acceptable salt or an ionized form thereof.

Compounds described herein may exist in their tautomeric form (for example, as an amide or imino ether). All such tautomeric forms are contemplated herein as part of the present disclosure.

All stereoisomers of the compounds of the present disclosure (for example, those which may exist due to asymmetric carbons on various substituents), including enantiomeric forms and diastereomeric forms, are contemplated within the scope of this invention. Individual stereoisomers of the compounds of the disclosure may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

### Screening Methods.

The present disclosure provides methods for identifying a compound that binds to a biomolecular target (e.g. RyR1). In some embodiments, the methods described herein can include screening a library of three-dimensional compound structures to identify ligands that fit a binding pocket of the biomolecular target such as RyR1.

In some embodiments, provided is a method of identifying a compound having RyR1 modulatory activity, the method comprising: (a) determining an open probability (Pₒ) of a RyR1 protein; (b) contacting the RyR1 protein with a test compound; (c) determining an open probability (Pₒ) of the RyR1 protein in the presence of the test compound; and (d) determining a difference between the Pₒ of the RyR1 protein in the presence and absence of the test compound; wherein a reduction in the Pₒ of the RyR1 protein in the presence of the test compound is indicative of the compound having RyR1 modulatory activity. In some embodiments, the RyR1 protein is a leaky RyR1. In some embodiments, wherein the RyR1 protein is a mutated RyR1 protein. In some embodiments, the RyR1 protein is a post-translationally modified RyR1 protein. In some embodiments, the RyR1 protein is a mutated and post-translationally modified RyR1 protein. In some embodiments, the test compound preferentially binds to a mutated RyR1 relative to wild-type RyR1. In some embodiments, the test compound preferentially binds to post-translationally modified RyR1 relative to wild-type RyR1. In some embodiments, test compound preferentially binds to a mutant and post-translationally modified RyR1 relative to a wild-type RyR1. In some embodiments, determining the open probability (Pₒ) of the RyR1 protein comprises recording a single channel Ca²⁺ current.

In some embodiments, provided is a method for identifying a compound that preferentially binds to leaky RyR1, comprising: (a) determining binding affinity of a test compound to a first RyR1 protein, wherein the first RyR1 protein is a wild-type RyR1 protein; (b) determining binding affinity of a test compound to a second RyR1 protein, wherein second RyR1 protein is a leaky RyR1, the leaky RyR comprising mutant RyR1 protein, post-translationally modified RyR1 protein, or a combination thereof, and (c) selecting a compound having a higher binding affinity to the second RyR1 protein relative to the first RyR1 protein. In some embodiments, the second RyR1 protein is a mutated RyR1 protein. In some embodiments, the second RyR1 protein is a post-translationally modified RyR1 protein. In some embodiments, the second RyR1 protein is a post-translationally modified RyR1 protein. In some embodiments, the second RyR1 protein is a mutated and post-translationally modified RyR1 protein. In some embodiments, wherein the test compound preferentially binds to a mutated RyR1 protein relative to wild-type RyR1 protein. In some embodiments, the test compound preferentially binds to post-translationally modified RyR1 protein relative to wild-type RyR1 protein. In some embodiments, the test compound preferentially binds to a mutant and post-translationally modified RyR1 relative to a wild-type RyR1 protein.

In some embodiments, the present disclosure provides a method comprising:
(a) determining open probability (Pₒ) of a first RyR1 protein, wherein the first RyR1 protein is treated with both an agent capable of phosphorylating, nitrosylating or oxidizing the first RyR1 protein and a test compound; and
(b) determining open probability (Pₒ) of a second RyR1 protein, wherein the second RyR1 protein is treated with the agent and not treated with the test compound.

In some embodiments, the method further comprises (c) determining open probability (Pₒ) of a third RyR1 protein, wherein the third RyR1 protein is neither treated with the agent nor treated with the test compound.

In some embodiments, the present disclosure provides a method comprising:
(a) determining open probability (Pₒ) of a first RyR1 protein, wherein the first RyR1 protein is a mutated RyR1 protein or a post-translationally modified RyR1 protein, and wherein the first RyR1 protein is treated with a test compound; and
(b) determining open probability (Pₒ) of a second RyR1 protein, wherein the second RyR1 protein is a mutated RyR1 protein or a post-translationally modified RyR1 protein, and wherein the second RyR1 protein is not treated with the test compound.

In some embodiments, determining the open probability (Pₒ) of the first RyR1 protein and the second RyR1 protein comprises recording a single channel Ca²⁺ current.

In some embodiments, the method further comprises determining a difference between the Pₒ of the first RyR1 protein and Pₒ of the second RyR1 protein. In some embodiments, the method further comprises determining the difference between the Pₒ of the first RyR1 protein and Pₒ of the third RyR1 protein.

In some embodiments, the method further comprises identifying the test compound as a target for further analysis based on the difference between the Pₒ of the first RyR1 protein and Pₒ of the second RyR1 protein.

In some embodiments, the method further comprises performing an analogous assay where another compound is used in place of the test compound, wherein the analogous assay provides a difference between:
(a) an open probability (Pₒ) of a fourth RyR1 protein, wherein the fourth RyR1 protein is treated with both an agent capable of phosphorylating, nitrosylating or oxidizing the first RyR1 protein and the other compound; and
(b) an open probability (Pₒ) of a fifth RyR1 protein, wherein the fifth RyR1 protein is treated with the agent and not treated with the other compound,
wherein the test compound is prioritized over the other compound for the further analysis based on a comparison of:
(i) the difference between the Pₒ of the first RyR1 protein and Pₒ of the second RyR1 protein; with
(ii) a difference between the Pₒ of the fourth RyR1 protein and Pₒ of the fifth RyR1 protein.

In some embodiments, the difference is subtractive.

In some embodiments, the agent is an oxidant. In some embodiments, the agent is H₂O₂.

In some embodiments, the present disclosure provides a method comprising:
(a) contacting a first RyR1 protein with an agent capable of phosphorylating, nitrosylating or oxidizing the first RyR1 protein and a test compound;
(b) contacting a second RyR1 protein with the agent and not with the test compound;
(c) subsequent to the contacting the first RyR1 protein with the agent and the test compound, measuring an open probability (Pₒ) of the first RyR1 protein; and
(d) subsequent to the contacting the first RyR1 protein with the agent and the test compound, measuring an open probability (Pₒ) of the second RyR1 protein.

In some embodiments, the method further comprises (e) determining open probability (Pₒ) of a third RyR1 protein without contacting the third RyR1 protein with the agent and without contacting the third RyR1 protein with the test compound.

In some embodiments, each of the determining the open probability (Pₒ) of the first RyR1 protein and the determining the open probability (Pₒ) of second RyR1 protein comprises recording a single channel Ca²⁺ current.

In some embodiments, the method further comprises determining a difference between the Pₒ of the first RyR1 protein and the Pₒ of the second RyR1 protein. In some embodiments, the method further comprises determining a difference between the Pₒ of the first RyR1 protein and the Pₒ of the third RyR1 protein.

In some embodiments, the method further comprises identifying the test compound as a target for further analysis based on the difference between the Pₒ of the first RyR1 protein and the Pₒ of the second RyR1 protein.

In some embodiments, the method further comprises performing an analogous assay where another compound is used in place of the test compound, wherein the analogous assay provides a difference between:
(a) an open probability (Pₒ) of a fourth RyR1 protein, wherein the fourth RyR1 protein is treated with both an agent capable of phosphorylating, nitrosylating or oxidizing the first RyR1 protein and the other compound; and
(b) an open probability (Pₒ) of a fifth RyR1 protein, wherein the fifth RyR1 protein is treated with the agent and not treated with the other compound,
wherein the test compound is prioritized over the other compound for the further analysis based on a comparison of:
(i) the difference between the Pₒ of the first RyR1 protein and Pₒ of the second RyR1 protein; with
(ii) a difference between the Pₒ of the fourth RyR1 protein and Pₒ of the fifth RyR1 protein.

In some embodiments, each difference is a subtractive difference.

In some embodiments, the method further comprises: subsequent to the contacting the first RyR1 protein with the agent and the test compound, fusing a first microsome containing the first RyR1 protein to a first planar lipid bilayer, and subsequent to the contacting the second RyR1 protein with the agent, fusing a second microsome containing the second RyR1 protein to a second planar lipid bilayer.

In some embodiments, the agent capable of post-translationally modifying the RyR1 (e.g., phosphorylating, nitrosylating or oxidizing) is an oxidant. In some embodiments, the agent is a nitrosylating agent. In some embodiments, the agent is a phosphorylating agent (e.g., PKA and/or CaMKII).

In some embodiments, the agent is an oxidant. In some embodiments, the oxidant is a solution containing H₂O₂. In some embodiments, the oxidant is a solution containing about 0.5 to about 10 mM H₂O₂.

In some embodiments, instead of, or in addition to treatment of RyR1 with an agent capable of post-translationally modifying the RyR1 (e.g., phosphorylating, nitrosylating or oxidizing agent), the present methods can utilize a mutant RyR1. In some embodiments, the mutant RyR1 is in a primed state having a higher open probability (Pₒ) as compared to RyR1 channel in a closed or resting state.

In some embodiments, each RyR1 protein is a wild type RyR1 protein. In some embodiments, each RyR1 protein is a C906A mutant. In some embodiments, each RyR1 protein is a W882A mutant.

In some embodiments, the initial test compound is a compound of Formula (I), (I-a), (I-b), (I-c), (I-d), (I-e), (I-f), (I-g), (I-h), (I-i), (I-j), (I-k), (I-k-1), (I-l), (I-l-1), (I-m), (I-m-1), (I-n), (I-o), (I-p), (II) or (III).

### Computational Methods.

In some embodiments, a cryo-EM model disclosed herein can be used as a tool to screen for ryanodine receptor modulator compounds that bind RyR1. In some embodiments, a cryo-EM model disclosed herein can be used as a tool to screen for ryanodine receptor modulator compounds which preferentially bind leaky RyR1 (e.g., mutated RyR1, or post-translationally modified RyR1 (e.g., phosphorylated, oxidized and/or nitrosylated RyR1)) and stabilize the closed state of the RyR channel.

Structures of compounds (e.g., Compound 1) and biomolecular targets (e.g. RyR1) provided herein can be used in computational methods for identifying ligands that bind to a biomolecular target (e.g. RyR1). Such methods can include, for example, screening a library of three-dimensional compound structures to identify ligands that fit a binding pocket of the biomolecular target via a molecular docking system (e.g. Glide, DOCK, AutoDock, AutoDock Vina, FRED, and EnzyDock); de-novo generation of a structure of a ligand that binds the biomolecular target via a ligand structure prediction system (e.g., CHARMM, AMBER, or GROMACS); optimization of known ligands (e.g., Compound 1) by evaluating binding of proposed analogs within the binding cavity of the biomolecular target, and combinations of the preceding.

Structures of compounds (e.g., Compound 1) and biomolecular targets (e.g. RyR1) provided herein can be used in computational methods of predicting a docked position of a target ligand in a binding site of a biomolecule, such as the use of a computer to assist in predicting a docked position of a target ligand in a binding site of a biomolecule that is capable of undergoing an induced fit as disclosed in US20210193273A1, which is incorporated herein by reference in its entirety.

In some embodiments, the present disclosure provides a method for predicting a docked position of a target ligand in a binding site of a biomolecule, the method comprising:
receiving a template ligand-biomolecule structure, the template ligand-biomolecule structure comprising a template ligand docked in the binding site of the biomolecule;
comparing a pharmacophore model of the template ligand to a pharmacophore model of the target ligand;
overlapping the pharmacophore model of the target ligand with the pharmacophore model of the template ligand while the template ligand is in the binding site of the biomolecule; and
predicting the docked position of the target ligand in the binding site of the biomolecule based on a position of the pharmacophore model of the target ligand when overlapped with the pharmacophore model of the template ligand, wherein the biomolecule is a RYT&2 domain of RyR1, wherein the template ligand-biomolecule structure is obtained by a process comprising subjecting a complex of the biomolecule and the template ligand to single-particle cryogenic electron microscopy analysis.

In some embodiments, the RYT&2 domain comprises a structure according to TABLE 2. In some embodiments, the template ligand has a three-dimensional conformation according to TABLE 3. In some embodiments, the RYT&2 domain further comprises second binding site. In some embodiments, the second binding site is an ATP-binding site. In some embodiments, the RYT&2 domain further comprises a nucleoside-containing molecule. In some embodiments, the nucleoside-containing molecule is an ATP molecule. In some embodiments the target ligand cooperatively binds the RYT&2 domain with the ATP molecule. In some embodiments, the ATP molecule has a three-dimensional conformation according to TABLE 4. In some embodiments, the target ligand cooperatively binds the RYT&2 domain with the ATP molecule. In some embodiments, the target ligand forms a pi-stacking interaction with W882 of the protein.

In some embodiments, the target ligand is a compound of Formula (I), (I-a), (I-b), (I-c), (I-d), (I-e), (I-f), (I-g), (I-h), (I-i), (I-j), (I-k), (I-k-1), (I-l), (I-l-1), (I-m), (I-m-1), (I-n), (I-o), (I-p), (II) or (III). In some embodiments, the target ligand and the template ligand are each independently a compound of Formula (I), (I-a), (I-b), (I-c), (I-d), (I-e), (I-f), (I-g), (I-h), (I-i), (I-j), (I-k), (I-k-1), (I-l), (I-l-1), (I-m), (I-m-1), (I-n), (I-o), (I-p), (II) or (III).

In some embodiments, the template ligand is

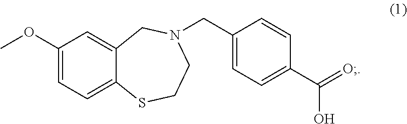 [SMILES: COc1ccc2c(c1)CN(Cc1ccc(C(=O)O)cc1)CCS2]

In some embodiments, the template ligand is

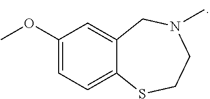

In some embodiments, the template ligand-biomolecule structure obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

In some embodiments, the method further comprises selecting the target ligand from a plurality of ligand candidates, each of the ligand candidates being different from the template ligand, and wherein selecting the target ligand comprises comparing the pharmacophore model of the template ligand to a pharmacophore model of each respective one of the plurality of ligand candidates.

In some embodiments, the method further comprises receiving a plurality of template ligand-biomolecule structures, each template ligand-biomolecule structure having a different template ligand docked in the binding site of the biomolecule, and generating the pharmacophore model of the template ligand by combining information from each of the template ligands from the plurality of template ligand-biomolecule structures.

In some embodiments, the target ligand has more than one structural conformation in the unbound state, and the docked position of the target ligand in the binding site of the biomolecule is predicted by enumerating a set of potential target ligand conformations and overlapping a respective pharmacophore model of the target ligand for each of the potential target ligand conformations with the pharmacophore model of the template ligand while the template ligand is in the binding site of the biomolecule.

In some embodiments, predicting the docked position of the target ligand in the binding site of the biomolecule comprises ignoring at least one clash between the target ligand conformation's atomic coordinates and the biomolecule's atomic coordinates.

In some embodiments, the method further comprises, for each target ligand conformation, modifying atomic coordinates of the biomolecule to reduce clashes between the docked target ligand conformation's atomic coordinates and the biomolecule's atomic coordinates, thereby creating an altered ligand-biomolecule structure comprising the docked target ligand and an altered biomolecule.

In some embodiments, the method further comprises predicting a re-docked position of each target ligand conformation by predicting each target ligand conformation's position in the binding site of the altered biomolecule; and for each target ligand conformation, modifying atomic coordinates of the altered biomolecule to reduce clashes between the atomic coordinates of the target ligand conformation's re-docked position and the atomic coordinates of the altered biomolecule, thereby creating are-altered ligand-biomolecule structure comprising a re-docked target ligand and a re-altered biomolecule.

In some embodiments, the method further comprises ranking each altered and re-altered ligand-biomolecule structure using a scoring function.

In some embodiments, the method further comprises identifying a subset of high-ranking target ligands corresponding to target ligands having a threshold value for an empirical activity.

Structures of compounds (e.g., Compound 1) and biomolecular targets (e.g. RyR1) provided herein can be used in systems, devices, and methods that can generate lead compounds on the basis of known structure and activity of a lead compound (e.g., Compound 1) and the structure of a binding site for the lead compound, such as the systems, devices, and methods provided in US20210217500A1, which is incorporated herein by reference in its entirety.

In some embodiments, the present disclosure provides a method of identifying a plurality of potential lead compounds, the method comprising the steps of:
(a) analyzing, using a computer system, an initial lead compound known to bind to a biomolecular target, the analyzing comprising partitioning, by providing a database of known reactions, the initial lead compound into atoms defining partitioned lead compound comprising a lead compound core and atoms defining a lead compound non-core, wherein the initial lead compound is partitioned using a computational retrosynthetic analysis of the initial lead compound;
(b) identifying, using the computer system, a plurality of alternative cores to replace the lead compound core in the initial lead compound, thereby generating a plurality of potential lead compounds each having a respective one of the plurality of alternative cores;
(c) calculating, using the computer system, a difference in binding free energy between the partitioned lead compound and each potential lead compound;
(d) predicting, using the computer system, whether each potential lead compound binds to the biomolecular target and identifying a predicted active set of potential lead compounds based on the prediction;
(e) obtaining a synthesized set of at least some of the potential leads of the predicted active set to establish a first of potential lead compounds; and
(f) determining, empirically, an activity of each of the first set of synthesized potential lead compounds,
wherein the biomolecular target is a RYT&2 domain of RyR1, and the structure of the biomolecular target used in the predicting of (d) is obtained by a process comprising subjecting a complex of the biomolecular target and the initial lead compound to single-particle cryogenic electron microscopy analysis.

In some embodiments, the structure of the biomolecular target obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

In some embodiments, the initial lead compound is a compound of Formula (I), (I-a), (I-b), (I-c), (I-d), (I-e), (I-f), (I-g), (I-h), (I-i), (I-j), (I-k), (I-k-1), (I-l), (I-l-1), (I-m), (I-m-1), (I-n), (I-o), (I-p), (II) or (III).

In some embodiments, the initial lead compound is

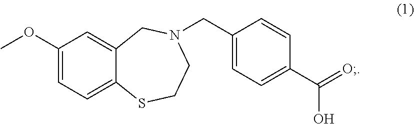 [SMILES: COc1ccc2c(c1)CN(Cc1ccc(C(=O)O)cc1)CCS2]

In some embodiments, the initial lead compound is s

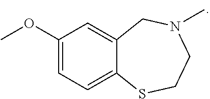

In some embodiments, the RYT&2 domain comprises a structure according to TABLE 2. In some embodiments, the RYT&2 domain contains an ATP molecule. In some embodiments, the ATP molecule has a three-dimensional conformation according to TABLE 4.

In some embodiments, the method further comprises obtaining a synthesized set of at least some of the potential lead compounds predicted to not bind with the biomolecular target to establish a second set of potential lead compounds and empirically determining an activity of each of the second set of synthesized potential lead compounds.

In some embodiments, the method further comprises comparing the empirically determined activity of each of the first set of synthesized potential lead compounds with a threshold activity level.

In some embodiments, the method further comprises comparing the empirically determined activity of each of the second set of synthesized potential lead compounds with a pre-determined activity level.

In some embodiments, the plurality of alternative cores are chosen from a database of synthetically feasible cores.

In some embodiments, the difference in binding free energy is calculated using a free energy perturbation technique.

In some embodiments, the generation of at least one potential lead compound comprises creating an additional covalent bond or annihilating an existing covalent bond, or both creating an additional first covalent bond and annihilating an existing second covalent bond different from the first covalent bond.

In some embodiments, the free energy perturbation technique uses a soft bond potential to calculate a bonded stretch interaction energy of existing covalent bonds for annihilation and additional covalent bonds for creation.

In some embodiments, the present disclosure provides a method for pharmaceutical drug discovery, comprising:
identifying an initial lead compound for binding to a biomolecular target; using a method to identify a predicted active set of potential lead compounds for binding to the biomolecular target based on the initial lead compound, comprising:
   (a) analyzing, using a computer system, an initial lead compound known to bind to a biomolecular target, the analyzing comprising partitioning, by providing a database of known reactions, the initial lead compound into atoms defining partitioned lead compound comprising a lead compound core and atoms defining a lead compound non-core, wherein the initial lead compound is partitioned using a computational retrosynthetic analysis of the initial lead compound;
   (b) identifying, using the computer system, a plurality of alternative cores to replace the lead compound core in the initial lead compound, thereby generating a plurality of potential lead compounds each having a respective one of the plurality of alternative cores;
   (c) calculating, using the computer system, a difference in binding free energy between the partitioned lead compound and each potential lead compound;
   (d) predicting, using the computer system, whether each potential lead compound binds to the biomolecular target and identifying a predicted active set of potential lead compounds based on the prediction;
   (e) obtaining a synthesized set of at least some of the potential leads of the predicted active set to establish a first of potential lead compounds; and
   (f) determining, empirically, an activity of each of the first set of synthesized potential lead compounds,
   (g) selecting one or more of the predicted active set of potential lead compounds for synthesis; and
   (h) assaying the one or more synthesized selected compounds to assess each synthesized selected compounds suitability for in vivo use as a pharmaceutical compound,
wherein the biomolecular target is a RY1&2 domain of RyR1, and the structure of the biomolecular target used in the predicting of (d) is obtained by a process comprising subjecting a complex of the biomolecular target and the initial lead compound to single-particle cryogenic electron microscopy analysis.

In some embodiments, the structure of the biomolecular target obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

In some embodiments, the initial lead compound is a compound of Formula (I), (I-a), (I-b), (I-c), (I-d), (I-e), (I-f), (I-g), (I-h), (I-i), (I-j), (I-k), (I-k-1), (I-l), (I-l-1), (I-m), (I-m-1), (I-n), (I-o), (I-p), (II) or (III).

In some embodiments, the initial lead compound is

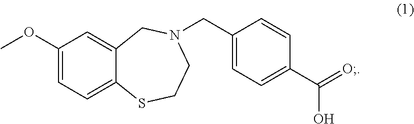 [SMILES: COc1ccc2c(c1)CN(Cc1ccc(C(=O)O)cc1)CCS2]

In some embodiments, the initial lead compound is

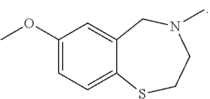

In some embodiments, the RY1&2 domain comprises a structure according to TABLE 2. In some embodiments, the RY1&2 domain contains an ATP molecule. In some embodiments, the ATP molecule has a three-dimensional conformation according to TABLE 4.

Structures of compounds (e.g., Compound 1) and biomolecular targets (e.g. RyR1) provided herein can be used in methods that estimate binding affinity between a ligand and a receptor molecule, including the systems and methods disclosed in U.S. Pat. No. 8,160,820B2, which is incorporated by reference herein in its entirety.

In some embodiments, the present disclosure provides a computer-implemented method of quantifying binding affinity between a ligand and a receptor molecule domain, the method comprising:
receiving by one or more computers, data representing a ligand molecule,
receiving by one or more computers, data representing a receptor molecule domain,
using the data representing the ligand molecule and the data representing the receptor molecule domain in computer analysis to identify ring structure within the ligand, the ring structure being an entire ring or a fused ring;
using the data representative of the identified ligand ring structure to designate a first ring face and a second ring face opposite to the first ring face, and classifying the ring structure by:
a) determining proximity of receptor atoms to atoms on the first face of the ligand ring; and
b) determining proximity of receptor atoms to atoms on the second face of the ligand ring;
c) determining solvation of the first face of the ligand ring and solvation of the second face of the ligand ring;
classifying the identified ligand ring structure as buried, solvent exposed, or having a single face exposed to solvent based on receptor atom proximity to and solvation of the first ring face and receptor atom proximity to and solvation of the second ring face; quantifying the binding affinity between the ligand and the receptor molecule domain based at least in part on the classification of the ring structure; and
displaying, via computer, information related to the classification of the ring structure, wherein the receptor molecule domain is a RYT&2 domain of RyR1, wherein the data representing a ligand molecule and the data representing a receptor molecule domain are obtained by a process comprising subjecting a complex comprising the ligand molecule and the receptor molecule domain to single-particle cryogenic electron microscopy analysis.

In some embodiments, the structure of the receptor molecule domain obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

In some embodiments, ligand molecule is a compound of Formula (I), (I-a), (I-b), (I-c), (I-d), (I-e), (I-f), (I-g), (I-h), (I-i), (I-j), (I-k), (I-k-1), (I-l), (I-l-1), (I-m), (I-m-1), (I-n), (I-o), (I-p), (II) or (III).

In some embodiments, the ligand molecule is

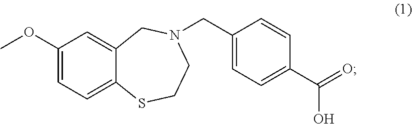 [SMILES: COc1ccc2c(c1)CN(Cc1ccc(C(=O)O)cc1)CCS2]

or a pharmaceutically-acceptable salt or ionized form thereof.

In some embodiments, the ligand molecule is

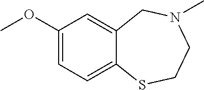

or a pharmaceutically-acceptable salt or ionized form thereof.

In some embodiments, the complex further comprises a RyR1 protein, wherein the RY1&2 domain is a domain of the RyR1 protein.

In some embodiments, the data representing the receptor molecule domain represents a three-dimensional structure of the receptor molecule according to TABLE 2. In some embodiments, the data representing a ligand molecule represents a three-dimensional structure of the ligand molecule according to TABLE 3.

In some embodiments, the receptor molecule domain contains an ATP molecule. In some embodiments, the data representing the receptor molecule domain further comprises data representing a three-dimensional structure of the ATP molecule according to TABLE 4.

In some embodiments, quantifying the binding affinity includes a step that scores hydrophobic interactions between one or more ligand atoms and one or more receptor atoms by awarding a bonus for the presence of hydrophobic enclosure of one or more atoms of said ligand by the receptor molecule domain, said bonus being indicative of enhanced binding affinity between said ligand and said receptor molecule domain.

In some embodiments, the method further comprises calculating an initial binding affinity and then adjusting the initial binding affinity based on the classification of the ring structure as buried, solvent exposed, or solvent exposed on one face.

In some embodiments, the classification of a ring structure as buried, solvent exposed, or solvent exposed on one surface, includes using a parameter substantially correlated with the number of close contacts on both sides of the ring structure or part thereof with the receptor molecule domain.

In some embodiments, the number of close contacts at different distances between receptor atoms and the two ring faces are determined, an initial classification of the ring is made based on the numbers of these contacts, and this initial classification is then followed by calculation of a scoring function, said scoring function comprising identifying a first ring shell and a second ring shell, and calculating the number of water molecules in the first shell and in the second shell, or calculating the number of water molecules in the first and second shell combined.

In some embodiments, the scoring function for classification of the ring structure as buried, solvent exposed, or solvent exposed on one surface, includes using a parameter substantially correlated with the lipophilic-lipophilic pair score between the ring structure or part thereof and the receptor molecule domain.

In some embodiments, the scoring function used to classify a ring structure as buried, solvent exposed, or solvent exposed on one surface, includes calculating the degree of enclosure of each atom of the ring structure by atoms of the receptor.

In some embodiments, the scoring function used to classify a ring structure as buried, solvent exposed, or solvent exposed on one surface, includes using a parameter that is substantially correlated with the degree of enclosure of each atom of the ring structure by atoms of the receptor.

In some embodiments, the scoring function enabling classification of the ring structure as buried, solvent exposed, or solvent exposed on one surface, includes the use of a parameter corresponding to a hydrophobic interaction of the ring structure or part thereof with the receptor molecule domain.

In some embodiments, the information displayed by computer includes a depiction of at least one of the degree to which the ring structure is enclosed by atoms of the receptor molecule domain; water molecules surrounding the ring structure in a first shell or a second shell or both the first and the second shell of the ligand; a value of a lipophilic-lipophilic pair score of the ring structure; and a number of close contacts of a face of the ring structure with the receptor molecule domain.

In some embodiments, solvent exposed ring structures in the ligand, if any, are substantially ignored in quantifying the component of the binding affinity between the ligand and the receptor molecule domains, other than to recognize hydrogen bonds and other parameters that are independent of the classification of ring structure.

In some embodiments, hydrophobic contribution to binding affinity from ring structures classified as solvent exposed, if any, is substantially ignored in quantifying the component of the binding affinity.

In some embodiments, a ring structure is classified as buried, and the method further comprises identifying a quantity representative of a strain energy induced in the ligand-receptor complex by the buried ring structure, in which the quantification of the component of binding affinity is further based in part on strain energy.

In some embodiments, the method further comprises identifying a quantity representative of a strain energy induced in the ligand-receptor complex by the aggregate of the ring structures identified as buried; identifying a quantity representative of a total neutral-neutral hydrogen bond energy; and quantifying the component of binding affinity between the ligand and the receptor molecule domain based at least in part on the quantity representative of the strain energy induced in the receptor by the aggregate of the buried ring structures, and on the quantity representative of the total neutral-neutral hydrogen bond energy.

In some embodiments, quantifying the component of binding affinity further comprises identifying a hydrogen bond capping energy associated with the entire ligand, and the component of binding affinity is quantified based on a greater of the hydrogen bond capping energy and the quantity representative of the strain energy induced in the receptor by the aggregate of the identified structures.

In some embodiments, the method further comprises identifying a binding motif of the receptor molecule domain with respect to the ligand; identifying a reorganization energy of the receptor molecule domain based on the binding motif; and identifying a first ring structure as contributing to the reorganization energy, the quantity representative of strain energy being identified independently of the classification of the first ring structure.

In some embodiments, the component of binding affinity attributable to strain is quantified using at least one of: molecular dynamics, molecule mechanics, conformational searching and minimization.

In some embodiments, the information displayed by computer includes a depiction of solvent exposure, if any, of the ring structure.

In some embodiments, the information displayed by computer includes a depiction of burial, if any, of the ring structure.

In some embodiments, the information displayed by computer includes a depiction of at least one of: the degree to which the ring structure is enclosed by atoms of the receptor molecule domain; water molecules surrounding the ring structure in a first shell or a second shell or both the first and the second shell of the ligand; a value of a lipophilic-lipophilic pair score of the ring structure; and a number of close contacts of a face of the ring structure with the receptor molecule domain.

In some embodiments, the method further comprises performing a test on a physical sample that includes the ligand and the receptor molecule domain, test components being selected based at least in part on the binding affinity between the ligand or part thereof and the receptor molecule, or on the component of such binding affinity.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively, or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which is also referred to or described as a program, software, a software application, an app, a module, a software module, a script, or code, can be written in any form of programming language, including compiled or interpreted languages, or declarative or procedural languages; and can be deployed in any form, including as a stand-alone program or as a module, component, subroutine, or other unit suitable for use in a computing environment. A program can, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit receives instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer can also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

### EMBODIMENTS

The following non-limiting embodiments provide illustrative examples of the invention, but do not limit the scope of the invention.

Embodiment 1. A composition comprising water, a protein, and a synthetic compound, wherein the protein is a ryanodine receptor 1 protein (RyR1) or a mutant thereof.

Embodiment 2. The composition of embodiment 1, further comprising calmodulin.

Embodiment 3. The composition of embodiment 2, wherein the calmodulin is human calmodulin.

Embodiment 4. The composition of any one of embodiments 1-3, further comprising a buffering agent.

Embodiment 5. The composition of embodiment 4, wherein the buffering agent is HEPES.

Embodiment 6. The composition of embodiment 4, wherein the buffering agent is EGTA.

Embodiment 7. The composition of any one of embodiments 1-6, further comprising a phospholipid.

Embodiment 8. The composition of embodiment 7, wherein the phospholipid is DOPS.

Embodiment 9. The composition of any one of embodiments 1-8, further comprising a zwitterionic surfactant.

Embodiment 10. The composition of embodiment 9, wherein the zwitterionic surfactant is CHAPS.

Embodiment 11. The composition of any one of embodiments 1-10, further comprising a disulfide-reducing agent.

Embodiment 12. The composition of embodiment 11, wherein the disulfide-reducing agent is TCEP.

Embodiment 13. The composition of any one of embodiments 1-12, further comprising a protease inhibitor.

Embodiment 14. The composition of embodiment 13, wherein the protease inhibitor is AEBSF.

Embodiment 15. The composition of embodiment 13, wherein the protease inhibitor is benzamidine hydrochloride.

Embodiment 16. The composition of any one of embodiments 1-15, further comprising caffeine.

Embodiment 17. The composition of embodiment 16, wherein caffeine is present at a concentration from about 3 mM to about 7 mM.

Embodiment 18. The composition of embodiment 16, wherein caffeine is present at a concentration of about 5 mM.

Embodiment 19. The composition of any one of embodiments 1-18, further comprising dissolved Ca²⁺.

Embodiment 20. The composition of embodiment 19, wherein dissolved Ca²⁺ is present at a concentration from about 5 μM to about 100 μM.

Embodiment 21. The composition of embodiment 19, wherein dissolved Ca²⁺ is present at a concentration from about 20 μM to about 40 μM.

Embodiment 22. The composition of embodiment 19, wherein dissolved Ca²⁺ is present at a concentration of about 30 μM.

Embodiment 23. The composition of any one of embodiments 1-22, wherein the protein is present at a concentration from about 1 μM to about 100 μM.

Embodiment 24. The composition of embodiment 23, wherein the protein is present at a concentration from about 1 μM to about 45 μM.

Embodiment 25. The composition of embodiment 23, wherein the protein is present at a concentration of about 15 μM.

Embodiment 26. The composition of any one of embodiments 1-25, further comprising sodium adenosine triphosphate (NaATP).

Embodiment 27. The composition of embodiment 26, wherein the NaATP is present at a concentration from about 3 mM to about 15 nM.

Embodiment 28. The composition of embodiment 26, wherein the NaATP is present at a concentration from about 10 mM.

Embodiment 29. The composition of any one of embodiments 1-28, wherein the aqueous solution is substantially free of cellular membrane.

Embodiment 30. A composition comprising a complex suspended in a solid medium, wherein the complex comprises a protein and a synthetic compound, wherein the protein is a ryanodine receptor 1 protein (RyR1) or mutant thereof.

Embodiment 31. The composition of embodiment 30, wherein the composition is prepared by a process comprising vitrifying an aqueous solution applied to an electron microscopy grid, wherein the aqueous solution comprises the protein and the synthetic compound.

Embodiment 32. The composition of embodiment 31, wherein, prior to the vitrifying, the aqueous solution is applied to the electron microscopy grid, and excess aqueous solution is removed from the electron microscopy grid by blotting the excess aqueous solution.

Embodiment 33. The composition of embodiment 31, wherein the vitrifying comprises plunge freezing the aqueous solution applied to the electron microscopy grid into liquid ethane chilled with liquid nitrogen.

Embodiment 34. The composition of any one of embodiments 31-33, wherein the aqueous solution further comprises a buffering agent.

Embodiment 35. The composition of embodiment 34, wherein the buffering agent is HEPES.

Embodiment 36. The composition of embodiment 34, wherein the buffering agent is EGTA.

Embodiment 37. The composition of any one of embodiments 31-36, wherein the aqueous solution further comprises a phospholipid.

Embodiment 38. The composition of embodiment 37, wherein the phospholipid is DOPS.

Embodiment 39. The composition of any one of embodiments 31-38, wherein the aqueous solution further comprises a zwitterionic surfactant.

Embodiment 40. The composition of embodiment 39, wherein the zwitterionic surfactant is CHAPS.

Embodiment 41. The composition of any one of embodiments 31-40, wherein the aqueous solution further comprises a disulfide-reducing agent.

Embodiment 42. The composition of embodiment 41, wherein the disulfide-reducing agent is TCEP.

Embodiment 43. The composition of any one of embodiments 31-42, wherein the aqueous solution further comprises a protease inhibitor.

Embodiment 44. The composition of embodiment 43, wherein the protease inhibitor is AEBSF.

Embodiment 45. The composition of embodiment 43, wherein the protease inhibitor is benzamidine hydrochloride.

Embodiment 46. The composition of any one of embodiments 31-45, wherein the aqueous solution further comprises caffeine.

Embodiment 47. The composition of embodiment 46, wherein the caffeine is present at a concentration from about 3 mM to about 7 mM.

Embodiment 48. The composition of embodiment 46, wherein the caffeine is present at a concentration of about 5 mM.

Embodiment 49. The composition of any one of embodiments 31-48, wherein the aqueous solution further comprises dissolved Ca²⁺.

Embodiment 50. The composition of embodiment 49, wherein the dissolved Ca²⁺ is present at a concentration from about 5 μM to about 100 μM.

Embodiment 51. The composition of embodiment 49, wherein the dissolved Ca²⁺ is present at a concentration from about 20 μM to about 40 μM.

Embodiment 52. The composition of embodiment 49, wherein the dissolved Ca²⁺ is present at a concentration of about 30 μM.

Embodiment 53. The composition of any one of embodiments 31-52, wherein the protein is present at a concentration from about 1 μM to about 100 μM.

Embodiment 54. The composition of any one of embodiments 31-52, wherein the protein is present at a concentration from about 1 μM to about 45 μM.

Embodiment 55. The composition of any one of embodiments 31-52, wherein the protein is present at a concentration of about 15 μM.

Embodiment 56. The composition of any one of embodiments 31-55, wherein the aqueous solution further comprises sodium adenosine triphosphate (NaATP).

Embodiment 57. The composition of embodiment 56, wherein the NaATP is present at a concentration from about 3 mM to about 15 nM.

Embodiment 58. The composition of embodiment 56, wherein the NaATP is present at a concentration of about 10 mM.

Embodiment 59. The composition of any one of embodiments 31-58, wherein the aqueous solution further comprises calmodulin.

Embodiment 60. The composition of embodiment 59, wherein the calmodulin is human calmodulin.

Embodiment 61. The composition of any one of embodiments 30-55, wherein the complex further comprises a nucleoside-containing molecule.

Embodiment 62. The composition of embodiment 61, wherein the nucleoside-containing molecule and the synthetic compound bind a RYR domain of the protein.

Embodiment 63. The composition of embodiment 62, wherein the RYR domain is a RY1&2 domain.

Embodiment 64. The composition of embodiment 63, wherein the RY1&2 domain has a three-dimensional structure according to TABLE 2.

Embodiment 65. The composition of any one of embodiments 61-64, wherein the nucleoside-containing molecule is a purine nucleoside-containing molecule.

Embodiment 66. The composition of any one of embodiments 61-65, wherein the nucleoside-containing molecule is a nucleotide or nucleoside polyphosphate.

Embodiment 67. The composition of any one of embodiments 61-66, wherein the nucleoside-containing molecule is an adenosine triphosphate (ATP) molecule.

Embodiment 68. The composition of embodiment 67, wherein the ATP molecule forms a pi-stacking interaction with W996 of the protein.

Embodiment 69. The composition of embodiment 67 or embodiment 68, wherein the ATP molecule has a three-dimensional conformation according to TABLE 4.

Embodiment 70. The composition of any one of embodiments 67-69, wherein the ATP molecule cooperatively binds the protein with the synthetic compound.

Embodiment 71. The composition of any one of embodiments 67-69, wherein the ATP molecule forms a pi-stacking interaction with the synthetic compound.

Embodiment 72. The composition of any one of embodiments 61-66, wherein the nucleoside-containing molecule is an adenosine diphosphate (ADP) molecule.

Embodiment 73. The composition of embodiment 72, wherein the complex further comprises a second ADP molecule, wherein both ADP molecules bind a common RYR domain of the protein.

Embodiment 74. The composition of any one of embodiments 61-71, wherein the complex further comprises a second nucleoside-containing molecule.

Embodiment 75. The composition of embodiment 74, wherein the second nucleoside-containing molecule binds a C-terminal domain of the RyR1 protein.

Embodiment 76. The composition of embodiment 74 or embodiment 75, wherein the second nucleoside-containing molecule is a nucleotide or nucleoside polyphosphate.

Embodiment 77. The composition of any one of embodiments 74-76, wherein the second nucleoside-containing molecule is a second ATP molecule.

Embodiment 78. The composition of any one of embodiments 30-55 and 61-73, wherein the complex further comprises calmodulin.

Embodiment 79. The composition of embodiment 78, wherein the calmodulin is human calmodulin.

Embodiment 80. The composition of any one of embodiments 30-79, wherein the complex further comprises calstabin.

Embodiment 81. The composition of embodiment 80, wherein the calstabin is rabbit calstabin.

Embodiment 82. The composition of embodiment 80, wherein the calstabin is human calstabin.

Embodiment 83. The composition of any one of embodiments 30-83, wherein the complex further comprises a caffeine molecule.

Embodiment 84. The composition of any one of embodiments 30-83, wherein the complex further comprises a Ca²⁺ ion.

Embodiment 85. The composition of any one of embodiments 30-84, wherein the RyR1 protein is in the closed state.

Embodiment 86. The composition of any one of embodiments 30-85, wherein the composition is substantially free of cellular membrane.

Embodiment 87. The composition of any one of embodiments 30-86, wherein the solid medium comprises vitreous ice.

Embodiment 88. The composition of embodiment 87, wherein the solid medium is substantially free of crystalline ice.

Embodiment 89. The composition of any one of embodiments 30-88, wherein the composition further comprises additional complexes, wherein each of the additional complexes independently comprises the protein and the synthetic compound.

Embodiment 90. The composition of any one of embodiments 1-61, wherein the synthetic compound binds a RYR domain of the protein.

Embodiment 91. The composition of embodiment 90, wherein the RYR domain is a RY1&2 domain.

Embodiment 92. The composition of any one of embodiments 1-91, wherein the synthetic compound forms a pi-stacking interaction with W882 of the protein.

Embodiment 93. The composition of any one of embodiments 1-92, wherein the synthetic compound forms a salt bridge with H879 of the protein.

Embodiment 94. The composition of any one of embodiments 1-93, wherein the protein is wild type RyR1.

Embodiment 95. The composition of any one of embodiments 1-93, wherein the protein is mutant RyR1.

Embodiment 96. The composition of embodiment 95, wherein the mutant RyR1 is W882A RyR1, W882A RyR1, or C906A RyR1.

Embodiment 97. The composition of any one of embodiments 1-96, wherein the protein is human RyR1.

Embodiment 98. The composition of any one of embodiments 1-96, wherein the protein is rabbit RyR1.

Embodiment 99. The composition of any one of embodiments 1-94, wherein the protein is a tetramer of rabbit RyR1 monomers, wherein each rabbit RyR1 monomer is a peptide according to SEQ ID NO: 3.

Embodiment 100. The composition of any one of embodiments 1-99, wherein the synthetic compound comprises a benzazepane, benzothiazepane, or benzodiazepane moiety.

Embodiment 101. The composition of any one of embodiments 1-100, wherein the synthetic compound comprises a benzothiazepane moiety.

Embodiment 102. The composition of embodiment 92, wherein the synthetic compound comprises a benzothiazepane moiety, wherein the benzothiazepane moiety forms the pi-stacking interaction with W882 of the protein.

Embodiment 103. The composition of any one of embodiments 1-102, wherein the synthetic compound is a compound of Formula (I):

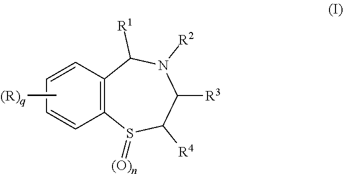

wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹.
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4,
or a pharmaceutically-acceptable salt thereof.

Embodiment 104. The composition of any one of embodiments 1-103, wherein the synthetic compound is a compound of Formula (I-k):

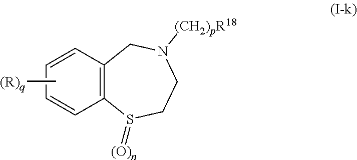

wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹⁸ is alkyl, aryl, cycloalkyl, or heterocyclyl, each of which is independently substituted or unsubstituted; or —NR¹⁵R¹⁶, —C(═O)NR¹⁵R¹⁶, —(C═O)OR¹⁵, or —OR¹⁵;
q is 0, 1, 2, 3, or 4;
p is 1, 2, 3, 4, 5, 6, 7, 8 9, or 10; and
n is 0, 1, or 2,
or a pharmaceutically-acceptable salt thereof.

Embodiment 105. The composition of embodiment 103 or embodiment 104, wherein each R is independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2.

Embodiment 106. The composition of any one of embodiments 103-105, wherein R¹⁸ is —NR¹⁵R¹⁶, —(C═O)OR¹⁵, —OR¹⁵, alkyl that is substituted or unsubstituted, or aryl that is substituted or unsubstituted.

Embodiment 107. The composition of any one of embodiments 1-103, wherein the synthetic compound is a compound of Formula (I-o):

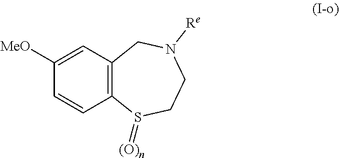

wherein:
R^{e} is —(C₁-C₆ alkyl)-phenyl, —(C₁-C₆ alkyl)-C(O)R^{b}, or substituted or unsubstituted —C₁-C₆ alkyl; and
R^{b} is —OH or —O—(C₁-C₆ alkyl),
wherein the phenyl or the substituted alkyl is substituted with one or more of halogen, hydroxyl, —C₁-C₆ alkyl, —O—(C₁-C₆ alkyl), —NH₂, —NH(C₁-C₆ alkyl), —N(C₁-C₆ alkyl)₂, cyano, or dioxolane,
or a pharmaceutically acceptable salt thereof.

Embodiment 108. The composition of any one of embodiments 1-107, wherein the synthetic compound is:

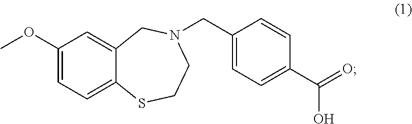 [SMILES: COc1ccc2c(c1)CN(Cc1ccc(C(=O)O)cc1)CCS2]

or an ionized form thereof.

Embodiment 109. The composition of any one of embodiments 1-107, wherein the synthetic compound is:

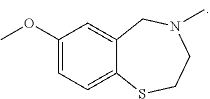

Embodiment 110. The composition of embodiment 108, wherein the synthetic compound has a three-dimensional conformation according to TABLE 3.

Embodiment 111. A vessel containing the composition of any one of embodiments 1-29.

Embodiment 112. The vessel of embodiment 111, wherein the vessel is a vial, ampule, test tube, or microwell plate.

Embodiment 113. A method of determining a binding site of a synthetic compound in a protein, the method comprising subjecting a composition of any one of embodiments 30-89 to single-particle cryogenic electron microscopy analysis.

Embodiment 114. A method for predicting a docked position of a target ligand in a binding site of a biomolecule, the method comprising:
receiving a template ligand-biomolecule structure, the template ligand-biomolecule structure comprising a template ligand docked in the binding site of the biomolecule; comparing a pharmacophore model of the template ligand to a pharmacophore model of the target ligand;
overlapping the pharmacophore model of the target ligand with the pharmacophore model of the template ligand while the template ligand is in the binding site of the biomolecule; and
predicting the docked position of the target ligand in the binding site of the biomolecule based on a position of the pharmacophore model of the target ligand when overlapped with the pharmacophore model of the template ligand,
wherein the biomolecule is a RY1&2 domain of RyR1, wherein the template ligand-biomolecule structure is obtained by a process comprising subjecting a complex of the biomolecule and the template ligand to single-particle cryogenic electron microscopy analysis.

Embodiment 115. The method of embodiment 114, wherein the RY1&2 domain comprises a structure according to TABLE 2.

Embodiment 116. The method of embodiment 114, wherein the template ligand has a three-dimensional conformation according to TABLE 3.

Embodiment 117. The method of embodiment 114, wherein the RY1&2 domain contains a nucleoside-containing molecule.

Embodiment 118. The method of embodiment 117, wherein the nucleoside-containing molecule is an ATP molecule.

Embodiment 119. The method of embodiment 118, wherein the ATP molecule has a three-dimensional conformation according to TABLE 4.

Embodiment 120. The method of embodiment 117 or embodiment 118, wherein the target ligand cooperatively binds the RY1&2 domain with the ATP molecule.

Embodiment 121. The method of any one of embodiments 117-120, wherein the target ligand forms a pi-stacking interaction with W882 of the protein.

Embodiment 122. The method of any one of embodiments 117-121, wherein the target ligand forms a pi-stacking interaction with W882 of the protein.

Embodiment 123. The method of embodiment 114, wherein the target ligand and the template ligand are each independently a compound of Formula (I):

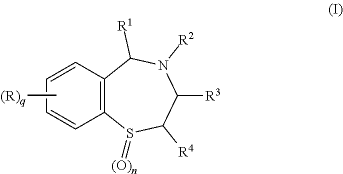

wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —N₀₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘR¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹.
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4.

Embodiment 124. The method of any one of embodiments 114-124, wherein the template ligand is:

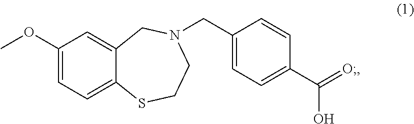 [SMILES: COc1ccc2c(c1)CN(Cc1ccc(C(=O)O)cc1)CCS2]

or a pharmaceutically-acceptable salt or an ionized form thereof.

Embodiment 125. The method of any one of embodiments 114-124, wherein the template ligand is

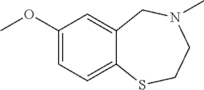

or a pharmaceutically-acceptable salt or ionized form thereof.

Embodiment 126. The method of embodiment 114, further comprising selecting the target ligand from a plurality of ligand candidates, each of the ligand candidates being different from the template ligand, and wherein selecting the target ligand comprises comparing the pharmacophore model of the template ligand to a pharmacophore model of each respective one of the plurality of ligand candidates.

Embodiment 127. The method of embodiment 114, further comprising receiving a plurality of template ligand-biomolecule structures, each template ligand-biomolecule structure having a different template ligand docked in the binding site of the biomolecule, and generating the pharmacophore model of the template ligand by combining information from each of the template ligands from the plurality of template ligand-biomolecule structures.

Embodiment 128. The method of embodiment 114, wherein the target ligand has more than one structural conformation in an unbound state, and the docked position of the target ligand in the binding site of the biomolecule is predicted by enumerating a set of potential target ligand conformations and overlapping a respective pharmacophore model of the target ligand for each of the potential target ligand conformations with the pharmacophore model of the template ligand while the template ligand is in the binding site of the biomolecule.

Embodiment 129. The method of embodiment 128, wherein predicting the docked position of the target ligand in the binding site of the biomolecule comprises ignoring at least one clash between the target ligand conformation's atomic coordinates and the biomolecule's atomic coordinates.

Embodiment 130. The method of embodiment 129, further comprising, for each target ligand conformation, modifying atomic coordinates of the biomolecule to reduce clashes between the docked target ligand conformation's atomic coordinates and the biomolecule's atomic coordinates, thereby creating an altered ligand-biomolecule structure comprising the docked target ligand and an altered biomolecule.

Embodiment 131. The method of embodiment 130, further comprising, predicting a re-docked position of each target ligand conformation by predicting each target ligand conformation's position in the binding site of the altered biomolecule; and

for each target ligand conformation, modifying atomic coordinates of the altered biomolecule to reduce clashes between the atomic coordinates of the target ligand conformation's re-docked position and the atomic coordinates of the altered biomolecule, thereby creating a re-altered ligand-biomolecule structure comprising a re-docked target ligand and a re-altered biomolecule.

Embodiment 132. The method of embodiment 131, further comprising ranking each altered and re-altered ligand-biomolecule structure using a scoring function.

Embodiment 133. The method of embodiment 132, further comprising identifying a subset of high-ranking target ligands corresponding to target ligands having a threshold value for an empirical activity.

Embodiment 134. A method of identifying a plurality of potential lead compounds, the method comprising the steps of:
(a) analyzing, using a computer system, an initial lead compound known to bind to a biomolecular target, the analyzing comprising partitioning, by providing a database of known reactions, the initial lead compound into atoms defining partitioned lead compound comprising a lead compound core and atoms defining a lead compound non-core, wherein the initial lead compound is partitioned using a computational retrosynthetic analysis of the initial lead compound;
(b) identifying, using the computer system, a plurality of alternative cores to replace the lead compound core in the initial lead compound, thereby generating a plurality of potential lead compounds each having a respective one of the plurality of alternative cores;
(c) calculating, using the computer system, a difference in binding free energy between the partitioned lead compound and each potential lead compound; (d) predicting, using the computer system, whether each potential lead compound will bind to the biomolecular target and identifying a predicted active set of potential lead compounds based on the prediction;
(e) obtaining a synthesized set of at least some of the potential leads of the predicted active set to establish a first of potential lead compounds; and
(f) determining, empirically, an activity of each of the first set of synthesized potential lead compounds,
wherein the biomolecular target is a RY1&2 domain of RyR1, and the structure of the biomolecular target used in the predicting of (d) is obtained by a process comprising subjecting a complex of the biomolecular target and the initial lead compound to single-particle cryogenic electron microscopy analysis.

Embodiment 135. The method of embodiment 134, wherein the structure of the of the biomolecular target obtained by single-particle cryogenic electron microscopy analysis has a resolution from about 2 Å to about 3.5 Å, from about 2 Å to about 3.4 Å, from about 2 Å to about 3.3 Å, from about 2 Å to about 3.2 Å, from about 2 Å to about 3.1 Å, from about 2 Å to about 3 Å, from about 2 Å to about 2.9 Å, from about 2 Å to about 2.8 Å, from about 2 Å to about 2.7 Å, from about 2 Å to about 2.6 Å, from about 2 Å to about 2.5 Å, from about 2.1 Å to about 2.5 Å, from about 2.2 Å to about 2.5 Å, from about 2.3 Å to about 2.5 Å, or from about 2.4 Å to about 2.5 Å.

Embodiment 136. The method of embodiment 134, wherein the initial lead compound is a compound of Formula (I):

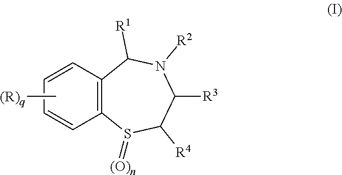

wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹.
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4.

Embodiment 137. The method of embodiment 134, wherein the initial lead compound is

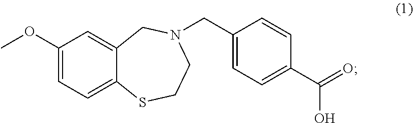 [SMILES: COc1ccc2c(c1)CN(Cc1ccc(C(=O)O)cc1)CCS2]

or an ionized form thereof.

Embodiment 138. The method of embodiment 134, wherein the initial lead compound is

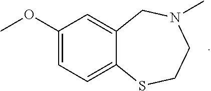

Embodiment 139. The method of embodiment 134, wherein the RY1&2 domain comprises a structure according to TABLE 2.

Embodiment 140. The method of embodiment 134, wherein the RY1&2 domain contains an ATP molecule.

Embodiment 141. The method of embodiment 140, wherein the ATP molecule has a three-dimensional conformation according to TABLE 4.

Embodiment 142. The method of embodiment 134, further comprising obtaining a synthesized set of at least some of the potential lead compounds predicted to not bind with the biomolecular target to establish a second set of potential lead compounds and empirically determining an activity of each of the second set of synthesized potential lead compounds.

Embodiment 143. The method of embodiment 134, further comprising comparing the empirically determined activity of each of the first set of synthesized potential lead compounds with a threshold activity level.

Embodiment 144. The method of embodiment 135, further comprising comparing the empirically determined activity of each of the second set of synthesized potential lead compounds with a pre-determined activity level.

Embodiment 145. The method of embodiment 134, wherein the plurality of alternative cores are chosen from a database of synthetically feasible cores.

Embodiment 146. The method of embodiment 134, wherein the difference in binding free energy is calculated using a free energy perturbation technique.

Embodiment 147. The method of embodiment 142, wherein the generation of at least one potential lead compound comprises creating an additional covalent bond or annihilating an existing covalent bond, or both creating an additional first covalent bond and annihilating an existing second covalent bond different from the first covalent bond.

Embodiment 148. The method of embodiment 143, wherein the free energy perturbation technique uses a soft bond potential to calculate a bonded stretch interaction energy of existing covalent bonds for annihilation and additional covalent bonds for creation.

Embodiment 149. A method for pharmaceutical drug discovery, comprising:
identifying an initial lead compound for binding to a biomolecular target; using the method of embodiment 134 to identify a predicted active set of potential lead compounds for binding to the biomolecular target based on the initial lead compound;
selecting one or more of the predicted active set of potential lead compounds for synthesis; and
assaying the one or more synthesized selected compounds to assess each synthesized selected compounds suitability for in vivo use as a pharmaceutical compound,
wherein the biomolecular target is a RY1&2 domain of RyR1, and the structure of the biomolecular target used in the predicting of (d) is obtained by a process comprising subjecting a complex of the biomolecular target and the initial lead compound to single-particle cryogenic electron microscopy analysis.

Embodiment 150. The method of embodiment 149, wherein the initial lead compound is compound of Formula (I):

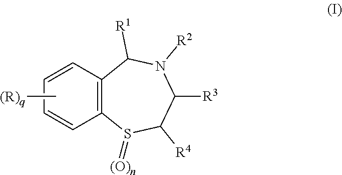

wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, —(CH₂)ₘR¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹.
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4.

Embodiment 151. The method of embodiment 149, wherein the initial lead compound is

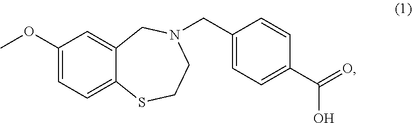 [SMILES: COc1ccc2c(c1)CN(Cc1ccc(C(=O)O)cc1)CCS2]

or an ionized form thereof.

Embodiment 152. The method of embodiment 149, wherein the initial lead compound is

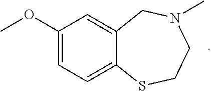

Embodiment 153. The method of embodiment 149, wherein the RY1&2 domain comprises a structure according to TABLE 2.

Embodiment 154. The method of embodiment 149, wherein the RY1&2 domain contains an ATP molecule.

Embodiment 155. The method of embodiment 153, wherein the ATP molecule has a three-dimensional conformation according to TABLE 4.

Embodiment 156. A computer-implemented method of quantifying binding affinity between a ligand and a receptor molecule, the method comprising:
receiving by one or more computers, data representing a ligand molecule,
receiving by one or more computers, data representing a receptor molecule domain, using the data representing the ligand molecule and the data representing the receptor molecule domain in computer analysis to identify ring structure within the ligand, the ring structure being an entire ring or a fused ring;
using the data representative of the identified ligand ring structure to designate a first ring face and a second ring face opposite to the first ring face, and classifying the ring structure by:
a) determining proximity of receptor atoms to atoms on the first face of the ligand ring; and
b) determining proximity of receptor atoms to atoms on the second face of the ligand ring;
c) determining solvation of the first face of the ligand ring and solvation of the second face of the ligand ring;
classifying the identified ligand ring structure as buried, solvent exposed or having a single face exposed to solvent based on receptor atom proximity to and solvation of the first ring face and receptor atom proximity to and solvation of the second ring face; quantifying the binding affinity between the ligand and the receptor molecule domain based at least in part on the classification of the ring structure; and displaying, via computer, information related to the classification of the ring structure,
wherein the receptor molecule domain is a RY1&2 domain of RyR1, wherein the data representing a ligand molecule and the data representing a receptor molecule domain are obtained by a process comprising subjecting a complex comprising the ligand molecule and the receptor molecule domain to single-particle cryogenic electron microscopy analysis.

Embodiment 157. The method of embodiment 156, wherein the initial lead compound is compound of Formula (I):

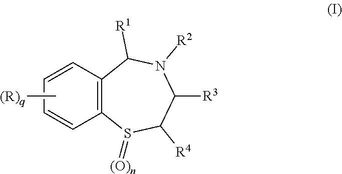

wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4.

Embodiment 158. The method of embodiment 156, wherein the ligand molecule is:

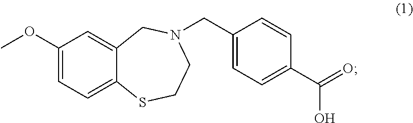 [SMILES: COc1ccc2c(c1)CN(Cc1ccc(C(=O)O)cc1)CCS2]

or an ionized form thereof.

Embodiment 159. The method of embodiment 156, wherein the ligand molecule is

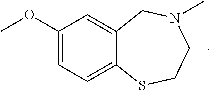

Embodiment 160. The method of embodiment 156, wherein the complex further comprises a RyR1 protein, wherein the RYT&2 domain is a domain of the RyR1 protein.

Embodiment 161. The method of embodiment 156, wherein the data representing the receptor molecule domain represents a three-dimensional structure of the receptor molecule according to TABLE 2.

Embodiment 162. The method of embodiment 156, wherein the data representing a ligand molecule represents a three-dimensional structure of the ligand molecule according to TABLE 3.

Embodiment 163. The method of embodiment 156, wherein the receptor molecule domain contains an ATP molecule.

Embodiment 164. The method of embodiment 163, wherein the data representing the receptor molecule domain further comprises data representing a three-dimensional structure of the ATP molecule according to TABLE 4.

Embodiment 165. The method of embodiment 156, wherein quantifying the binding affinity includes a step that scores hydrophobic interactions between one or more ligand atoms and one or more receptor atoms by awarding a bonus for the presence of hydrophobic enclosure of one or more atoms of said ligand by the receptor molecule domain, said bonus being indicative of enhanced binding affinity between said ligand and said receptor molecule domain.

Embodiment 166. The method of embodiment 156, further comprising calculating an initial binding affinity and then adjusting the initial binding affinity based on the classification of the ring structure as buried, solvent exposed or solvent exposed on one face.

Embodiment 167. The method of embodiment 156, wherein the classification of a ring structure as buried, solvent exposed, or solvent exposed on one surface, includes using a parameter substantially correlated with the number of close contacts on both sides of the ring structure or part thereof with the receptor molecule domain.

Embodiment 168. The method of embodiment 156, wherein the number of close contacts at different distances between receptor atoms and the two ring faces are determined, an initial classification of the ring is made based on the numbers of these contacts, and this initial classification is then followed by calculation of a scoring function, said scoring function comprising identifying a first ring shell and a second ring shell, and calculating the number of water molecules in the first shell and in the second shell, or calculating the number of water molecules in the first and second shell combined.

Embodiment 169. The method of embodiment 168, wherein the scoring function allowing classification of the ring structure as buried, solvent exposed, or solvent exposed on one surface, includes using a parameter substantially correlated with the lipophilic-lipophilic pair score between the ring structure or part thereof and the receptor molecule domain.

Embodiment 170. The method of embodiment 168, wherein the scoring function used to classify a ring structure as buried, solvent exposed, or solvent exposed on one surface, includes calculating the degree of enclosure of each atom of the ring structure by atoms of the receptor.

Embodiment 171. The method of embodiment 168, wherein the scoring function used to classify a ring structure as buried, solvent exposed, or solvent exposed on one surface, includes using a parameter that is substantially correlated with the degree of enclosure of each atom of the ring structure by atoms of the receptor.

Embodiment 172. The method of embodiment 156 or embodiment 168, wherein the scoring function allowing classification of the ring structure as buried, solvent exposed, or solvent exposed on one surface, includes the use of a parameter corresponding to a hydrophobic interaction of the ring structure or part thereof with the receptor molecule domain.

Embodiment 173. The method of embodiment 172, wherein the information displayed by computer includes a depiction of at least one of:
the degree to which the ring structure is enclosed by atoms of the receptor molecule domain;
water molecules surrounding the ring structure in a first shell or a second shell or both the first and the second shell of the ligand;
a value of a lipophilic-lipophilic pair score of the ring structure; and
a number of close contacts of a face of the ring structure with the receptor molecule domain.

Embodiment 174. The method of embodiment 156, wherein solvent exposed ring structures in the ligand, if any, are substantially ignored in quantifying the component of the binding affinity between the ligand and the receptor molecule domains, other than to recognize hydrogen bonds and other parameters that are independent of the classification of ring structure.

Embodiment 175. The method of embodiment 156, wherein hydrophobic contribution to binding affinity from ring structures classified as solvent exposed, if any, is substantially ignored in quantifying the component of the binding affinity.

Embodiment 176. The method of embodiment 156, wherein a ring structure is classified as buried, and the method further comprises:
identifying a quantity representative of a strain energy induced in the ligand-receptor complex by the buried ring structure, in which the quantification of the component of binding affinity is further based in part on strain energy.

Embodiment 177. The method of embodiment 176, further comprising
identifying a quantity representative of a strain energy induced in the ligand-receptor complex by the aggregate of the ring structures identified as buried;
identifying a quantity representative of a total neutral-neutral hydrogen bond energy; and
quantifying the component of binding affinity between the ligand and the receptor molecule domain based at least in part on the quantity representative of the strain energy induced in the receptor by the aggregate of the buried ring structures, and on the quantity representative of the total neutral-neutral hydrogen bond energy.

Embodiment 178. The method of embodiment 177, wherein
quantifying the component of binding affinity further comprises identifying a hydrogen bond capping energy associated with the entire ligand, and
the component of binding affinity is quantified based on a greater of the hydrogen bond capping energy and the quantity representative of the strain energy induced in the receptor by the aggregate of the identified structures.

Embodiment 179. The method of embodiment 177, further comprising:
identifying a binding motif of the receptor molecule domain with respect to the ligand;
identifying a reorganization energy of the receptor molecule domain based on the binding motif; and
identifying a first ring structure as contributing to the reorganization energy,
the quantity representative of strain energy being identified independently of the classification of the first ring structure.

Embodiment 180. The method of embodiment 176, wherein the component of binding affinity attributable to strain is quantified using at least one of molecular dynamics, molecule mechanics, conformational searching and minimization.

Embodiment 181. The method of embodiment 156, wherein the information displayed by computer includes a depiction of solvent exposure, if any, of the ring structure.

Embodiment 182. The method of embodiment 156, wherein the information displayed by computer includes a depiction of burial, if any, of the ring structure.

Embodiment 183. The method of embodiment 156, wherein the information displayed by computer includes a depiction of at least one of:
the degree to which the ring structure is enclosed by atoms of the receptor molecule domain;
water molecules surrounding the ring structure in a first shell or a second shell or both the first and the second shell of the ligand;
a value of a lipophilic-lipophilic pair score of the ring structure; and
a number of close contacts of a face of the ring structure with the receptor molecule domain.

Embodiment 184. The method of embodiment 156, further comprising,
performing a test on a physical sample that includes the ligand and the receptor molecule domain, test components being selected based at least in part on the binding affinity between the ligand or part thereof and the receptor molecule, or on the component of such binding affinity.

Embodiment 185. A method comprising:
(a) determining open probability (Pₒ) of a first RyR1 protein, wherein the first RyR1 protein is treated with both an agent capable of phosphorylating, nitrosylating or oxidizing the first RyR1 protein and a test compound; and
(b) determining open probability (Pₒ) of a second RyR1 protein, wherein the second RyR1 protein is treated with the agent and not treated with the test compound.

Embodiment 186. The method of embodiment 185, further comprising (c) determining open probability (Pₒ) of a third RyR1 protein, wherein the third RyR1 protein is neither treated with the agent nor treated with the test compound.

Embodiment 187. The method of embodiment 185 or embodiment 186, wherein determining the open probability (Pₒ) of the first RyR1 protein and the second RyR1 protein comprises recording a single channel Ca²⁺ current.

Embodiment 188. The method of any one of embodiments 185-187, further comprising determining a difference between the Pₒ of the first RyR1 protein and Pₒ of the second RyR1 protein.

Embodiment 189. The method of any one of embodiments 185-188, further comprising determining the difference between the Pₒ of the first RyR1 protein and Pₒ of the third RyR1 protein.

Embodiment 190. The method of embodiment 188, further comprising identifying the test compound as a target for further analysis based on the difference between the Pₒ of the first RyR1 protein and Pₒ of the second RyR1 protein.

Embodiment 191. The method of embodiment 190, further comprising performing an analogous assay where another compound is used in place of the test compound, wherein the analogous assay provides a difference between:
(a) an open probability (Pₒ) of a fourth RyR1 protein, wherein the fourth RyR1 protein is treated with both an agent capable of phosphorylating, nitrosylating or oxidizing the first RyR1 protein and the other compound; and
(b) an open probability (Pₒ) of a fifth RyR1 protein, wherein the fifth RyR1 protein is treated with the agent and not treated with the other compound, wherein the test compound is prioritized over the other compound for the further analysis based on a comparison of:
(i) the difference between the Pₒ of the first RyR1 protein and Pₒ of the second RyR1 protein; with
(ii) a difference between the Pₒ of the fourth RyR1 protein and Pₒ of the fifth RyR1 protein.

Embodiment 192. The method of any one of embodiments 188-191, wherein the difference is subtractive.

Embodiment 193. The method of any one of embodiments 185-191, wherein the agent is an oxidant. In some embodiments, the oxidant is H₂O₂.

Embodiment 194. A method comprising:
(a) contacting a first RyR1 protein with an agent capable of phosphorylating, nitrosylating or oxidizing the first RyR1 protein and a test compound;
(b) contacting a second RyR1 protein with the agent and not with the test compound;
(c) subsequent to the contacting the first RyR1 protein with the agent and the test compound, measuring an open probability (Pₒ) of the first RyR1 protein; and
(d) subsequent to the contacting the first RyR1 protein with the agent and the test compound, measuring an open probability (Pₒ) of the second RyR1 protein.

Embodiment 195. The method of embodiment 194, further comprising (e) determining open probability (Pₒ) of a third RyR1 protein without contacting the third RyR1 protein with the agent and without contacting the third RyR1 protein with the test compound.

Embodiment 196. The method of embodiment 194 or embodiment 195, wherein each of the determining the open probability (Pₒ) of the first RyR1 protein and the determining the open probability (Pₒ) of second RyR1 protein comprises recording a single channel Ca²⁺ current.

Embodiment 197. The method of any one of embodiments 194-196, further comprising determining a difference between the Pₒ of the first RyR1 protein and the Pₒ of the second RyR1 protein.

Embodiment 198. The method of any one of embodiments 194-197, further comprising determining a difference between the Pₒ of the first RyR1 protein and the Pₒ of the third RyR1 protein.

Embodiment 199. The method of embodiment 198, further comprising identifying the test compound as a target for further analysis based on the difference between the Pₒ of the first RyR1 protein and the Pₒ of the second RyR1 protein.

Embodiment 200. The method of embodiment 199, further comprising performing an analogous assay where another compound is used in place of the test compound, wherein the analogous assay provides a difference between:
(a) an open probability (Pₒ) of a fourth RyR1 protein, wherein the fourth RyR1 protein is treated with both an agent capable of phosphorylating, nitrosylating or oxidizing the first RyR1 protein and the other compound; and
(b) an open probability (Pₒ) of a fifth RyR1 protein, wherein the fifth RyR1 protein is treated with the agent and not treated with the other compound,
wherein the test compound is prioritized over the other compound for the further analysis based on a comparison of:
(i) the difference between the Pₒ of the first RyR1 protein and Pₒ of the second RyR1 protein; with
(ii) a difference between the Pₒ of the fourth RyR1 protein and Pₒ of the fifth RyR1 protein.

Embodiment 201. The method of any one of embodiments 197-200, wherein each difference is a subtractive difference.

Embodiment 202. The method of any one of embodiments 194-200, further comprising: subsequent to the contacting the first RyR1 protein with the agent and the test compound, fusing a first microsome containing the first RyR1 protein to a first planar lipid bilayer, and subsequent to the contacting the second RyR1 protein with the agent, fusing a second microsome containing the second RyR1 protein to a second planar lipid bilayer.

Embodiment 203. The method of any one of embodiments 194-202, wherein the agent is an oxidant. In some embodiments, the oxidant is a solution containing H₂O₂.

Embodiment 204. The method of any one of embodiments 194-203, wherein the oxidant is a solution containing about 0.5 to about 10 mM H₂O₂.

Embodiment 205. The method of any one of embodiments 185-204, wherein each RyR1 protein is a wild type RyR1 protein.

Embodiment 206. The method of any one of embodiments 185-204, wherein each RyR1 protein is a C906A mutant.

Embodiment 207. The method of any one of embodiments 185-204, wherein each RyR1 protein is a W882A mutant.

Embodiment 208. A method of identifying a compound having RyR1 modulatory activity, the method comprising:
(a) determining an open probability (Pₒ) of a RyR1 protein;
(b) contacting the RyR1 protein with a test compound;
(c) determining an open probability (Pₒ) of the RyR1 protein in the presence of the test compound; and
(d) determining a difference between the Pₒ of the RyR1 protein in the presence and absence of the test compound;
wherein a reduction in the Pₒ of the RyR1 protein in the presence of the test compound is indicative of the compound having RyR1 modulatory activity.

Embodiment 209. The method of embodiment 208, wherein the RyR1 protein is a leaky RyR1.

Embodiment 210. The method of embodiment 208 or embodiment 209, wherein the RyR1 protein is a mutated RyR1 protein.

Embodiment 211. The method of any one of embodiments 208-210, wherein the RyR1 protein is a post-translationally modified RyR1 protein.

Embodiment 212. The method of any one of embodiments 208-211, wherein the RyR1 protein is a mutated and post-translationally modified RyR1 protein.

Embodiment 213. The method of any one of embodiments 208-212, wherein the test compound preferentially binds to a mutated RyR1 relative to wild-type RyR1.

Embodiment 214. The method of any one of embodiments 208-213, wherein the test compound preferentially binds to post-translationally modified RyR1 relative to wild-type RyR1.

Embodiment 215. The method of any one of embodiments 208-215, wherein the test compound preferentially binds to a mutant and post-translationally modified RyR1 relative to a wild-type RyR1.

Embodiment 216. The method of any one of embodiments 208-215, wherein determining the open probability (Pₒ) of the RyR1 protein comprises recording a single channel Ca²⁺ current.

Embodiment 217. A method for identifying a compound having RyR1 modulatory activity, comprising:
(a) contacting a RyR1 protein with a ligand having known RyR1 modulatory activity to create a mixture, wherein the RyR1 protein is a leaky RyR1, the leaky RyR1 comprising mutant RyR1 protein, post-translationally modified RyR1 protein, or a combination thereof;
(b) contacting the mixture of step (a) with a test compound; and
(c) determining the ability of the test compound to displace the ligand from the RyR1 protein.

Embodiment 218. The method of embodiment 217, wherein the ligand is radiolabeled.

Embodiment 219. The method of embodiment 217 or embodiment 218, wherein determining the ability of the test compound to displace the ligand from the RyR1 protein comprises determining a radioactive signal in the RyR1 protein.

Embodiment 220. The method of any one of embodiments 217-219, wherein the RyR1 protein is a mutated RyR1 protein.

Embodiment 221. The method of any one of embodiments 217-220, wherein the RyR1 protein is a post-translationally modified RyR1 protein.

Embodiment 222. The method of any one of embodiments 217-221, wherein the RyR1 protein is a mutated and post-translationally modified RyR1 protein.

Embodiment 223. The method of any one of embodiments 217-222, wherein the test compound preferentially binds to a mutated RyR1 relative to wild-type RyR1.

Embodiment 224. The method of any one of embodiments 217-223, wherein the test compound preferentially binds to post-translationally modified RyR1 relative to wild-type RyR1.

Embodiment 225. The method of any one of embodiments 217-224, wherein the test compound preferentially binds to a mutant and post-translationally modified RyR1 relative to a wild-type RyR1.

Embodiment 226. A method for identifying a compound that preferentially binds to leaky RyR1, comprising:
(a) determining a binding affinity of a test compound to a first RyR1 protein, wherein the first RyR1 protein is a wild-type RyR1 protein;
(b) determining a binding affinity of a test compound to a second RyR1 protein, wherein second RyR1 protein is a leaky RyR1, the leaky RyR comprising mutant RyR1 protein, post-translationally modified RyR1 protein, or a combination thereof; and
(c) selecting a compound having a higher binding affinity to the second RyR1 protein relative to the first RyR1 protein.

Embodiment 227. The method of embodiment 226, wherein the second RyR1 protein is a mutated RyR1 protein.

Embodiment 228. The method of embodiment 226 or embodiment 227, wherein the second RyR1 protein is a post-translationally modified RyR1 protein.

Embodiment 229. The method of any one of embodiments 226-228, wherein the second RyR1 protein is a mutated and post-translationally modified RyR1 protein.

Embodiment 230. The method of any one of embodiments 226-229, wherein the test compound preferentially binds to a mutated RyR1 relative to wild-type RyR1.

Embodiment 231. The method of any one of embodiments 226-230, wherein the test compound preferentially binds to post-translationally modified RyR1 relative to wild-type RyR1.

Embodiment 232. The method of any one of embodiments 226-231, wherein the test compound preferentially binds to a mutant and post-translationally modified RyR1 relative to a wild-type RyR1.

Embodiment 233. The method of any one of embodiments 185-232, wherein the test compound contains a benzothiazepane moiety.

Embodiment 234. The method of any one of embodiments 185-233, wherein the test compound is a compound of Formula (I):

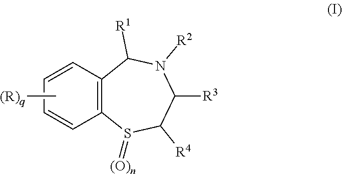

wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —N₀₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, —(CH₂)ₘ—R¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹.
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4,
or any other compound herein, or a pharmaceutically acceptable salt thereof.

### EXAMPLES

### Example 1: Purification of Recombinant Calmodulin and TEV Protease

All purification steps were performed on ice unless otherwise stated. Recombinant Homo sapiens calmodulin (CaM) was expressed in BL21 (DE3) E. coli cells with a N-terminal 6-histidine tag and a tobacco etch virus (TEV) protease cleavage site. Protein expression was induced with 0.8 mM IPTG added to E. coli at an OD₆₀₀ of 0.8 with overnight incubation at 18° C. prior to centrifugation for 10 min at 6500×g and storage at 80° C. CaM was purified using a two-step HisTrap™ (5 mL, GE Healthcare Life Sciences) column purification. In brief, the pellets were resuspended in buffer A (20 mM HEPES pH 7.5, 150 mM NaCl, 20 mM Imidazole, 5 mM BME, 0.5 mM AEBSF) and lysed using an emulsiflex (Avestin EmulsiFlex-C₃). The lysate was pelleted by centrifugation for 10 min at 100 kxg. The supernatant was then loaded over a HisTrap™ FF column and washed with 5 CV of buffer A to remove contaminants prior to elution using a linear gradient from buffer A to buffer B (buffer A containing 500 mM Imidazole). Fractions containing CaM were pooled, 1-2 mg of purified TEV protease was added, and the mixture was dialyzed overnight at 4° C. into buffer C (buffer A with no imidazole). CaM was then loaded onto a HisTrap™ HP column with the flowthrough collected and the wash fractionated to retain fractions containing CaM prior to elution of TEV and any remaining contaminants with a linear gradient from buffer C to buffer B. The flowthrough and any fractions containing CaM were pooled, concentrated to >2 mM, determined by spectroscopy using a NanoDrop® 1000 (ThermoFisher) with abs @ 280 nm and the extinction coefficient of CaM. CaM was stored at 20° C. TEV protease was purified in the same manner with the exception of using an uncleavable his-tag and thus ending after the first HisTrap column wherein the purified protease was stored at 80° C. in buffer C with 10% glycerol.

### Example 2: Purification of Native RyR1

All purification steps were performed on ice unless otherwise stated. RyR1 was purified from rabbit skeletal muscle with modifications to the previously published methodology. Rabbit back and thigh muscle tissue were harvested and snap frozen in liquid nitrogen immediately following euthanasia prior to shipping on dry ice and storage at −80° C. (BioIVT). 20 g of frozen rabbit muscle was resuspended and lysed in 200 mL buffer A (10 mM tris maleate pH 6.8, 1 mM EGTA, 1 mM benzamidine hydrochloride, 0.5 mM AEBSF) via blending with a Waring blender. The resulting suspension was pelleted by centrifugation for ten minutes at 11,000×g. The supernatant was filtered through cheesecloth to remove debris and the membranes were then pelleted by centrifugation for thirty minutes at 36,000×g.

The membranes were solubilized in buffer B (10 mM HEPES pH 7.4, 0.8 M NaCl, 1% CHAPS, 0.1% phosphatidylcholine, 1 mM EGTA, 2 mM DTT, 0.5 mM AEBSF, 1 mM benzamidine hydrochloride, 1 protease inhibitor tablet (Pierce)) prior to homogenization using a glass tissue grinder (Kontes). Homogenization was repeated following the addition of buffer C (buffer B with no NaCl) at a 1:1 ratio with buffer B. The resulting homogenate was submitted to centrifugation for thirty minutes at 100 kxg. The supernatant was then vacuum filtered and incubated with excess, purified CaM (prepared according to EXAMPLE 1) for thirty minutes prior to loading onto a HiTrap Q HP column (5 mL, GE Healthcare Life Sciences) at 1 mL/mm. This column was pre-equilibrated with buffer D (10 mM HEPES pH 7.4, 400 mM NaCl, 1.0% CHAPS, 1 mM EGTA, 0.5 mM TCEP, 0.5 mM AEBSF, 1 mM benzamidine hydrochloride, 0.01% 1,2-dioleoyl-sn-glycero-3-phosphocholine (DOPC, Avanti, Cat #850375C)).

DOPC, dissolved in chloroform, was evaporated under nitrogen gas and resuspended in buffer D. Contaminating proteins were washed away with six column volumes (CV) of buffer D prior to elution of RyR1 with a linear gradient from 480 to 550 mM NaCl using buffers D and E (buffer D with 600 mM NaCl). RyR1-containing fractions were pooled and concentrated to 10 mg/mL using 100,000 kDa cut-off centrifugation filters (MilliporeSigma) prior to addition of 10 mM NaATP, 0.5 mM Compound 1, 5 mM caffeine, and 30 μM Ca²⁺ free. Free Ca²⁺ concentrations were calculated using MaxChelator. Final RyR1 concentration was 8.4 mg/mL (15 μM), determined by spectroscopy using a NanoDrop® 1000 Spectrophotometer (ThermoFisher, 1 abs @ 280 nm=1 mg/mL).

### Example 3: Single Particle Cryogenic Electron Microscopy Analysis of RyR1

Using cryogenic electron microscopy, the structure of RyR1 at 2.45 Å was resolved, revealing a binding site in in the RY1&2 domain (3.10 Å local resolution). The binding site was determined to be formed by a cleft in the RY1&2 domain that binded to both Compound 1 (4-[(7-methoxy-2,3-dihydro-1,4-benzothiazepin-4(5H)-yl)methyl]benzoic acid) and adenosine triphosphate (ATP).

### Grid Preparation.

UltrAuFoil holey gold grids (Quantifoil R 0.6/1.0, Au 300) were plasma cleaned with H₂ and 02 (Gatan). 3 μL of the purified native RyR1 sample prepared in EXAMPLE 2 was applied to each grid. Grids were then blotted for 6.5 sec at blot force 3, with a wait time of thirty seconds and no drain time prior to vitrification by plunge freezing into liquid ethane chilled with liquid nitrogen with a Vitrobot Mark IV operated at 4° C. with 100% relative humidity. Ashless filter paper was used to limit Ca²⁺ contamination.

### Cryo-EM Data Collection & Processing.

Prepared grids were screened in-house on a Glacios Cryo-TEM (ThermoFisher) microscope with a 200-kV x-FEG source and a Falcon 3EC direct electron detector (ThermoFisher). Microscope operations and data collection were carried out using EPU software (ThermoFisher). High resolution data collection was performed at Columbia University on a Titan Krios 300-kV (ThermoFisher) microscope equipped with an energy filter (slit width 20 eV) and a K3 direct electron detector (Gatan). Data were collected using Leginon and at a nominal magnification of 105,000× in electron counting mode, corresponding to a pixel size of 0.826 Å. The electron dose rate was set to 16 e⁻/pixel/sec with 2.5 second exposures for a total dose of 57.65 e/Å².

CryoEM data processing was performed in cryoSPARC™ with image stacks aligned using Patch motion, defocus value estimation by Patch CTF estimation. Particle picking was performed using the template picker with pre-existing templates. 333,010 particles were initially picked from 6,862 micrographs and these were subjected to 2D classification in cryoSPARC™ with 50 classes. 154,000 particles from the highest-resolution classes were pooled for ab initio 3D reconstruction with a single class followed by homogenous refinement with C₄ symmetry imposed. Symmetry expansion was performed prior to local refinement with three separate masks. The first mask was composed of the N-terminal domains, the SPRY domains, the RY1&2 domain, calstabin, and calmodulin. The second mask surrounded the bridging-solenoid, and the third mask surrounded the pore of the RyR. Only the pore mask utilized C₄ symmetry. The resulting maps were combined in Chimera to generate a composite map prior to calibration of the pixel size using correlation coefficients with a map generated from the crystal structure of the N-terminal domain (2XOA). The pixel size was altered by 0.001 Å per step, up to 10 steps in each direction with an initial and final pixel size of 0.826 Å and 0.833 Å, respectively. Model building was performed in Coot. Real-space refinement was performed in Phenix. Figures of the final structure were created using ChimeraX. Quantification of the pore radius was calculated using HOLE.

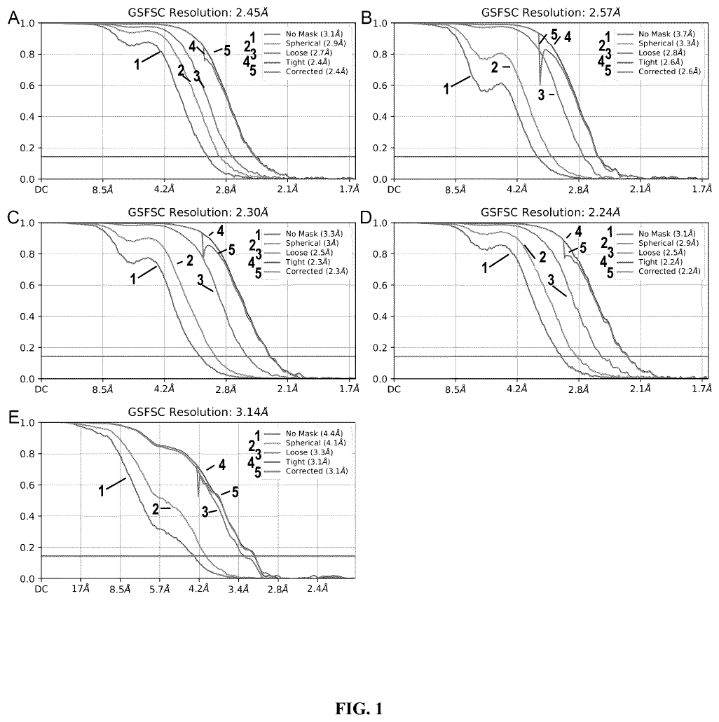

CryoEM statistics are summarized in TABLE 1. FIG. 1 provides GSFSC curves of RyR1 with Compound 1 & ATP. FIG. 1, shows global resolution (Panel A), resolution of local refinement with the NTD mask (Panel B), resolution of local refinement with the BrSol mask (Panel C), and resolution of local refinement of the pore, without symmetry expansion (Panel D), and local resolution of the RY1&2 domain (Panel E). TABLE 1. CryoEM statistics. Map resolution range represents the range determined by local refinement in cryoSPARC using the local masks.

TABLE 1
CryoEM statistics. Map resolution range represents the range determined
by local refinement in cryoSPARC using the local masks.
Data collection

| Microscope | FEI Titan Krios |
|---|---|
| Detector | Gatan K3 |
| Voltage (kV) | 300 |
| Magnification | 105,000 |
| Exposure (e⁻/Å²) | 57.65 |
| Defocus range (um) | −1 to −2 |
| Pixel size (Å) | 0.833 |

Processing

| | |
|---|---|
| Software | cryoSPARC |
| Symmetry | C4 |
| Initial particles (N) | 333,010 |
| Final particles (N) | 153,840 |
| Map resolution (Å) | 2.45^{a} |
| Map resolution | 2.24-2.57^{b} |
| range (Å) | |

Model Composition

| | |
|---|---|
| Peptide chains | 12 |
| Nonhydrogen | 149,472 |
| Protein residues | 18,644 |
| Ligands | 44 |

Mean B factors (Å²)

| | |
|---|---|
| Protein | 78.20 |
| Ligands | 89.69 |

R.m.s. deviations

| | |
|---|---|
| Bond length (Å) | 0.003 |
| Bond angles (°) | 0.496 |

Ramachandran

| | |
|---|---|
| Favored (%) | 97.52 |
| Allowed (%) | 2.48 |
| Disallowed (%) | 0.00 |

Validation^{c}

| | |
|---|---|
| MolProbity score | 1.58 |
| Clashscore | 5.12 |
| Rotamer outliers (%) | 1.9 |
| PDB ID | 7TZC |
| EMDB ID | 26205 |
| EMPIAR ID | 10997 |

^{a}Map resolution is the result of refinement in cryoSPARC before symmetry expansion and local refinement.
^{b}Map resolution range represents the range of the averages determined by local refinement in cryoSPARC.
^{c}EMDB: Electron Microscopy Data Bank; PDB: Protein Data Bank; RMSD: root-mean-square deviation.

### Results.

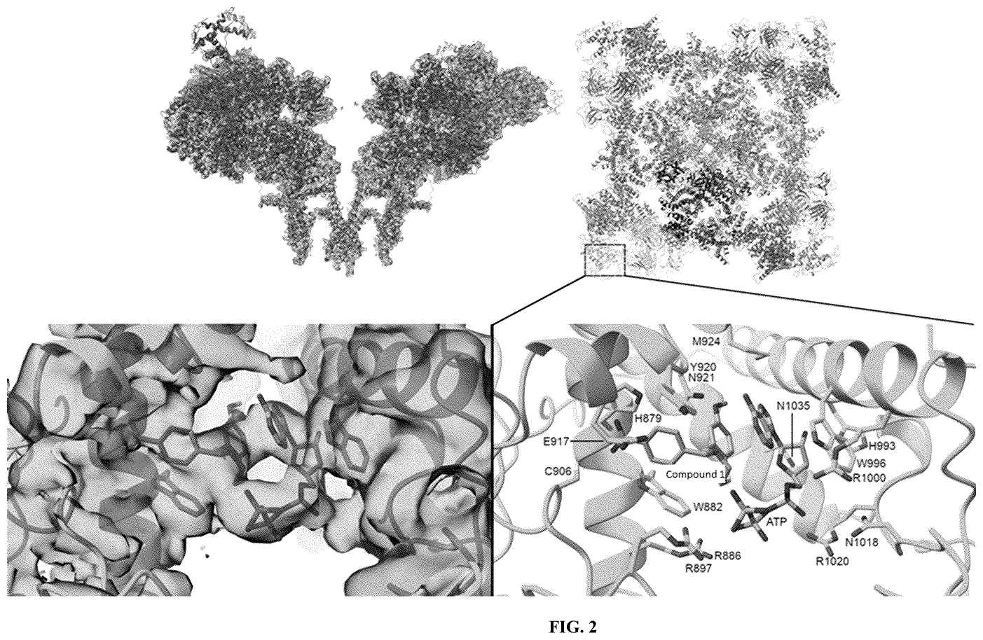

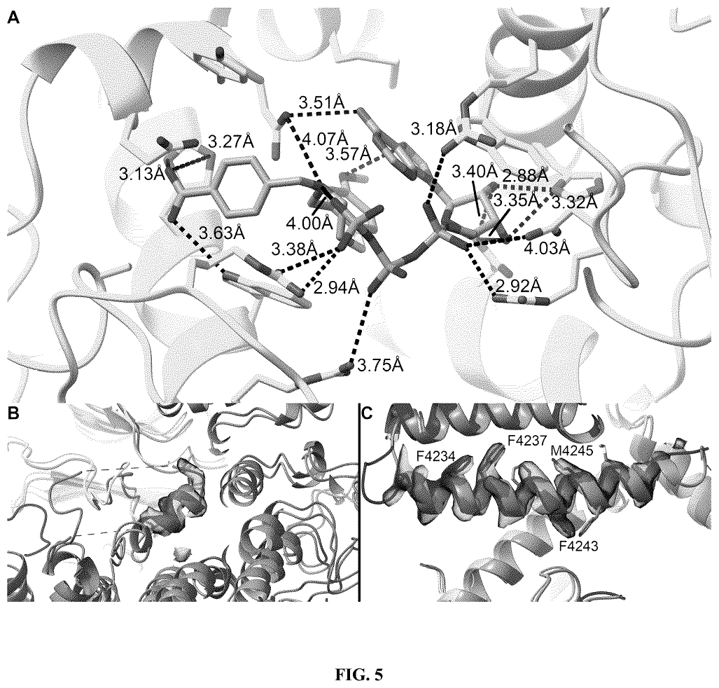

The data revealed that Compound 1 simultaneously occupies a binding site in the RY1&2 domain of RyR1 with a single molecule of ATP. ATP was bound on the interior, pi-stacking with W996, while Compound 1 was bound on the periphery, pi-stacking with W882 and ATP. The triphosphate tail of ATP was further supported by salt bridges with residues H993, R1000, N1018, R1020, and potentially R866 and R897. The ribose ring was also supported by N1035. Several potential interactions existed for the benzoic acid tail of Compound 1, including a salt bridge with H879 and potentially N921. The residues that were close enough to form hydrophobic or hydrogen bonding interactions are highlighted in FIG. 2 and the potential salt bridges are shown in FIG. 5, Panel A.

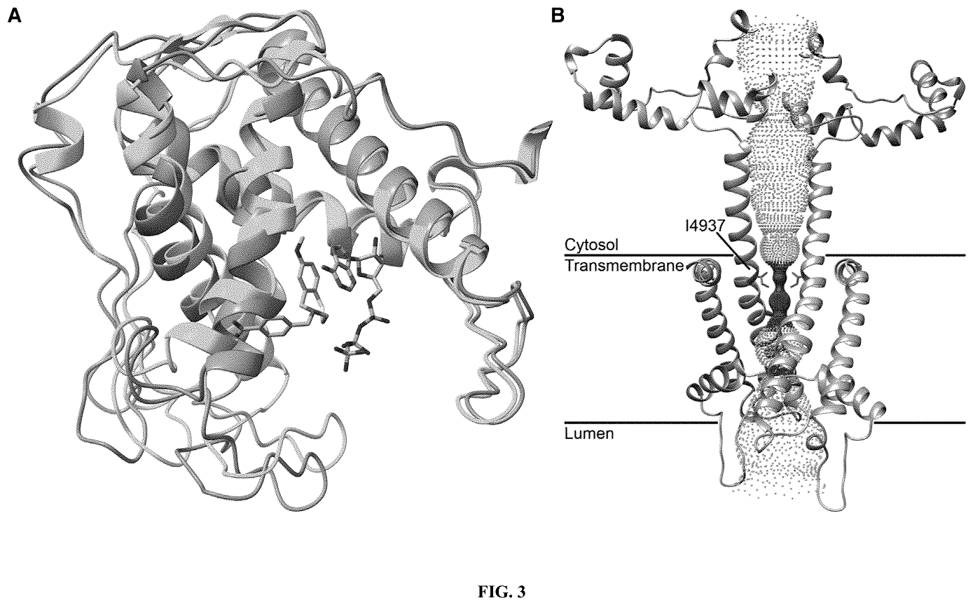

Compound 1 and ATP binding to RyR1 caused a conformation change in the RY1&2 domain. FIG. 3, Panel A depicts the RY1&2 domain in the presence (light grey) and absence (dark grey) of Compound 1. This conformational change resulted in a global shift that radiates outward to other domains. In the unbound state, the RY1&2 domain is open, whereas the ATP- and Compound 1-bound state showed the periphery of the domain closing around the aforementioned ligands, with the exception of the top-most helix, which bent outward to accommodate Compound 1. Sequence alignments indicate that proximate residues of the RY1&2 domain are also conserved between RyR1, 2, and 3, as well as across different sources, including W882, W996, C906, and the many arginine residues that support the phosphate tail of ATP.

The pore of the channel was also found to be closed despite the presence of Ca²⁺, ATP, and caffeine. FIG. 3, Panel B depicts the pore of the channel, which was found to be in the closed conformation. The transmembrane pore (residues 4,820-5,037) are depicted as a ribbon diagram of 2 protomers with hydrophobic gate residue 14937. The dotted representation of the accessible inner surface of the channel is light grey where the radius exceeds 4 Å and dark grey where the radius is less than 4 Å.

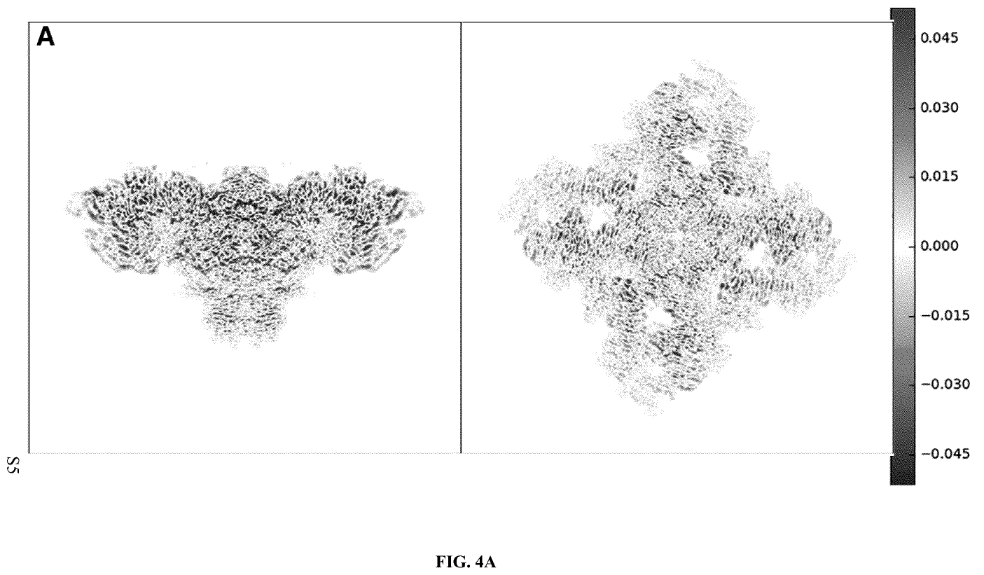

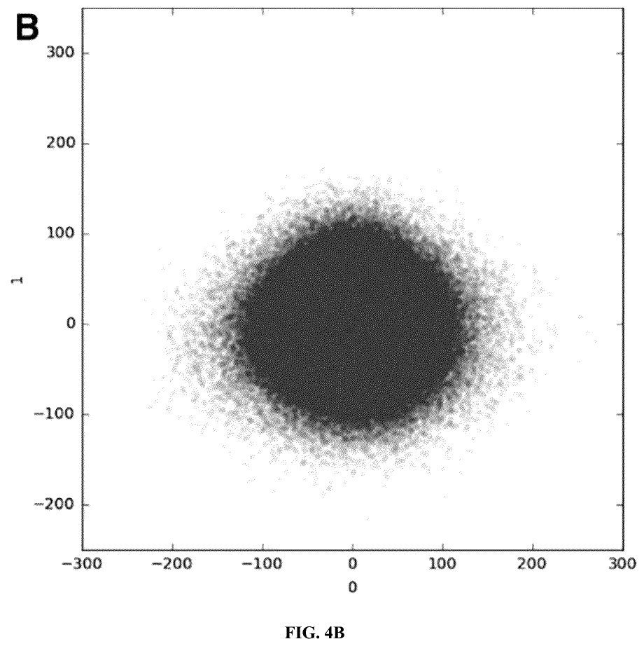

The presence of Ca²⁺, ATP, and caffeine ligands were sufficient to push the channel into a primed state, with a proportion (30%-60%) of the channels being in the open state. However, no channels were found to be in the open state in the presence of Compound 1. 3D variability slices show no variation in the pore (FIG. 4A) in the presence of Compound 1 (indicated by the lack of grey shading in the center), and the reaction coordinate scatterplot of the eigenvectors (FIG. 4B) shows that only one state (closed) is present. No significant conformation changes were found in other domains as a result of ryanodine receptor modulator binding, although the closing of the RY1&2 domain was accompanied by improved resolution, making it possible to resolve ligand binding.

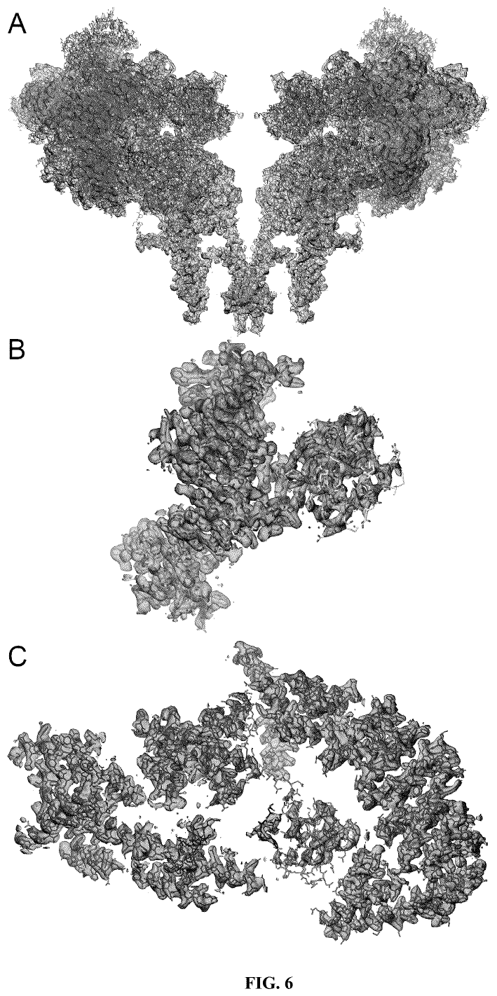

The improved resolution also allowed for additional assignments in numerous unstructured loops in addition to corrections made in the bridging and central solenoids, namely the addition of a short helix (residues 3,472-3,479, FIG. 5, Panel B) and the correcting of a mismodeled helix (residues 4224-4254, FIG. 5, Panel C), which was evident by the side-chain density of three phenylalanine residues (residues 4,234, 4,237, and 4,243). Representative side-chain densities for two protomers and for the SPRY domains and calstabin, as well as BrSol and CaM, are shown in FIG. 6. FIG. 6 provides a sideview of two protomers, (Panel A), SPRY domain beta-sheets and calstabin (Panel B), and bridging solenoid helices and calmodulin (Panel C). Connecting loops and select helices have been omitted from Panel B and Panel C for clarity.

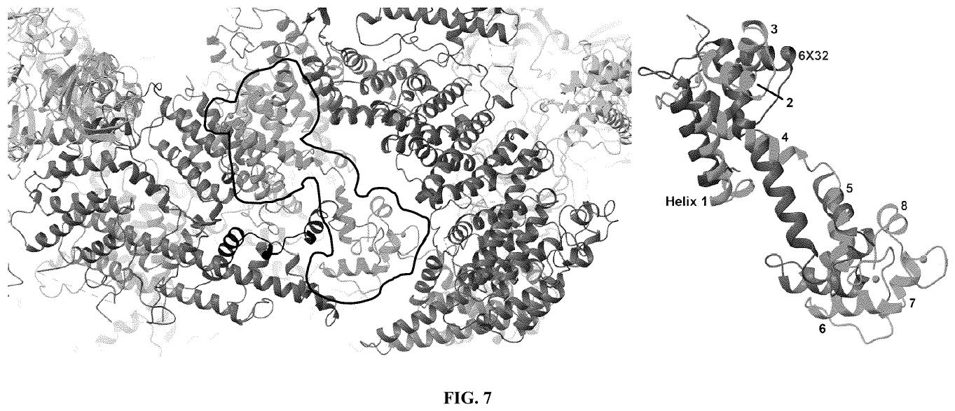

Changes in the conformation of CaM were attributed to Ca²⁺ binding to CaM along with significantly improved resolution, particularly regarding the c-lobe. The binding site of calstabin was unchanged. FIG. 7 shows the calmodulin binding site (left panel) and conformation change (right panel). In FIG. 7, left panel, calmodulin is outlined with a bold line. In FIG. 7, right panel, calmodulin is shown in light grey and aligned with PDB structure 6=32 in dark grey (Wt pig RyR1 in complex with apoCaM, EGTA condition) to show the conformation change as a result of Ca²⁺ binding.

The three dimensional atomic coordinates as determined by cryoEM for the RY1&2 domain of RyR1 (residues 855-1,037), Compound 1, and ATP in the binding site are provided in TABLE 2, TABLE 3, and TABLE 4, respectively.

TABLE 2
Three-dimensional atomic coordinates of RY1&2 domain.

| Id¹ | type_symbol² | label_atom_id³ | label_comp_id⁴ | label_seq_id⁵ | Cartn_x⁶ | Cartn_y⁷ | Cartn_z⁸ | B_iso_or_equiv⁹ |
|---|---|---|---|---|---|---|---|---|
| 11875 | N | N | ARG | 835 | 211.061 | 89.179 | 215.615 | 62.67 |
| 11876 | C | CA | ARG | 835 | 211.106 | 90.415 | 214.848 | 62.67 |
| 11877 | C | C | ARG | 835 | 211.679 | 91.587 | 215.634 | 62.67 |
| 11878 | O | O | ARG | 835 | 211.785 | 92.688 | 215.085 | 62.67 |
| 11879 | C | CB | ARG | 835 | 209.703 | 90.773 | 214.348 | 62.67 |
| 11880 | C | CG | ARG | 835 | 208.994 | 89.636 | 213.632 | 62.67 |
| 11881 | C | CD | ARG | 835 | 207.66 | 90.08 | 213.053 | 62.67 |
| 11882 | N | NE | ARG | 835 | 207.825 | 90.973 | 211.912 | 62.67 |
| 11883 | C | CZ | ARG | 835 | 207.74 | 92.295 | 211.975 | 62.67 |
| 11884 | N | NH1 | ARG | 835 | 207.482 | 92.919 | 213.113 | 62.67 |
| 11885 | N | NH2 | ARG | 835 | 207.915 | 93.009 | 210.867 | 62.67 |
| 11886 | N | N | GLY | 836 | 212.047 | 91.382 | 216.894 | 57.82 |
| 11887 | C | CA | GLY | 836 | 212.582 | 92.441 | 217.713 | 57.82 |
| 11888 | C | C | GLY | 836 | 212.355 | 92.182 | 219.187 | 57.82 |
| 11889 | O | O | GLY | 836 | 211.765 | 91.17 | 219.577 | 57.82 |
| 11890 | N | N | PRO | 837 | 212.824 | 93.092 | 220.037 | 51.4 |
| 11891 | C | CA | PRO | 837 | 212.646 | 92.908 | 221.482 | 51.4 |
| 11892 | C | C | PRO | 837 | 211.175 | 92.902 | 221.87 | 51.4 |
| 11893 | O | O | PRO | 837 | 210.358 | 93.63 | 221.305 | 51.4 |
| 11894 | C | CB | PRO | 837 | 213.383 | 94.109 | 222.085 | 51.4 |
| 11895 | C | CG | PRO | 837 | 213.398 | 95.127 | 220.997 | 51.4 |
| 11896 | C | CD | PRO | 837 | 213.509 | 94.355 | 219.719 | 51.4 |
| 11897 | N | N | HIS | 838 | 210.847 | 92.067 | 222.85 | 49.63 |
| 11898 | C | CA | HIS | 838 | 209.493 | 91.943 | 223.368 | 49.63 |
| 11899 | C | C | HIS | 838 | 209.413 | 92.586 | 224.743 | 49.63 |
| 11900 | O | O | HIS | 838 | 210.225 | 92.282 | 225.623 | 49.63 |
| 11901 | C | CB | HIS | 838 | 209.067 | 90.476 | 223.459 | 49.63 |
| 11902 | C | CG | HIS | 838 | 208.599 | 89.898 | 222.162 | 49.63 |
| 11903 | N | ND1 | HIS | 838 | 208.531 | 90.635 | 221 | 49.63 |
| 11904 | C | CD2 | HIS | 838 | 208.169 | 88.654 | 221.846 | 49.63 |
| 11905 | C | CE1 | HIS | 838 | 208.081 | 89.868 | 220.022 | 49.63 |
| 11906 | N | NE2 | HIS | 838 | 207.855 | 88.662 | 220.509 | 49.63 |
| 11907 | N | N | LEU | 839 | 208.439 | 93.471 | 224.925 | 47.46 |
| 11908 | C | CA | LEU | 839 | 208.156 | 94.019 | 226.241 | 47.46 |
| 11909 | C | C | LEU | 839 | 207.358 | 93.009 | 227.051 | 47.46 |
| 11910 | O | O | LEU | 839 | 206.435 | 92.371 | 226.539 | 47.46 |
| 11911 | C | CB | LEU | 839 | 207.388 | 95.333 | 226.123 | 47.46 |
| 11912 | C | CG | LEU | 839 | 208.112 | 96.443 | 225.363 | 47.46 |
| 11913 | C | CD1 | LEU | 839 | 207.249 | 97.687 | 225.281 | 47.46 |
| 11914 | C | CD2 | LEU | 839 | 209.441 | 96.756 | 226.022 | 47.46 |
| 11915 | N | N | VAL | 840 | 207.726 | 92.856 | 228.319 | 50.1 |
| 11916 | C | CA | VAL | 840 | 207.13 | 91.858 | 229.196 | 50.1 |
| 11917 | C | C | VAL | 840 | 206.578 | 92.561 | 230.425 | 50.1 |
| 11918 | O | O | VAL | 840 | 207.276 | 93.368 | 231.05 | 50.1 |
| 11919 | C | CB | VAL | 840 | 208.148 | 90.776 | 229.605 | 50.1 |
| 11920 | C | CG1 | VAL | 840 | 207.484 | 89.727 | 230.476 | 50.1 |
| 11921 | C | CG2 | VAL | 840 | 208.764 | 90.139 | 228.374 | 50.1 |
| 11922 | N | N | GLY | 841 | 205.33 | 92.258 | 230.769 | 54.81 |
| 11923 | C | CA | GLY | 841 | 204.732 | 92.79 | 231.967 | 54.81 |
| 11924 | C | C | GLY | 841 | 205.122 | 91.992 | 233.189 | 54.81 |
| 11925 | O | O | GLY | 841 | 206.002 | 91.123 | 233.157 | 54.81 |
| 11926 | N | N | PRO | 842 | 204.461 | 92.293 | 234.305 | 63.67 |
| 11927 | C | CA | PRO | 842 | 204.724 | 91.545 | 235.538 | 63.67 |
| 11928 | C | C | PRO | 842 | 204.417 | 90.067 | 235.358 | 63.67 |
| 11929 | O | O | PRO | 842 | 203.544 | 89.672 | 234.584 | 63.67 |
| 11930 | C | CB | PRO | 842 | 203.779 | 92.192 | 236.556 | 63.67 |
| 11931 | C | CG | PRO | 842 | 203.511 | 93.55 | 236.011 | 63.67 |
| 11932 | C | CD | PRO | 842 | 203.509 | 93.394 | 234.521 | 63.67 |
| 11933 | N | N | SER | 843 | 205.169 | 89.243 | 236.077 | 74.59 |
| 11934 | C | CA | SER | 843 | 205.046 | 87.795 | 235.994 | 74.59 |
| 11935 | C | C | SER | 843 | 204.27 | 87.284 | 237.197 | 74.59 |
| 11936 | O | O | SER | 843 | 204.544 | 87.685 | 238.333 | 74.59 |
| 11937 | C | CB | SER | 843 | 206.422 | 87.131 | 235.93 | 74.59 |
| 11938 | O | OG | SER | 843 | 207.181 | 87.431 | 237.088 | 74.59 |
| 11939 | N | N | ARG | 844 | 203.301 | 86.409 | 236.945 | 80.18 |
| 11940 | C | CA | ARG | 844 | 202.552 | 85.798 | 238.032 | 80.18 |
| 11941 | C | C | ARG | 844 | 203.468 | 84.883 | 238.834 | 80.18 |
| 11942 | O | O | ARG | 844 | 204.149 | 84.021 | 238.272 | 80.18 |
| 11943 | C | CB | ARG | 844 | 201.363 | 85.013 | 237.482 | 80.18 |
| 11944 | C | CG | ARG | 844 | 200.441 | 85.826 | 236.588 | 80.18 |
| 11945 | C | CD | ARG | 844 | 199.242 | 85.004 | 236.144 | 80.18 |
| 11946 | N | NE | ARG | 844 | 198.357 | 85.753 | 235.26 | 80.18 |
| 11947 | C | CZ | ARG | 844 | 198.432 | 85.736 | 233.936 | 80.18 |
| 11948 | N | NH1 | ARG | 844 | 199.345 | 85.018 | 233.304 | 80.18 |
| 11949 | N | NH2 | ARG | 844 | 197.567 | 86.457 | 233.228 | 80.18 |
| 11950 | N | N | CYS | 845 | 203.485 | 85.072 | 240.149 | 83.44 |
| 11951 | C | CA | CYS | 845 | 204.356 | 84.315 | 241.032 | 83.44 |
| 11952 | C | C | CYS | 845 | 203.537 | 83.641 | 242.122 | 83.44 |
| 11953 | O | O | CYS | 845 | 202.56 | 84.202 | 242.627 | 83.44 |
| 11954 | C | CB | CYS | 845 | 205.424 | 85.214 | 241.666 | 83.44 |
| 11955 | S | SG | CYS | 845 | 206.59 | 84.342 | 242.737 | 83.44 |
| 11956 | N | N | LEU | 846 | 203.945 | 82.428 | 242.477 | 70.84 |
| 11957 | C | CA | LEU | 846 | 203.286 | 81.691 | 243.54 | 70.84 |
| 11958 | C | C | LEU | 846 | 203.695 | 82.242 | 244.902 | 70.84 |
| 11959 | O | O | LEU | 846 | 204.665 | 82.991 | 245.04 | 70.84 |
| 11960 | C | CB | LEU | 846 | 203.618 | 80.205 | 243.449 | 70.84 |
| 11961 | C | CG | LEU | 846 | 203.11 | 79.476 | 242.207 | 70.84 |
| 11962 | C | CD1 | LEU | 846 | 203.557 | 78.03 | 242.232 | 70.84 |
| 11963 | C | CD2 | LEU | 846 | 201.596 | 79.57 | 242.119 | 70.84 |
| 11964 | N | N | SER | 847 | 202.934 | 81.858 | 245.921 | 64.56 |
| 11965 | C | CA | SER | 847 | 203.203 | 82.256 | 247.291 | 64.56 |
| 11966 | C | C | SER | 847 | 203.378 | 81.011 | 248.146 | 64.56 |
| 11967 | O | O | SER | 847 | 202.963 | 79.912 | 247.771 | 64.56 |
| 11968 | C | CB | SER | 847 | 202.08 | 83.134 | 247.856 | 64.56 |
| 11969 | O | OG | SER | 847 | 202.333 | 83.472 | 249.208 | 64.56 |
| 11970 | N | N | HIS | 848 | 204.01 | 81.195 | 249.307 | 60.85 |
| 11971 | C | CA | HIS | 848 | 204.199 | 80.074 | 250.22 | 60.85 |
| 11972 | C | C | HIS | 848 | 202.869 | 79.547 | 250.741 | 60.85 |
| 11973 | O | O | HIS | 848 | 202.784 | 78.387 | 251.159 | 60.85 |
| 11974 | C | CB | HIS | 848 | 205.105 | 80.486 | 251.379 | 60.85 |
| 11975 | C | CG | HIS | 848 | 204.61 | 81.674 | 252.142 | 60.85 |
| 11976 | N | ND1 | HIS | 848 | 204.649 | 82.954 | 251.633 | 60.85 |
| 11977 | C | CD2 | HIS | 848 | 204.072 | 81.777 | 253.379 | 60.85 |
| 11978 | C | CE1 | HIS | 848 | 204.151 | 83.794 | 252.522 | 60.85 |
| 11979 | N | NE2 | HIS | 848 | 203.794 | 83.106 | 253.591 | 60.85 |
| 11980 | N | N | THR | 849 | 201.826 | 80.378 | 250.724 | 65.17 |
| 11981 | C | CA | THR | 849 | 200.497 | 79.934 | 251.121 | 65.17 |
| 11982 | C | C | THR | 849 | 199.812 | 79.101 | 250.047 | 65.17 |
| 11983 | O | O | THR | 849 | 198.858 | 78.38 | 250.357 | 65.17 |
| 11984 | C | CB | THR | 849 | 199.622 | 81.14 | 251.467 | 65.17 |
| 11985 | O | OG1 | THR | 849 | 199.492 | 81.981 | 250.314 | 65.17 |
| 11986 | C | CG2 | THR | 849 | 200.245 | 81.939 | 252.599 | 65.17 |
| 11987 | N | N | ASP | 850 | 200.274 | 79.182 | 248.798 | 66.31 |
| 11988 | C | CA | ASP | 850 | 199.679 | 78.406 | 247.717 | 66.31 |
| 11989 | C | C | ASP | 850 | 199.944 | 76.914 | 247.845 | 66.31 |
| 11990 | O | O | ASP | 850 | 199.29 | 76.127 | 247.153 | 66.31 |
| 11991 | C | CB | ASP | 850 | 200.194 | 78.903 | 246.366 | 66.31 |
| 11992 | C | CG | ASP | 850 | 199.771 | 80.327 | 246.071 | 66.31 |
| 11993 | O | OD1 | ASP | 850 | 199.065 | 80.924 | 246.909 | 66.31 |
| 11994 | O | OD2 | ASP | 850 | 200.144 | 80.849 | 245 | 66.31 |
| 11995 | N | N | PHE | 851 | 200.881 | 76.507 | 248.697 | 63.64 |
| 11996 | C | CA | PHE | 851 | 201.176 | 75.101 | 248.927 | 63.64 |
| 11997 | C | C | PHE | 851 | 200.494 | 74.561 | 250.175 | 63.64 |
| 11998 | O | O | PHE | 851 | 200.782 | 73.434 | 250.588 | 63.64 |
| 11999 | C | CB | PHE | 851 | 202.687 | 74.889 | 249.018 | 63.64 |
| 12000 | C | CG | PHE | 851 | 203.408 | 75.138 | 247.727 | 63.64 |
| 12001 | C | CD1 | PHE | 851 | 203.804 | 76.416 | 247.376 | 63.64 |
| 12002 | C | CD2 | PHE | 851 | 203.685 | 74.095 | 246.864 | 63.64 |
| 12003 | C | CE1 | PHE | 851 | 204.464 | 76.647 | 246.189 | 63.64 |
| 12004 | C | CE2 | PHE | 851 | 204.346 | 74.322 | 245.677 | 63.64 |
| 12005 | C | CZ | PHE | 851 | 204.735 | 75.599 | 245.338 | 63.64 |
| 12006 | N | N | VAL | 852 | 199.603 | 75.337 | 250.784 | 66.56 |
| 12007 | C | CA | VAL | 852 | 198.864 | 74.915 | 251.969 | 66.56 |
| 12008 | C | C | VAL | 852 | 197.453 | 74.538 | 251.524 | 66.56 |
| 12009 | O | O | VAL | 852 | 196.696 | 75.424 | 251.094 | 66.56 |
| 12010 | C | CB | VAL | 852 | 198.83 | 76.015 | 253.036 | 66.56 |
| 12011 | C | CG1 | VAL | 852 | 198.097 | 75.528 | 254.273 | 66.56 |
| 12012 | C | CG2 | VAL | 852 | 200.24 | 76.457 | 253.384 | 66.56 |
| 12013 | N | N | PRO | 853 | 197.066 | 73.266 | 251.588 | 74.56 |
| 12014 | C | CA | PRO | 853 | 195.69 | 72.898 | 251.234 | 74.56 |
| 12015 | C | C | PRO | 853 | 194.693 | 73.549 | 252.18 | 74.56 |
| 12016 | O | O | PRO | 853 | 194.929 | 73.65 | 253.386 | 74.56 |
| 12017 | C | CB | PRO | 853 | 195.689 | 71.371 | 251.365 | 74.56 |
| 12018 | C | CG | PRO | 853 | 196.798 | 71.076 | 252.315 | 74.56 |
| 12019 | C | CD | PRO | 853 | 197.851 | 72.107 | 252.041 | 74.56 |
| 12020 | N | N | CYS | 854 | 193.568 | 73.993 | 251.622 | 90 |
| 12021 | C | CA | CYS | 854 | 192.524 | 74.685 | 252.378 | 90 |
| 12022 | C | C | CYS | 854 | 191.172 | 74.059 | 252.061 | 90 |
| 12023 | O | O | CYS | 854 | 190.362 | 74.638 | 251.325 | 90 |
| 12024 | C | CB | CYS | 854 | 192.527 | 76.184 | 252.074 | 90 |
| 12025 | S | SG | CYS | 854 | 192.611 | 76.599 | 250.316 | 90 |
| 12026 | N | N | PRO | 855 | 190.894 | 72.876 | 252.602 | 94.76 |
| 12027 | C | CA | PRO | 855 | 189.581 | 72.263 | 252.383 | 94.76 |
| 12028 | C | C | PRO | 855 | 188.483 | 73.04 | 253.088 | 94.76 |
| 12029 | O | O | PRO | 855 | 188.714 | 73.72 | 254.091 | 94.76 |
| 12030 | C | CB | PRO | 855 | 189.742 | 70.861 | 252.979 | 94.76 |
| 12031 | C | CG | PRO | 855 | 190.784 | 71.03 | 254.024 | 94.76 |
| 12032 | C | CD | PRO | 855 | 191.741 | 72.06 | 253.489 | 94.76 |
| 12033 | N | N | VAL | 856 | 187.268 | 72.933 | 252.543 | 111 |
| 12034 | C | CA | VAL | 856 | 186.13 | 73.621 | 253.137 | 111 |
| 12035 | C | C | VAL | 856 | 185.826 | 73.011 | 254.497 | 111 |
| 12036 | O | O | VAL | 856 | 186.132 | 71.841 | 254.763 | 111 |
| 12037 | C | CB | VAL | 856 | 184.909 | 73.56 | 252.202 | 111 |
| 12038 | C | CG1 | VAL | 856 | 184.192 | 72.224 | 252.329 | 111 |
| 12039 | C | CG2 | VAL | 856 | 183.959 | 74.719 | 252.48 | 111 |
| 12040 | N | N | ASP | 857 | 185.233 | 73.814 | 255.375 | 129.2 |
| 12041 | C | CA | ASP | 857 | 184.992 | 73.379 | 256.743 | 129.2 |
| 12042 | C | C | ASP | 857 | 183.884 | 72.335 | 256.785 | 129.2 |
| 12043 | O | O | ASP | 857 | 182.822 | 72.512 | 256.18 | 129.2 |
| 12044 | C | CB | ASP | 857 | 184.625 | 74.573 | 257.62 | 129.2 |
| 12045 | C | CG | ASP | 857 | 184.52 | 74.206 | 259.084 | 129.2 |
| 12046 | O | OD1 | ASP | 857 | 185.573 | 73.974 | 259.713 | 129.2 |
| 12047 | O | OD2 | ASP | 857 | 183.387 | 74.145 | 259.605 | 129.2 |
| 12048 | N | N | THR | 858 | 184.136 | 71.242 | 257.504 | 136.89 |
| 12049 | C | CA | THR | 858 | 183.164 | 70.171 | 257.684 | 136.89 |
| 12050 | C | C | THR | 858 | 182.897 | 69.901 | 259.162 | 136.89 |
| 12051 | O | O | THR | 858 | 182.52 | 68.79 | 259.538 | 136.89 |
| 12052 | C | CB | THR | 858 | 183.629 | 68.895 | 256.983 | 136.89 |
| 12053 | O | OG1 | THR | 858 | 184.924 | 68.523 | 257.47 | 136.89 |
| 12054 | C | CG2 | THR | 858 | 183.696 | 69.107 | 255.475 | 136.89 |
| 12055 | N | N | VAL | 859 | 183.097 | 70.909 | 260.014 | 137.99 |
| 12056 | C | CA | VAL | 859 | 182.819 | 70.75 | 261.439 | 137.99 |
| 12057 | C | C | VAL | 859 | 181.333 | 70.518 | 261.677 | 137.99 |
| 12058 | O | O | VAL | 859 | 180.948 | 69.724 | 262.545 | 137.99 |
| 12059 | C | CB | VAL | 859 | 183.337 | 71.976 | 262.216 | 137.99 |
| 12060 | C | CG1 | VAL | 859 | 182.827 | 71.965 | 263.642 | 137.99 |
| 12061 | C | CG2 | VAL | 859 | 184.856 | 72.011 | 262.203 | 137.99 |
| 12062 | N | N | GLN | 860 | 180.476 | 71.187 | 260.905 | 139.33 |
| 12063 | C | CA | GLN | 860 | 179.029 | 71.035 | 261.042 | 139.33 |
| 12064 | C | C | GLN | 860 | 178.646 | 69.656 | 260.513 | 139.33 |
| 12065 | O | O | GLN | 860 | 178.207 | 69.485 | 259.373 | 139.33 |
| 12066 | C | CB | GLN | 860 | 178.294 | 72.142 | 260.3 | 139.33 |
| 12067 | C | CG | GLN | 860 | 179.193 | 73.24 | 259.752 | 139.33 |
| 12068 | C | CD | GLN | 860 | 179.719 | 74.169 | 260.831 | 139.33 |
| 12069 | O | OE1 | GLN | 860 | 178.989 | 74.556 | 261.745 | 139.33 |
| 12070 | N | NE2 | GLN | 860 | 180.995 | 74.529 | 260.732 | 139.33 |
| 12071 | N | N | ILE | 861 | 178.823 | 68.65 | 261.365 | 148.41 |
| 12072 | C | CA | ILE | 861 | 178.521 | 67.28 | 260.975 | 148.41 |
| 12073 | C | C | ILE | 861 | 177.06 | $6.985 | 261.291 | 148.41 |
| 12074 | O | O | ILE | 861 | 176.737 | 66.373 | 262.314 | 148.41 |
| 12075 | C | CB | ILE | 861 | 179.476 | 66.287 | 261.667 | 148.41 |
| 12076 | C | CG1 | ILE | 861 | 179.653 | 66.627 | 263.15 | 148.41 |
| 12077 | C | CG2 | ILE | 861 | 180.831 | 66.292 | 260.98 | 148.41 |
| 12078 | C | CD1 | ILE | 861 | 180.631 | 65.71 | 263.862 | 148.41 |
| 12079 | N | N | VAL | 862 | 176.166 | 67.424 | 260.405 | 155.65 |
| 12080 | C | CA | VAL | 862 | 174.742 | 67.137 | 260.513 | 155.65 |
| 12081 | C | C | VAL | 862 | 174.22 | 66.713 | 259.149 | 155.65 |
| 12082 | O | O | VAL | 862 | 173.868 | 67.558 | 258.317 | 155.65 |
| 12083 | C | CB | VAL | 862 | 173.968 | 68.355 | 261.048 | 155.65 |
| 12084 | C | CG1 | VAL | 862 | 173.956 | 68.344 | 262.56 | 155.65 |
| 12085 | C | CG2 | VAL | 862 | 174.592 | 69.648 | 260.532 | 155.65 |
| 12086 | N | N | LEU | 863 | 174.165 | 65.41 | 258.903 | 160.19 |
| 12087 | C | CA | LEU | 863 | 173.628 | 64.94 | 257.638 | 160.19 |
| 12088 | C | C | LEU | 863 | 172.173 | 64.518 | 257.824 | 160.19 |
| 12089 | O | O | LEU | 863 | 171.792 | 64.063 | 258.906 | 160.19 |
| 12090 | C | CB | LEU | 863 | 174.454 | 63.773 | 257.09 | 160.19 |
| 12091 | C | CG | LEU | 863 | 175.968 | 63.982 | 256.986 | 160.19 |
| 12092 | C | CD1 | LEU | 863 | 176.668 | 63.502 | 258.251 | 160.19 |
| 12093 | C | CD2 | LEU | 863 | 176.549 | 63.301 | 255.752 | 160.19 |
| 12094 | N | N | PRO | 864 | 171.331 | 64.69 | 256.807 | 169.53 |
| 12095 | C | CA | PRO | 864 | 169.966 | 64.19 | 256.904 | 169.53 |
| 12096 | C | C | PRO | 864 | 169.963 | 62.681 | 257.028 | 169.53 |
| 12097 | O | O | PRO | 864 | 170.867 | 61.997 | 256.512 | 169.53 |
| 12098 | C | CB | PRO | 864 | 169.327 | 64.651 | 255.586 | 169.53 |
| 12099 | C | CG | PRO | 864 | 170.157 | 65.81 | 255.152 | 169.53 |
| 12100 | C | CD | PRO | 864 | 171.549 | 65.467 | 255.577 | 169.53 |
| 12101 | N | N | PRO | 865 | 168.968 | 62.113 | 257.718 | 169.73 |
| 12102 | C | CA | PRO | 865 | 168.96 | 60.652 | 257.914 | 169.73 |
| 12103 | C | C | PRO | 865 | 168.932 | 59.869 | 256.614 | 169.73 |
| 12104 | O | O | PRO | 865 | 169.544 | 58.797 | 256.527 | 169.73 |
| 12105 | C | CB | PRO | 865 | 167.693 | 60.422 | 258.75 | 169.73 |
| 12106 | C | CG | PRO | 865 | 166.828 | 61.61 | 258.464 | 169.73 |
| 12107 | C | CD | PRO | 865 | 167.775 | 62.757 | 258.292 | 169.73 |
| 12108 | N | N | HIS | 866 | 168.236 | 60.377 | 255.596 | 172.44 |
| 12109 | C | CA | HIS | 866 | 168.25 | 59.714 | 254.297 | 172.44 |
| 12110 | C | C | HIS | 866 | 169.622 | 59.817 | 253.643 | 172.44 |
| 12111 | O | O | HIS | 866 | 170.042 | 58.908 | 252.918 | 172.44 |
| 12112 | C | CB | HIS | 866 | 167.17 | 60.302 | 253.392 | 172.44 |
| 12113 | C | CG | HIS | 866 | 166.976 | 61.777 | 253.556 | 172.44 |
| 12114 | N | ND1 | HIS | 866 | 166.162 | 62.316 | 254.528 | 172.44 |
| 12115 | C | CD2 | HIS | 866 | 167.482 | 62.826 | 252.865 | 172.44 |
| 12116 | C | CE1 | HIS | 866 | 166.178 | 63.633 | 254.433 | 172.44 |
| 12117 | N | NE2 | HIS | 866 | 166.971 | 63.968 | 253.431 | 172.44 |
| 12118 | N | N | LEU | 867 | 170.33 | 60.925 | 253.873 | 173.45 |
| 12119 | C | CA | LEU | 867 | 171.698 | 61.037 | 253.38 | 173.45 |
| 12120 | C | C | LEU | 867 | 172.654 | 60.176 | 254.198 | 173.45 |
| 12121 | O | O | LEU | 867 | 173.626 | 59.634 | 253.659 | 173.45 |
| 12122 | C | CB | LEU | 867 | 172.149 | 62.499 | 253.393 | 173.45 |
| 12123 | C | CG | LEU | 867 | 172.054 | 63.248 | 252.061 | 173.45 |
| 12124 | C | CD1 | LEU | 867 | 170.614 | 63.352 | 251.584 | 173.45 |
| 12125 | C | CD2 | LEU | 867 | 172.688 | 64.627 | 252.17 | 173.45 |
| 12126 | N | N | GLU | 868 | 172.4 | 60.046 | 255.503 | 170.8 |
| 12127 | C | CA | GLU | 868 | 173.282 | 59.259 | 256.361 | 170.8 |
| 12128 | C | C | GLU | 868 | 173.294 | 57.793 | 255.947 | 170.8 |
| 12129 | O | O | GLU | 868 | 174.357 | 57.169 | 255.856 | 170.8 |
| 12130 | C | CB | GLU | 868 | 172.857 | 59.4 | 257.823 | 170.8 |
| 12131 | C | CG | GLU | 868 | 173.117 | 60.772 | 258.418 | 170.8 |
| 12132 | C | CD | GLU | 868 | 172.623 | 60.893 | 259.846 | 170.8 |
| 12133 | O | OE1 | GLU | 868 | 171.929 | 59.968 | 260.315 | 170.8 |
| 12134 | O | OE2 | GLU | 868 | 172.93 | 61.913 | 260.499 | 170.8 |
| 12135 | N | N | ARG | 869 | 172.113 | 57.222 | 255.698 | 178.66 |
| 12136 | C | CA | ARG | 869 | 172.042 | 55.819 | 255.303 | 178.66 |
| 12137 | C | C | ARG | 869 | 172.655 | 55.599 | 253.925 | 178.66 |
| 12138 | O | O | ARG | 869 | 173.335 | 54.592 | 253.695 | 178.66 |
| 12139 | C | CB | ARG | 869 | 170.591 | 55.334 | 255.342 | 178.66 |
| 12140 | C | CG | ARG | 869 | 169.613 | 56.183 | 254.54 | 178.66 |
| 12141 | C | CD | ARG | 869 | 169.332 | 55.574 | 253.174 | 178.66 |
| 12142 | N | NE | ARG | 869 | 168.835 | 56.564 | 252.225 | 178.66 |
| 12143 | C | CZ | ARG | 869 | 167.556 | 56.87 | 252.059 | 178.66 |
| 12144 | N | NH1 | ARG | 869 | 166.607 | 56.284 | 252.771 | 178.66 |
| 12145 | N | NH2 | ARG | 869 | 167.22 | 57.786 | 251.156 | 178.66 |
| 12146 | N | N | ILE | 870 | 172.431 | 56.532 | 252.996 | 177.89 |
| 12147 | C | CA | ILE | 870 | 172.955 | 56.37 | 251.645 | 177.89 |
| 12148 | C | C | ILE | 870 | 174.435 | 56.724 | 251.595 | 177.89 |
| 12149 | O | O | ILE | 870 | 175.114 | 56.451 | 250.597 | 177.89 |
| 12150 | C | CB | ILE | 870 | 172.131 | 57.212 | 250.653 | 177.89 |
| 12151 | C | CG1 | ILE | 870 | 172.138 | 56.56 | 249.27 | 177.89 |
| 12152 | C | CG2 | ILE | 870 | 172.66 | 58.637 | 250.585 | 177.89 |
| 12153 | C | CD1 | ILE | 870 | 171.5 | 55.189 | 249.248 | 177.89 |
| 12154 | N | N | ARG | 871 | 174.957 | 57.34 | 252.659 | 172.79 |
| 12155 | C | CA | ARG | 871 | 176.379 | 57.662 | 252.714 | 172.79 |
| 12156 | C | C | ARG | 871 | 177.233 | 56.402 | 252.677 | 172.79 |
| 12157 | O | O | ARG | 871 | 178.237 | 56.344 | 251.957 | 172.79 |
| 12158 | C | CB | ARG | 871 | 176.674 | 58.478 | 253.974 | 172.79 |
| 12159 | C | CG | ARG | 871 | 178.053 | 58.254 | 254.569 | 172.79 |
| 12160 | C | CD | ARG | 871 | 178.128 | 58.818 | 255.979 | 172.79 |
| 12161 | N | NE | ARG | 871 | 179.372 | 58.459 | 256.649 | 172.79 |
| 12162 | C | CZ | ARG | 871 | 179.541 | 57.363 | 257.375 | 172.79 |
| 12163 | N | NH1 | ARG | 871 | 178.563 | 56.488 | 257.544 | 172.79 |
| 12164 | N | NH2 | ARG | 871 | 180.722 | 57.136 | 257.943 | 172.79 |
| 12165 | N | N | GLU | 872 | 176.845 | 55.379 | 253.44 | 179.39 |
| 12166 | C | CA | GLU | 872 | 177.595 | 54.128 | 253.431 | 179.39 |
| 12167 | C | C | GLU | 872 | 177.44 | 53.408 | 252.097 | 179.39 |
| 12168 | O | O | GLU | 872 | 178.389 | 52.788 | 251.603 | 179.39 |
| 12169 | C | CB | GLU | 872 | 177.138 | 53.241 | 254.59 | 179.39 |
| 12170 | C | CG | GLU | 872 | 178.119 | 52.142 | 254.968 | 179.39 |
| 12171 | C | CD | GLU | 872 | 178.008 | 50.921 | 254.079 | 179.39 |
| 12172 | O | OE1 | GLU | 872 | 176.936 | 50.723 | 253.469 | 179.39 |
| 12173 | O | OE2 | GLU | 872 | 178.994 | 50.16 | 253.989 | 179.39 |
| 12174 | N | N | LYS | 873 | 176.248 | 53.478 | 251.5 | 176.84 |
| 12175 | C | CA | LYS | 873 | 176.033 | 52.848 | 250.201 | 176.84 |
| 12176 | C | C | LYS | 873 | 176.85 | 53.532 | 249.112 | 176.84 |
| 12177 | O | O | LYS | 873 | 177.25 | 52.892 | 248.132 | 176.84 |
| 12178 | C | CB | LYS | 873 | 174.545 | 52.862 | 249.851 | 176.84 |
| 12179 | C | CG | LYS | 873 | 173.891 | 51.492 | 249.905 | 176.84 |
| 12180 | C | CD | LYS | 873 | 172.475 | 51.575 | 250.445 | 176.84 |
| 12181 | C | CE | LYS | 873 | 172.474 | 52.036 | 251.893 | 176.84 |
| 12182 | N | NZ | LYS | 873 | 171.098 | 52.147 | 252.447 | 176.84 |
| 12183 | N | N | LEU | 874 | 177.1 | 54.835 | 249.261 | 176 |
| 12184 | C | CA | LEU | 874 | 177.958 | 55.534 | 248.309 | 176 |
| 12185 | C | C | LEU | 874 | 179.375 | 54.977 | 248.34 | 176 |
| 12186 | O | O | LEU | 874 | 180.023 | 54.847 | 247.295 | 176 |
| 12187 | C | CB | LEU | 874 | 177.963 | 57.034 | 248.605 | 176 |
| 12188 | C | CG | LEU | 874 | 178.764 | 57.906 | 247.634 | 176 |
| 12189 | C | CD1 | LEU | 874 | 178.167 | 57.84 | 246.235 | 176 |
| 12190 | C | CD2 | LEU | 874 | 178.83 | 59.343 | 248.128 | 176 |
| 12191 | N | N | ALA | 875 | 179.874 | 54.645 | 249.534 | 182.74 |
| 12192 | C | CA | ALA | 875 | 181.201 | 54.049 | 249.644 | 182.74 |
| 12193 | C | C | ALA | 875 | 181.256 | 52.695 | 248.948 | 182.74 |
| 12194 | O | O | ALA | 875 | 182.289 | 52.324 | 248.378 | 182.74 |
| 12195 | C | CB | ALA | 875 | 181.598 | 53.914 | 251.113 | 182.74 |
| 12196 | N | N | GLU | 876 | 180.157 | 51.938 | 248.992 | 180.89 |
| 12197 | C | CA | GLU | 876 | 180.113 | 50.658 | 248.294 | 180.89 |
| 12198 | C | C | GLU | 876 | 180.274 | 50.843 | 246.791 | 180.89 |
| 12199 | O | O | GLU | 876 | 181.02 | 50.101 | 246.141 | 180.89 |
| 12200 | C | CB | GLU | 876 | 178.799 | 49.938 | 248.596 | 180.89 |
| 12201 | C | CG | GLU | 876 | 178.499 | 49.759 | 250.072 | 180.89 |
| 12202 | C | CD | GLU | 876 | 177.146 | 49.119 | 250.311 | 180.89 |
| 12203 | O | OE1 | GLU | 876 | 176.334 | 49.074 | 249.363 | 180.89 |
| 12204 | O | OE2 | GLU | 876 | 176.894 | 48.659 | 251.444 | 180.89 |
| 12205 | N | N | ASN | 877 | 179.583 | 51.833 | 246.222 | 182.05 |
| 12206 | C | CA | ASN | 877 | 179.593 | 52.002 | 244.774 | 182.05 |
| 12207 | C | C | ASN | 877 | 180.933 | 52.535 | 244.282 | 182.05 |
| 12208 | O | O | ASN | 877 | 181.461 | 52.061 | 243.27 | 182.05 |
| 12209 | C | CB | ASN | 877 | 178.455 | 52.93 | 244.35 | 182.05 |
| 12210 | C | CG | ASN | 877 | 177.858 | 52.542 | 243.014 | 182.05 |
| 12211 | O | OD1 | ASN | 877 | 177.169 | 51.529 | 242.904 | 182.05 |
| 12212 | N | ND2 | ASN | 877 | 178.114 | 53.349 | 241.992 | 182.05 |
| 12213 | N | N | ILE | 878 | 181.5 | 53.519 | 244.985 | 182.27 |
| 12214 | C | CA | ILE | 878 | 182.766 | 54.101 | 244.549 | 182.27 |
| 12215 | C | C | ILE | 878 | 183.885 | 53.07 | 244.631 | 182.27 |
| 12216 | O | O | ILE | 878 | 184.81 | 53.075 | 243.809 | 182.27 |
| 12217 | C | CB | ILE | 878 | 183.095 | 55.369 | 245.36 | 182.27 |
| 12218 | C | CG1 | ILE | 878 | 183.262 | 55.047 | 246.847 | 182.27 |
| 12219 | C | CG2 | ILE | 878 | 182.019 | 56.426 | 245.156 | 182.27 |
| 12220 | C | CD1 | ILE | 878 | 183.607 | 56.251 | 247.698 | 182.27 |
| 12221 | N | N | HIS | 879 | 183.828 | 52.177 | 245.622 | 183.52 |
| 12222 | C | CA | HIS | 879 | 184.82 | 51.111 | 245.708 | 183.52 |
| 12223 | C | C | HIS | 879 | 184.752 | 50.201 | 244.489 | 183.52 |
| 12224 | O | O | HIS | 879 | 185.788 | 49.817 | 243.935 | 183.52 |
| 12225 | C | CB | HIS | 879 | 184.621 | 50.305 | 246.991 | 183.52 |
| 12226 | C | CG | HIS | 879 | 185.012 | 51.043 | 248.232 | 183.52 |
| 12227 | N | ND1 | HIS | 879 | 184.412 | 50.819 | 249.452 | 183.52 |
| 12228 | C | CD2 | HIS | 879 | 185.945 | 52.002 | 248.442 | 183.52 |
| 12229 | C | CE1 | HIS | 879 | 184.957 | 51.609 | 250.359 | 183.52 |
| 12230 | N | NE2 | HIS | 879 | 185.89 | 52.336 | 249.773 | 183.52 |
| 12231 | N | N | GLU | 880 | 183.54 | 49.846 | 244.055 | 186.64 |
| 12232 | C | CA | GLU | 880 | 183.402 | 49.039 | 242.848 | 186.64 |
| 12233 | C | C | GLU | 880 | 183.897 | 49.792 | 241.62 | 186.64 |
| 12234 | O | O | GLU | 880 | 184.599 | 49.221 | 240.778 | 186.64 |
| 12235 | C | CB | GLU | 880 | 181.949 | 48.602 | 242.669 | 186.64 |
| 12236 | C | CG | GLU | 880 | 181.594 | 47.341 | 243.439 | 186.64 |
| 12237 | C | CD | GLU | 880 | 180.133 | 46.967 | 243.307 | 186.64 |
| 12238 | O | OE1 | GLU | 880 | 179.277 | 47.867 | 243.427 | 186.64 |
| 12239 | O | OE2 | GLU | 880 | 179.841 | 45.773 | 243.086 | 186.64 |
| 12240 | N | N | LEU | 881 | 183.552 | 51.078 | 241.503 | 183.88 |
| 12241 | C | CA | LEU | 881 | 184.077 | 51.877 | 240.399 | 183.88 |
| 12242 | C | C | LEU | 881 | 185.594 | 51.976 | 240.471 | 183.88 |
| 12243 | O | O | LEU | 881 | 186.282 | 51.855 | 239.45 | 183.88 |
| 12244 | C | CB | LEU | 881 | 183.452 | 53.272 | 240.401 | 183.88 |
| 12245 | C | CG | LEU | 881 | 182.205 | 53.494 | 239.54 | 183.88 |
| 12246 | C | CD1 | LEU | 881 | 182.372 | 52.857 | 238.167 | 183.88 |
| 12247 | C | CD2 | LEU | 881 | 180.955 | 52.979 | 240.231 | 183.88 |
| 12248 | N | N | TRP | 882 | 186.134 | 52.197 | 241.671 | 185.87 |
| 12249 | C | CA | TRP | 882 | 187.582 | 52.17 | 241.848 | 185.87 |
| 12250 | C | C | TRP | 882 | 188.141 | 50.786 | 241.544 | 185.87 |
| 12251 | O | O | TRP | 882 | 189.207 | 50.659 | 240.931 | 185.87 |
| 12252 | C | CB | TRP | 882 | 187.941 | 52.614 | 243.268 | 185.87 |
| 12253 | C | CG | TRP | 882 | 188.898 | 51.71 | 243.983 | 185.87 |
| 12254 | C | CD1 | TRP | 882 | 188.582 | 50.638 | 244.765 | 185.87 |
| 12255 | C | CD2 | TRP | 882 | 190.327 | 51.81 | 243.998 | 185.87 |
| 12256 | N | NE1 | TRP | 882 | 189.724 | 50.059 | 245.258 | 185.87 |
| 12257 | C | CE2 | TRP | 882 | 190.81 | 50.76 | 244.803 | 185.87 |
| 12258 | C | CE3 | TRP | 882 | 191.244 | 52.683 | 243.407 | 185.87 |
| 12259 | C | CZ2 | TRP | 882 | 192.169 | 50.558 | 245.031 | 185.87 |
| 12260 | C | CZ3 | TRP | 882 | 192.592 | 52.482 | 243.636 | 185.87 |
| 12261 | C | CH2 | TRP | 882 | 193.042 | 51.428 | 244.44 | 185.87 |
| 12262 | N | N | ALA | 883 | 187.434 | 49.735 | 241.968 | 191.55 |
| 12263 | C | CA | ALA | 883 | 187.844 | 48.379 | 241.618 | 191.55 |
| 12264 | C | C | ALA | 883 | 187.71 | 48.131 | 240.121 | 191.55 |
| 12265 | O | O | ALA | 883 | 188.562 | 47.469 | 239.516 | 191.55 |
| 12266 | C | CB | ALA | 883 | 187.021 | 47.36 | 242.405 | 191.55 |
| 12267 | N | N | LEU | 884 | 186.641 | 48.647 | 239.507 | 191.76 |
| 12268 | C | CA | LEU | 884 | 186.443 | 48.453 | 238.074 | 191.76 |
| 12269 | C | C | LEU | 884 | 187.563 | 49.097 | 237.268 | 191.76 |
| 12270 | O | O | LEU | 884 | 188.083 | 48.492 | 236.322 | 191.76 |
| 12271 | C | CB | LEU | 884 | 185.089 | 49.017 | 237.645 | 191.76 |
| 12272 | C | CG | LEU | 884 | 183.987 | 48.011 | 237.303 | 191.76 |
| 12273 | C | CD1 | LEU | 884 | 183.524 | 47.249 | 238.538 | 191.76 |
| 12274 | C | CD2 | LEU | 884 | 182.819 | 48.709 | 236.624 | 191.76 |
| 12275 | N | N | THR | 885 | 187.95 | 50.323 | 237.628 | 194.73 |
| 12276 | C | CA | THR | 885 | 189.042 | 50.988 | 236.923 | 194.73 |
| 12277 | C | C | THR | 885 | 190.347 | 50.223 | 237.091 | 194.73 |
| 12278 | O | O | THR | 885 | 191.183 | 50.195 | 236.18 | 194.73 |
| 12279 | C | CB | THR | 885 | 189.196 | 52.426 | 237.42 | 194.73 |
| 12280 | O | OG1 | THR | 885 | 189.344 | 52.43 | 238.846 | 194.73 |
| 12281 | C | CG2 | THR | 885 | 187.981 | 53.257 | 237.036 | 194.73 |
| 12282 | N | N | ARG | 886 | 190.542 | 49.598 | 238.254 | 193.5 |
| 12283 | C | CA | ARG | 886 | 191.715 | 48.755 | 238.448 | 193.5 |
| 12284 | C | C | ARG | 886 | 191.649 | 47.51 | 237.572 | 193.5 |
| 12285 | O | O | ARG | 886 | 192.683 | 46.999 | 237.128 | 193.5 |
| 12286 | C | CB | ARG | 886 | 191.851 | 48.378 | 239.922 | 193.5 |
| 12287 | C | CG | ARG | 886 | 192.215 | 49.542 | 240.829 | 193.5 |
| 12288 | C | CD | ARG | 886 | 193.346 | 50.374 | 240.24 | 193.5 |
| 12289 | N | NE | ARG | 886 | 194.067 | 51.117 | 241.267 | 193.5 |
| 12290 | C | CZ | ARG | 886 | 194.865 | 52.149 | 241.029 | 193.5 |
| 12291 | N | NH1 | ARG | 886 | 195.048 | 52.614 | 239.804 | 193.5 |
| 12292 | N | NH2 | ARG | 886 | 195.49 | 52.734 | 242.047 | 193.5 |
| 12293 | N | N | ILE | 887 | 190.439 | 47.006 | 237.313 | 201.15 |
| 12294 | C | CA | ILE | 887 | 190.289 | 45.865 | 236.413 | 201.15 |
| 12295 | C | C | ILE | 887 | 190.711 | 46.249 | 235 | 201.15 |
| 12296 | O | O | ILE | 887 | 191.287 | 45.436 | 234.266 | 201.15 |
| 12297 | C | CB | ILE | 887 | 188.847 | 45.322 | 236.465 | 201.15 |
| 12298 | C | CG1 | ILE | 887 | 188.84 | 43.889 | 236.999 | 201.15 |
| 12299 | C | CG2 | ILE | 887 | 188.182 | 45.359 | 235.094 | 201.15 |
| 12300 | C | CD1 | ILE | 887 | 189.536 | 42.897 | 236.091 | 201.15 |
| 12301 | N | N | GLU | 888 | 190.441 | 47.495 | 234.596 | 198.16 |
| 12302 | C | CA | GLU | 888 | 190.956 | 47.977 | 233.32 | 198.16 |
| 12303 | C | C | GLU | 888 | 192.477 | 48.044 | 233.338 | 198.16 |
| 12304 | O | O | GLU | 888 | 193.134 | 47.742 | 232.335 | 198.16 |
| 12305 | C | CB | GLU | 888 | 190.359 | 49.344 | 232.985 | 198.16 |
| 12306 | C | CG | GLU | 888 | 189.039 | 49.278 | 232.229 | 198.16 |
| 12307 | C | CD | GLU | 888 | 187.863 | 48.93 | 233.12 | 198.16 |
| 12308 | O | OE1 | GLU | 888 | 187.483 | 49.773 | 233.958 | 198.16 |
| 12309 | O | OE2 | GLU | 888 | 187.319 | 47.814 | 232.982 | 198.16 |
| 12310 | N | N | GLN | 889 | 193.056 | 48.437 | 234.474 | 197.81 |
| 12311 | C | CA | GLN | 889 | 194.505 | 48.447 | 234.622 | 197.81 |
| 12312 | C | C | GLN | 889 | 195.092 | 47.048 | 234.759 | 197.81 |
| 12313 | O | O | GLN | 889 | 196.318 | 46.905 | 234.72 | 197.81 |
| 12314 | C | CB | GLN | 889 | 194.903 | 49.297 | 235.828 | 197.81 |
| 12315 | C | CG | GLN | 889 | 194.561 | 50.771 | 235.688 | 197.81 |
| 12316 | C | CD | GLN | 889 | 195.053 | 51.595 | 236.859 | 197.81 |
| 12317 | O | OE1 | GLN | 889 | 195.557 | 51.056 | 237.845 | 197.81 |
| 12318 | N | NE2 | GLN | 889 | 194.912 | 52.912 | 236.758 | 197.81 |
| 12319 | N | N | GLY | 890 | 194.258 | 46.025 | 234.918 | 199.32 |
| 12320 | C | CA | GLY | 890 | 194.727 | 44.661 | 235.019 | 199.32 |
| 12321 | C | C | GLY | 890 | 194.7 | 44.052 | 236.402 | 199.32 |
| 12322 | O | O | GLY | 890 | 195.217 | 42.942 | 236.576 | 199.32 |
| 12323 | N | N | TRP | 891 | 194.124 | 44.735 | 237.39 | 200.33 |
| 12324 | C | CA | TRP | 891 | 194.066 | 44.181 | 238.735 | 200.33 |
| 12325 | C | C | TRP | 891 | 193.125 | 42.984 | 238.785 | 200.33 |
| 12326 | O | O | TRP | 891 | 192.089 | 42.955 | 238.116 | 200.33 |
| 12327 | C | CB | TRP | 891 | 193.608 | 45.24 | 239.739 | 200.33 |
| 12328 | C | CG | TRP | 891 | 194.622 | 46.307 | 240.027 | 200.33 |
| 12329 | C | CD1 | TRP | 891 | 195.609 | 46.75 | 239.196 | 200.33 |
| 12330 | C | CD2 | TRP | 891 | 194.75 | 47.062 | 241.238 | 200.33 |
| 12331 | N | NE1 | TRP | 891 | 196.339 | 47.738 | 239.812 | 200.33 |
| 12332 | C | CE2 | TRP | 891 | 195.832 | 47.947 | 241.067 | 200.33 |
| 12333 | C | CE3 | TRP | 891 | 194.052 | 47.076 | 242.45 | 200.33 |
| 12334 | C | CZ2 | TRP | 891 | 196.232 | 48.837 | 242.061 | 200.33 |
| 12335 | C | CZ3 | TRP | 891 | 194.451 | 47.96 | 243.436 | 200.33 |
| 12336 | C | CH2 | TRP | 891 | 195.532 | 48.828 | 243.235 | 200.33 |
| 12337 | N | N | THR | 892 | 193.496 | 41.989 | 239.587 | 202.86 |
| 12338 | C | CA | THR | 892 | 192.669 | 40.819 | 239.828 | 202.86 |
| 12339 | C | C | THR | 892 | 192.502 | 40.63 | 241.328 | 202.86 |
| 12340 | O | O | THR | 892 | 193.088 | 41.357 | 242.134 | 202.86 |
| 12341 | C | CB | THR | 892 | 193.269 | 39.55 | 239.2 | 202.86 |
| 12342 | O | OG1 | THR | 892 | 194.608 | 39.361 | 239.675 | 202.86 |
| 12343 | C | CG2 | THR | 892 | 193.282 | 39.658 | 237.683 | 202.86 |
| 12344 | N | N | TYR | 893 | 191.682 | 39.65 | 241.699 | 195.44 |
| 12345 | C | CA | TYR | 893 | 191.474 | 39.352 | 243.109 | 195.44 |
| 12346 | C | C | TYR | 893 | 192.742 | 38.781 | 243.729 | 195.44 |
| 12347 | O | O | TYR | 893 | 193.451 | 37.986 | 243.106 | 195.44 |
| 12348 | C | CB | TYR | 893 | 190.315 | 38.373 | 243.281 | 195.44 |
| 12349 | C | CG | TYR | 893 | 190.193 | 37.821 | 244.682 | 195.44 |
| 12350 | C | CD1 | TYR | 893 | 189.742 | 38.617 | 245.726 | 195.44 |
| 12351 | C | CD2 | TYR | 893 | 190.528 | 36.503 | 244.961 | 195.44 |
| 12352 | C | CE1 | TYR | 893 | 189.63 | 38.118 | 247.008 | 195.44 |
| 12353 | C | CE2 | TYR | 893 | 190.419 | 35.994 | 246.24 | 195.44 |
| 12354 | C | CZ | TYR | 893 | 189.969 | 36.806 | 247.259 | 195.44 |
| 12355 | O | OH | TYR | 893 | 189.857 | 36.306 | 248.536 | 195.44 |
| 12356 | N | N | GLY | 894 | 193.027 | 39.192 | 244.962 | 201.46 |
| 12357 | C | CA | GLY | 894 | 194.181 | 38.706 | 245.679 | 201.46 |
| 12358 | C | C | GLY | 894 | 194.131 | 39.049 | 247.153 | 201.46 |
| 12359 | O | O | GLY | 894 | 193.636 | 40.108 | 247.553 | 201.46 |
| 12360 | N | N | PRO | 895 | 194.636 | 38.143 | 247.996 | 199.44 |
| 12361 | C | CA | PRO | 895 | 194.689 | 38.443 | 249.438 | 199.44 |
| 12362 | C | C | PRO | 895 | 195.543 | 39.655 | 249.765 | 199.44 |
| 12363 | O | O | PRO | 895 | 195.234 | 40.394 | 250.708 | 199.44 |
| 12364 | C | CB | PRO | 895 | 195.264 | 37.153 | 250.039 | 199.44 |
| 12365 | C | CG | PRO | 895 | 196.007 | 36.506 | 248.914 | 199.44 |
| 12366 | C | CD | PRO | 895 | 195.228 | 36.833 | 247.676 | 199.44 |
| 12367 | N | N | VAL | 896 | 196.612 | 39.879 | 249.008 | 197.42 |
| 12368 | C | CA | VAL | 896 | 197.476 | 41.035 | 249.214 | 197.42 |
| 12369 | C | C | VAL | 896 | 197.019 | 42.17 | 248.31 | 197.42 |
| 12370 | O | O | VAL | 896 | 196.564 | 41.944 | 247.183 | 197.42 |
| 12371 | C | CB | VAL | 896 | 198.946 | 40.659 | 248.952 | 197.42 |
| 12372 | C | CG1 | VAL | 896 | 199.497 | 39.848 | 250.113 | 197.42 |
| 12373 | C | CG2 | VAL | 896 | 199.067 | 39.87 | 247.656 | 197.42 |
| 12374 | N | N | ARG | 897 | 197.13 | 43.402 | 248.806 | 192.89 |
| 12375 | C | CA | ARG | 897 | 196.7 | 44.595 | 248.082 | 192.89 |
| 12376 | C | C | ARG | 897 | 197.939 | 45.342 | 247.602 | 192.89 |
| 12377 | O | O | ARG | 897 | 198.497 | 46.165 | 248.336 | 192.89 |
| 12378 | C | CB | ARG | 897 | 195.832 | 45.484 | 248.971 | 192.89 |
| 12379 | C | CG | ARG | 897 | 195.015 | 46.518 | 248.216 | 192.89 |
| 12380 | C | CD | ARG | 897 | 195.297 | 47.923 | 248.717 | 192.89 |
| 12381 | N | NE | ARG | 897 | 196.569 | 48.431 | 248.218 | 192.89 |
| 12382 | C | CZ | ARG | 897 | 196.702 | 49.166 | 247.123 | 192.89 |
| 12383 | N | NH1 | ARG | 897 | 195.655 | 49.512 | 246.391 | 192.89 |
| 12384 | N | NH2 | ARG | 897 | 197.915 | 49.567 | 246.754 | 192.89 |
| 12385 | N | N | ASP | 898 | 198.364 | 45.059 | 246.372 | 195.71 |
| 12386 | C | CA | ASP | 898 | 199.552 | 45.677 | 245.8 | 195.71 |
| 12387 | C | C | ASP | 898 | 199.234 | 46.203 | 244.41 | 195.71 |
| 12388 | O | O | ASP | 898 | 198.53 | 45.548 | 243.636 | 195.71 |
| 12389 | C | CB | ASP | 898 | 200.722 | 44.687 | 245.722 | 195.71 |
| 12390 | C | CG | ASP | 898 | 200.501 | 43.458 | 246.577 | 195.71 |
| 12391 | O | OD1 | ASP | 898 | 199.637 | 42.633 | 246.213 | 195.71 |
| 12392 | O | OD2 | ASP | 898 | 201.188 | 43.316 | 247.61 | 195.71 |
| 12393 | N | N | ASP | 899 | 199.756 | 47.392 | 244.1 | 197.06 |
| 12394 | C | CA | ASP | 899 | 199.545 | 47.972 | 242.778 | 197.06 |
| 12395 | C | C | ASP | 899 | 200.328 | 47.214 | 241.712 | 197.06 |
| 12396 | O | O | ASP | 899 | 199.788 | 46.879 | 240.651 | 197.06 |
| 12397 | C | CB | ASP | 899 | 199.935 | 49.45 | 242.786 | 197.06 |
| 12398 | C | CG | ASP | 899 | 199.124 | 50.26 | 243.779 | 197.06 |
| 12399 | O | OD1 | ASP | 899 | 199.371 | 50.131 | 244.996 | 197.06 |
| 12400 | O | OD2 | ASP | 899 | 198.236 | 51.022 | 243.342 | 197.06 |
| 12401 | N | N | ASN | 900 | 201.607 | 46.935 | 241.978 | 200.46 |
| 12402 | C | CA | ASN | 900 | 202.431 | 46.238 | 240.995 | 200.46 |
| 12403 | C | C | ASN | 900 | 201.964 | 44.802 | 240.796 | 200.46 |
| 12404 | O | O | ASN | 900 | 201.922 | 44.309 | 239.663 | 200.46 |
| 12405 | C | CB | ASN | 900 | 203.899 | 46.27 | 241.42 | 200.46 |
| 12406 | C | CG | ASN | 900 | 204.831 | 45.798 | 240.321 | 200.46 |
| 12407 | O | OD1 | ASN | 900 | 204.486 | 45.841 | 239.14 | 200.46 |
| 12408 | N | ND2 | ASN | 900 | 206.019 | 45.346 | 240.705 | 200.46 |
| 12409 | N | N | LYS | 901 | 201.612 | 44.114 | 241.883 | 198.27 |
| 12410 | C | CA | LYS | 901 | 201.126 | 42.745 | 241.766 | 198.27 |
| 12411 | C | C | LYS | 901 | 199.718 | 42.686 | 241.191 | 198.27 |
| 12412 | O | O | LYS | 901 | 199.282 | 41.613 | 240.757 | 198.27 |
| 12413 | C | CB | LYS | 901 | 201.172 | 42.056 | 243.131 | 198.27 |
| 12414 | C | CG | LYS | 901 | 201.035 | 40.548 | 243.066 | 198.27 |
| 12415 | C | CD | LYS | 901 | 202.095 | 39.944 | 242.165 | 198.27 |
| 12416 | C | CE | LYS | 901 | 201.637 | 38.603 | 241.633 | 198.27 |
| 12417 | N | NZ | LYS | 901 | 200.383 | 38.766 | 240.85 | 198.27 |
| 12418 | N | N | ARG | 902 | 199.01 | 43.817 | 241.163 | 199.91 |
| 12419 | C | CA | ARG | 902 | 197.66 | 43.901 | 240.603 | 199.91 |
| 12420 | C | C | ARG | 902 | 196.713 | 42.914 | 241.283 | 199.91 |
| 12421 | O | O | ARG | 902 | 195.895 | 42.26 | 240.634 | 199.91 |
| 12422 | C | CB | ARG | 902 | 197.675 | 43.691 | 239.086 | 199.91 |
| 12423 | C | CG | ARG | 902 | 198.872 | 44.316 | 238.378 | 199.91 |
| 12424 | C | CD | ARG | 902 | 198.519 | 44.811 | 236.986 | 199.91 |
| 12425 | N | NE | ARG | 902 | 197.848 | 46.105 | 237.032 | 199.91 |
| 12426 | C | CZ | ARG | 902 | 198.476 | 47.269 | 237.121 | 199.91 |
| 12427 | N | NH1 | ARG | 902 | 199.796 | 47.34 | 237.175 | 199.91 |
| 12428 | N | NH2 | ARG | 902 | 197.762 | 48.39 | 237.162 | 199.91 |
| 12429 | N | N | LEU | 903 | 196.831 | 42.799 | 242.604 | 198.64 |
| 12430 | C | CA | LEU | 903 | 195.943 | 41.974 | 243.411 | 198.64 |
| 12431 | C | C | LEU | 903 | 195.179 | 42.87 | 244.375 | 198.64 |
| 12432 | O | O | LEU | 903 | 195.776 | 43.721 | 245.042 | 198.64 |
| 12433 | C | CB | LEU | 903 | 196.72 | 40.902 | 244.18 | 198.64 |
| 12434 | C | CG | LEU | 903 | 197.408 | 39.821 | 243.345 | 198.64 |
| 12435 | C | CD1 | LEU | 903 | 197.989 | 38.74 | 244.242 | 198.64 |
| 12436 | C | CD2 | LEU | 903 | 196.44 | 39.223 | 242.338 | 198.64 |
| 12437 | N | N | HIS | 904 | 193.859 | 42.681 | 244.442 | 197.54 |
| 12438 | C | CA | HIS | 904 | 193.004 | 43.515 | 245.272 | 197.54 |
| 12439 | C | C | HIS | 904 | 191.889 | 42.662 | 245.87 | 197.54 |
| 12440 | O | O | HIS | 904 | 191.115 | 42.043 | 245.118 | 197.54 |
| 12441 | C | CB | HIS | 904 | 192.415 | 44.67 | 244.457 | 197.54 |
| 12442 | C | CG | HIS | 904 | 191.816 | 45.758 | 245.292 | 197.54 |
| 12443 | N | ND1 | HIS | 904 | 192.178 | 45.976 | 246.604 | 197.54 |
| 12444 | C | CD2 | HIS | 904 | 190.881 | 46.693 | 245.001 | 197.54 |
| 12445 | C | CE1 | HIS | 904 | 191.492 | 46.997 | 247.085 | 197.54 |
| 12446 | N | NE2 | HIS | 904 | 190.698 | 47.45 | 246.132 | 197.54 |
| 12447 | N | N | PRO | 905 | 191.785 | 42.595 | 247.201 | 197.87 |
| 12448 | C | CA | PRO | 905 | 190.672 | 41.841 | 247.806 | 197.87 |
| 12449 | C | C | PRO | 905 | 189.304 | 42.39 | 247.441 | 197.87 |
| 12450 | O | O | PRO | 905 | 188.334 | 41.626 | 247.366 | 197.87 |
| 12451 | C | CB | PRO | 905 | 190.954 | 41.954 | 249.311 | 197.87 |
| 12452 | C | CG | PRO | 905 | 191.817 | 43.169 | 249.449 | 197.87 |
| 12453 | C | CD | PRO | 905 | 192.667 | 43.193 | 248.217 | 197.87 |
| 12454 | N | N | CYS | 906 | 189.198 | 43.7 | 247.212 | 195.97 |
| 12455 | C | CA | CYS | 906 | 187.917 | 44.299 | 246.857 | 195.97 |
| 12456 | C | C | CYS | 906 | 187.464 | 43.919 | 245.451 | 195.97 |
| 12457 | O | O | CYS | 906 | 186.288 | 44.104 | 245.122 | 195.97 |
| 12458 | C | CB | CYS | 906 | 188.001 | 45.822 | 246.983 | 195.97 |
| 12459 | S | SG | CYS | 906 | 186.448 | 46.709 | 246.697 | 195.97 |
| 12460 | N | N | LEU | 907 | 188.357 | 43.376 | 244.622 | 197.34 |
| 12461 | C | CA | LEU | 907 | 188.035 | 43.093 | 243.222 | 197.34 |
| 12462 | C | C | LEU | 907 | 187.275 | 41.769 | 243.115 | 197.34 |
| 12463 | O | O | LEU | 907 | 187.635 | 40.86 | 242.366 | 197.34 |
| 12464 | C | CB | LEU | 907 | 189.301 | 43.064 | 242.377 | 197.34 |
| 12465 | C | CG | LEU | 907 | 189.435 | 44.101 | 241.26 | 197.34 |
| 12466 | C | CD1 | LEU | 907 | 188.096 | 44.328 | 240.572 | 197.34 |
| 12467 | C | CD2 | LEU | 907 | 190.009 | 45.404 | 241.791 | 197.34 |
| 12468 | N | N | VAL | 908 | 186.208 | 41.662 | 243.907 | 198.54 |
| 12469 | C | CA | VAL | 908 | 185.256 | 40.566 | 243.76 | 198.54 |
| 12470 | C | C | VAL | 908 | 183.84 | 41.127 | 243.687 | 198.54 |
| 12471 | O | O | VAL | 908 | 183.113 | 40.882 | 242.716 | 198.54 |
| 12472 | C | CB | VAL | 908 | 185.401 | 39.534 | 244.896 | 198.54 |
| 12473 | C | CG1 | VAL | 908 | 185.652 | 40.223 | 246.231 | 198.54 |
| 12474 | C | CG2 | VAL | 908 | 184.172 | 38.634 | 244.96 | 198.54 |
| 12475 | N | N | ASN | 909 | 183.446 | 41.891 | 244.705 | 195.89 |
| 12476 | C | CA | ASN | 909 | 182.116 | 42.479 | 244.789 | 195.89 |
| 12477 | C | C | ASN | 909 | 182.1 | 43.451 | 245.959 | 195.89 |
| 12478 | O | O | ASN | 909 | 182.942 | 43.377 | 246.858 | 195.89 |
| 12479 | C | CB | ASN | 909 | 181.032 | 41.408 | 244.96 | 195.89 |
| 12480 | C | CG | ASN | 909 | 179.635 | 41.95 | 244.73 | 195.89 |
| 12481 | O | OD1 | ASN | 909 | 179.45 | 42.94 | 244.023 | 195.89 |
| 12482 | N | ND2 | ASN | 909 | 178.642 | 41.302 | 245.329 | 195.89 |
| 12483 | N | N | PHE | 910 | 181.13 | 44.369 | 245.937 | 196.1 |
| 12484 | C | CA | PHE | 910 | 181.025 | 45.352 | 247.012 | 196.1 |
| 12485 | C | C | PHE | 910 | 180.674 | 44.684 | 248.337 | 196.1 |
| 12486 | O | O | PHE | 910 | 181.222 | 45.036 | 249.388 | 196.1 |
| 12487 | C | CB | PHE | 910 | 180 | 46.431 | 246.651 | 196.1 |
| 12488 | C | CG | PHE | 910 | 178.571 | 45.968 | 246.712 | 196.1 |
| 12489 | C | CD1 | PHE | 910 | 178.022 | 45.227 | 245.679 | 196.1 |
| 12490 | C | CD2 | PHE | 910 | 177.774 | 46.286 | 247.799 | 196.1 |
| 12491 | C | CE1 | PHE | 910 | 176.708 | 44.804 | 245.734 | 196.1 |
| 12492 | C | CE2 | PHE | 910 | 176.46 | 45.867 | 247.859 | 196.1 |
| 12493 | C | CZ | PHE | 910 | 175.926 | 45.125 | 246.825 | 196.1 |
| 12494 | N | N | HIS | 911 | 179.755 | 43.715 | 248.309 | 195.26 |
| 12495 | C | CA | HIS | 911 | 179.415 | 42.996 | 249.532 | 195.26 |
| 12496 | C | C | HIS | 911 | 180.521 | 42.025 | 249.927 | 195.26 |
| 12497 | O | O | HIS | 911 | 180.827 | 41.875 | 251.116 | 195.26 |
| 12498 | C | CB | HIS | 911 | 178.084 | 42.266 | 249.362 | 195.26 |
| 12499 | C | CG | HIS | 911 | 176.904 | 43.045 | 249.853 | 195.26 |
| 12500 | N | ND1 | HIS | 911 | 177.029 | 44.157 | 250.658 | 195.26 |
| 12501 | C | CD2 | HIS | 911 | 175.575 | 42.872 | 249.655 | 195.26 |
| 12502 | C | CE1 | HIS | 911 | 175.83 | 44.635 | 250.935 | 195.26 |
| 12503 | N | NE2 | HIS | 911 | 174.93 | 43.874 | 250.338 | 195.26 |
| 12504 | N | N | SER | 912 | 181.132 | 41.358 | 248.945 | 196.23 |
| 12505 | C | CA | SER | 912 | 182.244 | 40.463 | 249.247 | 196.23 |
| 12506 | C | C | SER | 912 | 183.474 | 41.245 | 249.692 | 196.23 |
| 12507 | O | O | SER | 912 | 184.38 | 40.687 | 250.322 | 196.23 |
| 12508 | C | CB | SER | 912 | 182.565 | 39.595 | 248.031 | 196.23 |
| 12509 | O | OG | SER | 912 | 183.598 | 38.67 | 248.325 | 196.23 |
| 12510 | N | N | LEU | 913 | 183.527 | 42.532 | 249.364 | 193.04 |
| 12511 | C | CA | LEU | 913 | 184.565 | 43.395 | 249.905 | 193.04 |
| 12512 | C | C | LEU | 913 | 184.438 | 43.452 | 251.426 | 193.04 |
| 12513 | O | O | LEU | 913 | 183.33 | 43.625 | 251.949 | 193.04 |
| 12514 | C | CB | LEU | 913 | 184.454 | 44.8 | 249.307 | 193.04 |
| 12515 | C | CG | LEU | 913 | 184.867 | 46.005 | 250.155 | 193.04 |
| 12516 | C | CD1 | LEU | 913 | 186.378 | 46.122 | 250.271 | 193.04 |
| 12517 | C | CD2 | LEU | 913 | 184.27 | 47.282 | 249.581 | 193.04 |
| 12518 | N | N | PRO | 914 | 185.537 | 43.285 | 252.162 | 189.69 |
| 12519 | C | CA | PRO | 914 | 185.454 | 43.329 | 253.627 | 189.69 |
| 12520 | C | C | PRO | 914 | 184.903 | 44.66 | 254.117 | 189.69 |
| 12521 | O | O | PRO | 914 | 185.238 | 45.724 | 253.594 | 189.69 |
| 12522 | C | CB | PRO | 914 | 186.907 | 43.123 | 254.068 | 189.69 |
| 12523 | C | CG | PRO | 914 | 187.537 | 42.378 | 252.939 | 189.69 |
| 12524 | C | CD | PRO | 914 | 186.878 | 42.895 | 251.693 | 189.69 |
| 12525 | N | N | GLU | 915 | 184.04 | 44.584 | 255.129 | 187.9 |
| 12526 | C | CA | GLU | 915 | 183.457 | 45.797 | 255.695 | 187.9 |
| 12527 | C | C | GLU | 915 | 184.491 | 46.738 | 256.306 | 187.9 |
| 12528 | O | O | GLU | 915 | 184.396 | 47.952 | 256.053 | 187.9 |
| 12529 | C | CB | GLU | 915 | 182.376 | 45.427 | 256.72 | 187.9 |
| 12530 | C | CG | GLU | 915 | 180.965 | 45.811 | 256.299 | 187.9 |
| 12531 | C | CD | GLU | 915 | 180.762 | 47.314 | 256.24 | 187.9 |
| 12532 | O | OE1 | GLU | 915 | 181.457 | 48.039 | 256.982 | 187.9 |
| 12533 | O | OE2 | GLU | 915 | 179.91 | 47.771 | 255.449 | 187.9 |
| 12534 | N | N | PRO | 916 | 185.463 | 46.283 | 257.112 | 186.35 |
| 12535 | C | CA | PRO | 916 | 186.494 | 47.227 | 257.582 | 186.35 |
| 12536 | C | C | PRO | 916 | 187.258 | 47.891 | 256.451 | 186.35 |
| 12537 | O | O | PRO | 916 | 187.575 | 49.084 | 256.542 | 186.35 |
| 12538 | C | CB | PRO | 916 | 187.405 | 46.345 | 258.448 | 186.35 |
| 12539 | C | CG | PRO | 916 | 186.53 | 45.233 | 258.896 | 186.35 |
| 12540 | C | CD | PRO | 916 | 185.627 | 44.955 | 257.733 | 186.35 |
| 12541 | N | N | GLU | 917 | 187.561 | 47.152 | 255.381 | 185.87 |
| 12542 | C | CA | GLU | 917 | 188.178 | 47.77 | 254.213 | 185.87 |
| 12543 | C | C | GLU | 917 | 187.211 | 48.734 | 253.538 | 185.87 |
| 12544 | O | O | GLU | 917 | 187.618 | 49.79 | 253.039 | 185.87 |
| 12545 | C | CB | GLU | 917 | 188.645 | 46.696 | 253.23 | 185.87 |
| 12546 | C | CG | GLU | 917 | 189.384 | 47.24 | 252.016 | 185.87 |
| 12547 | C | CD | GLU | 917 | 190.717 | 47.87 | 252.372 | 185.87 |
| 12548 | O | OE1 | GLU | 917 | 191.288 | 47.51 | 253.423 | 185.87 |
| 12549 | O | OE2 | GLU | 917 | 191.194 | 48.729 | 251.6 | 185.87 |
| 12550 | N | N | ARG | 918 | 185.924 | 48.382 | 253.509 | 185.54 |
| 12551 | C | CA | ARG | 918 | 184.914 | 49.303 | 253.002 | 185.54 |
| 12552 | C | C | ARG | 918 | 184.826 | 50.549 | 253.873 | 185.54 |
| 12553 | O | O | ARG | 918 | 184.604 | 51.656 | 253.369 | 185.54 |
| 12554 | C | CB | ARG | 918 | 183.56 | 48.599 | 252.931 | 185.54 |
| 12555 | C | CG | ARG | 918 | 182.508 | 49.333 | 252.122 | 185.54 |
| 12556 | C | CD | ARG | 918 | 181.178 | 48.605 | 252.195 | 185.54 |
| 12557 | N | NE | ARG | 918 | 181.339 | 47.166 | 252.028 | 185.54 |
| 12558 | C | CZ | ARG | 918 | 180.35 | 46.286 | 252.107 | 185.54 |
| 12559 | N | NH1 | ARG | 918 | 179.104 | 46.666 | 252.34 | 185.54 |
| 12560 | N | NH2 | ARG | 918 | 180.619 | 44.993 | 251.951 | 185.54 |
| 12561 | N | N | ASN | 919 | 184.999 | 50.387 | 255.187 | 185.11 |
| 12562 | C | CA | ASN | 919 | 184.935 | 51.519 | 256.103 | 185.11 |
| 12563 | C | C | ASN | 919 | 186.167 | 52.412 | 256.021 | 185.11 |
| 12564 | O | O | ASN | 919 | 186.176 | 53.485 | 256.633 | 185.11 |
| 12565 | C | CB | ASN | 919 | 184.745 | 51.023 | 257.537 | 185.11 |
| 12566 | C | CG | ASN | 919 | 183.399 | 50.36 | 257.749 | 185.11 |
| 12567 | O | OD1 | ASN | 919 | 182.402 | 50.745 | 257.137 | 185.11 |
| 12568 | N | ND2 | ASN | 919 | 183.364 | 49.357 | 258.617 | 185.11 |
| 12569 | N | N | TYR | 920 | 187.205 | 51.993 | 255.291 | 174.44 |
| 12570 | C | CA | TYR | 920 | 188.397 | 52.826 | 255.156 | 174.44 |
| 12571 | C | C | TYR | 920 | 188.07 | 54.157 | 254.491 | 174.44 |
| 12572 | O | O | TYR | 920 | 188.55 | 55.211 | 254.925 | 174.44 |
| 12573 | C | CB | TYR | 920 | 189.472 | 52.082 | 254.363 | 174.44 |
| 12574 | C | CG | TYR | 920 | 190.567 | 51.489 | 255.219 | 174.44 |
| 12575 | C | CD1 | TYR | 920 | 190.374 | 50.294 | 255.899 | 174.44 |
| 12576 | C | CD2 | TYR | 920 | 191.795 | 52.123 | 255.343 | 174.44 |
| 12577 | C | CE1 | TYR | 920 | 191.374 | 49.749 | 256.681 | 174.44 |
| 12578 | C | CE2 | TYR | 920 | 192.801 | 51.586 | 256.123 | 174.44 |
| 12579 | C | CZ | TYR | 920 | 192.585 | 50.399 | 256.789 | 174.44 |
| 12580 | O | OH | TYR | 920 | 193.584 | 49.86 | 257.567 | 174.44 |
| 12581 | N | N | ASN | 921 | 187.256 | 54.131 | 253.438 | 175.01 |
| 12582 | C | CA | ASN | 921 | 186.814 | 55.343 | 252.765 | 175.01 |
| 12583 | C | C | ASN | 921 | 185.465 | 55.835 | 253.271 | 175.01 |
| 12584 | O | O | ASN | 921 | 184.943 | 56.823 | 252.745 | 175.01 |
| 12585 | C | CB | ASN | 921 | 186.755 | 55.121 | 251.253 | 175.01 |
| 12586 | C | CG | ASN | 921 | 188.119 | 54.869 | 250.648 | 175.01 |
| 12587 | O | OD1 | ASN | 921 | 189.147 | 55.147 | 251.266 | 175.01 |
| 12588 | N | ND2 | ASN | 921 | 188.137 | 54.349 | 249.426 | 175.01 |
| 12589 | N | N | LEU | 922 | 184.888 | 55.168 | 254.275 | 171.93 |
| 12590 | C | CA | LEU | 922 | 183.626 | 55.637 | 254.84 | 171.93 |
| 12591 | C | C | LEU | 922 | 183.787 | 57.018 | 255.459 | 171.93 |
| 12592 | O | O | LEU | 922 | 182.873 | 57.848 | 255.39 | 171.93 |
| 12593 | C | CB | LEU | 922 | 183.105 | 54.641 | 255.876 | 171.93 |
| 12594 | C | CG | LEU | 922 | 181.742 | 54.006 | 255.589 | 171.93 |
| 12595 | C | CD1 | LEU | 922 | 180.747 | 55.057 | 255.122 | 171.93 |
| 12596 | C | CD2 | LEU | 922 | 181.86 | 52.882 | 254.573 | 171.93 |
| 12597 | N | N | GLN | 923 | 184.941 | 57.279 | 256.077 | 165.29 |
| 12598 | C | CA | GLN | 923 | 185.239 | 58.631 | 256.536 | 165.29 |
| 12599 | C | C | GLN | 923 | 185.272 | 59.605 | 255.367 | 165.29 |
| 12600 | O | O | GLN | 923 | 184.687 | 60.692 | 255.438 | 165.29 |
| 12601 | C | CB | GLN | 923 | 186.568 | 58.65 | 257.29 | 165.29 |
| 12602 | C | CG | GLN | 923 | 186.476 | 58.191 | 258.735 | 165.29 |
| 12603 | C | CD | GLN | 923 | 185.877 | 59.247 | 259.645 | 165.29 |
| 12604 | O | OE1 | GLN | 923 | 185.682 | 60.394 | 259.241 | 165.29 |
| 12605 | N | NE2 | GLN | 923 | 185.586 | 58.866 | 260.883 | 165.29 |
| 12606 | N | N | MET | 924 | 185.949 | 59.231 | 254.279 | 162.31 |
| 12607 | C | CA | MET | 924 | 185.94 | 60.062 | 253.08 | 162.31 |
| 12608 | C | C | MET | 924 | 184.549 | 60.123 | 252.461 | 162.31 |
| 12609 | O | O | MET | 924 | 184.11 | 61.189 | 252.012 | 162.31 |
| 12610 | C | CB | MET | 924 | 186.955 | 59.536 | 252.067 | 162.31 |
| 12611 | C | CG | MET | 924 | 188.403 | 59.747 | 252.473 | 162.31 |
| 12612 | S | SD | MET | 924 | 188.844 | 61.492 | 252.569 | 162.31 |
| 12613 | C | CE | MET | 924 | 188.513 | 62.019 | 250.889 | 162.31 |
| 12614 | N | N | SER | 925 | 183.841 | 58.991 | 252.429 | 164.42 |
| 12615 | C | CA | SER | 925 | 182.497 | 58.972 | 251.861 | 164.42 |
| 12616 | C | C | SER | 925 | 181.551 | 59.861 | 252.658 | 164.42 |
| 12617 | O | O | SER | 925 | 180.67 | 60.513 | 252.085 | 164.42 |
| 12618 | C | CB | SER | 925 | 181.967 | 57.54 | 251.804 | 164.42 |
| 12619 | O | OG | SER | 925 | 180.67 | 57.498 | 251.235 | 164.42 |
| 12620 | N | N | GLY | 926 | 181.713 | 59.892 | 253.981 | 161.12 |
| 12621 | C | CA | GLY | 926 | 180.91 | 60.794 | 254.79 | 161.12 |
| 12622 | C | C | GLY | 926 | 181.162 | 62.251 | 254.457 | 161.12 |
| 12623 | O | O | GLY | 926 | 180.236 | 63.065 | 254.447 | 161.12 |
| 12624 | N | N | GLU | 927 | 182.42 | 62.6 | 254.179 | 155.43 |
| 12625 | C | CA | GLU | 927 | 182.742 | 63.978 | 253.827 | 155.43 |
| 12626 | C | C | GLU | 927 | 182.187 | 64.346 | 252.457 | 155.43 |
| 12627 | O | O | GLU | 927 | 181.823 | 65.504 | 252.223 | 155.43 |
| 12628 | C | CB | GLU | 927 | 184.255 | 64.195 | 253.868 | 155.43 |
| 12629 | C | CG | GLU | 927 | 184.91 | 63.814 | 255.188 | 155.43 |
| 12630 | C | CD | GLU | 927 | 184.351 | 64.581 | 256.371 | 155.43 |
| 12631 | O | OE1 | GLU | 927 | 183.97 | 65.757 | 256.196 | 155.43 |
| 12632 | O | OE2 | GLU | 927 | 184.29 | 64.006 | 257.478 | 155.43 |
| 12633 | N | N | THR | 928 | 182.117 | 63.379 | 251.538 | 155.4 |
| 12634 | C | CA | THR | 928 | 181.656 | 63.674 | 250.184 | 155.4 |
| 12635 | C | C | THR | 928 | 180.231 | 64.212 | 250.193 | 155.4 |
| 12636 | O | O | THR | 928 | 179.925 | 65.204 | 249.522 | 155.4 |
| 12637 | C | CB | THR | 928 | 181.75 | 62.424 | 249.308 | 155.4 |
| 12638 | O | OG1 | THR | 928 | 181.095 | 61.329 | 249.96 | 155.4 |
| 12639 | C | CG2 | THR | 928 | 183.203 | 62.063 | 249.046 | 155.4 |
| 12640 | N | N | LEU | 929 | 179.343 | 63.569 | 250.952 | 153.49 |
| 12641 | C | CA | LEU | 929 | 177.996 | 64.107 | 251.112 | 153.49 |
| 12642 | C | C | LEU | 929 | 178.006 | 65.35 | 251.989 | 153.49 |
| 12643 | O | O | LEU | 929 | 177.235 | 66.288 | 251.757 | 153.49 |
| 12644 | C | CB | LEU | 929 | 177.067 | 63.044 | 251.694 | 153.49 |
| 12645 | C | CG | LEU | 929 | 176.9 | 61.78 | 250.852 | 153.49 |
| 12646 | C | CD | LEU | 929 | 175.757 | 60.939 | 251.384 | 153.49 |
| 12647 | C | CD2 | LEU | 929 | 176.673 | 62.133 | 249.393 | 153.49 |
| 12648 | N | N | LYS | 930 | 178.873 | 65.373 | 253.003 | 148.29 |
| 12649 | C | CA | LYS | 930 | 178.954 | 66.531 | 253.887 | 148.29 |
| 12650 | C | C | LYS | 930 | 179.426 | 67.768 | 253.135 | 148.29 |
| 12651 | O | O | LYS | 930 | 178.936 | 68.877 | 253.379 | 148.29 |
| 12652 | C | CB | LYS | 930 | 179.888 | 66.226 | 255.056 | 148.29 |
| 12653 | C | CG | LYS | 930 | 179.613 | 67.041 | 256.304 | 148.29 |
| 12654 | C | CD | LYS | 930 | 180.683 | 66.8 | 257.355 | 148.29 |
| 12655 | C | CE | LYS | 930 | 180.974 | 65.316 | 257.524 | 148.29 |
| 12656 | N | NZ | LYS | 930 | 179.783 | 64.559 | 257.992 | 148.29 |
| 12657 | N | N | THR | 931 | 180.384 | 67.6 | 252.219 | 148.25 |
| 12658 | C | CA | THR | 931 | 180.882 | 68.739 | 251.454 | 148.25 |
| 12659 | C | C | THR | 931 | 179.796 | 69.323 | 250.56 | 148.25 |
| 12660 | O | O | THR | 931 | 179.815 | 70.52 | 250.251 | 148.25 |
| 12661 | C | CB | THR | 931 | 182.098 | 68.332 | 250.624 | 148.25 |
| 12662 | O | OG1 | THR | 931 | 181.843 | 67.077 | 249.98 | 148.25 |
| 12663 | C | CG2 | THR | 931 | 183.325 | 68.21 | 251.513 | 148.25 |
| 12664 | N | N | LEU | 932 | 178.843 | 68.494 | 250.129 | 146.66 |
| 12665 | C | CA | LEU | 932 | 177.701 | 69.015 | 249.387 | 146.66 |
| 12666 | C | C | LEU | 932 | 176.921 | 70.014 | 250.23 | 146.66 |
| 12667 | O | O | LEU | 932 | 176.562 | 71.098 | 249.757 | 146.66 |
| 12668 | C | CB | LEU | 932 | 176.799 | 67.865 | 248.94 | 146.66 |
| 12669 | C | CG | LEU | 932 | 177.392 | 66.928 | 247.89 | 146.66 |
| 12670 | C | CD1 | LEU | 932 | 176.56 | 65.666 | 247.772 | 146.66 |
| 12671 | C | CD2 | LEU | 932 | 177.478 | 67.641 | 246.553 | 146.66 |
| 12672 | N | N | LEU | 933 | 176.658 | 69.666 | 251.491 | 147.38 |
| 12673 | C | CA | LEU | 933 | 176.038 | 70.617 | 252.405 | 147.38 |
| 12674 | C | C | LEU | 933 | 176.984 | 71.77 | 252.719 | 147.38 |
| 12675 | O | O | LEU | 933 | 176.554 | 72.924 | 252.827 | 147.38 |
| 12676 | C | CB | LEU | 933 | 175.612 | 69.905 | 253.687 | 147.38 |
| 12677 | C | CG | LEU | 933 | 174.576 | 68.793 | 253.522 | 147.38 |
| 12678 | C | CD1 | LEU | 933 | 174.398 | 68.029 | 254.823 | 147.38 |
| 12679 | C | CD2 | LEU | 933 | 173.251 | 69.371 | 253.055 | 147.38 |
| 12680 | N | N | ALA | 934 | 178.276 | 71.473 | 252.875 | 143.59 |
| 12681 | C | CA | ALA | 934 | 179.245 | 72.515 | 253.202 | 143.59 |
| 12682 | C | C | ALA | 934 | 179.376 | 73.528 | 252.072 | 143.59 |
| 12683 | O | O | ALA | 934 | 179.464 | 74.737 | 252.318 | 143.59 |
| 12684 | C | CB | ALA | 934 | 180.601 | 71.889 | 253.521 | 143.59 |
| 12685 | N | N | LEU | 935 | 179.393 | 73.056 | 250.823 | 144.48 |
| 12686 | C | CA | LEU | 935 | 179.517 | 73.964 | 249.689 | 144.48 |
| 12687 | C | C | LEU | 935 | 178.264 | 74.804 | 249.478 | 144.48 |
| 12688 | O | O | LEU | 935 | 178.296 | 75.758 | 248.694 | 144.48 |
| 12689 | C | CB | LEU | 935 | 179.84 | 73.18 | 248.415 | 144.48 |
| 12690 | C | CG | LEU | 935 | 181.316 | 73.043 | 248.027 | 144.48 |
| 12691 | C | CD1 | LEU | 935 | 182.142 | 72.456 | 249.162 | 144.48 |
| 12692 | C | CD2 | LEU | 935 | 181.46 | 72.2 | 246.769 | 144.48 |
| 12693 | N | N | GLY | 936 | 177.168 | 74.476 | 250.158 | 150.57 |
| 12694 | C | CA | GLY | 936 | 175.926 | 75.206 | 250.019 | 150.57 |
| 12695 | C | C | GLY | 936 | 174.896 | 74.549 | 249.13 | 150.57 |
| 12696 | O | O | GLY | 936 | 173.816 | 75.121 | 248.936 | 150.57 |
| 12697 | N | N | CYS | 937 | 175.192 | 73.374 | 248.583 | 162.55 |
| 12698 | C | CA | CYS | 937 | 174.234 | 72.683 | 247.733 | 162.55 |
| 12699 | C | C | CYS | 937 | 173.042 | 72.2 | 248.549 | 162.55 |
| 12700 | O | O | CYS | 937 | 173.194 | 71.685 | 249.66 | 162.55 |
| 12701 | C | CB | CYS | 937 | 174.906 | 71.503 | 247.033 | 162.55 |
| 12702 | S | SG | CYS | 937 | 173.758 | 70.276 | 246.375 | 162.55 |
| 12703 | N | N | HIS | 938 | 171.848 | 72.372 | 247.988 | 161.04 |
| 12704 | C | CA | HIS | 938 | 170.624 | 71.876 | 248.6 | 161.04 |
| 12705 | C | C | HIS | 938 | 170.348 | 70.465 | 248.1 | 161.04 |
| 12706 | O | O | HIS | 938 | 170.102 | 70.259 | 246.907 | 161.04 |
| 12707 | C | CB | HIS | 938 | 169.448 | 72.799 | 248.279 | 161.04 |
| 12708 | C | CG | HIS | 938 | 168.108 | 72.18 | 248.524 | 161.04 |
| 12709 | N | ND1 | HIS | 938 | 167.3 | 71.728 | 247.502 | 161.04 |
| 12710 | C | CD2 | HIS | 938 | 167.431 | 71.941 | 249.672 | 161.04 |
| 12711 | C | CE1 | HIS | 938 | 166.184 | 71.237 | 248.011 | 161.04 |
| 12712 | N | NE2 | HIS | 938 | 166.239 | 71.353 | 249.325 | 161.04 |
| 12713 | N | N | VAL | 939 | 170.393 | 69.497 | 249.013 | 166.12 |
| 12714 | C | CA | VAL | 939 | 170.246 | 68.09 | 248.669 | 166.12 |
| 12715 | C | C | VAL | 939 | 169.271 | 67.44 | 249.64 | 166.12 |
| 12716 | O | O | VAL | 939 | 169.318 | 67.673 | 250.851 | 166.12 |
| 12717 | C | CB | VAL | 939 | 171.609 | 67.356 | 248.674 | 166.12 |
| 12718 | C | CG1 | VAL | 939 | 172.366 | 67.64 | 249.961 | 166.12 |
| 12719 | C | CG2 | VAL | 939 | 171.418 | 65.859 | 248.481 | 166.12 |
| 12720 | N | N | GLY | 940 | 168.372 | 66.627 | 249.091 | 172.75 |
| 12721 | C | CA | GLY | 940 | 167.414 | 65.888 | 249.891 | 172.75 |
| 12722 | C | C | GLY | 940 | 166.979 | 64.641 | 249.157 | 172.75 |
| 12723 | O | O | GLY | 940 | 167.3 | 64.437 | 247.984 | 172.75 |
| 12724 | N | N | MET | 941 | 166.235 | 63.797 | 249.872 | 178.41 |
| 12725 | C | CA | MET | 941 | 165.727 | 62.545 | 249.311 | 178.41 |
| 12726 | C | C | MET | 941 | 164.506 | 62.866 | 248.453 | 178.41 |
| 12727 | O | O | MET | 941 | 163.353 | 62.661 | 248.841 | 178.41 |
| 12728 | C | CB | MET | 941 | 165.398 | 61.551 | 250.416 | 178.41 |
| 12729 | C | CG | MET | 941 | 165.021 | 60.166 | 249.914 | 178.41 |
| 12730 | S | SD | MET | 941 | 164.582 | 59.038 | 251.249 | 178.41 |
| 12731 | C | CE | MET | 941 | 163.193 | 59.902 | 251.978 | 178.41 |
| 12732 | N | N | ALA | 942 | 164.777 | 63.395 | 247.257 | 180.73 |
| 12733 | C | CA | ALA | 942 | 163.697 | 63.737 | 246.339 | 180.73 |
| 12734 | C | C | ALA | 942 | 162.947 | 62.494 | 245.879 | 180.73 |
| 12735 | O | O | ALA | 942 | 161.713 | 62.499 | 245.796 | 180.73 |
| 12736 | C | CB | ALA | 942 | 164.249 | 64.501 | 245.137 | 180.73 |
| 12737 | N | N | ASP | 943 | 163.671 | 61.419 | 245.578 | 184.69 |
| 12738 | C | CA | ASP | 943 | 163.081 | 60.187 | 245.075 | 184.69 |
| 12739 | C | C | ASP | 943 | 163.534 | 59.012 | 245.929 | 184.69 |
| 12740 | O | O | ASP | 943 | 164.733 | 58.843 | 246.177 | 184.69 |
| 12741 | C | CB | ASP | 943 | 163.464 | 59.952 | 243.611 | 184.69 |
| 12742 | C | CG | ASP | 943 | 162.877 | 60.995 | 242.68 | 184.69 |
| 12743 | O | OD1 | ASP | 943 | 163.286 | 62.172 | 242.768 | 184.69 |
| 12744 | O | OD2 | ASP | 943 | 162.008 | 60.637 | 241.858 | 184.69 |
| 12745 | N | N | GLU | 944 | 162.572 | 58.206 | 246.375 | 188.57 |
| 12746 | C | CA | GLU | 944 | 162.855 | 56.957 | 247.069 | 188.57 |
| 12747 | C | C | GLU | 944 | 162.595 | 55.734 | 246.203 | 188.57 |
| 12748 | O | O | GLU | 944 | 163.288 | 54.722 | 246.349 | 188.57 |
| 12749 | C | CB | GLU | 944 | 162.017 | 56.862 | 248.351 | 188.57 |
| 12750 | C | CG | GLU | 944 | 162.238 | 55.601 | 249.182 | 188.57 |
| 12751 | C | CD | GLU | 944 | 163.486 | 55.663 | 250.049 | 188.57 |
| 12752 | O | OE1 | GLU | 944 | 164.486 | 56.285 | 249.632 | 188.57 |
| 12753 | O | OE2 | GLU | 944 | 163.464 | 55.087 | 251.156 | 188.57 |
| 12754 | N | N | LYS | 945 | 161.615 | 55.813 | 245.299 | 185.13 |
| 12755 | C | CA | LYS | 945 | 161.359 | 54.712 | 244.377 | 185.13 |
| 12756 | C | C | LYS | 945 | 162.521 | 54.52 | 243.411 | 185.13 |
| 12757 | O | O | LYS | 945 | 162.786 | 53.398 | 242.962 | 185.13 |
| 12758 | C | CB | LYS | 945 | 160.057 | 54.955 | 243.612 | 185.13 |
| 12759 | C | CG | LYS | 945 | 159.355 | 56.268 | 243.947 | 185.13 |
| 12760 | C | CD | LYS | 945 | 159.934 | 57.446 | 243.17 | 185.13 |
| 12761 | C | CE | LYS | 945 | 159.285 | 58.755 | 243.592 | 185.13 |
| 12762 | N | NZ | LYS | 945 | 159.874 | 59.923 | 242.88 | 185.13 |
| 12763 | N | N | ALA | 946 | 163.222 | 55.606 | 243.073 | 188.97 |
| 12764 | C | CA | ALA | 946 | 164.381 | 55.496 | 242.193 | 188.97 |
| 12765 | C | C | ALA | 946 | 165.483 | 54.663 | 242.835 | 188.97 |
| 12766 | O | O | ALA | 946 | 166.158 | 53.882 | 242.154 | 188.97 |
| 12767 | C | CB | ALA | 946 | 164.9 | 56.887 | 241.828 | 188.97 |
| 12768 | N | N | GLU | 947 | 165.689 | 54.824 | 244.144 | 187.7 |
| 12769 | C | CA | GLU | 947 | 166.666 | 53.998 | 244.846 | 187.7 |
| 12770 | C | C | GLU | 947 | 166.252 | 52.532 | 244.841 | 187.7 |
| 12771 | O | O | GLU | 947 | 167.096 | 51.641 | 244.692 | 187.7 |
| 12772 | C | CB | GLU | 947 | 166.844 | 54.501 | 246.279 | 187.7 |
| 12773 | C | CG | GLU | 947 | 167.713 | 53.607 | 247.152 | 187.7 |
| 12774 | C | CD | GLU | 947 | 167.986 | 54.211 | 248.515 | 187.7 |
| 12775 | O | OE1 | GLU | 947 | 167.614 | 55.384 | 248.73 | 187.7 |
| 12776 | O | OE2 | GLU | 947 | 168.572 | 53.515 | 249.371 | 187.7 |
| 12777 | N | N | ASP | 948 | 164.953 | 52.264 | 245.002 | 189.72 |
| 12778 | C | CA | ASP | 948 | 164.48 | 50.886 | 245.076 | 189.72 |
| 12779 | C | C | ASP | 948 | 164.746 | 50.13 | 243.78 | 189.72 |
| 12780 | O | O | ASP | 948 | 165.189 | 48.976 | 243.806 | 189.72 |
| 12781 | C | CB | ASP | 948 | 162.989 | 50.864 | 245.411 | 189.72 |
| 12782 | C | CG | ASP | 948 | 162.668 | 51.609 | 246.692 | 189.72 |
| 12783 | O | OD1 | ASP | 948 | 163.531 | 51.646 | 247.594 | 189.72 |
| 12784 | O | OD2 | ASP | 948 | 161.551 | 52.158 | 246.796 | 189.72 |
| 12785 | N | N | ASN | 949 | 164.488 | 50.76 | 242.636 | 188.29 |
| 12786 | C | CA | ASN | 949 | 164.633 | 50.115 | 241.336 | 188.29 |
| 12787 | C | C | ASN | 949 | 165.672 | 50.861 | 240.513 | 188.29 |
| 12788 | O | O | ASN | 949 | 165.478 | 52.035 | 240.179 | 188.29 |
| 12789 | C | CB | ASN | 949 | 163.295 | 50.069 | 240.595 | 188.29 |
| 12790 | C | CG | ASN | 949 | 163.425 | 49.504 | 239.195 | 188.29 |
| 12791 | O | OD1 | ASN | 949 | 163.608 | 50.245 | 238.229 | 188.29 |
| 12792 | N | ND2 | ASN | 949 | 163.334 | 48.185 | 239.078 | 188.29 |
| 12793 | N | N | LEU | 950 | 166.761 | 50.174 | 240.175 | 187.01 |
| 12794 | C | CA | LEU | 950 | 167.81 | 50.738 | 239.339 | 187.01 |
| 12795 | C | C | LEU | 950 | 168.642 | 49.603 | 238.759 | 187.01 |
| 12796 | O | O | LEU | 950 | 168.844 | 48.571 | 239.404 | 187.01 |
| 12797 | C | CB | LEU | 950 | 168.692 | 51.719 | 240.125 | 187.01 |
| 12798 | C | CG | LEU | 950 | 169.095 | 51.352 | 241.557 | 187.01 |
| 12799 | C | CD1 | LEU | 950 | 170.301 | 50.422 | 241.59 | 187.01 |
| 12800 | C | CD2 | LEU | 950 | 169.366 | 52.61 | 242.364 | 187.01 |
| 12801 | N | N | LYS | 951 | 169.112 | 49.799 | 237.531 | 183.4 |
| 12802 | C | CA | LYS | 951 | 169.935 | 48.789 | 236.883 | 183.4 |
| 12803 | C | C | LYS | 951 | 171.317 | 48.734 | 237.525 | 183.4 |
| 12804 | O | O | LYS | 951 | 171.806 | 49.717 | 238.087 | 183.4 |
| 12805 | C | CB | LYS | 951 | 170.065 | 49.077 | 235.388 | 183.4 |
| 12806 | C | CG | LYS | 951 | 169.932 | 47.843 | 234.51 | 183.4 |
| 12807 | C | CD | LYS | 951 | 170.816 | 47.932 | 233.277 | 183.4 |
| 12808 | C | CE | LYS | 951 | 172.28 | 47.754 | 233.643 | 183.4 |
| 12809 | N | NZ | LYS | 951 | 173.171 | 47.821 | 232.453 | 183.4 |
| 12810 | N | N | LYS | 952 | 171.945 | 47.564 | 237.442 | 185.22 |
| 12811 | C | CA | LYS | 952 | 173.282 | 47.342 | 237.98 | 185.22 |
| 12812 | C | C | LYS | 952 | 174.216 | 46.913 | 236.856 | 185.22 |
| 12813 | O | O | LYS | 952 | 174.048 | 45.831 | 236.284 | 185.22 |
| 12814 | C | CB | LYS | 952 | 173.259 | 46.285 | 239.085 | 185.22 |
| 12815 | C | CG | LYS | 952 | 172.789 | 46.802 | 240.432 | 185.22 |
| 12816 | C | CD | LYS | 952 | 173.74 | 47.86 | 240.967 | 185.22 |
| 12817 | C | CE | LYS | 952 | 173.383 | 48.26 | 242.388 | 185.22 |
| 12818 | N | NZ | LYS | 952 | 173.509 | 47.114 | 243.332 | 185.22 |
| 12819 | N | N | THR | 953 | 175.196 | 47.758 | 236.545 | 188.55 |
| 12820 | C | CA | THR | 953 | 176.24 | 47.375 | 235.605 | 188.55 |
| 12821 | C | C | THR | 953 | 177.181 | 46.369 | 236.258 | 188.55 |
| 12822 | O | O | THR | 953 | 177.597 | 46.54 | 237.405 | 188.55 |
| 12823 | C | CB | THR | 953 | 177.02 | 48.607 | 235.137 | 188.55 |
| 12824 | O | OG1 | THR | 953 | 176.2 | 49.391 | 234.261 | 188.55 |
| 12825 | C | CG2 | THR | 953 | 178.294 | 48.205 | 234.402 | 188.55 |
| 12826 | N | N | LYS | 954 | 177.504 | 45.305 | 235.526 | 195.24 |
| 12827 | C | CA | LYS | 954 | 178.403 | 44.273 | 236.015 | 195.24 |
| 12828 | C | C | LYS | 954 | 179.589 | 44.13 | 235.074 | 195.24 |
| 12829 | O | O | LYS | 954 | 179.473 | 44.352 | 233.865 | 195.24 |
| 12830 | C | CB | LYS | 954 | 177.694 | 42.919 | 236.166 | 195.24 |
| 12831 | C | CG | LYS | 954 | 176.93 | 42.758 | 237.47 | 195.24 |
| 12832 | C | CD | LYS | 954 | 176.559 | 41.303 | 237.714 | 195.24 |
| 12833 | C | CE | LYS | 954 | 175.981 | 41.105 | 239.107 | 195.24 |
| 12834 | N | NZ | LYS | 954 | 175.757 | 39.667 | 239.427 | 195.24 |
| 12835 | N | N | LEU | 955 | 180.732 | 43.764 | 235.645 | 198.53 |
| 12836 | C | CA | LEU | 955 | 181.925 | 43.553 | 234.841 | 198.53 |
| 12837 | C | C | LEU | 955 | 181.701 | 42.396 | 233.871 | 198.53 |
| 12838 | O | O | LEU | 955 | 181.058 | 41.402 | 234.228 | 198.53 |
| 12839 | C | CB | LEU | 955 | 183.134 | 43.26 | 235.73 | 198.53 |
| 12840 | C | CG | LEU | 955 | 183.954 | 44.466 | 236.186 | 198.53 |
| 12841 | C | CD1 | LEU | 955 | 185.098 | 44.033 | 237.091 | 198.53 |
| 12842 | C | CD2 | LEU | 955 | 184.481 | 45.226 | 234.98 | 198.53 |
| 12843 | N | N | PRO | 956 | 182.2 | 42.496 | 232.641 | 200.65 |
| 12844 | C | CA | PRO | 956 | 182.055 | 41.384 | 231.696 | 200.65 |
| 12845 | C | C | PRO | 956 | 182.746 | 40.13 | 232.21 | 200.65 |
| 12846 | O | O | PRO | 956 | 183.767 | 40.196 | 232.897 | 200.65 |
| 12847 | C | CB | PRO | 956 | 182.721 | 41.913 | 230.42 | 200.65 |
| 12848 | C | CG | PRO | 956 | 182.667 | 43.401 | 230.553 | 200.65 |
| 12849 | C | CD | PRO | 956 | 182.824 | 43.676 | 232.018 | 200.65 |
| 12850 | N | N | LYS | 957 | 182.163 | 38.977 | 231.876 | 198.41 |
| 12851 | C | CA | LYS | 957 | 182.756 | 37.703 | 232.268 | 198.41 |
| 12852 | C | C | LYS | 957 | 184.141 | 37.529 | 231.658 | 198.41 |
| 12853 | O | O | LYS | 957 | 185.031 | 36.934 | 232.278 | 198.41 |
| 12854 | C | CB | LYS | 957 | 181.838 | 36.548 | 231.864 | 198.41 |
| 12855 | C | CG | LYS | 957 | 180.975 | 36.833 | 230.642 | 198.41 |
| 12856 | C | CD | LYS | 957 | 179.586 | 37.312 | 231.045 | 198.41 |
| 12857 | C | CE | LYS | 957 | 178.847 | 37.927 | 229.868 | 198.41 |
| 12858 | N | NZ | LYS | 957 | 179.512 | 39.171 | 229.39 | 198.41 |
| 12859 | N | N | THR | 958 | 184.341 | 38.036 | 230.439 | 200.24 |
| 12860 | C | CA | THR | 958 | 185.667 | 37.995 | 229.832 | 200.24 |
| 12861 | C | C | THR | 958 | 186.664 | 38.831 | 230.627 | 200.24 |
| 12862 | C | O | THR | 958 | 187.815 | 38.421 | 230.819 | 200.24 |
| 12863 | C | CB | THR | 958 | 185.599 | 38.47 | 228.378 | 200.24 |
| 12864 | O | OG1 | THR | 958 | 186.92 | 38.755 | 227.902 | 200.24 |
| 12865 | C | CG2 | THR | 958 | 184.731 | 39.715 | 228.252 | 200.24 |
| 12866 | N | N | TYR | 959 | 186.244 | 40.005 | 231.098 | 200.56 |
| 12867 | C | CA | TYR | 959 | 187.106 | 40.865 | 231.911 | 200.56 |
| 12868 | C | C | TYR | 959 | 186.881 | 40.549 | 233.39 | 200.56 |
| 12869 | O | O | TYR | 959 | 186.36 | 41.349 | 234.17 | 200.56 |
| 12870 | C | CB | TYR | 959 | 186.843 | 42.333 | 231.599 | 200.56 |
| 12871 | C | CG | TYR | 959 | 188.099 | 43.173 | 231.532 | 200.56 |
| 12872 | C | CD1 | TYR | 959 | 189.322 | 42.661 | 231.945 | 200.56 |
| 12873 | C | CD2 | TYR | 959 | 188.062 | 44.477 | 231.055 | 200.56 |
| 12874 | C | CE1 | TYR | 959 | 190.473 | 43.422 | 231.884 | 200.56 |
| 12875 | C | CE2 | TYR | 959 | 189.21 | 45.247 | 230.991 | 200.56 |
| 12876 | C | CZ | TYR | 959 | 190.411 | 44.714 | 231.407 | 200.56 |
| 12877 | O | OH | TYR | 959 | 191.557 | 45.473 | 231.346 | 200.56 |
| 12878 | N | N | MET | 960 | 187.3 | 39.345 | 233.768 | 200.52 |
| 12879 | C | CA | MET | 960 | 187.121 | 38.834 | 235.118 | 200.52 |
| 12880 | C | C | MET | 960 | 188.432 | 38.907 | 235.89 | 200.52 |
| 12881 | O | O | MET | 960 | 189.519 | 38.91 | 235.306 | 200.52 |
| 12882 | C | CB | MET | 960 | 186.623 | 37.387 | 235.1 | 200.52 |
| 12883 | C | CG | MET | 960 | 187.623 | 36.405 | 234.51 | 200.52 |
| 12884 | S | SD | MET | 960 | 187.67 | 34.831 | 235.387 | 200.52 |
| 12885 | C | CE | MET | 960 | 189.015 | 34.011 | 234.534 | 200.52 |
| 12886 | N | N | MET | 961 | 188.315 | 38.972 | 237.213 | 203.31 |
| 12887 | C | CA | MET | 961 | 189.477 | 38.944 | 238.085 | 203.31 |
| 12888 | C | C | MET | 961 | 189.83 | 37.5 | 238.438 | 203.31 |
| 12889 | O | O | MET | 961 | 189.331 | 36.543 | 237.839 | 203.31 |
| 12890 | C | CB | MET | 961 | 189.22 | 39.767 | 239.345 | 203.31 |
| 12891 | C | CG | MET | 961 | 188.196 | 40.872 | 239.169 | 203.31 |
| 12892 | S | SD | MET | 961 | 186.557 | 40.206 | 238.83 | 203.31 |
| 12893 | C | CE | MET | 961 | 186.42 | 38.996 | 240.144 | 203.31 |
| 12894 | N | N | SER | 962 | 190.712 | 37.344 | 239.428 | 203.53 |
| 12895 | C | CA | SER | 962 | 191.068 | 36.011 | 239.903 | 203.53 |
| 12896 | C | C | SER | 962 | 189.87 | 35.304 | 240.525 | 203.53 |
| 12897 | O | O | SER | 962 | 189.686 | 34.096 | 240.339 | 203.53 |
| 12898 | C | CB | SER | 962 | 192.213 | 36.106 | 240.909 | 203.53 |
| 12899 | O | OG | SER | 962 | 192.095 | 35.112 | 241.914 | 203.53 |
| 12900 | N | N | ASN | 963 | 189.049 | 36.041 | 241.277 | 200.07 |
| 12901 | C | CA | ASN | 963 | 187.896 | 35.428 | 241.929 | 200.07 |
| 12902 | C | C | ASN | 963 | 186.879 | 34.937 | 240.906 | 200.07 |
| 12903 | O | O | ASN | 963 | 186.26 | 33.883 | 241.092 | 200.07 |
| 12904 | C | CB | ASN | 963 | 187.253 | 36.418 | 242.901 | 200.07 |
| 12905 | C | CG | ASN | 963 | 186.67 | 35.734 | 244.124 | 200.07 |
| 12906 | O | OD1 | ASN | 963 | 186.573 | 36.331 | 245.197 | 200.07 |
| 12907 | N | ND2 | ASN | 963 | 186.269 | 34.48 | 243.964 | 200.07 |
| 12908 | N | N | GLY | 964 | 186.693 | 35.688 | 239.82 | 200.61 |
| 12909 | C | CA | GLY | 964 | 185.777 | 35.309 | 238.767 | 200.61 |
| 12910 | C | C | GLY | 964 | 184.372 | 35.855 | 238.902 | 200.61 |
| 12911 | O | O | GLY | 964 | 183.574 | 35.7 | 237.969 | 200.61 |
| 12912 | N | N | TYR | 965 | 184.041 | 36.486 | 240.025 | 199.62 |
| 12913 | C | CA | TYR | 965 | 182.712 | 37.047 | 240.204 | 199.62 |
| 12914 | C | C | TYR | 965 | 182.572 | 38.348 | 239.416 | 199.62 |
| 12915 | O | O | TYR | 965 | 183.548 | 38.918 | 238.921 | 199.62 |
| 12916 | C | CB | TYR | 965 | 182.43 | 37.286 | 241.689 | 199.62 |
| 12917 | C | CG | TYR | 965 | 180.98 | 37.574 | 242.006 | 199.62 |
| 12918 | C | CD1 | TYR | 965 | 180.02 | 36.573 | 241.926 | 199.62 |
| 12919 | C | CD2 | TYR | 965 | 180.572 | 38.844 | 242.39 | 199.62 |
| 12920 | C | CE1 | TYR | 965 | 178.692 | 36.832 | 242.215 | 199.62 |
| 12921 | C | CE2 | TYR | 965 | 179.248 | 39.112 | 242.681 | 199.62 |
| 12922 | C | CZ | TYR | 965 | 178.313 | 38.104 | 242.593 | 199.62 |
| 12923 | O | OH | TYR | 965 | 176.995 | 38.371 | 242.883 | 199.62 |
| 12924 | N | N | LYS | 966 | 181.329 | 38.814 | 239.29 | 197.66 |
| 12925 | C | CA | LYS | 966 | 181.047 | 40.046 | 238.572 | 197.66 |
| 12926 | C | C | LYS | 966 | 180.661 | 41.134 | 239.559 | 197.66 |
| 12927 | O | O | LYS | 966 | 179.544 | 41.093 | 240.099 | 197.66 |
| 12928 | C | CB | LYS | 966 | 179.925 | 39.829 | 237.563 | 197.66 |
| 12929 | C | CG | LYS | 966 | 180.198 | 38.717 | 236.564 | 197.66 |
| 12930 | C | CD | LYS | 966 | 178.941 | 38.339 | 235.795 | 197.66 |
| 12931 | C | CE | LYS | 966 | 178.422 | 39.499 | 234.96 | 197.66 |
| 12932 | N | NZ | LYS | 966 | 179.371 | 39.867 | 233.875 | 197.66 |
| 12933 | N | N | PRO | 967 | 181.531 | 42.104 | 239.836 | 198.3 |
| 12934 | C | CA | PRO | 967 | 181.123 | 43.24 | 240.67 | 198.3 |
| 12935 | C | C | PRO | 967 | 180 | 44.019 | 240.007 | 198.3 |
| 12936 | O | O | PRO | 967 | 179.926 | 44.122 | 238.781 | 198.3 |
| 12937 | C | CB | PRO | 967 | 182.399 | 44.085 | 240.776 | 198.3 |
| 12938 | C | CG | PRO | 967 | 183.513 | 43.138 | 240.472 | 198.3 |
| 12939 | C | CD | PRO | 967 | 182.957 | 42.163 | 239.477 | 198.3 |
| 12940 | N | N | ALA | 968 | 179.119 | 44.584 | 240.837 | 192.86 |
| 12941 | C | CA | ALA | 968 | 177.891 | 45.223 | 240.369 | 192.86 |
| 12942 | C | C | ALA | 968 | 177.797 | 46.652 | 240.894 | 192.86 |
| 12943 | O | O | ALA | 968 | 177.034 | 46.93 | 241.832 | 192.86 |
| 12944 | C | CB | ALA | 968 | 176.663 | 44.414 | 240.785 | 192.86 |
| 12945 | N | N | PRO | 969 | 178.556 | 47.584 | 240.32 | 188.4 |
| 12946 | C | CA | PRO | 969 | 178.314 | 49.001 | 240.604 | 188.4 |
| 12947 | C | C | PRO | 969 | 177.015 | 49.474 | 239.968 | 188.4 |
| 12948 | O | O | PRO | 969 | 176.487 | 48.867 | 239.034 | 188.4 |
| 12949 | C | CB | PRO | 969 | 179.526 | 49.703 | 239.983 | 188.4 |
| 12950 | C | CG | PRO | 969 | 180.005 | 48.765 | 238.937 | 188.4 |
| 12951 | C | CD | PRO | 969 | 179.755 | 47.393 | 239.486 | 188.4 |
| 12952 | N | N | LEU | 970 | 176.498 | 50.578 | 240.501 | 182.02 |
| 12953 | C | CA | LEU | 970 | 175.232 | 51.118 | 240.024 | 182.02 |
| 12954 | C | C | LEU | 970 | 175.353 | 51.573 | 238.573 | 182.02 |
| 12955 | O | O | LEU | 970 | 176.331 | 52.217 | 238.185 | 182.02 |
| 12956 | C | CB | LEU | 970 | 174.792 | 52.284 | 240.914 | 182.02 |
| 12957 | C | CG | LEU | 970 | 173.301 | 52.612 | 241.034 | 182.02 |
| 12958 | C | CD1 | LEU | 970 | 173.052 | 53.434 | 242.286 | 182.02 |
| 12959 | C | CD2 | LEU | 970 | 172.784 | 53.355 | 239.812 | 182.02 |
| 12960 | N | N | ASP | 971 | 174.348 | 51.23 | 237.77 | 177.85 |
| 12961 | C | CA | ASP | 971 | 174.3 | 51.632 | 236.37 | 177.85 |
| 12962 | C | C | ASP | 971 | 173.63 | 52.996 | 236.266 | 177.85 |
| 12963 | O | O | ASP | 971 | 172.449 | 53.142 | 236.602 | 177.85 |
| 12964 | C | CB | ASP | 971 | 173.548 | 50.601 | 235.531 | 177.85 |
| 12965 | C | CG | ASP | 971 | 173.481 | 50.984 | 234.067 | 177.85 |
| 12966 | O | OD1 | ASP | 971 | 174.538 | 50.972 | 233.401 | 177.85 |
| 12967 | O | OD2 | ASP | 971 | 172.374 | 51.301 | 233.582 | 177.85 |
| 12968 | N | N | LEU | 972 | 174.38 | 53.988 | 235.791 | 167.78 |
| 12969 | C | CA | LEU | 972 | 173.919 | 55.369 | 235.734 | 167.78 |
| 12970 | C | C | LEU | 972 | 174.138 | 55.98 | 234.353 | 167.78 |
| 12971 | O | O | LEU | 972 | 174.436 | 57.17 | 234.232 | 167.78 |
| 12972 | C | CB | LEU | 972 | 174.609 | 56.198 | 236.816 | 167.78 |
| 12973 | C | CG | LEU | 972 | 175.941 | 55.611 | 237.293 | 167.78 |
| 12974 | C | CD1 | LEU | 972 | 177.101 | 56.117 | 236.444 | 167.78 |
| 12975 | C | CD2 | LEU | 972 | 176.172 | 55.886 | 238.773 | 167.78 |
| 12976 | N | N | SER | 973 | 173.998 | 55.169 | 233.301 | 163.48 |
| 12977 | C | CA | SER | 973 | 174.115 | 55.69 | 231.943 | 163.48 |
| 12978 | C | C | SER | 973 | 172.988 | 56.665 | 231.627 | 163.48 |
| 12979 | O | O | SER | 973 | 173.2 | 57.675 | 230.947 | 163.48 |
| 12980 | C | CB | SER | 973 | 174.127 | 54.538 | 230.938 | 163.48 |
| 12981 | O | OG | SER | 973 | 172.943 | 53.766 | 231.033 | 163.48 |
| 12982 | N | N | HIS | 974 | 171.778 | 56.375 | 232.112 | 162.89 |
| 12983 | C | CA | HIS | 974 | 170.645 | 57.265 | 231.877 | 162.89 |
| 12984 | C | C | HIS | 974 | 170.813 | 58.589 | 232.615 | 162.89 |
| 12985 | O | O | HIS | 974 | 170.141 | 59.576 | 232.294 | 162.89 |
| 12986 | C | CB | HIS | 974 | 169.349 | 56.575 | 232.299 | 162.89 |
| 12987 | C | CG | HIS | 974 | 169.467 | 55.8 | 233.574 | 162.89 |
| 12988 | N | ND1 | HIS | 974 | 169.736 | 54.448 | 233.6 | 162.89 |
| 12989 | C | CD2 | HIS | 974 | 169.36 | 56.186 | 234.867 | 162.89 |
| 12990 | C | CE1 | HIS | 974 | 169.785 | 54.035 | 234.853 | 162.89 |
| 12991 | N | NE2 | HIS | 974 | 169.561 | 55.07 | 235.642 | 162.89 |
| 12992 | N | N | VAL | 975 | 171.699 | 58.626 | 233.613 | 161.53 |
| 12993 | C | CA | VAL | 975 | 171.892 | 59.84 | 234.394 | 161.53 |
| 12994 | C | C | VAL | 975 | 172.626 | 60.889 | 233.567 | 161.53 |
| 12995 | O | O | VAL | 975 | 173.63 | 60.601 | 232.904 | 161.53 |
| 12996 | C | CB | VAL | 975 | 172.656 | 59.518 | 235.689 | 161.53 |
| 12997 | C | CG1 | VAL | 975 | 173.035 | 60.796 | 236.413 | 161.53 |
| 12998 | C | CG | VAL | 975 | 171.821 | 58.616 | 236.586 | 161.53 |
| 12999 | N | N | ARG | 976 | 172.118 | 62.121 | 233.605 | 155.63 |
| 13000 | C | CA | ARG | 976 | 172.748 | 63.257 | 232.95 | 155.63 |
| 13001 | C | C | ARG | 976 | 172.956 | 64.369 | 233.968 | 155.63 |
| 13002 | O | O | ARG | 976 | 172.189 | 64.505 | 234.926 | 155.63 |
| 13003 | C | CB | ARG | 976 | 171.908 | 63.776 | 231.774 | 155.63 |
| 13004 | C | CG | ARG | 976 | 171.694 | 62.772 | 230.647 | 155.63 |
| 13005 | C | CD | ARG | 976 | 172.889 | 62.689 | 229.701 | 155.63 |
| 13006 | N | NE | ARG | 976 | 173.937 | 61.798 | 230.186 | 155.63 |
| 13007 | C | CZ | ARG | 976 | 175.097 | 62.202 | 230.685 | 155.63 |
| 13008 | N | NH1 | ARG | 976 | 175.399 | 63.486 | 230.78 | 155.63 |
| 13009 | N | NH2 | ARG | 976 | 175.976 | 61.294 | 231.1 | 155.63 |
| 13010 | N | N | LEU | 977 | 173.999 | 65.167 | 233.752 | 153.15 |
| 13011 | C | CA | LEU | 977 | 174.368 | 66.248 | 234.655 | 153.15 |
| 13012 | C | C | LEU | 977 | 174.276 | 67.582 | 233.93 | 153.15 |
| 13013 | O | O | LEU | 977 | 174.704 | 67.7 | 232.777 | 153.15 |
| 13014 | C | CB | LEU | 977 | 175.783 | 66.054 | 235.208 | 153.15 |
| 13015 | C | CG | LEU | 977 | 175.967 | 65.026 | 236.325 | 153.15 |
| 13016 | C | CD1 | LEU | 977 | 177.407 | 65.029 | 236.81 | 153.15 |
| 13017 | C | CD2 | LEU | 977 | 175.01 | 65.304 | 237.473 | 153.15 |
| 13018 | N | N | THR | 978 | 173.719 | 68.58 | 234.61 | 144.98 |
| 13019 | C | CA | THR | 978 | 173.669 | 69.923 | 234.061 | 144.98 |
| 13020 | C | C | THR | 978 | 175.068 | 70.539 | 234.058 | 144.98 |
| 13021 | O | O | THR | 978 | 175.945 | 70.104 | 234.81 | 144.98 |
| 13022 | C | CB | THR | 978 | 172.719 | 70.799 | 234.874 | 144.98 |
| 13023 | O | OG1 | THR | 978 | 173.319 | 71.109 | 236.138 | 144.98 |
| 13024 | C | CG2 | THR | 978 | 171.405 | 70.073 | 235.114 | 144.98 |
| 13025 | N | N | PRO | 979 | 175.309 | 71.543 | 233.208 | 142.53 |
| 13026 | C | CA | PRO | 979 | 176.623 | 72.211 | 233.233 | 142.53 |
| 13027 | C | C | PRO | 979 | 176.974 | 72.794 | 234.59 | 142.53 |
| 13028 | O | O | PRO | 979 | 178.151 | 72.798 | 234.973 | 142.53 |
| 13029 | C | CB | PRO | 979 | 176.476 | 73.3 | 232.163 | 142.53 |
| 13030 | C | CG | PRO | 979 | 175.444 | 72.769 | 231.232 | 142.53 |
| 13031 | C | CD | PRO | 979 | 174.472 | 72.015 | 232.091 | 142.53 |
| 13032 | N | N | ALA | 980 | 175.981 | 73.296 | 235.329 | 144.12 |
| 13033 | C | CA | ALA | 980 | 176.239 | 73.775 | 236.682 | 144.12 |
| 13034 | C | C | ALA | 980 | 176.673 | 72.635 | 237.595 | 144.12 |
| 13035 | O | O | ALA | 980 | 177.548 | 72.813 | 238.451 | 144.12 |
| 13036 | C | CB | ALA | 980 | 174.997 | 74.469 | 237.24 | 144.12 |
| 13037 | N | N | GLN | 981 | 176.067 | 71.456 | 237.432 | 146.53 |
| 13038 | C | CA | GLN | 981 | 176.451 | 70.307 | 238.246 | 146.53 |
| 13039 | C | C | GLN | 981 | 177.877 | 69.866 | 237.945 | 146.53 |
| 13040 | O | O | GLN | 981 | 178.591 | 69.397 | 238.84 | 146.53 |
| 13041 | C | CB | GLN | 981 | 175.474 | 69.152 | 238.024 | 146.53 |
| 13042 | C | CG | GLN | 981 | 174.124 | 69.345 | 238.695 | 146.53 |
| 13043 | C | CD | GLN | 981 | 174.22 | 69.353 | 240.208 | 146.53 |
| 13044 | O | OE1 | GLN | 981 | 173.417 | 69.99 | 240.888 | 146.53 |
| 13045 | N | NE2 | GLN | 981 | 175.202 | 68.636 | 240.743 | 146.53 |
| 13046 | N | N | THR | 982 | 178.308 | 69.997 | 236.688 | 139.99 |
| 13047 | C | CA | THR | 982 | 179.68 | 69.644 | 236.339 | 139.99 |
| 13048 | C | C | THR | 982 | 180.677 | 70.529 | 237.074 | 139.99 |
| 13049 | O | O | THR | 982 | 181.741 | 70.061 | 237.497 | 139.99 |
| 13050 | C | CB | THR | 982 | 179.883 | 69.748 | 234.827 | 139.99 |
| 13051 | O | OG1 | THR | 982 | 179.622 | 71.09 | 234.4 | 139.99 |
| 13052 | C | CG2 | THR | 982 | 178.944 | 68.797 | 234.098 | 139.99 |
| 13053 | N | N | THR | 983 | 180.351 | 71.814 | 237.234 | 134.32 |
| 13054 | C | CA | THR | 983 | 181.197 | 72.7 | 238.026 | 134.32 |
| 13055 | C | C | THR | 983 | 181.252 | 72.245 | 239.479 | 134.32 |
| 13056 | O | O | THR | 983 | 182.298 | 72.346 | 240.132 | 134.32 |
| 13057 | C | CB | THR | 983 | 180.687 | 74.138 | 237.93 | 134.32 |
| 13058 | O | OG1 | THR | 983 | 180.565 | 74.511 | 236.552 | 134.32 |
| 13059 | C | CG2 | THR | 983 | 181.647 | 75.095 | 238.621 | 134.32 |
| 13060 | N | N | LEU | 984 | 180.132 | 71.74 | 240.002 | 132.05 |
| 13061 | C | CA | LEU | 984 | 180.119 | 71.214 | 241.363 | 132.05 |
| 13062 | C | C | LEU | 984 | 181.058 | 70.024 | 241.502 | 132.05 |
| 13063 | O | O | LEU | 984 | 181.747 | 69.883 | 242.52 | 132.05 |
| 13064 | C | CB | LEU | 984 | 178.696 | 70.822 | 241.76 | 132.05 |
| 13065 | C | CG | LEU | 984 | 178.52 | 70.195 | 243.144 | 132.05 |
| 13066 | C | CD1 | LEU | 984 | 177.876 | 71.181 | 244.107 | 132.05 |
| 13067 | C | CD2 | LEU | 984 | 177.708 | 68.913 | 243.053 | 132.05 |
| 13068 | N | N | VAL | 985 | 181.098 | 69.156 | 240.489 | 129.21 |
| 13069 | C | CA | VAL | 985 | 182.001 | 68.008 | 240.524 | 129.21 |
| 13070 | C | C | VAL | 985 | 183.449 | 68.473 | 240.594 | 129.21 |
| 13071 | O | O | VAL | 985 | 184.274 | 67.881 | 241.301 | 129.21 |
| 13072 | C | CB | VAL | 985 | 181.754 | 67.098 | 239.306 | 129.21 |
| 13073 | C | CG1 | VAL | 985 | 182.694 | 65.904 | 239.334 | 129.21 |
| 13074 | C | CG2 | VAL | 985 | 180.306 | 66.639 | 239.274 | 129.21 |
| 13075 | N | N | ASP | 986 | 183.781 | 69.541 | 239.864 | 123.63 |
| 13076 | C | CA | ASP | 986 | 185.134 | 70.084 | 239.923 | 123.63 |
| 13077 | C | C | ASP | 986 | 185.465 | 70.588 | 241.321 | 123.63 |
| 13078 | O | O | ASP | 986 | 186.569 | 70.356 | 241.828 | 123.63 |
| 13079 | C | CB | ASP | 986 | 185.293 | 71.206 | 238.898 | 123.63 |
| 13080 | C | CG | ASP | 986 | 184.774 | 70.821 | 237.527 | 123.63 |
| 13081 | O | OD1 | ASP | 986 | 184.697 | 69.609 | 237.238 | 123.63 |
| 13082 | O | OD2 | ASP | 986 | 184.44 | 71.731 | 236.74 | 123.63 |
| 13083 | N | N | ARG | 987 | 184.522 | 71.285 | 241.96 | 120.92 |
| 13084 | C | CA | ARG | 987 | 184.751 | 71.757 | 243.321 | 120.92 |
| 13085 | C | C | ARG | 987 | 184.833 | 70.596 | 244.303 | 120.92 |
| 13086 | O | O | ARG | 987 | 185.628 | 70.629 | 245.249 | 120.92 |
| 13087 | C | CB | ARG | 987 | 183.653 | 72.74 | 243.728 | 120.92 |
| 13088 | C | CG | ARG | 987 | 183.846 | 74.136 | 243.155 | 120.92 |
| 13089 | C | CD | ARG | 987 | 182.752 | 75.094 | 243.599 | 120.92 |
| 13090 | N | NE | ARG | 987 | 181.616 | 75.099 | 242.684 | 120.92 |
| 13091 | C | CZ | ARG | 987 | 180.516 | 74.377 | 242.847 | 120.92 |
| 13092 | N | NH1 | ARG | 987 | 180.365 | 73.571 | 243.885 | 120.92 |
| 13093 | N | NH2 | ARG | 987 | 179.542 | 74.468 | 241.946 | 120.92 |
| 13094 | N | N | LEU | 988 | 184.015 | 69.561 | 244.1 | 117.34 |
| 13095 | C | CA | LEU | 988 | 184.09 | 68.384 | 244.96 | 117.34 |
| 13096 | C | C | LEU | 988 | 185.427 | 67.672 | 244.806 | 117.34 |
| 13097 | O | O | LEU | 988 | 186.02 | 67.226 | 245.795 | 117.34 |
| 13098 | C | CB | LEU | 988 | 182.938 | 67.43 | 244.651 | 117.34 |
| 13099 | C | CG | LEU | 988 | 181.541 | 67.882 | 245.072 | 117.34 |
| 13100 | C | CD1 | LEU | 988 | 180.516 | 66.824 | 244.704 | 117.34 |
| 13101 | C | CD2 | LEU | 988 | 181.499 | 68.179 | 246.562 | 117.34 |
| 13102 | N | N | ALA | 989 | 185.914 | 67.549 | 243.569 | 107.78 |
| 13103 | C | CA | ALA | 989 | 187.208 | 66.915 | 243.344 | 107.78 |
| 13104 | C | C | ALA | 989 | 188.333 | 67.716 | 243.986 | 107.78 |
| 13105 | O | O | ALA | 989 | 189.262 | 67.141 | 244.565 | 107.78 |
| 13106 | C | CB | ALA | 989 | 187.456 | 66.743 | 241.846 | 107.78 |
| 13107 | N | N | GLU | 990 | 188.269 | 69.045 | 243.888 | 99.15 |
| 13108 | C | CA | GLU | 990 | 189.299 | 69.884 | 244.491 | 99.15 |
| 13109 | C | C | GLU | 990 | 189.319 | 69.726 | 246.005 | 99.15 |
| 13110 | O | O | GLU | 990 | 190.389 | 69.62 | 246.617 | 99.15 |
| 13111 | C | CB | GLU | 990 | 189.075 | 71.345 | 244.105 | 99.15 |
| 13112 | C | CG | GLU | 990 | 190.084 | 72.301 | 244.713 | 99.15 |
| 13113 | C | CD | GLU | 990 | 189.914 | 73.722 | 244.219 | 99.15 |
| 13114 | O | OE1 | GLU | 990 | 188.829 | 74.045 | 243.692 | 99.15 |
| 13115 | O | OE2 | GLU | 990 | 190.868 | 74.517 | 244.355 | 99.15 |
| 13116 | N | N | ASN | 991 | 188.14 | 69.712 | 246.63 | 98.5 |
| 13117 | C | CA | ASN | 991 | 188.077 | 69.556 | 248.079 | 98.5 |
| 13118 | C | C | ASN | 991 | 188.571 | 68.183 | 248.514 | 98.5 |
| 13119 | O | O | ASN | 991 | 189.292 | 68.066 | 249.511 | 98.5 |
| 13120 | C | CB | ASN | 991 | 186.652 | 69.786 | 248.574 | 98.5 |
| 13121 | C | CG | ASN | 991 | 186.516 | 69.559 | 250.063 | 98.5 |
| 13122 | O | OD1 | ASN | 991 | 186.308 | 68.434 | 250.515 | 98.5 |
| 13123 | N | ND2 | ASN | 991 | 186.644 | 70.627 | 250.835 | 98.5 |
| 13124 | N | N | GLY | 992 | 188.185 | 67.134 | 247.785 | 96.19 |
| 13125 | C | CA | GLY | 992 | 188.601 | 65.793 | 248.161 | 96.19 |
| 13126 | C | C | GLY | 992 | 190.107 | 65.624 | 248.144 | 96.19 |
| 13127 | O | O | GLY | 992 | 190.678 | 64.97 | 249.019 | 96.19 |
| 13128 | N | N | HIS | 993 | 190.77 | 66.206 | 247.143 | 94.12 |
| 13129 | C | CA | HIS | 993 | 192.226 | 66.166 | 247.101 | 94.12 |
| 13130 | C | C | HIS | 993 | 192.832 | 66.942 | 248.263 | 94.12 |
| 13131 | O | O | HIS | 993 | 193.811 | 66.497 | 248.874 | 94.12 |
| 13132 | C | CB | HIS | 993 | 192.725 | 66.719 | 245.767 | 94.12 |
| 13133 | C | CG | HIS | 993 | 194.205 | 66.605 | 245.583 | 94.12 |
| 13134 | N | ND1 | HIS | 993 | 194.85 | 65.392 | 245.476 | 94.12 |
| 13135 | C | CD2 | HIS | 993 | 195.167 | 67.553 | 245.486 | 94.12 |
| 13136 | C | CE1 | HIS | 993 | 196.145 | 65.597 | 245.322 | 94.12 |
| 13137 | N | NE2 | HIS | 993 | 196.364 | 66.899 | 245.325 | 94.12 |
| 13138 | N | N | ASN | 994 | 192.262 | 68.106 | 248.585 | 88.4 |
| 13139 | C | CA | ASN | 994 | 192.799 | 68.918 | 249.672 | 88.4 |
| 13140 | C | C | ASN | 994 | 192.628 | 68.228 | 251.019 | 88.4 |
| 13141 | O | O | ASN | 994 | 193.513 | 68.304 | 251.878 | 88.4 |
| 13142 | C | CB | ASN | 994 | 192.131 | 70.291 | 249.679 | 88.4 |
| 13143 | C | CG | ASN | 994 | 192.504 | 71.122 | 248.471 | 88.4 |
| 13144 | O | OD1 | ASN | 994 | 192.996 | 70.599 | 247.473 | 88.4 |
| 13145 | N | ND2 | ASN | 994 | 192.273 | 72.426 | 248.555 | 88.4 |
| 13146 | N | N | VAL | 995 | 191.493 | 67.557 | 251.226 | 89.79 |
| 13147 | C | CA | VAL | 995 | 191.291 | 66.813 | 252.467 | 89.79 |
| 13148 | C | C | VAL | 995 | 192.33 | 65.71 | 252.595 | 89.79 |
| 13149 | O | O | VAL | 995 | 192.921 | 65.51 | 253.663 | 89.79 |
| 13150 | C | CB | VAL | 995 | 189.858 | 66.252 | 252.53 | 89.79 |
| 13151 | C | CG1 | VAL | 995 | 189.711 | 65.3 | 253.705 | 89.79 |
| 13152 | C | CG2 | VAL | 995 | 188.851 | 67.382 | 252.635 | 89.79 |
| 13153 | N | N | TRP | 996 | 192.574 | 64.979 | 251.506 | 98.91 |
| 13154 | C | CA | TRP | 996 | 193.617 | 63.96 | 251.517 | 98.91 |
| 13155 | C | C | TRP | 996 | 194.994 | 64.583 | 251.704 | 98.91 |
| 13156 | O | O | TRP | 996 | 195.849 | 64.021 | 252.399 | 98.91 |
| 13157 | C | CB | TRP | 996 | 193.566 | 63.147 | 250.224 | 98.91 |
| 13158 | C | CG | TRP | 996 | 194.721 | 62.214 | 250.058 | 98.91 |
| 13159 | C | CD1 | TRP | 996 | 194.849 | 60.966 | 250.589 | 98.91 |
| 13160 | C | CD2 | TRP | 996 | 195.913 | 62.453 | 249.302 | 98.91 |
| 13161 | N | NE1 | TRP | 996 | 196.048 | 60.412 | 250.214 | 98.91 |
| 13162 | C | CE2 | TRP | 996 | 196.72 | 61.306 | 249.423 | 98.91 |
| 13163 | C | CE3 | TRP | 996 | 196.378 | 63.526 | 248.537 | 98.91 |
| 13164 | C | CZ2 | TRP | 996 | 197.964 | 61.201 | 248.808 | 98.91 |
| 13165 | C | CZ3 | TRP | 996 | 197.612 | 63.42 | 247.926 | 98.91 |
| 13166 | C | CH2 | TRP | 996 | 198.391 | 62.266 | 248.065 | 98.91 |
| 13167 | N | N | ALA | 997 | 195.229 | 65.742 | 251.085 | 87.3 |
| 13168 | C | CA | ALA | 997 | 196.539 | 66.379 | 251.172 | 87.3 |
| 13169 | C | C | ALA | 997 | 196.857 | 66.802 | 252.6 | 87.3 |
| 13170 | O | O | ALA | 997 | 197.94 | 66.508 | 253.119 | 87.3 |
| 13171 | C | CB | ALA | 997 | 196.6 | 67.58 | 250.229 | 87.3 |
| 13172 | N | N | ARG | 998 | 195.921 | 67.49 | 253.257 | 83.63 |
| 13173 | C | CA | ARG | 998 | 196.184 | 67.976 | 254.608 | 83.63 |
| 13174 | C | C | ARG | 998 | 196.273 | 66.823 | 255.599 | 83.63 |
| 13175 | O | O | ARG | 998 | 196.978 | 66.917 | 256.61 | 83.63 |
| 13176 | C | CB | ARG | 998 | 195.112 | 68.982 | 255.028 | 83.63 |
| 13177 | C | CG | ARG | 998 | 193.709 | 68.418 | 255.168 | 83.63 |
| 13178 | C | CD | ARG | 998 | 193.359 | 68.167 | 256.627 | 83.63 |
| 13179 | N | NE | ARG | 998 | 191.938 | 67.901 | 256.81 | 83.63 |
| 13180 | C | CZ | ARG | 998 | 191.031 | 68.834 | 257.061 | 83.63 |
| 13181 | N | NH1 | ARG | 998 | 191.363 | 70.109 | 257.173 | 83.63 |
| 13182 | N | NH2 | ARG | 998 | 189.757 | 68.478 | 257.205 | 83.63 |
| 13183 | N | N | ASP | 999 | 195.558 | 65.729 | 255.333 | 83.34 |
| 13184 | C | CA | ASP | 999 | 195.692 | 64.543 | 256.171 | 83.34 |
| 13185 | C | C | ASP | 999 | 197.095 | 63.958 | 256.072 | 83.34 |
| 13186 | O | O | ASP | 999 | 197.675 | 63.547 | 257.083 | 83.34 |
| 13187 | C | CB | ASP | 999 | 194.632 | 63.509 | 255.784 | 83.34 |
| 13188 | C | CG | ASP | 999 | 195.164 | 62.086 | 255.789 | 83.34 |
| 13189 | O | OD1 | ASP | 999 | 195.814 | 61.684 | 254.799 | 83.34 |
| 13190 | O | OD2 | ASP | 999 | 194.934 | 61.369 | 256.785 | 83.34 |
| 13191 | N | N | ARG | 1000 | 197.657 | 63.917 | 254.862 | 80.66 |
| 13192 | C | CA | ARG | 1000 | 199.01 | 63.399 | 254.69 | 80.66 |
| 13193 | C | C | ARG | 1000 | 200.046 | 64.326 | 255.315 | 80.66 |
| 13194 | O | O | ARG | 1000 | 201.053 | 63.859 | 255.859 | 80.66 |
| 13195 | C | CB | ARG | 1000 | 199.306 | 63.187 | 253.207 | 80.66 |
| 13196 | C | CG | ARG | 1000 | 198.569 | 62.013 | 252.589 | 80.66 |
| 13197 | C | CD | ARG | 1000 | 198.942 | 60.712 | 253.277 | 80.66 |
| 13198 | N | NE | ARG | 1000 | 198.406 | 59.551 | 252.577 | 80.66 |
| 13199 | C | CZ | ARG | 1000 | 199.041 | 58.903 | 251.611 | 80.66 |
| 13200 | N | NH1 | ARG | 1000 | 200.242 | 59.278 | 251.2 | 80.66 |
| 13201 | N | NH2 | ARG | 1000 | 198.457 | 57.853 | 251.041 | 80.66 |
| 13202 | N | N | VAL | 1001 | 199.821 | 65.64 | 255.24 | 74.1 |
| 13203 | C | CA | VAL | 1001 | 200.755 | 66.589 | 255.84 | 74.1 |
| 13204 | C | C | VAL | 1001 | 200.826 | 66.385 | 257.347 | 74.1 |
| 13205 | O | O | VAL | 1001 | 201.91 | 66.415 | 257.942 | 74.1 |
| 13206 | C | CB | VAL | 1001 | 200.36 | 68.032 | 255.48 | 74.1 |
| 13207 | C | CG1 | VAL | 1001 | 201.228 | 69.024 | 256.233 | 74.1 |
| 13208 | C | CG2 | VAL | 1001 | 200.486 | 68.252 | 253.989 | 74.1 |
| 13209 | N | N | ALA | 1002 | 199.673 | 66.176 | 257.987 | 74.87 |
| 13210 | C | CA | ALA | 1002 | 199.665 | 65.896 | 259.419 | 74.87 |
| 13211 | C | C | ALA | 1002 | 200.424 | 64.614 | 259.734 | 74.87 |
| 13212 | O | O | ALA | 1002 | 200.983 | 64.466 | 260.826 | 74.87 |
| 13213 | C | CB | ALA | 1002 | 198.228 | 65.809 | 259.929 | 74.87 |
| 13214 | N | N | GLN | 1003 | 200.456 | 63.676 | 258.789 | 75.47 |
| 13215 | C | CA | GLN | 1003 | 201.204 | 62.438 | 258.959 | 75.47 |
| 13216 | C | C | GLN | 1003 | 202.679 | 62.583 | 258.608 | 75.47 |
| 13217 | O | O | GLN | 1003 | 203.437 | 61.624 | 258.79 | 75.47 |
| 13218 | C | CB | GLN | 1003 | 200.578 | 61.328 | 258.112 | 75.47 |
| 13219 | C | CG | GLN | 1003 | 199.197 | 60.905 | 258.578 | 75.47 |
| 13220 | C | CD | GLN | 1003 | 198.565 | 59.873 | 257.667 | 75.47 |
| 13221 | O | OE1 | GLN | 1003 | 198.757 | 59.901 | 256.451 | 75.47 |
| 13222 | N | NE2 | GLN | 1003 | 197.81 | 58.951 | 258.251 | 75.47 |
| 13223 | N | N | GLY | 1004 | 203.104 | 63.741 | 258.115 | 73.99 |
| 13224 | C | CA | GLY | 1004 | 204.494 | 63.977 | 257.794 | 73.99 |
| 13225 | C | C | GLY | 1004 | 204.864 | 63.862 | 256.333 | 73.99 |
| 13226 | O | O | GLY | 1004 | 206.053 | 63.955 | 256.007 | 73.99 |
| 13227 | N | N | TRP | 1005 | 203.895 | 63.661 | 255.446 | 71.64 |
| 13228 | C | CA | TRP | 1005 | 204.19 | 63.537 | 254.027 | 71.64 |
| 13229 | C | C | TRP | 1005 | 204.51 | 64.897 | 253.416 | 71.64 |
| 13230 | O | O | TRP | 1005 | 204.008 | 65.936 | 253.854 | 71.64 |
| 13231 | C | CB | TRP | 1005 | 203.015 | 62.892 | 253.296 | 71.64 |
| 13232 | C | CG | TRP | 1005 | 202.853 | 61.446 | 253.621 | 71.64 |
| 13233 | C | CD1 | TRP | 1005 | 202.397 | 60.916 | 254.791 | 71.64 |
| 13234 | C | CD2 | TRP | 1005 | 203.149 | 60.338 | 252.766 | 71.64 |
| 13235 | N | NE1 | TRP | 1005 | 202.391 | 59.545 | 254.718 | 71.64 |
| 13236 | C | CE2 | TRP | 1005 | 202.848 | 59.165 | 253.484 | 71.64 |
| 13237 | C | CE3 | TRP | 1005 | 203.638 | 60.223 | 251.463 | 71.64 |
| 13238 | C | CZ2 | TRP | 1005 | 203.02 | 57.895 | 252.944 | 71.64 |
| 13239 | C | CZ3 | TRP | 1005 | 203.809 | 58.962 | 250.928 | 71.64 |
| 13240 | C | CH2 | TRP | 1005 | 203.501 | 57.815 | 251.667 | 71.64 |
| 13241 | N | N | SER | 1006 | 205.36 | 64.881 | 252.392 | 68.02 |
| 13242 | C | CA | SER | 1006 | 205.769 | 66.094 | 251.702 | 68.02 |
| 13243 | C | C | SER | 1006 | 205.847 | 65.819 | 250.208 | 68.02 |
| 13244 | O | O | SER | 1006 | 205.981 | 64.674 | 249.772 | 68.02 |
| 13245 | C | CB | SER | 1006 | 207.117 | 66.613 | 252.216 | 68.02 |
| 13246 | O | OG | SER | 1006 | 207.048 | 66.932 | 253.594 | 68.02 |
| 13247 | N | N | TYR | 1007 | 205.761 | 66.889 | 249.424 | 67.42 |
| 13248 | C | CA | TYR | 1007 | 205.809 | 66.772 | 247.975 | 67.42 |
| 13249 | C | C | TYR | 1007 | 207.223 | 66.475 | 247.498 | 67.42 |
| 13250 | O | O | TYR | 1007 | 208.203 | 66.969 | 248.063 | 67.42 |
| 13251 | C | CB | TYR | 1007 | 205.305 | 68.052 | 247.312 | 67.42 |
| 13252 | C | CG | TYR | 1007 | 205.593 | 68.108 | 245.83 | 67.42 |
| 13253 | C | CD1 | TYR | 1007 | 204.858 | 67.349 | 244.932 | 67.42 |
| 13254 | C | CD2 | TYR | 1007 | 206.606 | 68.914 | 245.33 | 67.42 |
| 13255 | C | CE1 | TYR | 1007 | 205.121 | 67.392 | 243.58 | 67.42 |
| 13256 | C | CE2 | TYR | 1007 | 206.876 | 68.963 | 243.979 | 67.42 |
| 13257 | C | CZ | TYR | 1007 | 206.13 | 68.201 | 243.109 | 67.42 |
| 13258 | O | OH | TYR | 1007 | 206.395 | 68.248 | 241.761 | 67.42 |
| 13259 | N | N | SER | 1008 | 207.322 | 65.663 | 246.45 | 67.04 |
| 13260 | C | CA | SER | 1008 | 208.583 | 65.398 | 245.776 | 67.04 |
| 13261 | C | C | SER | 1008 | 208.294 | 64.925 | 244.361 | 67.04 |
| 13262 | O | O | SER | 1008 | 207.324 | 64.199 | 244.133 | 67.04 |
| 13263 | C | CB | SER | 1008 | 209.413 | 64.345 | 246.516 | 67.04 |
| 13264 | O | OG | SER | 1008 | 210.594 | 64.031 | 245.8 | 67.04 |
| 13265 | N | N | ALA | 1009 | 209.14 | 65.339 | 243.415 | 66.11 |
| 13266 | C | CA | ALA | 1009 | 208.985 | 64.879 | 242.04 | 66.11 |
| 13267 | C | C | ALA | 1009 | 209.168 | 63.37 | 241.944 | 66.11 |
| 13268 | O | O | ALA | 1009 | 208.459 | 62.697 | 241.187 | 66.11 |
| 13269 | C | CB | ALA | 1009 | 209.976 | 65.601 | 241.127 | 66.11 |
| 13270 | N | N | VAL | 1010 | 210.111 | 62.824 | 242.702 | 70.19 |
| 13271 | C | CA | VAL | 1010 | 210.356 | 61.388 | 242.734 | 70.19 |
| 13272 | C | C | VAL | 1010 | 209.68 | 60.803 | 243.966 | 70.19 |
| 13273 | O | O | VAL | 1010 | 209.902 | 61.269 | 245.089 | 70.19 |
| 13274 | C | CB | VAL | 1010 | 211.863 | 61.088 | 242.738 | 70.19 |
| 13275 | C | CG1 | VAL | 1010 | 212.1 | 59.59 | 242.75 | 70.19 |
| 13276 | C | CG2 | VAL | 1010 | 212.534 | 61.734 | 241.535 | 70.19 |
| 13277 | N | N | GLN | 1011 | 208.853 | 59.783 | 243.758 | 75.37 |
| 13278 | C | CA | GLN | 1011 | 208.115 | 59.15 | 244.842 | 75.37 |
| 13279 | C | C | GLN | 1011 | 209.064 | 58.344 | 245.719 | 75.37 |
| 13280 | O | O | GLN | 1011 | 209.907 | 57.598 | 245.211 | 75.37 |
| 13281 | C | CB | GLN | 1011 | 207.012 | 58.248 | 244.287 | 75.37 |
| 13282 | C | CG | GLN | 1011 | 206.284 | 57.44 | 245.346 | 75.37 |
| 13283 | C | CD | GLN | 1011 | 205.179 | 56.581 | 244.768 | 75.37 |
| 13284 | O | OE1 | GLN | 1011 | 205.084 | 55.39 | 245.064 | 75.37 |
| 13285 | N | NE2 | GLN | 1011 | 204.332 | 57.183 | 243.942 | 75.37 |
| 13286 | N | N | ASP | 1012 | 208.926 | 58.502 | 247.034 | 75.3 |
| 13287 | C | CA | ASP | 1012 | 209.732 | 57.773 | 248.013 | 75.3 |
| 13288 | C | C | ASP | 1012 | 208.822 | 57.42 | 249.187 | 75.3 |
| 13289 | O | O | ASP | 1012 | 208.63 | 58.232 | 250.097 | 75.3 |
| 13290 | C | CB | ASP | 1012 | 210.935 | 58.599 | 248.461 | 75.3 |
| 13291 | C | CG | ASP | 1012 | 212.002 | 57.76 | 249.138 | 75.3 |
| 13292 | O | OD1 | ASP | 1012 | 211.647 | 56.869 | 249.937 | 75.3 |
| 13293 | O | OD2 | ASP | 1012 | 213.2 | 57.989 | 248.869 | 75.3 |
| 13294 | N | N | ILE | 1013 | 208.258 | 56.209 | 249.158 | 74.73 |
| 13295 | C | CA | ILE | 1013 | 207.333 | 55.799 | 250.218 | 74.73 |
| 13296 | C | C | ILE | 1013 | 208.007 | 55.75 | 251.582 | 74.73 |
| 13297 | O | O | ILE | 1013 | 207.433 | 56.278 | 252.547 | 74.73 |
| 13298 | C | CB | ILE | 1013 | 206.642 | 54.482 | 249.837 | 74.73 |
| 13299 | C | CG1 | ILE | 1013 | 205.924 | 54.628 | 248.493 | 74.73 |
| 13300 | C | CG2 | ILE | 1013 | 205.663 | 54.064 | 250.921 | 74.73 |
| 13301 | C | CD1 | ILE | 1013 | 204.874 | 55.715 | 248.476 | 74.73 |
| 13302 | N | N | PRO | 1014 | 209.19 | 55.142 | 251.756 | 70.95 |
| 13303 | C | CA | PRO | 1014 | 209.82 | 55.173 | 253.089 | 70.95 |
| 13304 | C | C | PRO | 1014 | 210.101 | 56.578 | 253.594 | 70.95 |
| 13305 | O | O | PRO | 1014 | 210.04 | 56.82 | 254.806 | 70.95 |
| 13306 | C | CB | PRO | 1014 | 211.112 | 54.373 | 252.879 | 70.95 |
| 13307 | C | CG | PRO | 1014 | 210.809 | 53.472 | 251.743 | 70.95 |
| 13308 | C | CD | PRO | 1014 | 209.94 | 54.275 | 250.829 | 70.95 |
| 13309 | N | N | ALA | 1015 | 210.408 | 57.512 | 252.7 | 71.05 |
| 13310 | C | CA | ALA | 1015 | 210.651 | 58.896 | 253.077 | 71.05 |
| 13311 | C | C | ALA | 1015 | 209.377 | 59.727 | 253.124 | 71.05 |
| 13312 | O | O | ALA | 1015 | 209.452 | 60.929 | 253.4 | 71.05 |
| 13313 | C | CB | ALA | 1015 | 211.651 | 59.54 | 252.113 | 71.05 |
| 13314 | N | N | ARG | 1016 | 208.22 | 59.116 | 252.862 | 71.25 |
| 13315 | C | CA | ARG | 1016 | 206.932 | 59.81 | 252.861 | 71.25 |
| 13316 | C | C | ARG | 1016 | 206.934 | 60.985 | 251.886 | 71.25 |
| 13317 | O | O | ARG | 1016 | 206.49 | 62.088 | 252.209 | 71.25 |
| 13318 | C | CB | ARG | 1016 | 206.543 | 60.265 | 254.269 | 71.25 |
| 13319 | C | CG | ARG | 1016 | 206.44 | 59.13 | 255.274 | 71.25 |
| 13320 | C | CD | ARG | 1016 | 205.756 | 59.583 | 256.552 | 71.25 |
| 13321 | N | NE | ARG | 1016 | 206.41 | 60.746 | 257.14 | 71.25 |
| 13322 | C | CZ | ARG | 1016 | 207.374 | 60.683 | 258.047 | 71.25 |
| 13323 | N | NH1 | ARG | 1016 | 207.827 | 59.525 | 258.497 | 71.25 |
| 13324 | N | NH2 | ARG | 1016 | 207.898 | 61.813 | 258.515 | 71.25 |
| 13325 | N | N | ARG | 1017 | 207.443 | 60.744 | 250.682 | 68.4 |
| 13326 | C | CA | ARG | 1017 | 207.451 | 61.729 | 249.61 | 68.4 |
| 13327 | C | C | ARG | 1017 | 206.515 | 61.256 | 248.509 | 68.4 |
| 13328 | O | O | ARG | 1017 | 206.622 | 60.115 | 248.049 | 68.4 |
| 13329 | C | CB | ARG | 1017 | 208.864 | 61.93 | 249.062 | 68.4 |
| 13330 | C | CG | ARG | 1017 | 209.91 | 62.215 | 250.126 | 68.4 |
| 13331 | C | CD | ARG | 1017 | 209.685 | 63.563 | 250.785 | 68.4 |
| 13332 | N | NE | ARG | 1017 | 209.877 | 64.663 | 249.85 | 68.4 |
| 13333 | C | CZ | ARG | 1017 | 211.053 | 65.207 | 249.569 | 68.4 |
| 13334 | N | NH1 | ARG | 1017 | 212.169 | 64.767 | 250.126 | 68.4 |
| 13335 | N | NH2 | ARG | 1017 | 211.111 | 66.216 | 248.705 | 68.4 |
| 13336 | N | N | ASN | 1018 | 205.6 | 62.129 | 248.089 | 75.61 |
| 13337 | C | CA | ASN | 1018 | 204.582 | 61.758 | 247.122 | 75.61 |
| 13338 | C | C | ASN | 1018 | 204.474 | 62.81 | 246.027 | 75.61 |
| 13339 | O | O | ASN | 1018 | 204.415 | 64.012 | 246.323 | 75.61 |
| 13340 | C | CB | ASN | 1018 | 203.219 | 61.583 | 247.803 | 75.61 |
| 13341 | C | CG | ASN | 1018 | 202.179 | 60.991 | 246.88 | 75.61 |
| 13342 | O | OD1 | ASN | 1018 | 201.492 | 61.71 | 246.158 | 75.61 |
| 13343 | N | ND2 | ASN | 1018 | 202.053 | 59.67 | 246.903 | 75.61 |
| 13344 | N | N | PRO | 1019 | 204.448 | 62.39 | 244.758 | 75.86 |
| 13345 | C | CA | PRO | 1019 | 204.324 | 63.359 | 243.658 | 75.86 |
| 13346 | C | C | PRO | 1019 | 203.01 | 64.117 | 243.642 | 75.86 |
| 13347 | O | O | PRO | 1019 | 202.951 | 65.196 | 243.039 | 75.86 |
| 13348 | C | CB | PRO | 1019 | 204.468 | 62.486 | 242.404 | 75.86 |
| 13349 | C | CG | PRO | 1019 | 205.139 | 61.237 | 242.878 | 75.86 |
| 13350 | C | CD | PRO | 1019 | 204.641 | 61.017 | 244.267 | 75.86 |
| 13351 | N | N | ARG | 1020 | 201.959 | 63.595 | 244.271 | 85.03 |
| 13352 | C | CA | ARG | 1020 | 200.658 | 64.246 | 244.284 | 85.03 |
| 13353 | C | C | ARG | 1020 | 200.385 | 65.003 | 245.575 | 85.03 |
| 13354 | C | O | ARG | 1020 | 199.258 | 65.467 | 245.777 | 85.03 |
| 13355 | C | CB | ARG | 1020 | 199.551 | 63.214 | 244.044 | 85.03 |
| 13356 | C | CG | ARG | 1020 | 199.521 | 62.666 | 242.63 | 85.03 |
| 13357 | C | CD | ARG | 1020 | 199.199 | 61.183 | 242.621 | 85.03 |
| 13358 | N | NE | ARG | 1020 | 200.256 | 60.401 | 243.25 | 85.03 |
| 13359 | C | CZ | ARG | 1020 | 200.262 | 59.078 | 243.332 | 85.03 |
| 13360 | N | NH1 | ARG | 1020 | 199.276 | 58.35 | 242.834 | 85.03 |
| 13361 | N | NH2 | ARG | 1020 | 201.283 | 58.469 | 243.928 | 85.03 |
| 13362 | N | N | LEU | 1021 | 201.382 | 65.148 | 246.449 | 76.98 |
| 13363 | C | CA | LEU | 1021 | 201.217 | 65.876 | 247.707 | 76.98 |
| 13364 | C | C | LEU | 1021 | 201.289 | 67.378 | 247.427 | 76.98 |
| 13365 | O | O | LEU | 1021 | 202.159 | 68.103 | 247.911 | 76.98 |
| 13366 | C | CB | LEU | 1021 | 202.271 | 65.443 | 248.715 | 76.98 |
| 13367 | C | CG | LEU | 1021 | 201.762 | 65.056 | 250.102 | 76.98 |
| 13368 | C | CD1 | LEU | 1021 | 200.939 | 66.18 | 250.706 | 76.98 |
| 13369 | C | CD2 | LEU | 1021 | 200.955 | 63.773 | 250.028 | 76.98 |
| 13370 | N | N | VAL | 1022 | 200.342 | 67.839 | 246.619 | 77.81 |
| 13371 | C | CA | VAL | 1022 | 200.29 | 69.236 | 246.197 | 77.81 |
| 13372 | C | C | VAL | 1022 | 198.832 | 69.674 | 246.192 | 77.81 |
| 13373 | O | O | VAL | 1022 | 197.921 | 68.844 | 246.049 | 77.81 |
| 13374 | C | CB | VAL | 1022 | 200.941 | 69.432 | 244.806 | 77.81 |
| 13375 | C | CG1 | VAL | 1022 | 199.923 | 69.254 | 243.687 | 77.81 |
| 13376 | C | CG2 | VAL | 1022 | 201.654 | 70.775 | 244.706 | 77.81 |
| 13377 | N | N | PRO | 1023 | 198.574 | 70.967 | 246.387 | 78.42 |
| 13378 | C | CA | PRO | 1023 | 197.199 | 71.462 | 246.267 | 78.42 |
| 13379 | C | C | PRO | 1023 | 196.627 | 71.209 | 244.88 | 78.42 |
| 13380 | O | O | PRO | 1023 | 197.351 | 71.142 | 243.885 | 78.42 |
| 13381 | C | CB | PRO | 1023 | 197.339 | 72.958 | 246.556 | 78.42 |
| 13382 | C | CG | PRO | 1023 | 198.481 | 73.023 | 247.495 | 78.42 |
| 13383 | C | CD | PRO | 1023 | 199.444 | 71.95 | 247.056 | 78.42 |
| 13384 | N | N | TYR | 1024 | 195.3 | 71.068 | 244.835 | 90.95 |
| 13385 | C | CA | TYR | 1024 | 194.622 | 70.619 | 243.621 | 90.95 |
| 13386 | C | C | TYR | 1024 | 194.893 | 71.546 | 242.442 | 90.95 |
| 13387 | O | O | TYR | 1024 | 195.028 | 71.085 | 241.303 | 90.95 |
| 13388 | C | CB | TYR | 1024 | 193.121 | 70.513 | 243.887 | 90.95 |
| 13389 | C | CG | TYR | 1024 | 192.319 | 69.874 | 242.777 | 90.95 |
| 13390 | C | CD1 | TYR | 1024 | 192.154 | 68.497 | 242.72 | 90.95 |
| 13391 | C | CD2 | TYR | 1024 | 191.709 | 70.647 | 241.799 | 90.95 |
| 13392 | C | CE1 | TYR | 1024 | 191.415 | 67.908 | 241.715 | 90.95 |
| 13393 | C | CE2 | TYR | 1024 | 190.969 | 70.066 | 240.79 | 90.95 |
| 13394 | C | CZ | TYR | 1024 | 190.825 | 68.697 | 240.753 | 90.95 |
| 13395 | O | OH | TYR | 1024 | 190.086 | 68.113 | 239.75 | 90.95 |
| 13396 | N | N | ARG | 1025 | 194.972 | 72.855 | 242.691 | 85.6 |
| 13397 | C | CA | ARG | 1025 | 195.19 | 73.795 | 241.596 | 85.6 |
| 13398 | C | C | ARG | 1025 | 196.568 | 73.614 | 240.971 | 85.6 |
| 13399 | O | O | ARG | 1025 | 196.743 | 73.827 | 239.766 | 85.6 |
| 13400 | C | CB | ARG | 1025 | 195.003 | 75.229 | 242.089 | 85.6 |
| 13401 | C | CG | ARG | 1025 | 195.611 | 75.505 | 243.448 | 85.6 |
| 13402 | C | CD | ARG | 1025 | 195.303 | 76.923 | 243.894 | 85.6 |
| 13403 | N | NE | ARG | 1025 | 193.912 | 77.278 | 243.637 | 85.6 |
| 13404 | C | CZ | ARG | 1025 | 192.903 | 76.991 | 244.448 | 85.6 |
| 13405 | N | NH1 | ARG | 1025 | 193.091 | 76.341 | 245.584 | 85.6 |
| 13406 | N | NH2 | ARG | 1025 | 191.672 | 77.365 | 244.108 | 85.6 |
| 13407 | N | N | LEU | 1026 | 197.558 | 73.225 | 241.77 | 80.85 |
| 13408 | C | CA | LEU | 1026 | 198.904 | 72.985 | 241.269 | 80.85 |
| 13409 | C | C | LEU | 1026 | 199.082 | 71.585 | 240.7 | 80.85 |
| 13410 | O | O | LEU | 1026 | 200.155 | 71.278 | 240.172 | 80.85 |
| 13411 | C | CB | LEU | 1026 | 199.931 | 73.218 | 242.38 | 80.85 |
| 13412 | C | CG | LEU | 1026 | 200.024 | 74.642 | 242.927 | 80.85 |
| 13413 | C | CD1 | LEU | 1026 | 201.086 | 74.735 | 244.009 | 80.85 |
| 13414 | C | CD2 | LEU | 1026 | 200.312 | 75.623 | 241.804 | 80.85 |
| 13415 | N | N | LEU | 1027 | 198.065 | 70.735 | 240.794 | 87.28 |
| 13416 | C | CA | LEU | 1027 | 198.172 | 69.377 | 240.289 | 87.28 |
| 13417 | C | C | LEU | 1027 | 198.105 | 69.366 | 238.765 | 87.28 |
| 13418 | O | O | LEU | 1027 | 197.473 | 70.223 | 238.141 | 87.28 |
| 13419 | C | CB | LEU | 1027 | 197.061 | 68.507 | 240.875 | 87.28 |
| 13420 | C | CG | LEU | 1027 | 197.231 | 66.992 | 240.776 | 87.28 |
| 13421 | C | CD1 | LEU | 1027 | 198.489 | 66.552 | 241.504 | 87.28 |
| 13422 | C | CD2 | LEU | 1027 | 196.009 | 66.283 | 241.336 | 87.28 |
| 13423 | N | N | ASP | 1028 | 198.776 | 68.385 | 238.166 | 91.18 |
| 13424 | C | CA | ASP | 1028 | 198.761 | 68.246 | 236.718 | 91.18 |
| 13425 | C | C | ASP | 1028 | 197.383 | 67.808 | 236.235 | 91.18 |
| 13426 | O | O | ASP | 1028 | 196.603 | 67.203 | 236.974 | 91.18 |
| 13427 | C | CB | ASP | 1028 | 199.823 | 67.246 | 236.262 | 91.18 |
| 13428 | C | CG | ASP | 1028 | 199.909 | 66.035 | 237.166 | 91.18 |
| 13429 | O | OD1 | ASP | 1028 | 198.854 | 65.449 | 237.48 | 91.18 |
| 13430 | O | OD2 | ASP | 1028 | 201.035 | 65.663 | 237.558 | 91.18 |
| 13431 | N | N | GLU | 1029 | 197.091 | 68.122 | 234.97 | 92.98 |
| 13432 | C | CA | GLU | 1029 | 195.758 | 67.872 | 234.43 | 92.98 |
| 13433 | C | C | GLU | 1029 | 195.46 | 66.38 | 234.331 | 92.98 |
| 13434 | O | O | GLU | 1029 | 194.3 | 65.966 | 234.438 | 92.98 |
| 13435 | C | CB | GLU | 1029 | 195.615 | 68.537 | 233.062 | 92.98 |
| 13436 | C | CG | GLU | 1029 | 195.772 | 70.048 | 233.089 | 92.98 |
| 13437 | C | CD | GLU | 1029 | 194.672 | 70.736 | 233.876 | 92.98 |
| 13438 | O | OE1 | GLU | 1029 | 193.559 | 70.177 | 233.962 | 92.98 |
| 13439 | O | OE2 | GLU | 1029 | 194.922 | 71.837 | 234.41 | 92.98 |
| 13440 | N | N | ALA | 1030 | 196.49 | 65.56 | 234.114 | 92.51 |
| 13441 | C | CA | ALA | 1030 | 196.275 | 64.123 | 233.974 | 92.51 |
| 13442 | C | C | ALA | 1030 | 195.679 | 63.528 | 235.244 | 92.51 |
| 13443 | O | O | ALA | 1030 | 194.702 | 62.771 | 235.191 | 92.51 |
| 13444 | C | CB | ALA | 1030 | 197.59 | 63.429 | 233.617 | 92.51 |
| 13445 | N | N | THR | 1031 | 196.252 | 63.864 | 236.402 | 96.56 |
| 13446 | C | CA | THR | 1031 | 195.684 | 63.4 | 237.664 | 96.56 |
| 13447 | C | C | THR | 1031 | 194.446 | 64.207 | 238.034 | 96.56 |
| 13448 | O | O | THR | 1031 | 193.527 | 63.688 | 238.678 | 96.56 |
| 13449 | C | CB | THR | 1031 | 196.73 | 63.481 | 238.776 | 96.56 |
| 13450 | O | OG1 | THR | 1031 | 197.95 | 62.876 | 238.329 | 96.56 |
| 13451 | C | CG2 | THR | 1031 | 196.247 | 62.755 | 240.022 | 96.56 |
| 13452 | N | N | LYS | 1032 | 194.406 | 65.479 | 237.632 | 95.85 |
| 13453 | C | CA | LYS | 1032 | 193.249 | 66.318 | 237.923 | 95.85 |
| 13454 | C | C | LYS | 1032 | 191.987 | 65.762 | 237.276 | 95.85 |
| 13455 | O | O | LYS | 1032 | 190.914 | 65.759 | 237.89 | 95.85 |
| 13456 | C | CB | LYS | 1032 | 193.515 | 67.748 | 237.453 | 95.85 |
| 13457 | C | CG | LYS | 1032 | 192.353 | 68.702 | 237.638 | 95.85 |
| 13458 | C | CD | LYS | 1032 | 192.75 | 70.121 | 237.272 | 95.85 |
| 13459 | C | CE | LYS | 1032 | 193.889 | 70.613 | 238.147 | 95.85 |
| 13460 | N | NZ | LYS | 1032 | 194.29 | 72.003 | 237.8 | 95.85 |
| 13461 | N | N | ARG | 1033 | 192.097 | 65.288 | 236.033 | 101.94 |
| 13462 | C | CA | ARG | 1033 | 190.947 | 64.683 | 235.369 | 101.94 |
| 13463 | C | C | ARG | 1033 | 190.555 | 63.368 | 236.03 | 101.94 |
| 13464 | O | O | ARG | 1033 | 189.367 | 63.03 | 236.101 | 101.94 |
| 13465 | C | CB | ARG | 1033 | 191.249 | 64.469 | 233.886 | 101.94 |
| 13466 | N | N | SER | 1034 | 191.541 | 62.608 | 236.514 | 102.5 |
| 13467 | C | CA | SER | 1034 | 191.248 | 61.317 | 237.131 | 102.5 |
| 13468 | C | C | SER | 1034 | 190.392 | 61.478 | 238.381 | 102.5 |
| 13469 | O | O | SER | 1034 | 189.439 | 60.718 | 238.591 | 102.5 |
| 13470 | C | CB | SER | 1034 | 192.549 | 60.588 | 237.463 | 102.5 |
| 13471 | O | OG | SER | 1034 | 192.289 | 59.362 | 238.122 | 102.5 |
| 13472 | N | N | ASN | 1035 | 190.719 | 62.457 | 239.226 | 113.46 |
| 13473 | C | CA | ASN | 1035 | 189.898 | 62.715 | 240.404 | 113.46 |
| 13474 | C | C | ASN | 1035 | 188.527 | 63.245 | 240.007 | 113.46 |
| 13475 | O | O | ASN | 1035 | 187.52 | 62.938 | 240.656 | 113.46 |
| 13476 | C | CB | ASN | 1035 | 190.605 | 63.701 | 241.334 | 113.46 |
| 13477 | C | CG | ASN | 1035 | 191.908 | 63.153 | 241.88 | 113.46 |
| 13478 | O | OD1 | ASN | 1035 | 191.935 | 62.526 | 242.939 | 113.46 |
| 13479 | N | ND2 | ASN | 1035 | 192.997 | 63.388 | 241.158 | 113.46 |
| 13480 | N | N | ARG | 1036 | 188.472 | 64.048 | 238.942 | 110.13 |
| 13481 | C | CA | ARG | 1036 | 187.202 | 64.603 | 238.488 | 110.13 |
| 13482 | C | C | ARG | 1036 | 186.251 | 63.504 | 238.03 | 110.13 |
| 13483 | O | O | ARG | 1036 | 185.048 | 63.557 | 238.312 | 110.13 |
| 13484 | C | CB | ARG | 1036 | 187.453 | 65.604 | 237.362 | 110.13 |
| 13485 | C | CG | ARG | 1036 | 186.699 | 66.911 | 237.501 | 110.13 |
| 13486 | C | CD | ARG | 1036 | 187.553 | 68.069 | 237.017 | 110.13 |
| 13487 | N | NE | ARG | 1036 | 188.181 | 67.779 | 235.733 | 110.13 |
| 13488 | C | CZ | ARG | 1036 | 189.078 | 68.559 | 235.145 | 110.13 |
| 13489 | N | NH1 | ARG | 1036 | 189.478 | 69.691 | 235.699 | 110.13 |
| 13490 | N | NH2 | ARG | 1036 | 189.587 | 68.192 | 233.973 | 110.13 |
| 13491 | N | N | ASP | 1037 | 186.771 | 62.503 | 237.317 | 119.26 |
| 13492 | C | CA | ASP | 1037 | 185.93 | 61.403 | 236.855 | 119.26 |
| 13493 | C | C | ASP | 1037 | 185.365 | 60.61 | 238.027 | 119.26 |
| 13494 | O | O | ASP | 1037 | 184.197 | 60.205 | 238.008 | 119.26 |
| 13495 | C | CB | ASP | 1037 | 186.728 | 60.491 | 235.924 | 119.26 |
| 13496 | C | CG | ASP | 1037 | 187.267 | 61.224 | 234.713 | 119.26 |
| 13497 | O | OD1 | ASP | 1037 | 186.624 | 62.2 | 234.273 | 119.26 |
| 13498 | O | OD2 | ASP | 1037 | 188.334 | 60.825 | 234.201 | 119.26 |

¹atom site ID number as assigned in mmCIF file for RCSB PBD structure 7TZC;
²element symbol representing atom species;
³atom identifier assigned in mmCIF file for RCSB PBD structure 7TZC;
⁴encompassing residue type;
⁵encompassing residue number;
⁶⁻⁸Atom-site coordinates in angstroms specified according to a set of orthogonal Cartesian axes related to the cell axes; Isotropic atomic displacement parameter, or equivalent isotropic atomic displacement parameter, B_{eq}, calculated from the anisotropic displacement parameters, where: B_{eq} = (1/3) sumᵢ[sumⱼ(B^{ij} Aᵢ Aⱼ a*ᵢ a*ⱼ)]; A = the real space cell lengths; a* = the reciprocal space cell lengths; and B^{ij} = 8 pi² U^{ij}.

TABLE 3
Three-dimensional atomic coordinates of Compound 1.

| Id¹ | type_symbol² | label_atom_id³ | Cartn_x⁶ | Cartn_y⁷ | Cartn_z⁸ | B_iso_or_equiv⁹ |
|---|---|---|---|---|---|---|
| 148715 | C | C01 | 187.936 | 59.258 | 246.449 | 168.47 |
| 148716 | C | C03 | 189.331 | 57.425 | 246.849 | 168.47 |
| 148717 | C | C04 | 189.69 | 56.271 | 247.524 | 168.47 |
| 148718 | C | C05 | 190.863 | 55.598 | 247.173 | 168.47 |
| 148719 | C | C06 | 191.646 | 56.092 | 246.149 | 168.47 |
| 148720 | C | C07 | 191.289 | 57.253 | 245.47 | 168.47 |
| 148721 | C | C08 | 190.125 | 57.916 | 245.823 | 168.47 |
| 148722 | C | C10 | 193.748 | 54.166 | 246.996 | 168.47 |
| 148723 | C | C11 | 193.541 | 54.847 | 248.343 | 168.47 |
| 148724 | C | C13 | 191.245 | 54.303 | 247.931 | 168.47 |
| 148725 | C | C14 | 192.55 | 53.166 | 249.615 | 168.47 |
| 148726 | C | C15 | 191.372 | 52.203 | 249.521 | 168.47 |
| 148727 | C | C16 | 190.374 | 52.235 | 250.483 | 168.47 |
| 148728 | C | C17 | 189.302 | 51.365 | 250.401 | 168.47 |
| 148729 | C | C18 | 189.252 | 50.456 | 249.36 | 168.47 |
| 148730 | C | C19 | 190.249 | 50.416 | 248.409 | 168.47 |
| 148731 | C | C20 | 191.317 | 51.282 | 248.491 | 168.47 |
| 148732 | C | C21 | 188.093 | 49.485 | 249.229 | 168.47 |
| 148733 | N | N12 | 192.289 | 54.43 | 248.934 | 168.47 |
| 148734 | O | O02 | 188.161 | 58.103 | 247.201 | 168.47 |
| 148735 | O | O22 | 187.869 | 48.962 | 248.112 | 168.47 |
| 148736 | O | O23 | 187.356 | 49.234 | 250.211 | 168.47 |
| 148737 | S | S09 | 193.179 | 55.242 | 245.66 | 168.47 |

^{1-3,6-9}See description for TABLE 2 above.

TABLE 4
Three-dimensional atomic coordinates of ATP.

| Id¹ | type_symbol² | label_atom_id³ | Cartn_x⁶ | Cartn_y⁷ | Cartn_z⁸ | B_iso_or_equiv⁹ |
|---|---|---|---|---|---|---|
| 148684 | P | PG | 197.186 | 53.295 | 248.546 | 142.43 |
| 148685 | O | O1G | 196.024 | 53.026 | 249.44 | 142.43 |
| 148686 | O | O2G | 198.212 | 54.263 | 249.146 | 142.43 |
| 148687 | O | O3G | 197.905 | 52.026 | 248.075 | 142.43 |
| 148688 | P | PB | 197.291 | 54.492 | 245.803 | 142.43 |
| 148689 | O | O1B | 196.72 | 53.732 | 244.673 | 142.43 |
| 148690 | O | O2B | 198.816 | 54.469 | 245.916 | 142.43 |
| 148691 | O | O3B | 196.705 | 54.006 | 247.199 | 142.43 |
| 148692 | P | PA | 197.257 | 57.362 | 246.526 | 142.43 |
| 148693 | O | O1A | 198.589 | 57.834 | 246.097 | 142.43 |
| 148694 | O | O2A | 197.115 | 57.104 | 248.026 | 142.43 |
| 148695 | O | O3A | 196.843 | 56.019 | 245.779 | 142.43 |
| 148696 | O | O5′ | 196.123 | 58.368 | 246.083 | 142.43 |
| 148697 | C | C5′ | 196.392 | 59.777 | 245.926 | 142.43 |
| 148698 | C | C4′ | 195.302 | 60.388 | 245.081 | 142.43 |
| 148699 | O | O4′ | 194.022 | 59.837 | 245.469 | 142.43 |
| 148700 | C | C3′ | 195.162 | 61.904 | 245.198 | 142.43 |
| 148701 | O | O3′ | 194.779 | 62.479 | 243.954 | 142.43 |
| 148702 | C | C2′ | 194.069 | 62.058 | 246.257 | 142.43 |
| 148703 | O | O2′ | 193.357 | 63.28 | 246.108 | 142.43 |
| 148704 | C | C1′ | 193.167 | 60.871 | 245.923 | 142.43 |
| 148705 | N | N9 | 192.388 | 60.373 | 247.053 | 142.43 |
| 148706 | C | C8 | 191.105 | 60.725 | 247.383 | 142.43 |
| 148707 | N | N7 | 190.642 | 60.124 | 248.452 | 142.43 |
| 148708 | C | C5 | 191.695 | 59.319 | 248.857 | 142.43 |
| 148709 | C | C6 | 191.847 | 58.424 | 249.934 | 142.43 |
| 148710 | N | N6 | 190.894 | 58.18 | 250.839 | 142.43 |
| 148711 | N | N1 | 193.028 | 57.778 | 250.053 | 142.43 |
| 148712 | C | C2 | 193.985 | 58.017 | 249.153 | 142.43 |
| 148713 | N | N3 | 193.957 | 58.836 | 248.1 | 142.43 |
| 148714 | C | C4 | 192.779 | 59.462 | 248.005 | 142.43 |

^{1-3,6-9}See description for TABLE 2 above.

### Example 4: Mutagenesis and Expression of Recombinant RyR1

Constructs expressing wild-type, W882A, W996A, and C906A RyR1 were formed by introducing the respective mutations into fragments of rabbit RyR1 using QuikChangeR II XL Site-Directed Mutagenesis Kit (Agilent) with an HpaI-HpaI fragment in a pBlueScript vector. Each fragment was subcloned into a full length RyR1 construct in pcDNA3.1 vector using an HpaI restriction enzyme. Mutagenesis was confirmed by sequencing and expressed in 293T/17 cells using Lipofectamine™ 2000 (Thermo Fisher Scientific, Cat #11668027). The primers used to introduce specific mutations (codons in parentheses, mutated nucleotides in bold) are as follows: rRyR1-W882A-F: GAACATCCATGAACTC(GCG)GCGCTGACGCGCATT (SEQ ID NO: 4), rRyR1-W996A-F GAATGGGCATAACGTG(GCG)GCACGAGACCGAGTG (SEQ ID NO: 5), and rRyR1-C906A-F: CAAGAGGCTGCACCCG(GCA)CTAGTGAACTTCCACAGCC (SEQ ID NO: 6). For each mutant, the second primer was the complementary reverse to the forward primer. HEK293 cells grown in DMEM supplemented with 10% (v/v) FBS (Invitrogen), 100 U/mL penicillin, 100 mg/mL streptomycin, and 2 mM L-glutamine were co-transfected with WT or mutant RyR1 cDNA using X-tremeGENE™ 9 DNA Transfection Reagent (Millipore Sigma, Cat #6365787001). Cells were collected as pellets 48 h after transfection.

### Example 5: Single-Channel Recordings of Wild-Type (WT) and Mutant RyR1 Reconstituted in Planar Lipid Bilayer

To characterize the role of the periphery of the RY1&2 in the binding and stabilizing effects of Compound 1, single-channel recordings of wild-type (WT) RyR1 and C906A and W882A mutants reconstituted in planar lipid bilayers were performed.

### SR Vesicle Preparation and Ryanodine Receptor Modulator Treatment.

HEK293 cell pellets prepared in EXAMPLE 4 were homogenized in 1 mM tris-maleate buffer (pH 7.4) in the presence of protease inhibitors (Roche), and spun by centrifuge at 8,000 rpm (5,900×g) for 20 min at 4° C. The supernatant was spun by ultracentrifuge at 32,000 rpm (100,000×g) for 45 minutes at 4° C. The final pellet containing microsomal fractions enriched in SR vesicles was resuspended and aliquoted in 300 mM sucrose and 5 mM Pipes (pH 7.0) containing protease inhibitors. Samples were frozen in liquid nitrogen and stored at −80° C. 10 μM S107 or Compound 1 was added to microsomes overnight at 4° C.

### Planar Lipid Bilayers.

Planar lipid bilayers were formed using a 3:1 mixture of phosphatidylethanolamine and phosphatidylcholine (Avanti Polar Lipids, Cat #441601G) suspended (30 mg/mL) in decane by painting the lipid/decane solution across a 200 μm aperture in a polysulfonate cup (Warner Instruments) separating two chambers. The trans chamber (1 mL) representing the intra-SR (luminal) compartment was connected to the headstage input of a bilayer voltage clamp amplifier (BC-525D, Warner Instruments) and the cis chamber (1 mL), representing the cytoplasmic compartment, was held at virtual ground. Solutions in both chambers were as follows: 1 mM EGTA, 250/125 mM Hepes/Tris, 50 mM KCl, 0.64 mM CaCl₂), pH 7.35 as cis solution and 250 mM Hepes, 53 mM Ca(OH)₂, 50 mM KCl, pH 7.35 as trans solution.

The concentration of free Ca²⁺ in the cis chamber was calculated using the WinMaxC program (version 2.50; www.stanford.edu/-cpatton/maxc.html). SR vesicles were added to the cis side, and fusion with the lipid bilayer was induced by making the cis side hyperosmotic by addition of 400-500 mM KCl. After the appearance of potassium and chloride channels, the cis compartment was perfused with the cis solution. Single-channel currents were recorded at 0 mV using a Bilayer Clamp BC-535 amplifier (Warner Instruments), filtered at 1 kHz, and digitized at 4 kHz. All experiments were performed at room temperature. Data acquisition was performed using Digidata 1440A and Axoscope 10.2 software, recordings were analyzed using Clampfit 10.2 (Molecular Devices). Open probability was identified by 50% threshold analyses using a minimum of 2 min of continuous record. For measurements with oxidized RyR1, microsomes were incubated with 1 mM H₂O₂ for 30 min at 37° C. to induce oxidation. At the conclusion of each experiment, 5 μM ryanodine was added to the cis chamber to confirm channels as RyR. Experiments were repeated at least 3 times (n≥30 cells per group).

### Results.

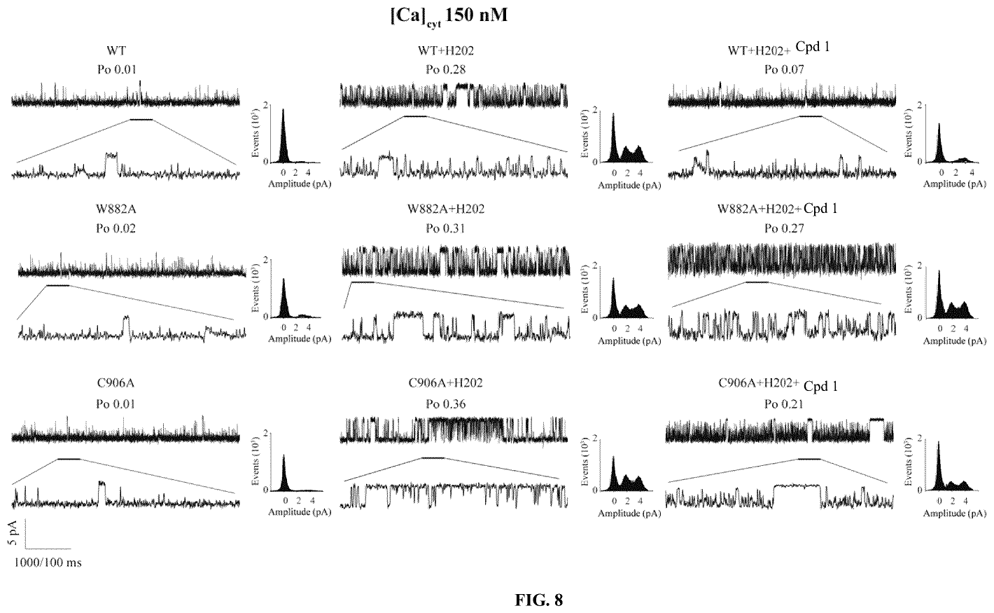

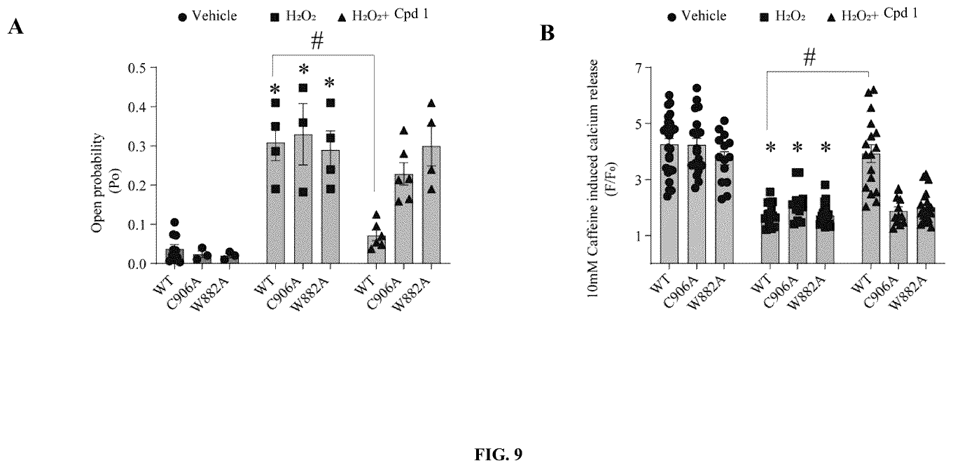

The resulting traces are provided in FIG. 8. In FIG. 8, sections of each trace are shown with expanded timescale to demonstrate subconductance states, and opening events are recorded as an upward deflection. Quantification of single channel current open probability (Pₒ) is provided in FIG. 9, Panel A (data are means±SEMs; 1-way-ANOVA shows *p<0.05 versus WT).

The open probability (Pₒ) of the mutant channels correlated with that of wild-type (WT) channels under resting conditions (150 nm Ca²⁺) and following treatment with H₂O₂ to trigger the oxidation-induced Ca²⁺ leak, indicating that these mutants remain functional; however, the Ca²⁺ leak was not rescued by the addition of Compound 1 in W882A and showed only a minor reduction for C₉₀₆A.

### Example 6: Ca²⁺ Imaging in HEK293 Cells Expressing WT and Mutant RyR1 Channels

To further confirm the results of EXAMPLE 5, Ca²⁺ release was measured in response to the caffeine-induced activation of RyR1.

### Methods.

Cytosolic Ca²⁺ measurements were performed with HEK293 cells expressing WT or mutant RyR1 (prepared according to EXAMPLE 4) grown on a glass-bottom dish for 26-30 h after plasmid transfection. Experiments were performed at 26° C. HEK293 cells were loaded with 4 μM fluo-4 AM in culture medium for 30 min at 37° C. and then incubated with Krebs solution (140 mM NaCl, 5 mM KCl, 2 mM CaCl₂), 1 mM MgCl₂, 11 mM glucose, and 5 mM HEPES, pH 7.4). For measurements with oxidized RyR1, transfected cells were incubated with 1 mM H₂O₂ for 30 min at 37° C. to induce oxidation. Confocal imaging was performed by excitation with a 488 nm light from the argon laser of a Zeiss LSM 800 inverted confocal microscope (40× oil immersion lens). Experiments were repeated at least 3 times (n>30 cells per group). Data were analyzed using Image J software.

### Results.

Quantification of caffeine-induced calcium release in response to 10 mM caffeine is provided in FIG. 9, Panel B. In this experiment, oxidation of RyR1 caused a leak that depleted intracellular Ca²⁺, blunting the response to caffeine-induced Ca²⁺ release. Both mutants were unaffected by Compound 1 and only the WT channels could be restored to stable conditions following oxidation. These results corroborate that the binding site of Compound 1 resides in the RY1&2 domain and indicate that residues in the periphery of the RY1&2 play a role in the allosteric regulation by Compound 1. In addition, the mutation of C906 does not confer protection against the oxidation-induced leak.

### Example 7: Ligand Binding Assay

Compound 1 binding was assayed with RyR1, oxidized and phosphorylated RyR1, and RyR1 mutants in the presence of radiolabeled ATP or ADP. The similarities between the adenine ring of ATP and the benzothiazepine moiety of ryanodine receptor modulator compounds provided sound reasoning to also test the RY1&2 domain for ryanodine receptor modulator binding using a radiolabeled form S107, which competes with Compound 1 for the binding site in RyR1.

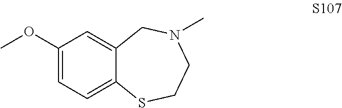

H₂O₂ Treatment and Phosphorylation of Recombinant RyR.

ER vesicles from HEK293 cells expressing RyR1-WT or RyR1-mutant from were prepared by homogenizing cell pellets obtained in EXAMPLE 4 on ice using a Teflon glass douncer (50 times) with two volumes of: 20 mM Tris-maleate pH 7.4, 1 mM EDTA, 1 mM DTT, and protease inhibitors (Roche). Homogenate was then spun by centrifuge at 4,000×g for 15 min at 4° C. The resulting supernatant was spun by centrifuge at 40,000×g for 30 min at 4° C. The final pellet, containing the ER fractions, was resuspended and aliquoted in 250 mM sucrose, 10 mM MOPS pH 7.4, 1 mM EDTA, 1 mM DTT and protease inhibitors. Samples were frozen in liquid nitrogen and stored at −80° C.

For PKA-phosphorylated channel experiments, −200 mg of microsomes were in vitro phosphorylated with 40 units of PKA catalytic subunit (SigmaAldrich, Cat #P2645) for 30 min at 30° C. in the presence of the following buffer: 50 mM Tris/PIPES pH 7.0, 8 mM MgCl₂, 1 mM MgATP, and 1 mM EGTA. The samples were then spun by centrifuge for 10 min at 100,000×g. The resulting pellets were washed four times with wash buffer (300 mM sucrose, 10 mM imidazole, pH 7.4) and aliquots were frozen in liquid nitrogen and stored at −80° C. Oxidation of RyR1 was induced by incubating microsomes with 1 mM H₂O₂ for 30 min at room temperature prior to washing.

### Binding Assay.

Titrative ³H-S107 binding, performed in the absence and presence of 10 mM NaATP, was initiated by addition of ³H-S107 (10-10,000 nM final concentration) to 0.1 mg skeletal sarcoplasmic reticulum (SR) microsomes in binding buffer (50 mM Tris-HCl, pH 7.5, 150 mM NaCl, 25 mM MgCl₂). For ATP and ADP competition, 5107 binding was assessed at a concentration of 1 mM. All samples were incubated at room temperature for 30 min ³H-S107 binding was stopped by addition of ice-cold binding buffer prior to filtration through GF/B Whatman filters pre-equilibrated with 0.015% PE. Filters were washed 3 times with 5 mL of wash buffer (10 mM MOPS, 200 mM NaCl, pH 7.4), dried, and counted. Data were normalized to ³H-ryanodine binding. Nonspecific binding was determined using 20-fold excess unlabeled S107. ³²P-ATP and ³²P-ADP binding were initiated by addition of the respective radioligands (100-50,000 nM) to 0.1 mg recombinant RyR1 microsomes in binding buffer. Samples were incubated at room temperature for 60 min and the reaction was stopped as previously described. ³H-S107 binding performed in native rabbit microsomes used only the endogenous ATP, while assays with recombinant RyR1 in HEK293 microsomes include the addition of 10 mM ATP or ADP. Data were normalized to ³H-ryanodine binding.

### Results.

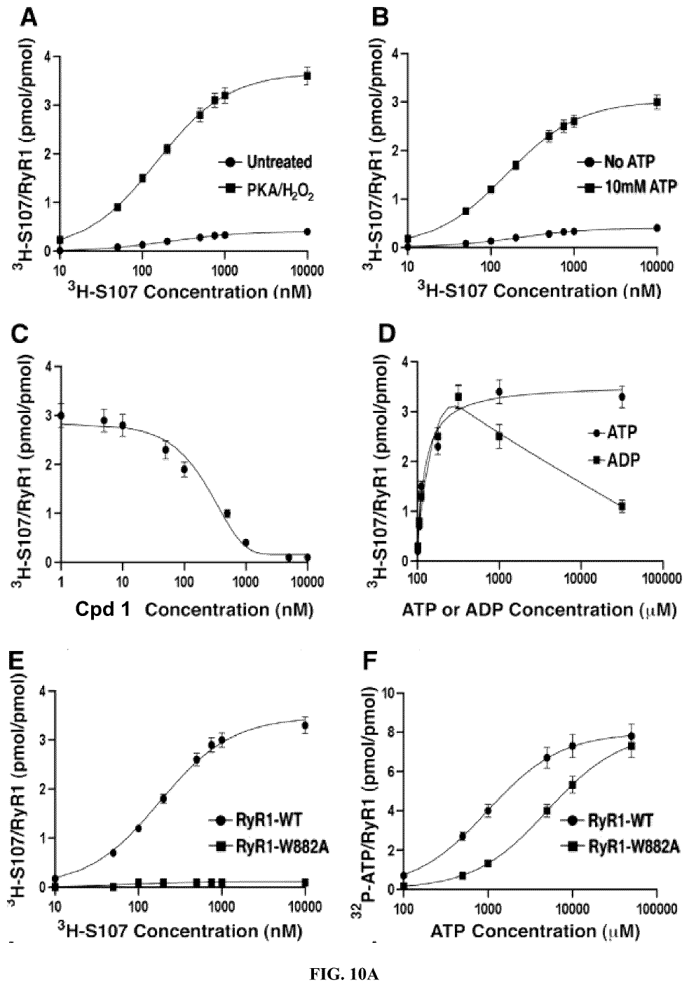

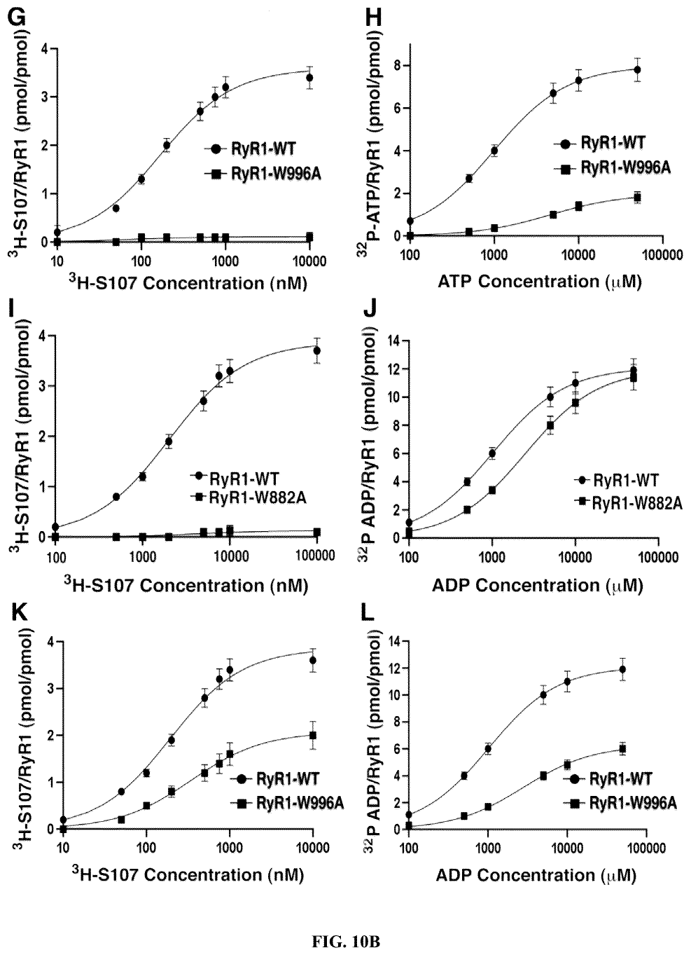

FIG. 10A, Panel A is a chart that illustrates the effects of PKA phosphorylation and H₂O₂ oxidation of RyR1 on S107 binding. Binding was performed with untreated microsomes and microsomes treated with PKA and 1.0 mM H₂O₂. FIG. 10A, Panel B is a chart that illustrates the effects of ATP on S107 binding to purified RyR1. FIG. 10A, Panel C illustrates S107-Compound 1 competition performed with PKA/H₂O₂ treated microsomes, 500 nM of ³H-S107, and varied concentrations (1-10,000 nM) of unlabeled Compound 1. FIG. 10A, Panel D illustrates S107 binding in the presence of increasing concentrations of ATP or ADP. FIG. 10A, Panel E illustrates S107 binding to recombinant RyR1-WT and RyR1-W882A mutant in microsomes treated with PKA and H₂02. FIG. 10A, Panel F illustrates ³²P-ATP binding to WT and W882A RyR1. FIG. 10B, Panel G illustrates S107 binding to recombinant RyR1-WT and RyR1-W996A. FIG. 10B, Panel H illustrates ³²P-ATP binding to WT and W996A RyR1. FIG. 10B, Panels I-L illustrate radioligand binding to WT and mutant channels with ADP in place of ATP. Expression of RyR1-W882A and W996A channels were confirmed by ³H-ryanodine binding comparable to RyR-WT microsomes. In FIG. 10A and FIG. 10B, error bars represent the SD of the mean from 4 replicates. Ligand binding affinities and stoichiometries for each assay are summarized in TABLE 5.

TABLE 5
Radioligand binding.

| ³H-S107 | Kd (nM) | Bmax (mol S107/mol RyR1) |
|---|---|---|
| Untreated | 150 ± 7 | 0.4 ± 0.1 |
| PKA/H₂O₂ | 155 ± 9 | 3.7 ± 0.2 |
| No ATP | 147 ± 6 | 0.4 ± 1.0 |
| 10 mM ATP | 152 ± 7 | 3.0 ± 0.2 |
| RyR1-WT | 200 ± 11 | 3.5 ± 0.3 |
| RyR1-W882A | No | |
| | detectable | |
| | binding | |
| RyR1-W996A | No | |
| | detectable | |
| | binding | |
| ³²P-ATP | Kd (mM) | Bmax |
| RyR1-WT | 1.0 ± 0.1 | 8.0 ± 0.6 |
| RyR1-W882A | 5.0 ± 0.4 | 7.5 ± 0.6 |
| RyR1-W996A | 4.5 ± 0.3 | 2.5 ± 0.4 |
| ³H-S107 | Kd (nM) | Bmax (mol S107/mol RyR1) |
| RyR1-WT | 188 ± 12 | 3.6 ± 0.3 |
| RyR1-W882A | No | |
| | detectable | |
| | binding | |
| RyR1-W996A | 250 ± 10 | 2.0 ± 0.2 |
| ³²P-ATP | Kd (mM) | Bmax |
| RyR1-WT | 1.0 ± 0.1 | 12.0 ± 1.1 |
| RyR1-W882A | 3.0 ± 0.3 | 11.6 ± 0.6 |
| RyR1-W996A | 2.5 ± 0.3 | 6.0 ± 0.6 |

Ryanodine receptor modulator binding (B-max) to RyR1 was increased approximately 10-fold by oxidation and phosphorylation of the channel (TABLE 5), mimicking the condition of RyR in disease states (FIG. 10A, Panel A), and ryanodine receptor modulator binding was increased by a similar degree in the presence of 10 mM ATP (FIG. 10A, Panel B). When equal concentrations of Compound 1 and ³H-S107 were present (500 nM each), ³H-S107 binding also decreased 2-fold. This result indicated the increased affinity of Compound 1 compared to S107 (FIG. 10A, Panel C). This follows in accordance with the structures of S107 and Compound 1, as the former is a scaffold lacking the benzoic acid tail of the latter and both bear similarities to the adenine ring of ATP, while the benzoic acid tail of Compound 1 resembles the ribose ring and tail.

Mutation of the primary binding residue (W882A) abolished ³H-S107 binding to the channel (FIG. 10A, Panel E). In contrast, ATP binding was maintained, although slightly reduced with RyR1-W882A, as evidenced by the decreased affinity of radiolabeled ATP (FIG. 10A, Panel F). However, ATP binding was significantly reduced in the W996 Å mutant (FIG. 10B, Panel G). In this instance, ATP binding was partially retained at the C-terminal binding site in RyR1. Finally, Compound 1 binding was also abolished in W996A, likely as a result of the loss of ATP binding in the RY1&2 binding pocket (FIG. 10B, Panel H).

These experiments were then repeated with ADP in place of ATP to compare ADP binding to this site (FIG. 10B, Panels I-L). ADP exhibited similar binding to WT-RyR1, with the exception of greater stoichiometry, with 12 molecules of ADP per channel compared to a maximum of 8 with ATP, wherein the C-terminal site is occupied by one molecule of either ligand. Likewise, ADP binding remained greater than one molecule per channel in RyR1-W996A. This result indicated the binding of two molecules of ADP to the peripheral binding site, whereas only a single ATP binds to this site. To confirm this observation and to assess the competition between ADP and ryanodine receptor modulator binding, S107 binding was measured in the presence of increasing concentrations of ATP or ADP (FIG. 10A, Panel D). In this experiment, no competition was observed in the presence of ATP. However, high concentrations of ADP were found to have a significant inhibitory effect on the binding of S107. This effect required higher than physiological concentrations, as ryanodine receptor modulators exhibit a much higher affinity. These data indicate that the additional occupancy of ADP is in the same ryanodine receptor modulator-binding site and that the binding properties of ATP and ADP are not identical.

1.-209. (canceled)

210. A composition comprising a complex suspended in a solid medium, the solid medium comprising vitreous ice, wherein the complex comprises a protein and a synthetic compound, wherein the protein is a ryanodine receptor 1 protein (RyR1) or mutant thereof.

211. The composition of claim 210, wherein the composition is prepared by a process comprising vitrifying an aqueous solution applied to an electron microscopy grid, wherein the aqueous solution comprises the protein and the synthetic compound.

212. The composition of claim 211, wherein the aqueous solution includes one or more of caffeine, a Ca²⁺ ion, sodium adenosine triphosphate (NaATP), or calmodulin.

213. The composition of claim 210, wherein the solid medium further comprises a nucleoside-containing molecule, wherein the nucleoside-containing molecule and the synthetic compound bind a RYR domain of the protein.

214. The composition of claim 213, wherein the RYR domain is a RY1&2 domain.

215. The composition of claim 214, wherein the RY1&2 domain is comprised within a SPRY domain of the RyR1 protein.

216. The composition of claim 214, wherein the RY1&2 domain has a three-dimensional structure according to TABLE 2.

217. The composition of claim 213, wherein the nucleoside-containing molecule is a purine nucleoside-containing molecule, a nucleotide or nucleoside polyphosphate, an adenosine triphosphate (ATP) molecule, or an adenosine diphosphate (ADP) molecule.

218. The composition of claim 213, wherein the nucleoside-containing molecule is an adenosine triphosphate (ATP) molecule, wherein the ATP molecule forms a pi-stacking interaction with W996 of the protein.

219. The composition of claim 218, wherein the ATP molecule has a three-dimensional conformation according to TABLE 4.

220. The composition of claim 218, wherein the ATP molecule cooperatively binds the protein with the synthetic compound, or wherein the ATP molecule forms a pi-stacking interaction with the synthetic compound.

221. The composition of claim 213, wherein the complex comprises two adenosine diphosphate (ADP) molecules, wherein both ADP molecules bind a common RYR domain of the protein.

222. The composition of claim 213, wherein the complex further comprises a second nucleoside-containing molecule bound to a C-terminal domain of the RyR1 protein, wherein the second nucleoside-containing molecule is a second ATP molecule.

223. The composition of claim 210, wherein the complex further comprises one or more of calmodulin, calstabin, caffeine, or a Ca²⁺ ion.

224. The composition of claim 210, wherein the synthetic compound binds a RY 1&2 domain of the protein.

225. The composition of claim 210, wherein the synthetic compound forms a pi-stacking interaction with W882 of the protein, or a salt bridge with H879 of the protein.

226. The composition of claim 210, wherein the protein is mutant RyR1 or a post-translationally modified RyR1.

227. The composition of claim 210, wherein the synthetic compound comprises a benzazepane, benzothiazepane, benzothiazepine, or benzodiazepane moiety.

228. The composition of claim 210, wherein the synthetic compound is a compound of Formula (I): 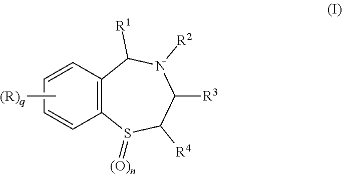
wherein:
each R is independently acyl, O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ R¹⁰;
R³ is acyl, O-acyl, alkyl, alkenyl, aryl, alkylaryl, cycloalkyl, heteroaryl, or heterocyclyl, each of which is independently substituted or substituted; or H, CO₂Y, or C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —N—HR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, —OH, —SO₂R¹¹, —NHSO₂R¹¹, C(═O)(R¹²), NHC═O(R¹²), —OC═O(R¹²), or —P(═O)R¹³R⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4,
or a pharmaceutically-acceptable salt thereof.

229. The composition of claim 210, wherein the synthetic compound is a compound of Formula (I-k): 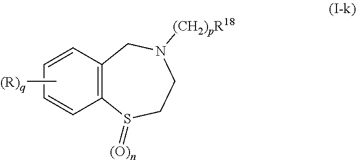
wherein:
each R is independently acyl, O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹⁸ is alkyl, aryl, cycloalkyl, or heterocyclyl, each of which is independently substituted or unsubstituted; or —NR¹⁵R¹⁶, —C(═O)NR¹⁵R¹⁶, —(C═O)OR¹⁵, or —OR¹⁵;
q is 0, 1, 2, 3, or 4;
p is 1, 2, 3, 4, 5, 6, 7, 8 9, or 10; and
n is 0, 1, or 2,
or a pharmaceutically-acceptable salt thereof.

230. The composition of claim 210, wherein the synthetic compound is: 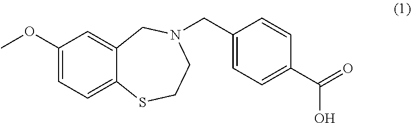 [SMILES: COc1ccc2c(c1)CN(Cc1ccc(C(=O)O)cc1)CCS2]
or an ionized form thereof.

231. The composition of claim 230, wherein the synthetic compound has a three-dimensional conformation according to TABLE 3.

232. A method for predicting a docked position of a target ligand in a binding site of a biomolecule, the method comprising:
receiving a template ligand-biomolecule structure, the template ligand-biomolecule structure comprising a template ligand docked in the binding site of the biomolecule;
comparing a pharmacophore model of the template ligand to a pharmacophore model of the target ligand;
overlapping the pharmacophore model of the target ligand with the pharmacophore model of the template ligand while the template ligand is in the binding site of the biomolecule; and
predicting the docked position of the target ligand in the binding site of the biomolecule based on a position of the pharmacophore model of the target ligand when overlapped with the pharmacophore model of the template ligand,
wherein the template ligand-biomolecule structure is obtained by a process comprising subjecting a complex of the biomolecule and the template ligand to single-particle cryogenic electron microscopy analysis,
wherein the biomolecule is a ryanodine receptor 1 protein (RyR1) or a mutant thereof and the template ligand is a synthetic compound, and
wherein the complex of the biomolecule and the template ligand is obtained by the process to prepare the composition of claim 211.

233. The method of claim 232, wherein the biomolecule is a RY1&2 domain of RyR1.

234. The method of claim 233, wherein the RY1&2 domain comprises a structure according to TABLE 2.

235. The method of claim 233, wherein the RY1&2 domain further comprises an ATP molecule.

236. The method of claim 235, wherein the ATP molecule has a three-dimensional conformation according to TABLE 4.

237. The method of claim 232, wherein the template ligand is 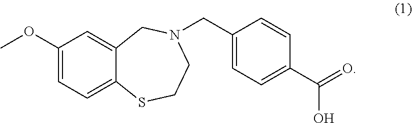 [SMILES: COc1ccc2c(c1)CN(Cc1ccc(C(=O)O)cc1)CCS2]
or an ionized form thereof.

238. A method of identifying a plurality of potential lead compounds, the method comprising the steps of:
(a) analyzing, using a computer system, an initial lead compound known to bind to a biomolecular target, the analyzing comprising partitioning, by providing a database of known reactions, the initial lead compound into atoms defining partitioned lead compound comprising a lead compound core and atoms defining a lead compound non-core, wherein the initial lead compound is partitioned using a computational retrosynthetic analysis of the initial lead compound;
(b) identifying, using the computer system, a plurality of alternative cores to replace the lead compound core in the initial lead compound, thereby generating a plurality of potential lead compounds each having a respective one of the plurality of alternative cores;
(c) calculating, using the computer system, a difference in binding free energy between the partitioned lead compound and each potential lead compound;
(d) predicting, using the computer system, whether each potential lead compound will bind to the biomolecular target and identifying a predicted active set of potential lead compounds based on the prediction;
(e) obtaining a synthesized set of at least some of the potential leads of the predicted active set to establish a first of potential lead compounds; and
(f) determining, empirically, an activity of each of the first set of synthesized potential lead compounds,
wherein the structure of the biomolecular target used in the predicting of (d) is obtained by a process comprising subjecting a complex of the biomolecular target and the initial lead compound to single-particle cryogenic electron microscopy analysis,
wherein the biomolecular target is a ryanodine receptor 1 protein (RyR1) or a mutant thereof and the initial lead compound is a synthetic compound, and
wherein the complex of the biomolecular target and the initial lead compound is obtained by the process to prepare the composition of claim 211.

239. A method for pharmaceutical drug discovery, comprising:
identifying an initial lead compound for binding to a biomolecular target;
using the method of claim 238 to identify a predicted active set of potential lead compounds for binding to the biomolecular target based on the initial lead compound;
selecting one or more of the predicted active set of potential lead compounds for synthesis; and
assaying the one or more synthesized selected compounds to assess each synthesized selected compounds suitability for in vivo use as a pharmaceutical compound,
wherein the biomolecular target is a RY1&2 domain of RyR1, and the structure of the biomolecular target used in the predicting of (d) is obtained by a process comprising subjecting a complex of the biomolecular target and the initial lead compound to single-particle cryogenic electron microscopy analysis.

240. A computer-implemented method of quantifying binding affinity between a ligand and a receptor molecule, the method comprising:
receiving by one or more computers, data representing a ligand molecule,
receiving by one or more computers, data representing a receptor molecule domain,
using the data representing the ligand molecule and the data representing the receptor molecule domain in computer analysis to identify a ring structure within the ligand, the ring structure being an entire ring or a fused ring;
using the data representative of the identified ligand ring structure to designate a first ring face and a second ring face opposite to the first ring face, and classifying the ring structure by:
a) determining proximity of receptor atoms to atoms on the first face of the ligand ring; and
b) determining proximity of receptor atoms to atoms on the second face of the ligand ring;
c) determining solvation of the first face of the ligand ring and solvation of the second face of the ligand ring;
classifying the identified ligand ring structure as buried, solvent exposed or having a single face exposed to solvent based on receptor atom proximity to and solvation of the first ring face and receptor atom proximity to and solvation of the second ring face;
quantifying the binding affinity between the ligand and the receptor molecule domain based at least in part on the classification of the ring structure; and
displaying, via computer, information related to the classification of the ring structure,
wherein the receptor molecule domain is a RY1&2 domain of RyR1 protein or a mutant thereof, wherein the data representing a ligand molecule and the data representing a receptor molecule domain are obtained by a process comprising subjecting a complex comprising the ligand molecule and the receptor molecule domain to single-particle cryogenic electron microscopy analysis,
wherein the ligand molecule is a synthetic compound, and wherein the complex is obtained by the process to prepare the composition of claim 211.

241. A method of identifying a compound having RyR1 modulatory activity, the method comprising:
(a) determining open probability (Pₒ) of a RyR1 protein, wherein the RyR1 protein is a mutant RyR protein, a post-translationally modified RyR1 protein, or a combination thereof,
(b) contacting the RyR1 protein with a test compound;
(c) determining open probability (Pₒ) of the RyR1 protein in the presence of the test compound; and
(d) determining a difference between the Pₒ of the RyR1 protein in the presence and absence of the test compound;
wherein a reduction in the Pₒ of the RyR1 protein in the presence of the test compound compared with the Pₒ of the RyR1 protein in the absence of the test compound is indicative of the compound having RyR1 modulatory activity.

242. The method of claim 241, wherein the RyR1 protein is a mutated or a post-translationally modified RyR1 protein, and wherein the test compound preferentially binds to a mutant or post-translationally modified RyR1 relative to a wild-type RyR1.

243. A method for identifying a compound having RyR1 modulatory activity, comprising:
(a) contacting a RyR1 protein with a ligand having known RyR1 modulatory activity to create a mixture, wherein the RyR1 protein is a mutant RyR1 protein, post-translationally modified RyR1 protein, or a combination thereof;
(b) contacting the mixture of step (a) with a test compound; and
(c) determining the ability of the test compound to displace the ligand from the RyR1 protein.

244. The method of claim 243, wherein the ligand is labeled and generates a signal, and wherein determining the ability of the test compound to displace the ligand from the RyR1 protein comprises determining a change in the signal.
